(19) United States
(12) Patent Application Publication
(10) Pub. No.: US 2024/0280561 A1
(43) Pub. Date: Aug. 22, 2024

(54) COMPOSITIONS AND METHODS FOR TREATING AND/OR IDENTIFYING AN AGENT FOR TREATING INTESTINAL CANCERS

(71) Applicant: Dana-Farber Cancer Institute, Inc., Boston, MA (US)

(72) Inventors: Nilay Sethi, Boston, MA (US); Sandor Spisak, Boston, MA (US)

(21) Appl. No.: 18/567,200

(22) PCT Filed: Jun. 8, 2022
(86) PCT No.: PCT/US2022/032639

§ 371 Date: Dec. 5, 2023

(51) Int. Cl. G01N 33/50; C12N 15/85; G01N 33/68

(52) U.S. Cl. CPC G01N 33/5011; C12N 15/85; G01N 33/68

(57) ABSTRACT

The present invention relates, in part, to compositions and methods for treating and/or identifying an agent for treating intestinal cancers, such as a colorectal cancer (CRC).

### RELATED APPLICATIONS

This application claims the benefit of U.S. Provisional Application No. 63/208,313, filed Jun. 8, 2021, which is hereby incorporated by reference in its entirety.

### BACKGROUND OF THE INVENTION

Intestinal stem cells and their immediate progeny replicate frequently in the crypt to sustain the most rapidly renewing epithelium in the human body. Wingless/integrated (Wnt) signaling is critical for maintaining stem cell reservoirs and crypt homeostasis. As the immediate progeny push beyond the crypt into the villus, they enter post-mitotic differentiation, giving rise to mature intestinal cells that provide absorptive, barrier, and endocrine functions. Simplistically, Wnt activity hinders whereas transforming growth factor (TGF-β)/bone morphogenetic protein (BMP) signaling supports differentiation of progenitors into mature enterocytes, establishing a crypt-villus gradient (Sancho et al. (2004) Annu Rev Cell Dev Biol 20: 695-723). Disrupting the balance between stem cell and differentiation programs is a defining property of colorectal cancer (CRC), which remains the third most common and second most deadly malignancy worldwide accounting for 1.8 million new cases and greater than 860,000 deaths each year, respectively (Siegel et al. (2018) Cancer statistics, 68: 7-30).

The near-universal initiating event in sporadic CRC involves genomic alterations that activate Wnt signaling, most often through loss-of-function APC mutations (Kinzler and Vogelstein (1996) Cell 87: 159-170). Aberrant Wnt activation leads to expansion of the stem cell compartment, shifting the homeostasis between the crypt and villus. As these early colonic lesions evolve, they often develop insensitivity to pro-differentiation cues by selecting for alterations in the TGF-β/BMP pathway. Alternatively, germline variants and mutations in components of the TGF-β/BMP pathway function as susceptibility loci for CRC and predispose to polyposis syndromes, respectively (Haramis et al. (2004) Science 303: 1684-1686; Broderick et al. (2007) Nat Genet 39: 1315-1317; Tomlinson et al. (2011) PLoS Genet 7: e1002105). As such, genomic alterations that hinder intestinal differentiation, either by activating stem cell-like programs or inactivating pro-differentiation pathways, are central to CRC development. Despite the intellectual clarity of this observation, critical regulators of aberrantly active stem cell-like programs or convert this understanding into efficacious treatment for CRC have yet to be uncovered.

SRY-Box transcription factor 9 (SOX9) is a key developmental transcription factor that guides cell fate decisions during developmental and adult homeostasis in diverse tissue including cartilage (Wagner et al. (1994) Cell 79: 1111-1120; Akiyama et al. (2005) 102: 14665-14670), testis (Moreno-Mendoza et al. (2004) Biol Reprod 70: 114-122; Jakob and Lovell-Badge (2011) FEBS J 278: 1002-1009), skin (Kadaja et al. (2014) Genes Dev 28: 328-341; Adam et al. (2015), Nature 521: 366-370), and breast (Guo et al. (2012) Cell 148: 1015-1028). In the intestines, biallelic genetic inactivation of SOX9 led to impaired Paneth cell differentiation in genetically engineered mouse models (Bastide et al. (2007) The Journal of cell biology 178: 635-648; Mori-Akiyama et al. (2007) Gastroenterology 133: 539-546). However, the role of SOX9 in CRC remains unclear as there is evidence for oncogenic and tumor suppressor functions (Lu et al. (2008) Am J Clin Pathol 130: 897-904; Cancer Genome Atlas (2012) Nature 487: 330-337; Matheu et al. (2012) Cancer Res 72: 1301-1315; Carrasco-Garcia et al. (2016) Sci Rep 6: 32350; Prevostel et al. (2016) Oncotarget 7: 82228-82243; Prevostel and Blache (2017) Eur J Cancer 86: 150-157; Hiramatsu et al. (2019) Proc Natl Acad Sci USA 116: 1704-1713; Vasaikar et al. (2019) Cell 177: 1035-1049 e1019). Thus, development of a new treatment regime that would enhance intestinal differentiation in cancer cells on a patient is much needed.

### SUMMARY OF THE INVENTION

The present invention is based, at least in part, on the discovery that genomic alterations that encourage stem cell activity and hinder proper maturation are central to the development of colorectal cancer (CRC). This CRC phenotype can be ameliorated by downregulating one or more biomarker listed in Table 1, such as the ones associated with Paneth and stem cell activity (e.g., Wnt, SOX9, and/or PROM1), such as by deleting one or both copies of the genes listed in Table 1, or by upregulating or enhancing the activity of one or more biomarker listed in Table 2, such as the ones associated with intestinal differentiation (e.g., KRT20), and/or by treatment with an agent that modifies the copy number, the expression level, and/or the activity of one or more biomarkers listed in Table 1 or 2, or a fragment thereof. In accordance with disclosures provided herein, various intestinal cancers (e.g., CRC, small intestine cancer, adenocarcinoma, and the like) and/or polyposis in a subject can be treated by administering to the subject an agent that modifies the copy number, the expression level, and/or the activity of one or more biomarkers listed in Table 1 or 2, or a fragment thereof. Such an administration can treat the intestinal cancers, including CRC, and/or polyposis by inducing intestinal differentiation of CRC cells into mature intestinal cells in the subject. Moreover, it was unexpectedly determined herein that modulators of the one or more biomarkers listed in Tables 1-2, such as Wnt, SOX9, PROM1, and/or KRT20, are useful for modulating the growth of cancer cells, particularly in patients afflicted with an intestinal cancer or a CRC, and represents a novel strategy for treating the intestinal cancer, CRC, and/or polyposis. In addition, the biomarkers provide a screening platform for identifying agents that modulate intestinal cell differentiation.

One aspect of the invention provides a method of treating a subject afflicted with an intestinal cancer and/or polyposis comprising administering to the subject a therapeutically effective amount of an agent that modifies the copy number, the expression level, and/or the activity of one or more biomarkers listed in Table 1 or 2, or a fragment thereof.

Numerous embodiments are further provided that can be applied to any aspect of the present invention and/or combined with any other embodiment described herein. For example, in one embodiment, the agent decreases the copy number, the expression level and/or the activity of one or more biomarkers listed in Table 1 or a fragment thereof. In another embodiment, the agent decreases the copy number, the expression level, and/or the activity of a Wnt polypeptide, a SOX9 polypeptide, and/or a PROM1 polypeptide, or polynucleotide encoding the respective polypeptides. In still another embodiment, the agent increases the copy number, the expression level and/or the activity of one or more biomarkers listed in Table 2 or a fragment thereof. In another embodiment, the agent increases the copy number, the expression level, and/or the activity of a KRT20 polypeptide and/or a CDH1 (Cadherin 1) polypeptide, or polynucleotide encoding the respective polypeptides. In yet another embodiment, the agent is a small molecule inhibitor, CRISPR single-guide RNA (sgRNA), RNA interfering agent, antisense oligonucleotide, peptide or peptidomimetic inhibitor, aptamer, antibody, or intrabody. In another embodiment, the RNA interfering agent is a small interfering RNA (siRNA), CRISPR RNA (crRNA), a small hairpin RNA (shRNA), a microRNA (miRNA), or a piwi-interacting RNA (piRNA). In still another embodiment, the shRNA comprises a nucleic acid sequence selected from the group consisting of nucleic acid sequence listed in Table 3. In yet another embodiment, the agent comprises an intrabody, or an antigen binding fragment thereof, that specifically binds to the one or more biomarkers and/or a substrate of the one or more biomarkers listed in Table 1 or 2. In another embodiment, wherein the intrabody, or antigen binding fragment thereof, is a murine, chimeric, humanized, composite, or human intrabody, or antigen binding fragment thereof. In another embodiment, the intrabody, or antigen binding fragment thereof, is detectably labeled, comprises an effector domain, comprises an Fc domain, and/or is selected from the group consisting of Fv, Fav, F(ab′)2, Fab′, dsFv, scFv, sc(Fv)2, and diabody fragments. In another embodiment, the agent increases the copy number, the expression level and/or the activity of one or more biomarkers listed in Table 2 or a fragment thereof. In still another embodiment, the agent increases the sensitivity of the cancer cells to an immunotherapy. In yet another embodiment, the immunotherapy is administered before, after, or concurrently with the agent. In still another embodiment, the immunotherapy comprises an anti-cancer vaccine and/or virus. In another embodiment, the immunotherapy is a cell-based immunotherapy, optionally wherein the cell-based immunotherapy is chimeric antigen receptor (CAR-T) therapy. In another embodiment, the method also comprises administering to the subject at least one additional cancer therapy or regimen. In yet another embodiment, the at least one additional cancer therapy or regimen is administered before, after, or concurrently with the agent and/or the immunotherapy. In yet another embodiment, the agent is administered in a pharmaceutically acceptable formulation. In still another embodiment, the intestinal cancer is CRC. In still another embodiment, the intestinal cancer is CRC, small intestine cancer, and/or adenocarcinoma. In some embodiments, the subject is at risk for an intestinal cancer. In some embodiments, the subject is afflicted with polyposis.

In another aspect, a method of promoting intestinal differentiation, such as promoting intestinal differentiation of an intestinal cancer cell toward a mature intestinal cell in a subject, the method comprising administering to the subject an effective amount of an agent that modifies the copy number, the expression level, and/or the activity of one or more biomarkers listed in Table 1 or 2 or a fragment thereof, is provided.

As described above, numerous embodiments are further provided that can be applied to any aspect of the present invention and/or combined with any other embodiment described herein. For example, in one embodiment, the agent inhibits or blocks a stem cell-like program in a subject, thereby promoting intestinal differentiation. In another embodiment, the agent decreases the copy number, the expression level and/or the activity of one or more biomarkers listed in Table 1 or a fragment thereof. Yet in another embodiment, the agent decreases the copy number, the expression level, and/or the activity of a Wnt polypeptide, a SOX9 polypeptide, and/or a PROM1 polypeptide, or polynucleotide encoding the respective polypeptides. In still another embodiment, the agent increases the copy number, the expression level and/or the activity of one or more biomarkers listed in Table 2 or a fragment thereof. In another embodiment, the agent increases the copy number, the expression level, and/or the activity of a KRT20 polypeptide and/or a CDH1 (Cadherin 1) polypeptide, or polynucleotide encoding the respective polypeptides. In yet another embodiment, the agent is a small molecule inhibitor, CRISPR single-guide RNA (sgRNA), RNA interfering agent, antisense oligonucleotide, peptide or peptidomimetic inhibitor, aptamer, antibody, or intrabody. In another embodiment, the RNA interfering agent is a small interfering RNA (siRNA), CRISPR RNA (crRNA), a small hairpin RNA (shRNA), a microRNA (miRNA), or a piwi-interacting RNA (piRNA). In still another embodiment, the shRNA comprises a nucleic acid sequence selected from the group consisting of nucleic acid sequence listed in Table 3. In yet another embodiment, the agent comprises an intrabody, or an antigen binding fragment thereof, that specifically binds to the one or more biomarkers and/or a substrate of the one or more biomarkers listed in Table 1 or 2. In another embodiment, wherein the intrabody, or antigen binding fragment thereof, is a murine, chimeric, humanized, composite, or human intrabody, or antigen binding fragment thereof. In another embodiment, the intrabody, or antigen binding fragment thereof, is detectably labeled, comprises an effector domain, comprises an Fc domain, and/or is selected from the group consisting of Fv, Fav, F(ab′)2, Fab′, dsFv, scFv, sc(Fv)2, and diabody fragments.

Another aspect of the present invention is a method of determining whether a subject afflicted with or at risk (e.g., a subject afflicted with polyposis) for developing an intestinal cancer and/or a CRC would benefit from a therapy modulating the copy number, amount, and/or activity of at least one biomarker listed in Table 1 or 2, the method comprising a) obtaining a biological sample from the subject; b) determining the copy number, amount, and/or activity of at least one biomarker listed in Table 1 or 2; c) determining the copy number, amount, and/or activity of the at least one biomarker in a control; and d) comparing the copy number, amount, and/or activity of the at least one biomarker detected in steps b) and c), wherein the presence of, or a significant increase in, the copy number, amount, and/or activity of at least one biomarker listed in Table 1 in the subject sample relative to the control copy number, amount, and/or activity of the at least one biomarker indicates that the subject afflicted with or at risk for developing the intestinal cancer would benefit from the therapy, or wherein the absence of, or a significant decrease in, the copy number, amount, and/or activity of at least one biomarker listed in Table 2 in the subject sample relative to the control copy number, amount, and/or activity of the at least one biomarker indicates that the subject afflicted with or at risk for developing the intestinal cancer would benefit from the therapy, is provided.

As described above, numerous embodiments are further provided that can be applied to any aspect of the present invention and/or combined with any other embodiment described herein. For example, in one embodiment, the method further comprises recommending, prescribing, or administering the agent that modifies the copy number, the expression level, and/or the activity of one or more biomarkers listed in Table 1 or 2 or a fragment thereof if the subject is determined to benefit from the agent. In another embodiment, the method further comprises recommending, prescribing, or administering at least one additional cancer therapy that is administered before, after, or concurrently with the agent that modifies the copy number, the expression level, and/or the activity of one or more biomarkers listed in Table 1 or 2 or a fragment thereof. In still another embodiment, the method further comprises recommending, prescribing, or administering cancer therapy other than the agent that modifies the copy number, the expression level, and/or the activity of one or more biomarkers listed in Table 1 or 2 or a fragment thereof if the subject is determined not to benefit from the agent. In yet another embodiment, the agent is a small molecule inhibitor, CRISPR single-guide RNA (sgRNA), RNA interfering agent, antisense oligonucleotide, peptide or peptidomimetic inhibitor, aptamer, antibody, or intrabody. In another embodiment, the RNA interfering agent is a small interfering RNA (siRNA), CRISPR RNA (crRNA), a small hairpin RNA (shRNA), a microRNA (miRNA), or a piwi-interacting RNA (piRNA). In still another embodiment, the shRNA comprises a nucleic acid sequence selected from the group consisting of nucleic acid sequence listed in Table 3. In yet another embodiment, the agent comprises an intrabody, or an antigen binding fragment thereof, that specifically binds to the one or more biomarkers and/or a substrate of the one or more biomarkers listed in Table 1 or 2. In another embodiment, wherein the intrabody, or antigen binding fragment thereof, is a murine, chimeric, humanized, composite, or human intrabody, or antigen binding fragment thereof. In another embodiment, the intrabody, or antigen binding fragment thereof, is detectably labeled, comprises an effector domain, comprises an Fc domain, and/or is selected from the group consisting of Fv, Fav, F(ab′)2, Fab′, dsFv, scFv, sc(Fv)2, and diabody fragments. In another embodiment, the control sample comprises cells.

Another aspect of the present invention is a method of identifying a subject afflicted with, or at risk for developing, an intestinal cancer or a CRC that can be treated by modulating the copy number, amount, and/or activity of at least one biomarker listed in Table 1 or 2, the method comprising detecting an increased or decreased expression level of at least one biomarker listed in Table 1 or 2 in a cell from the subject relative to a control, thereby identifying the subject afflicted with, or at risk of developing, a cancer that can be treated by modulating the copy number, amount, and/or activity of at least one biomarker listed in Table 1 or 2, optionally wherein a biological sample comprising the cell from the subject is obtained from the subject.

Numerous embodiments are further provided that can be applied to any aspect of the present invention and/or combined with any other embodiment described herein. In one embodiment, the agent decreases the copy number, amount, and/or activity of at least one biomarker listed in Table 1. In another embodiment, the method also comprises recommending, prescribing, or administering to the identified subject an agent that inhibits the at least one biomarker listed in Table 1. In yet another embodiment, the agent increases the copy number, amount, and/or activity of at least one biomarker listed in Table 2. In another embodiment, the method further comprises recommending, prescribing, or administering to the identified subject an immunotherapy. In one embodiment, the immunotherapy comprises an anti-cancer vaccine, an anti-cancer virus, and/or a checkpoint inhibitor. In another embodiment, the method further comprises recommending, prescribing, or administering to the subject a cancer therapy selected from the group consisting of targeted therapy, chemotherapy, radiation therapy, and/or hormonal therapy. In yet another embodiment, the control comprises a sample derived from a cancerous or non-cancerous sample from either the patient or a member of the same species to which the patient belongs. In still another embodiment, the control is a known reference value. In one embodiment, the intestinal cancer is CRC. In another embodiment, the intestinal cancer is CRC, small intestine cancer, and/or adenocarcinoma.

In yet another aspect, a method for predicting the clinical outcome of a subject afflicted with an intestinal cancer or a CRC to treatment with a down-regulator of SOX9 and/or PROM1 signaling, the method comprising a) determining the copy number, amount, and/or activity of at least one biomarker listed in Table 1 or 2 in a subject sample; b) determining the copy number, amount, and/or activity of the at least one biomarker in a control having a good clinical outcome; and c) comparing the copy number, amount, and/or activity of the at least one biomarker in the subject sample and in the control, wherein the presence of, or a significant change in the copy number, amount, and/or activity of at least one biomarker listed in Table 1 or 2 in the subject sample as compared to the copy number, amount and/or activity in the control, is an indication that the subject has a favorable clinical outcome, is provided.

In another aspect, a method for monitoring the efficacy of a down-regulator of SOX9 and/or PROM1 signaling in activating intestinal cell differentiation and/or treating an intestinal cancer or a CRC, wherein the subject is administered a therapeutically effective amount of the down-regulator of SOX9 and/or PROM1 signaling, the method comprising a) detecting in a subject sample at a first point in time the copy number, amount, and/or activity of at least one biomarker listed in Table 1 or 2; b) repeating step a) at a subsequent point in time; and c) comparing the amount or activity of at least one biomarker listed in Table 1 or 2 detected in steps a) and b) to monitor the progression of the cancer in the subject, wherein the absence of, or a significant decrease in the copy number, amount, and/or activity of, the at least one biomarker listed in Table 1 in the subject sample as compared to the copy number, amount and/or activity in the control, is an indication that the down-regulator of SOX9 and/or PROM1 signaling effectively treats the intestinal cancer and/or the CRC in the subject, or wherein the presence of, or a significant increase in the copy number, amount, and/or activity of, the at least one biomarker listed in Table 2 in the subject sample as compared to the copy number, amount and/or activity in the control, is an indication that the down-regulator of SOX9 and/or PROM1 signaling effectively treats the intestinal cancer and/or the CRC in the subject.

In still another aspect, a method of assessing the efficacy of an agent that inhibits the copy number, amount, and/or activity of at least one biomarker listed in Table 1 for activating intestinal cell differentiation, treating polyposis and/or treating an intestinal cancer or a CRC, comprising a) detecting in a sample at a first point in time the copy number, amount, and/or or activity of at least one biomarker listed in Table 1; b) repeating step a) during at least one subsequent point in time after contacting the sample with the agent; and c) comparing the copy number, amount, and/or activity detected in steps a) and b), wherein the absence of, or a significant decrease in, the copy number, amount, and/or activity of, the at least one biomarker listed in Table 1, in the subsequent sample as compared to the copy number, amount, and/or activity in the sample at the first point in time, indicates that the agent effectively activates intestinal cell differentiation, treats polyposis and/or treats the intestinal cancer or the CRC, optionally wherein the sample is from a subject, is provided.

In yet still another aspect, a method of assessing the efficacy of an agent that enhances the copy number, amount, and/or activity of at least one biomarker listed in Table 2 for activating intestinal cell differentiation, treats polyposis and/or treating an intestinal cancer or a CRC, comprising a) detecting in a sample at a first point in time the copy number, amount, and/or or activity of at least one biomarker listed in Table 2; b) repeating step a) during at least one subsequent point in time after contacting the sample with the agent; and c) comparing the copy number, amount, and/or activity detected in steps a) and b), wherein the presence of, or a significant increase in, the copy number, amount, and/or activity of, the at least one biomarker listed in Table 2, in the subsequent sample as compared to the copy number, amount, and/or activity in the sample at the first point in time, indicates that the agent effectively activates intestinal cell differentiation, treats polyposis and/or treats the intestinal cancer or the CRC, optionally wherein the sample is from a subject, is provided.

As described above, numerous embodiments are further provided that can be applied to any aspect of the present invention and/or combined with any other embodiment described herein. In one embodiment, between the first point in time and the subsequent point in time, the subject has undergone treatment, completed treatment, and/or is in remission for the cancer. In another embodiment, treatment comprises administering the agent to the subject. In yet another embodiment, the first and/or the subsequent sample comprises ex vivo or in vitro samples. In still another embodiment, the first and/or at least one subsequent sample is a portion of a single sample or pooled samples obtained from the subject. In another embodiment, the sample comprises cells, serum, peritumoral tissue, and/or intratumoral tissue obtained from the subject. In yet another embodiment, the one or more biomarkers listed in Table 1 or 2. In one embodiment, the cancer or cancer cell is a CRC. In another embodiment, the neuroendocrine cancer is CRC, small intestine cancer, and/or adenocarcinoma. In yet another embodiment, the cancer or cancer cell is in an animal model of the cancer. In still another embodiment, the animal model is a mouse model. In one embodiment, the cancer is in a mammalian subject. In another embodiment, the mammalian subject is a mouse or a human. In yet another embodiment, the mammal is a human.

In another aspect, a genetically modified cell, comprising: a first stably integrated endogenous reporter system expressing one or more biomarkers listed in Table 1 and a first signal, wherein an increased level of the first signal corresponds to an endogenous stem cell-like transcriptional activity within the cell; and/or a second stably integrated endogenous reporter system expressing one or more biomarkers listed in Table 2 and a second signal, wherein an increased level of the second signal corresponds to an endogenous intestinal differentiation activity within the cell, is provided.

As described above, numerous embodiments are further provided that can be applied to any aspect of the present invention and/or combined with any other embodiment described herein. In one embodiment, the one or more biomarkers listed in Table 1 comprises SOX9, PROM1, LGR5, and/or ASCL2 and the one or more biomarkers listed in Table 2 comprises KRT20, MUC2, and/or DPP4. In other embodiment, the genetically modified cell comprises one or more additional stably integrated endogenous reporter system expressing one or more biomarkers listed in Table 1 or 2 and an additional signal. Consistent with these embodiments, the first signal, the second signal, and the additional signal comprise a molecule, a probe, and/or a protein that are fluorescent or radioactive, and/or the first signal, the second signal, or the additional signal comprise a green fluorescent protein (GFP), a red fluorescent protein (RFP), a yellow fluorescent protein (YFP), and/or a cyan fluorescent protein (CFP), or a derivative or a fragment thereof, or the first signal is a GFP and the second signal is a RFP, or a derivative thereof, such as mKATE2. In yet another embodiment, the cell exhibits a phenotype of an intestinal cancer or a CRC, and/or the cell is derived from a model of an intestinal cancer or a CRC, and/or the cell is derived from a cancerous sample from either a subject or a member of the same species to which the subject belongs, and/or the cell expresses the first signal, the second signal, and/or the additional signal. In another embodiment, a stable cell line comprising a plurality of the cells as disclosed above and herein.

In another aspect, a method of identifying an agent for activating intestinal cell differentiation, treating polyposis and/or treating an intestinal cancer or a CRC, comprising: a) detecting at a first point in time the first signal and/or the second signal in a sample comprising the genetically modified cell or the stable cell line of the above; b) repeating step a) during at least one subsequent point in time after contacting the sample with the agent; and c) comparing the expression level of the first signal and/or the second signal detected in steps a) and b), wherein the absence of, or a significant decrease in, the expression level of the first signal in the subsequent sample as compared to the expression level of the first signal in the sample at the first point in time, indicates that the agent effectively activates intestinal cell differentiation, treats polyposis and/or treats the intestinal cancer or the CRC, and/or wherein the presence of, or a significant increase in, the expression level of the second signal in the subsequent sample as compared to the expression level of the second signal in the sample at the first point in time, indicates that the agent effectively activates intestinal cell differentiation, treats polyposis and/or treats the intestinal cancer or the CRC, optionally wherein the sample is from a subject, is provided.

As described above, numerous embodiments are further provided that can be applied to any aspect of the present invention and/or combined with any other embodiment described herein. In one embodiment, the agent is a small molecule, CRISPR single-guide RNA (sgRNA), RNA interfering agent, antisense oligonucleotide, peptide or peptidomimetic inhibitor, aptamer, antibody, or intrabody. In another embodiment, the RNA interfering agent is a small interfering RNA (siRNA), CRISPR RNA (crRNA), a small hairpin RNA (shRNA), a microRNA (miRNA), or a piwi-interacting RNA (piRNA).

In another aspect, a method of making an endogenous cell reporter system, comprising: integrating a first cassette comprising a first signal and an antibiotic resistance into a genomic locus of one or more biomarkers listed in Table 1 in a cell; and/or integrating a second cassette comprising a second signal and an antibiotic resistance into a genomic locus of one or more biomarkers listed in Table 2 in a cell, is provided.

As described above, numerous embodiments are further provided that can be applied to any aspect of the present invention and/or combined with any other embodiment described herein. In one embodiment, the one or more biomarkers listed in Table 1 comprises SOX9, PROM1, LGR5, and/or ASCL2 and the one or more biomarkers listed in Table 2 comprises KRT20, MUC2, and/or DPP4. In other embodiment, the method comprises integrating one or more additional cassettes comprising an additional signal and an antibiotic resistance into a genomic locus of one or more biomarkers listed in Table 1 or 2. In another embodiment, the method comprises selecting an antibiotic-resistant population of cells, wherein the antibiotic-resistant population of cells indicates successful integration of the first cassette, the second cassette and/or the one or more additional cassettes. Consistent with these embodiments, the first signal, the second signal, or the additional signal comprises a polynucleotide encoding a molecule, a probe, and/or a protein that are fluorescent or radioactive, and/or the first signal, the second signal, or the additional signal comprises a polynucleotide encoding a green fluorescent protein (GFP), a red fluorescent protein (RFP), a yellow fluorescent protein (YFP), and/or a cyan fluorescent protein (CFP), or a derivative or a fragment thereof, or the first signal is a polynucleotide encoding a GFP and the second signal is a polynucleotide encoding a RFP, or a derivative thereof, such as mKATE2. In yet another embodiment, the cell exhibits a phenotype of an intestinal cancer or a CRC, and/or the cell is derived from a model of an intestinal cancer or a CRC, and/or the cell is derived from a cancerous sample from either a subject or a member of the same species to which the subject belongs, and/or the cell expresses the first signal, the second signal, and/or the additional signal. In another embodiment, the method comprises developing a stable cell line comprising a plurality of the cells as disclosed above and herein.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIG. 1A-FIG. 1G show that SOX9 is required for CRC proliferation and primary tumor growth. FIG. 1A shows cell line dependency for SOX9 CRISPR KO (left) and RNAI (right) plotted against SOX9 mRNA expression in CCLE CRC cell lines; regression line indicated in blue; p-value calculated by Pearson correlation. FIG. 1B shows SOX9 and 3-actin protein expression in HT-115 cells stably expressing control or shRNAs targeting SOX9 by immunoblot (left panel). Adherent proliferation of indicated HT-115 cells over a six day period as determined by cell titer glo assay (middle panel). Area quantification of HT-115 control and indicated SOX9-shRNAs under ultra-low attachment culture (right panel). Data presented as mean±S.D. of three culture replicates; p-value calculated using two-sided Student's t-test. FIG. 1C shows Immunoblot and colony formation of HT-29 cells expressing doxycycline-inducible NTC, shRNA #1 or shRNA #5 in the presence and absence of 0.5 μg/ml doxycycline under adherent culture. Immunoblot showing protein levels of SOX9 and vincuclin (loading control). FIG. 1D shows soft-agar colony formation assay of COLO-205 cells expressing control or indicated shRNAs targeting SOX9. Quantification of colony number presented as mean±S.D. of three cell culture replicates and representative phase contrast images; p-value calculated by two-sided Student's t-test. FIG. 1E shows primary tumor growth of HT-29 cells stably expressing vector control or SOX9 shRNA #1 injected into the flanks of nude mice. Representative image of nude mouse xenografts and ex-vivo tumors at experiment end-point. Growth curve of primary tumors at different time points after subcutaneous injection. Data expressed as mean±S.D (n=5); P values calculated by two-sided Student's t-test where *p<0.05. FIG. 1F shows proportion charts representing distribution of WT/heterozygous, heterozygous, homozygous SOX9 inactivation in single cell clones generated from three CRC cell lines stably expressing a SOX9 sgRNA and Cas9 (left). Proliferation of indicated COLO-205 single clones measured by cell titerGlo (right). FIG. 1G shows LS180 cells that were nucleofected with Cas9/SOX9 sgRNA complex and then amplicon sequenced at cut-site to determine frequency of in-frame and frameshift indels.

FIG. 2A-FIG. 2E show that SOX9 blocks intestinal differentiation in human CRC. FIG. 2A shows Sox9 and Krt20 immunohistochemistry of normal mouse small intestines. Scale bar=100 μM. FIG. 2B shows V5-tagged proteins, KRT20 and GAPDH (loading control) protein expression in LS180 cells overexpressing V5-tagged GFP, WT or mutant SOX9 in the presence of 0.5 ug/mL doxycycline by immunoblot. FIG. 2C shows mRNA expression of SOX9, KRT20, MUC2, and CDX2 in LS180 cells overexpressing V5-tagged GFP, WT or mutant SOX9 in the presence of 0.5 ug/mL doxycycline by RT-PCR. Data expressed as mean±S.D of three technical replicates; p-values calculated by two-sided Student's t-test where *** p<0.005, and **** p<0.001. FIG. 2D shows SOX9, Keratin20 and GAPDH (loading control) protein expression in HT-115 cells inducibly expressing NTC or SOX9-sh #1 using 0.25 μg/ul doxycycline over time by immunoblot. FIG. 2E shows E-cadherin, Vimentin and GAPDH (loading control) protein expression in single cell clones generated from COLO-205 sgRNA #9 cell line. Phase contrast images of different clones cultured at Day 1. Scale bar=100 μM.

FIG. 3A-FIG. 3G show that SOX9 KD promotes intestinal differentiation in organoid models of CRC. FIG. 3A shows heat map illustrating mRNA expression of intestinal differentiation (blue) and stem cell (red) markers in human colon organoids stably expressing control, SOX9-sh #1 and SOX9-sh #5. FIG. 3B shows Sox9 and Vinculin (loading control) protein expression in mouse Apc^{KO}; Kras^{G12D} colon organoids expressing vector control, Sox9 sh #2 or Sox9 sh #5 by immunoblot. Normalized proliferation of organoids expressing vector control, shRNA #2, or shNA #5 determined by cell titer glo over 6 days in culture. Data expressed as mean±S.D of three replicates; p-values calculated by two-sided Student's t-test where *** p<0.005 and **** p<0.001. FIG. 3C shows Quantification of nuclear Ki67 staining from total nuclei count of fixed mouse Apc^{KO};Kras^{G12D} colon organoids stably expressing control or indicated SOX9 shRNAs. Data expressed as mean±S.D and p-values calculated by two-sided Student's t-test where *** p<0.005 and **** p<0.001. Scale bar=50 μM. FIG. 3D shows mRNA expression of Sox9; stem cell markers Lgr5, Lrig1, and Prom1; WNT pathway markers Axin2 and Ascl2; and intestinal differentiation marker Krt20 in engineered Apc^{KO}; Kras^{G12D} colon organoids by qRT-PCR. Data expressed as mean±S.D of three biological replicates; p-values calculated by two-sided Student's t-test where ** p<0.01, *** p<0.005 and **** p<0.001. FIG. 3E shows schematic indicating xenograft experimental design in which individual mice where injected with Apc^{KO} Kras^{G12D} control organoids on the left flank and one of two Sox9 shRNAs in the right flank (top left panel). Primary tumor xenograft growth curve shown as well as individual tumor volumes at experiment end-point at day 30 (bottom panel). Representative images of nude mice with subcutaneous tumors and excised tumors from the mice at experiment end-point (top right panel). FIG. 3F shows Sox9 and Vinculin (loading control) protein expression in xenograft tumors at experiment end-point validating gene knockdown. FIG. 3G shows Sox9, Ki67, and Muc2 immunohistochemistry and alcian blue staining of formalin fixed xenograft tumors. Scale bars=100 μM and 20 μM.

FIG. 4A-FIG. 4F show that SOX9 activates an enhancer-driven stem cell program in CRC. FIG. 4A shows heat map depicting gene expression profiles of LS180 CRC cells inducibly expressing GFP control, WT SOX9, and SOX9^{ΔC} in the presence or absence of doxycycline. Select gene groups are highlighted in boxes with red representing upregulated genes and blue representing downregulated genes. FIG. 4B shows distribution of genome-wide SOX9 binding sites among intergenic, intronic, promoter, and exonic regions in HT-115 cells. FIG. 4C shows gene ontology enrichment analysis in ‘Biological Process’ category for genes associated with SOX9-binding in HT-115 cells. Binominal false discovery rate q-value following Bonferroni correction. FIG. 4D shows transcription factor binding similarity (Giggle score) using publicly available CHIP-seq data of indicated transcription factors and V5-SOX9 CHIP-seq data. FIG. 4E shows gene track using integrative genomics viewer (IGV) snapshot showing indicated genes associated with stem cell function; signal tracks of H3K27ac-ChIP-seq data (red) and the V5-ChIP-seq data (blue) are derived from HT-115 cells inducibly overexpressing GFP, WT SOX9 and mutant SOX9 with the indicated doxycycline conditions; signal track for TCF4 (purple) was obtained from endogenous CHIP data using LS180 CRC cell line. FIG. 4F shows Kaplein-Mieir curve indicating disease-free survival of patients with CRC that have low, intermediate, or high expression of SOX9.

FIG. 5A-FIG. 5F show that SOX9 directly activates PROM1 via an WNT-responsive intronic enhancer. FIG. 5A shows Venn diagram (left panel) summarizing genes potentially regulated by SOX9 based on RNA-seq and indicated CHIP-seq data sets from HT-115 cells inducibly overexpressing GFP control or SOX9. Volcano plot (right panel) display log₂fold of differentially expressed genes (SOX/GFP) along x-axis (FDR<0.05). Blue points mark the genes significantly associated with SOX9-binding based on V5-ChIP-seq or differential H3K27ac-ChIP-seq. FIG. 5B shows Prom1 mRNA expression levels in control or Sox9 shRNA expressing Apc^{KO} Kras^{G12D} colon organoids by qRT-PCR. Data expressed as mean±S.D of three technical replicates; P values calculated by two-sided Student's t-test where **** p<0.001. FIG. 5C shows PROM1 mRNA expression levels in human CRC with WT, heterozygous, or homozygous SOX9 mutations. FIG. 5D shows PROM1 and GAPDH (loading control) protein expression in LS180 cells overexpressing GFP, WT SOX9 and mutant SOX9 in the presence and absence of 0.5 ug/mL doxycycline. FIG. 5E shows Gene track depicting intron 1 of PROM1 with signal tracks of H3K27ac-ChIP-seq data (in red) and the V5-ChIP-seq data (in blue) in the HT-115 cells overexpressing GFP, WT SOX9 and mutant SOX9 in the indicated doxycycline conditions. Peak scale indicated in upper right hand corner of respective CHIP-seq data. FIG. 5F shows PROM1 enhancer reporter assay. Schematic of reporter system in which 537-bp SOX9-binding site in the intron of PROM1 was placed upstream of a minimal promoter (MP) and GFP using Gibson cloning (left). Phase contrast and fluorescence images of HEK293T cells transiently transfected with indicated plasmids and/or treated with WNT3A (right).

FIG. 6A-FIG. 6J show that PROM1 blocks intestinal differentiation in CRC. FIG. 6A shows normalized proliferation and colony formation of HT-115 CRC cells inducibly expressing nontargeting control (NTC) or different shRNAs against PROM1 using 0.25 μg/mL doxycycline following 8 days of culture. Data expressed as mean±S.D. of three replicates; P values calculated by two-sided Student's t-test where *** p<0.005. FIG. 6B shows normalized proliferation and colony formation of LS180 CRC cells inducibly expressing NTC or different shRNAs against PROM1 using 0.25 μg/mL doxycycline following 8 days of culture. Data expressed as mean±S.D. of three replicates; P values calculated by two-sided Student's t-test where **** p<0.001. FIG. 6C shows primary tumor xenograft growth curve of NTC control or indicated PROM1 KD HT-29 cells by weekly tumor volume measurements (left) and tumor weights at experiment end-point (right). P values calculated by two-sided Student's t-test. FIG. 6D shows PROM1, KRT20, and GAPDH (loading control) protein expression in HT115 cells expressing NTC or inducible PROM1-shRNAs in the presence and absence of 0.5 ug/mL doxycycline for 5 days by immunoblot. FIG. 6E shows KRT20 and vinculin (loading control) protein expression in LS180 cells expressing NTC or shRNA #4 targeting PROM1 using 0.5 ug/mL doxycycline over indicated times by immunoblot. FIG. 6F shows KRT20, PROM1, SOX9 and vinculin (loading control) protein expression in LS180 cells expressing GFP control, PROM1, PROM1-V5, PROM1ΔC, and WT SOX9 by immunoblot. FIG. 6G shows KRT20, PROM1, SOX9, GAPDH, and vinculin (loading control) protein expression in HT-115 (left) and LS180 (right) cells expressing NTC or different shRNAs against PROM1. FIG. 6H shows KRT20, PROM1, SOX9, AXIN2, GAPDH, and vinculin (loading control) protein expression in HT-115 (left) and LS180 (right) cells inducible expressing PROM1 shRNA #4 followed by its release via doxycycline withdrawal over the indicated time period. FIG. 6I shows KRT20 mRNA expression in LS180 cells inducibly expressing either NTC or PROM11 sh #4 targeting 3′UTR and GFP or WT-SOX9 using 0.5 μg/ul dioxyline for 3 days by RT-PCR (top panel). KRT20, SOX9 and vinculin (loading control) protein expression in LS180 cells expressing the indicated vectors (bottom panel). Data expressed as mean±S.D. of three technical replicates; p-values calculated by two-sided Student's t-test where **** p<0.001. FIG. 6J shows KRT20 mRNA and protein expression in LS180 cells stably overexpressing GFP control, WT PROM1, or indicated truncated PROM1 proteins.

FIG. 7 shows that PROM1-SOX9 positive feedback loop blocks differentiation in CRC and further shows schematic summarizing the intergration of stem cell (Wnt) and differentiation (TGF-0) cues by SOX9, which then participates in a reinforcing positive feedback loop involving PROM1 to maintain stem cell activity in CRC.

FIG. 8A-FIG. 8J show that WT SOX9 is necessary for CRC proliferation and growth. FIG. 8A shows adherent proliferation and low-attachment colony formation of five CRC cell lines stably expressing vector control and SOX9 shRNA #1 as determined by cell titer glo assay measured at four days. Data expressed as mean±S.D and P values calculated by two-sided Student's t-test; # indicates no significant difference. FIG. 8B shows phase contrast images of COLO205 vector control and shRNA #1 colonies under low-attachment culture conditions. Quantification of colony number and size expressed as mean±S.D. of three cell culture replicates; p-values calculated by two-sided Student's t-test. FIG. 8C shows immunoblot (left panel), phase contrast images (middle panel), and proliferation (right panel) of HT-29 parental, vector control, and shRNA #1 cells. Immunoblot shows SOX9 and Vimentin expression relative to β-actin loading control. Phase contrast images of isogenic HT-29 cells growing under low-attachment or adherent conditions. Normalized proliferation determined by cell titer glo assay. Data expressed as mean±S.D. and p-values calculated by two-sided Student's t-test, *=P value <0.05. FIG. 8D shows normalized proliferation by cell titer glo and phase contrast images of HT-115 control and SOX9-shRNAs under ultra-low attachment culture. Data presented as mean S.D. of three cell culture replicates; P values calculated by two-sided Student's t-test. Representative phase contrast images of HT-115 control or shRNA #1 colonies under ultra-low attachment culture conditions. FIG. 8E shows immunoblot and proliferation of COLO-205 cells expressing control or indicated SOX9 shRNAs. Immunoblot showing SOX9, TAZ, and f-actin (loading control) expression. Proliferation determined by cell titer glo. Data presented as mean±S.D. of three cell culture replicates; p-values calculated by two-sided Student's t-test. FIG. 8F shows immunoblot and colony area quantification of COLO-205 cells expressing control or SOX9 sh #1 targeting 3′UTR and overexpression of GFP control or WT SOX9. Immunoblot showing protein levels of SOX9, TAZ and f-actin (loading control). Quantification of colony size presented as mean±S.D. of three cell culture replicates; p-value calculated by two-sided Student's t-test. FIG. 8G shows anti-V5 Immunoblot and proliferation of V5-tagged GFP control and SOX9 overexpression HT-29 cells as determined by cell titer glo. Data expressed as mean S.D. of three cell culture replicates; p-values calculated by two-sided Student's t-test, #=not significant difference. FIG. 8H shows anti-V5 Immunoblot and proliferation of V5-tagged GFP control and SOX9 overexpression COLO-205 cells as determined by cell titer glo. Data expressed as mean S.D. of three cell culture replicates; p-values calculated by two-sided Student's t-test, #=not significant difference. FIG. 8I shows quantification of soft agar colony formation in COLO-205 cell lines expressing doxycycline-inducible shRNA #1, shRNA #2, or shRNA #5 in the presence and absence of 1 ug/ul doxycycline. Data expressed as mean±S.D. of three cell culture replicates; p-values calculated by two-sided Student's t-test. FIG. 8J shows quantification of xenograft tumor volumes of HT-29 cells expressing a control or indicated inducible SOX9 shRNAs in nude mice given doxycycline 625 ppm chow for 36 days. Data expressed as mean±S.D. of indicated tumor number; p-value calculated by one-sided ANOVA.

FIG. 9A-FIG. 9B show that SOX9 KO impairs human CRC proliferation. FIG. 9A shows immunoblot and proliferation of COLO-205 cells stably expressing control and indicated sgRNAs targeting SOX9 under adherent culture. Immunoblot showing protein levels of SOX9 and β-actin (loading control). Data presented as mean±S.D. of three cell culture replicates; p-values calculated by two-sided Student's t-test. FIG. 9B shows most frequent DNA amplicon sequencing reads from LS180 CRC cells harboring an endogenous SOX9 heterozygous mutation nucleofected with an inactivating SOX9 sgRNA complexed with recombinant Cas9.

FIG. 10A-FIG. 10E show that SOX9 expression is inversely correlated with intestinal differentiation in human CRC. FIG. 10A shows E-cadherin, SOX9 and β-actin (loading control) protein expression in COLO-205 cells stably expressing GFP or SOX9. FIG. 10B shows mRNA expression levels of SOX9 and CDH1 in HT-29 CRC cells stably expressing vector control or indicated SOX9 shRNAs by qRT-PCR. Data expressed as mean±S.D of three technical replicates; p-values calculated by two-sided Student's t-test. FIG. 10C shows SOX9 and CDH1 mRNA expression in COLO-205 cells expressing nontargeting control (NTC) or indicated inducible SOX9 shRNAs in the presence and absence of 0.5 ug/ul doxycycline for 13 days by qRT-PCR. Data expressed as mean±S.D of three technical replicates; p-values calculated by two-sided Student's t-test where *p<0.05, ** p<0.01, and *** p<0.005. FIG. 10D shows SOX9, E-cadherin (CDH1), Vimentin and GAPDH (loading control) protein expression following inducible SOX9 KD with shRNA #1 over time in the presence and absence of 0.5 ug/ul doxycycline by immunoblot. FIG. 10E shows immunofluorescence of E-cadherin (red), Vimentin (green), and Hoechst (blue) in HT-115 cells following inducible SOX9 KD.

FIG. 11A-FIG. 11C shows that SOX9 suppression promotes intestinal differentiation in murine neoplastic organoid models. FIG. 11A shows Ki67 immunohistochemistry, Keratin20 immunofluorescence (green), and Alcian blue staining in fixed mouse ApcKO;KrasG12D colon organoids stably expressing control or indicated SOX9 shRNAs. Scale bar=50 μM. FIG. 11B shows immunoblot showing Sox9 and Vinculin (loading control) expression levels in mouse ApcKO colon organoids engineered to express inducible shRNAs targeting RFP control or SOX9; mRNA expression levels of Sox9 in engineered ApcKO colon organoids treated with 0.5, 1, or 2 ug/mL of doxycycline by qRT-PCR; Normalized proliferation of ApcKO colon organoids expressing shRFP, shSox9 #2, or shSox9 #5 determined by cell titer glo after 8 days in culture. Data expressed as mean±S.D of three technical replicates for qRT-PCR and three cell culture replicates for proliferation; P values calculated by two-sided Student's t-test where *** p<0.005 and **** p<0.001. FIG. 11C shows immunoblot showing Sox9 and Vinculin (loading control) expression levels in mouse ApcKO;KrasG12D colon organoids engineered to express inducible shRNAs targeting RFP control or SOX9; mRNA expression levels of Sox9 in engineered ApcKO;KrasG12D colon organoids treated with 0.5 ug/mL of doxycycline by qRT-PCR; mRNA expression levels of stem cell and WNT pathway markers Lrig1, Lgr5, Axin2, and Ascl2 in engineered ApcKO;KrasG12D colon organoids treated with 0.5 ug/mL of doxycycline by qRT-PCR; Normalized proliferation rate of ApcKO;KrasG12D colon organoids expressing shRFP, shSox9 #2, or shSox9 #5 determined by cell titer glo after 8 days in culture. Data expressed as mean±S.D of three technical replicates for qRT-PCR and three cell culture replicates for proliferation; P values calculated by two-sided Student's t-test where *** p<0.005 and **** p<0.001.

FIG. 12A-FIG. 12B shows that SOX9 KD induced intestinal differentiation is reversible and leads to impaired xenograft growth. FIG. 12A shows Krt20, Sox9 and 3-actin (loading control) protein expression levels in mouse ApcKO;KrasG12D colon organoids engineered to express inducible shRNAs targeting RFP or SOX9; schematic indicating duration of treatment with and release from 0.5 ug/mL doxycycline exposure in colon organoids. FIG. 12B shows primary tumor growth of ApcKO KrasG12D colon organoids expressing control or indicated Sox9 shRNAs injected into the flanks of nude mice (each mouse carried a control tumor on one flank and one of two Sox9 shRNAs in the other flank). Individual dots represent tumor volumes from indicated mice (using last two digits of mouse #) at day 30. Immunoblot using protein extracted from tumors at the experiment end-point. There were a subset of mice in which Sox9 KD was not successful (“escapers”) indicated in blue and a majority of mice in which Sox9 KD was confirmed (“validated”) indicated in blue. Data expressed as mean±S.D of control (n=15), shRNA #2 (n=7), and shRNA #5 (n=8) groups; P values calculated by two-sided Student's t-test where *p<0.05 and **** p<0.001.

FIG. 13A-FIG. 13G show that SOX9 directly regulates crypt-restricted transcriptional programs. FIG. 13A shows principle component analysis of RNA-seq transcriptional profiles from LS180 CRC cells expressing GFP control, WT SOX9, or SOX9ΔC in the presence or absence of doxycycline. FIG. 13B shows gene ontology analysis and associated p-values based on RNA-seq data from (A). FIG. 13C shows gene-set enrichment analysis (GSEA) using 200 gene Lgr5+ intestinal stem cell signature. FIG. 13D shows monomer and dimer transcription factor motif predictions using HOMER de novo motif analysis in the V5-SOX9-ChIP-seq data set. Statistical analysis was performed using the Fisher exact test. FIG. 13E shows Lyz gene track using integrative genomics viewer (IGV) snapshot showing signal from H3K27ac-ChIP-seq data (red) and the V5-ChIP-seq data (blue) derived from HT-115 cells inducibly overexpressing GFP, WT SOX9 and SOX9^{ΔC} with the indicated doxycycline conditions. FIG. 13F shows MSI1 and MSI2 gene tracks using IGV snapshot showing signal from H3K27ac-ChIP-seq data (red) and the V5-ChIP-seq data (blue) derived from HT-115 cells inducibly overexpressing GFP, WT SOX9 and SOX9ΔC with the indicated doxycycline conditions. FIG. 13G shows mRNA expression levels of indicated stem cell genes in human CRC samples expressing high and low SOX9 levels from TCGA cohort. Each point represents the expression value of an individual patient and the mean is indicated by the black line.

FIG. 14A-FIG. 14B show that SOX9 is regulated by positive signals from Wnt and negative cues from TGF-β signaling pathways. FIG. 14A shows schematic of Wnt pathway regulation of SOX9 based on these findings (left panel). AXIN2, SOX9 and 3-actin (loading control) protein expression in HT-115 (Apc Mutant) cells treated with DMSO, rWNT3A or indicated concentrations of WNT-inhibitor ICG-001 by immunoblot (middle panel). SOX9 and β-actin (loading control) protein expression in HT-115 cells expressing GFP or dominant-negative (dn) TCF4 (right panel); S.E.=short exposure, L.E.=long exposure. FIG. 14B shows schematic of TGF-β pathway regulation of SOX9 based on results disclosed herein. Protein expression of SOX9 and β-actin (loading control) in HT-115 cells treated with indicated concentration of rTGF-β or TGF-β inhibitor.

FIG. 15A-FIG. 15F show SOX9 directly activates intestinal stem cell gene PROM1 in CRC. FIG. 15A shows mRNA expression levels of Sox9 in control or Sox9 shRNA expressing ApcKO KrasG12D colon organoids by qRT-PCR. Data expressed as mean S.D of three technical replicates; p-values calculated by two-sided Student's t-test where **** p<0.001. FIG. 15B shows mRNA expression levels of PROM1 in control or SOX9 shRNA expressing normal human colon organoids by qRT-PCR. Data expressed as mean S.D of three technical replicates; p-values calculated by two-sided Student's t-test where **** p<0.001. FIG. 15C shows Prom1, Sox9, and Gadph (loading control) protein expression in ApcKO KrasG12D colon organoids expressing vector control or inducible Sox9 shRNA in the presence and absence of 0.5 ug/mL doxycycline by immunoblot. FIG. 15D shows mRNA expression levels of PROM1 in HT-115 CRC cells overexpressing GFP control, WT SOX9, or each of three truncated SOX9 proteins by qRT-PCR. Data expressed as mean±S.D of three technical replicates; p-values calculated by two-sided Student's t-test where *p<0.05 and *** p<0.005. FIG. 15E shows Prom1, Sox9, Vinculin (loading control) and Gadph (loading control) protein expression in HT-115 SOX9 shRNA #1 (targets 3′UTR) expressing GFP control, WT SOX9, or each of three truncated SOX9 proteins by immunoblot. FIG. 15F shows mRNA expression levels of SOX9 in LS180 CRC cells inducibly overexpressing GFP control, WT SOX9, or mutant SOX9^{ΔC} in the presence or absence of 0.5 ug/mL doxycycline by qRT-PCR. Data expressed as mean S.D of three technical replicates; p-values calculated by two-sided Student's t-test where **** p<0.001.

FIG. 16A-FIG. 16H show PROM1 is a functional regulator of a SOX9-mediated stem cell program in CRC. FIG. 16A shows mRNA expression levels of PROM1 in HT-115 CRC cells expressing inducible control or PROM1 targeting shRNAs in the presence or absence of 0.5 ug/mL doxycycline by qRT-PCR. Data expressed as mean±S.D of three technical replicates. FIG. 16B shows PROM1, SOX9, p-AKT, T-AKT, p-S6, and GAPDH (loading control) protein levels in HT-115 CRC expressing inducible control or PROM1-targeting shRNAs in the presence or absence of 0.5 ug/mL doxycycline by immunoblot. FIG. 16C shows normalized proliferation of HT-115 CRC cell lines inducibly expressing control or PROM1 targeting shRNAs. Data points shown as cells cultured in presence of 0.5 ug/mL doxycycline divided by cells cultured in the absence of doxycycline. Proliferation determined by cell titer glo. Data expressed as mean±S.D. of three technical replicates; P values calculated by two-sided Student's t-test where **** p<0.001. FIG. 16D shows crystal violet staining of HT115 and LS180 CRC cell lines inducibly expressing non-targeting control (NTC) or PROM1 targeting shRNAs in the presence or absence of 0.5 ug/mL doxycycline. FIG. 16E shows mRNA expression levels of PROM1 and KRT20 in HT-115 CRC cells inducibly expressing non-targeting control or PROM1 targeting shRNAs and constitutively over-expressing in the presence or absence of 0.5 ug/mL doxycycline measured by qRT-PCR. Data expressed as mean±S.D. of three technical replicates; P values calculated by two-sided Student's t-test where *** p<0.005. FIG. 16F shows SOX9, KRT20 and Vinculin (loading control) protein levels in LS180 cell lines inducibly expressing non-targeting control, PROM1-sh #2, or PROM1-sh #4 in the presence or absence of 0.5 ug/mL doxycycline by immunoblot. FIG. 16G shows mRNA expression levels of PROM1 and SOX9 in LS180 CRC cell lines inducibly overexpressing control, SOX9, or PROM1-shRNA #4 in the presence or absence of 0.5 ug/mL doxycycline by qRT-PCR. Data expressed as mean±S.D. of three technical replicates; P values calculated by two-sided Student's t-test where ** p<0.01, *** p<0.005. FIG. 16H shows KRT20 and GAPDH (loading control) protein levels in LS180 cell lines inducibly expressing non-targeting control or PROM1-shRNA #4 and GFP control or SOX9 in the presence or absence of 0.5 ug/mL doxycycline by immunoblot.

FIG. 17A-FIG. 17F show design, establishment, and validation of endogenous stem cell program reporter system. FIG. 17A shows visual representation of the SOX9 genomic locus, sgRNA location, homolog arm structure; E1-E3 represent SOX9 exons (top panel). Post-integration genomic locus of the T2A-GFP-P2A-Neo reporter cassette at the SOX9 locus (bottom panel). Validation primers F1, R1 and R2 are indicated by blue arrows. Primers F1 and R1 amplifies a 672 bp genomic region in the unmodified parental cell line and a 3048 bp product after successful integration of reporter cassette. Primers F1 and R2 verify proper location and orientation of integrated cassette by amplifying a 352 bp product. FIG. 17B shows DNA gel electrophoresis of PCR amplified products showing integration (3048 bp product) and proper orientation (352 bp product) of cassette in LS180^{SOX9-GFP} genome-edited cell line. FIG. 17C shows phase contrast and GFP images of parental LS180 and LS180^{SOX9-GFP} endogenous reporter cell line (top panel); quantification of GFP+ cells in parental LS180 and LS180^{SOX9-GFP} endogenous reporter cell line by flow cytometry. FIG. 17D shows quantification of GFP+ cells in parental, LS180^{SOX9-GFP} and two LS180^{SOx9-GFP} cell lines stably expressing two SOX9 specific shRNAs by flow cytometry. FIG. 17E shows schematic describing focused CRISPR screen using 76 sgRNAs in LS180^{SOX9-T2A-GFP} cell line (left). Following library infection, LS180^{SOX9-GFP} cells were divided into 3 sorted fractions based on GFP intensity. sgRNA abundance is shown in GFP low and negative sorted fractions (right). FIG. 17F shows Kernel density plot demonstrating the distribution of sgRNAs in the top 2.5% GFP+ relative to bottom 2.5% GFP+ sorted fractions; SOX9 and GFP sgRNAs are represented by blue and green dots, respectively, whereas the control sgRNAs are represented by gray dots.

FIG. 18A-FIG. 18D show validation of endogenous SOX9-GFP stem cell reporter. FIG. 18A shows flow cytometry showing distribution of GFP+ cells in LS180 parental versus LS180SOX9-GFP reporter cell line. FIG. 18B shows histogram showing distribution of GFP intensity in control and SOX9 KD LS180SOX9-GFP reporter cells. FIG. 18C shows distribution of GFPhigh, GFPlow, and GFPneg cells in control and SOX9 KD LS180SOX9-GFP reporter cells. FIG. 18D shows work flow and data impact of different normalizations methods to CRISPR screen data.

FIG. 19A-FIG. 19E show development of endogenous intestinal differentiation reporter system. FIG. 19A shows genomic locus of KRT20 in which E1 through E8 represent exons; diagram of HDR template (cassette) containing the GFP fluorescent reporter and neomycin antibiotic selection marker (yellow); modified genomic KRT20 locus after successful integration of the GFP fluorescent reporter cassette (bottom panel). FIG. 19B shows quantification of GFP+ cells in HT-29^{KRT20-GFP} control and SOX9 knockdown in HT-29^{KRT20-GFP} cell lines by flow cytometry. FIG. 19C shows schematic describing focused CRISPR screen using 76 sgRNAs in HT-29^{KRT20-GFP} cell line (left). Following library infection, HT-29^{KRT20-GFP} cells were divided into 2 sorted fractions based on GFP intensity.

FIG. 19D shows Kernel density plot demonstrating distribution of sgRNAs in the top 2.5% GFP+ relative to bottom 2.5% GFP+ sorted fractions; SOX9, GFP, KRT20 sgRNAs are represented by blue, green, and yellow dots, respectively, whereas the control sgRNAs are represented by gray dots. FIG. 19E shows probability density plots showing SOX9, GFP, KRT20 sgRNA distribution as Log₂ Fold Change relative to all control sgRNAs at day 3 and 7.

FIG. 20A-FIG. 20D show differentiation reporter systems. FIG. 20A shows immunohistochemical expression levels of Sox9 and Krt20 in the normal mouse intestines. FIG. 20B shows beta score enrichment of indicated sgRNA populations by comparing different top and bottom percentages. FIG. 20C shows SOX9 sgRNA abundance in HT-29KRT20-mKate2 differentiation and LS180SOX9-GFP stem reporter cells at day 3 and day 7. FIG. 20D shows heatmap of indicated individual sgRNAs relative abundance in HT-29KRT20-mKate2 at day 3 and day 7.

FIG. 21A-FIG. 21C show development of dual stem cell and differentiation endogenous reporter system. FIG. 21A shows schematic showing successful integration of mKate2-blasticidin cassette into the SOX9 locus and GFP-neomycin cassette into the KRT20 locus to establish a dual endogenous reporter that monitors stem cell and differentiation activity, respectively. FIG. 21B shows, after infection of focused CRISPR library in HT-29^{SOX9-mate2/KRT20-GFP}, sgRNA distribution as represented by rank plot where the x-axis shows the rank and the y-axis shows the Log₂ fold change of each sgRNAs (left) and kernel density plot as previously described (right); SOX9, GFP, KRT20, and mKate2 sgRNAs are represented by blue, green, yellow, and red dots, respectively, whereas the control sgRNAs are represented by gray dots. FIG. 21C shows probability density plots showing SOX9, GFP, KRT20, and mKate2 sgRNA distribution as Log₂ Fold Change relative to all control sgRNAs in focused library screen experiments involving HT-29 and HT-115 dual reporter cell lines (left panel); corresponding beta analysis showing gene level enrichment as an aggregate of the corresponding individual sgRNAs (right panel).

FIG. 22A-FIG. 22C show dual endogenous reporter system. FIG. 22A shows flow cytometry representation and gating strategy of GFP+ and K2+ cell populations in single reporter and dual reporter cell lines. FIG. 22B shows flow cytometry distribution of GFP+ and K2+ cell populations in parental versus dual endogenous reporter cell line. FIG. 22C shows SOX9, KRT20, GFP, and mK2 sgRNA abundance in HT-29^{KRT20-mate2/SOX9-GFP}, HT-115^{KRT20-mKate2/SOX9-GFP}, and LS180^{KRT20-mKate2/SOX9-GFP} dual endogenous reporter cell lines.

FIG. 23A-FIG. 23D show application of stem cell and differentiation endogenous reporter system to discover novel therapeutic agents. FIG. 23A shows a schematic describing focused 31 epigenetic inhibitor screen in LS180^{SOX9-GFP stem cell and HT-}115^{KRT2-mKate2} intestinal differentiation cell lines (left); venn diagram showing number of compounds that activated differentiation reporter, blocked stem cell activity, and reduced viability of three parental CRC cell lines. FIG. 23B shows dose-response curves of 11 epigenetic inhibitors relatively to DMSO in LS180^{SOX9-GFP} stem cell and HT-115^{KRT20-mKate2} differentiation reporter cell lines. FIG. 23C shows reporter activity in HT-115KRT20-mKate2 differentiation reporter cell line treated with indicated concentrations of Compound 6 or DMSO as measured by a plate reader. FIG. 23D shows KRT20, SOX9, and GAPDH protein expression in parental HT-115 cell line treated with indicated concentrations of Compound 6 by immunoblot.

FIG. 24A-FIG. 24C show epigenetic drug screen using stem cell and differentiation reporter system. FIG. 24A shows stem cell reporter (LS180^{SOX9-GFP}) differentiation reporter (HT-115^{KRT20-mKate2}), and viability (LS180, HT-29, HT-115 parental lines) outputs following 4 days of culture with 31 epigenetic inhibitors at 10 uM. FIG. 24B shows fluorescent image of GFP signal in HT-29^{KRT20-eGFP} differentiation reporter cell line treated with DMSO control or compound 11. FIG. 24C shows immunoblot of KRT20, E-cadherin, and GAPDH in HT-115 CRC cells treated with indicated concentration of compound 11.

FIG. 25A-FIG. 25E show that impaired differentiation is a conserved event in mouse models of intestinal neoplasia. FIG. 25A shows a schematic depicting WNT and BMP signaling gradient in the intestines. Stem cells and Paneth cells reside in the crypt whereas differentiated cell types are found in the villus. FIG. 25B shows relative mRNA expression of Wnt/stem cell (top) and differentiation genes (bottom) in intestinal tissue derived from indicated mice by qRT-PCR; mean±S.D. of three biological replicates. FIG. 25C shows representative images of intestinal lesions from Lgr5-Apc^{f/f}-tdT mice including hematoxylin and eosin (H&E) staining and tdTomato (tdT), Sox9, Krt20, and Muc2 immunohistochemistry (IHC); scale bar=250 μM. FIG. 25D shows representative images of intestinal lesions from AOM/DSS treated mice including H&E staining Sox9, Krt20, and Muc2 IHC. Normal and adenoma regions labeled; scale bar=250 μM. FIG. 25E shows representative images of intestinal lesions from MNU treated Lgr5^{eGFP} mice including H&E staining and eGFP (Lgr5 expressing cells), Sox9, Krt20, and Muc2 IHC. Normal, dysplastic and carcinoma regions labeled. Scale bar=250 μM.

FIG. 26A-FIG. 26E show that scRNA-seq of intestinal mouse adenomas reveals features of aberrant stem cell activity. FIG. 26A shows tdT+ intestinal epithelial cells isolated by fluorescence-activated cell sorting (FACS) from Lgr5-Apc^{f/+}-tdT (control) and Lgr5-Apc^{f/f}-tdT (experimental) mice 28 days following tamoxifen induction. FIG. 26B shows UMAP representation of single cell transcriptome profiling of tdT+ epithelial cells from Lgr5-Apc^{f/+}-tdT (control) and Lgr5-Apc^{f/f}-tdT (experimental) mice colored by cell type (top left panel) and sample identity (top right panel). UMAP of separated samples along with pie chart indicating distribution of cell types (bottom panel). SC=stem cell, TA=transit amplifying, EP=enterocyte progenitor, E=mature enterocyte, G=goblet cell, P=Paneth cell, EE=enteroendocrine, T=tuft cell, AbSC=aberrant stem cell-like. FIG. 26C shows distribution of eGFP+ cells (Lgr5 expression) from tdT+ cells isolated by FACS from Lgr5-Apc^{f/+}-tdT (control) and Lgr5-Apc^{f/f}-tdT (experimental) mice. FIG. 26D shows UMAP representation of intestinal stem cell signature (left) Sox9 (right). FIG. 26E shows representative images of intestinal lesions from Lgr5-Apc^{f/f}-tdT mice including H&E staining and tdT, eGFP, Sox9, Olfm4, Krt20, and Muc2 IHC; scale bars=250 m. FIG. 26F shows transcription factor perturbation gene ontology analysis (EnrichR) of top 100 genes upregulated in AbSCcluster.

FIG. 27A-FIG. 27J show reactivation of genes associated with fetal intestinal development upon Apc inactivation. FIG. 27A shows a volcano plot showing differentially expressed genes from AbSC cluster. Enterocyte (blue), stem cell (green) and fetal intestines (purple) genes are highlighted. FIG. 27B shows average expression of differentially upregulated (red) and downregulated (blue) genes across fetal intestines at indicated timepoints of mouse development by RNA-seq. Heatmap representing differentially expressed genes in E12 fetal intestines compared to adult intestines ranked by fold change; a normalized enrichment score of upregulated genes in AbSC is shown on the right (p<1×10-5). E=embryonic, number indicates day, Ad=adult. FIG. 27C shows a normalized fetal-like intestinal gene signature expression on UMAP plot. FIG. 27D shows normalized Ly6a/Sca-1 expression on UMAP plot. FIG. 27E shows chromatin accessibility at Ly6a genomic locus in tdT+ cells isolated by FACS from Lgr5-tdT and Lgr5-Apc^{f/f}-tdT mice by ATAC sequencing. FIG. 27F shows mRNA expression of Ly6a in tdT+ cells isolated by FACS from Lgr5-tdT and Lgr5-Apc^{f/f}-tdT mice by qRT-PCR. FIG. 27G shows representative images of H&E staining and Ly6a/Sca-1, Sox9, and tdT IHC from Lgr5-Apc^{f/f}-tdT mice; scale bars=100 μM and 20 μM. Ly6a Sox9 quantification expressed as a percentage of tdT+ lesion at day 14 and 21 following tamoxifen induction. FIG. 27H shows representative image of Ly6a/Sca-1 IHC of high-grade dysplastic lesion from AOM/DSS treated mice shown in FIG. 25D. FIG. 27I shows the percentage of tdT+ cells that are Sca-1+ by FACS in Lgr5-tdT and Lgr5-Apc^{f/f}-tdT mice in indicated conditions. FIG. 27J shows representative image (top) and quantification (bottom) of organoids formed on Day 6 after plating 1000 FACS-isolated tdT+/Sca-1+ and tdT+/Sca-1−cells from Lgr5-Apc^{f/f}-tdT mice; mean±S.D of three biological replicates; p-values calculated by two-sided Student's t-test.

FIG. 28A-FIG. 28K show characterization of human FAP adenomas by histopathology and scRNA-seq. FIG. 28A shows representative images of adenoma and normal adjacent tissue from patient with FAP patient including H&E staining Sox9, Krt20, and Muc2 IHC; scale bars=250 μM. FIG. 28B shows UMAP representation of scRNA-seq of human adenoma and paired normal indicating 4 distinct cell clusters; E=enterocyte, G=goblet, Int=intermediate, Ab=aberrant. FIG. 28C shows a violin plot indicated SOX9 expression in four different cell clusters. FIG. 28D shows a violin plot indicated expression of ISC gene signature in four different cell clusters. P-value calculated by Wilcoxon rank sum test with Bonferroni correction. FIG. 28E shows a violin plot indicated expression of AbSC gene signature in four different cell clusters. P-value calculated by Wilcoxon rank sum test with Bonferroni correction. FIG. 28F shows a violin plot indicated expression of IFN/fetal-like gene signature in four different cell clusters. P-value calculated by Wilcoxon rank sum test with Bonferroni correction. FIG. 28G shows representative images of organoids derived from adenoma and adjacent-normal tissue from patient with FAP including H&E and Alcian blue (AB) staining Sox9, Krt20, and Muc2 IHC; dotted line in paired normal sample indicates one of several crypts in the organoid. FIG. 28H shows mRNA expression of SOX9; stem cell marker LGR5; WNT pathway markers AXIN2 and ASCL2; AbSC markers LY6e, TROP2 and intestinal differentiation markers KRT20, DPP4 in FAP organoids by qRT-PCR. Data expressed as mean±S.D of three biological and two technical replicates. FIG. 28I shows a phase contrast and fluorescent images of intestinal organoids derived from Apc^{f/f-tdT} (control) and Lgr5− Apc^{f/f-tdT} mice (top). Immunoblot of Sox9, tdT, and Vinculin (loading control) protein levels in indicated organoids (bottom).

FIG. 28J shows a phase contrast and fluorescent images of intestinal organoids derived from Apc^{f/f-tdT} (control) and Lgr5− Apc^{f/f-tdT} mice grown in WRN and DMEM media. FIG. 28K shows expression of stem cell (red), differentiation (blue) and developmental (purple) genes in organoids derived from Apc^{f/f-tdT} (control) and Lgr5-Apc^{f/f-tdT} mice by bulk RNA-sequencing.

FIG. 29A-FIG. 29G show that Sox9 is required for Apc^{KO} adenomas and organoids. FIG. 29A shows representative images of H&E staining and tdT, Sox9, eGFP (Lgr5) and Krt20 IHC from Lgr5-Apc^{f/f}-tdT (control) and Lgr5-Apc^{f/f}-Sox9^{f/f}-tdT (experimental) mice; corresponding normalized mRNA expression of Sox9, Ascl2, Lgr5, and Krt20. Data expressed as mean±S.D of three biological replicates. FIG. 29B shows schematic of in vivo experimental design. Kaplan-Meier survival curve of Lgr5-Apc^{f/f}-tdT (n=10) and Lgr5− Apc^{f/f}-Sox9^{f/f}-tdT (n=9) mice using high-dose (HD) tamoxifen (TAM) for induction and maintenance. Log− rank P-value=0.0069. FIG. 29C shows representative images of tdT, Sox9, and Krt20 IHC from Lgr5-Apc^{f/f}-Sox9^{f/f}-tdT mice. Genomic DNA was extracted from tdT+ cells isolated by FACS and subjected to Sox9 recombination-specific PCR. PCR product indicating no recombination ˜1200 bp and recombination ˜300 bp. FIG. 29D shows organoids from Lgr5-Apc^{f/f}-Sox9^{f/f}-tdT mice were generated at experimental endpoint, treated with either AdGFP or Ad^{GFP-Cre} and subjected to Sox9 recombination-specific PCR and a proliferation assay by CTG. Data expressed as mean S.D of three biological replicates. P-values calculated by two-sided Student's t-test. FIG. 29E shows proliferation of organoids derived from tdT, Apc^{f/f}-tdT, and Apc^{f/f}-Sox9^{f/f}-tdT mice and infected with AdGFP-Cre at indicated time points by CTG. Data expressed as mean±S.D of three biological replicates. FIG. 29F shows organoids derived from tdT, Apc^{f/f}-tdT, and Apc^{f/f}-Sox9^{f/f}-tdT mice were infected with Ad^{GFP-Cre} and percent tdT+ was quantified by FACS using the experimental approach in the schematic. Data expressed as mean±. S.D of two independent experiments. P-values calculated by two-sided Student's t-test. FIG. 29G shows organoids derived from tdT, Apc^{f/f}-tdT, and Apc^{f/f}-Sox9^{f/f}-tdT mice were infected with AdGFP or AdGFP-Cre, formalin fixed, and then processed for histopathology. Representative images of H&E staining and tdT, Sox9, and Ki67 IHC. Quantification of % Ki67 positivity in 5-6 organoids per condition. P-values calculated by two-sided Student's t-test.

FIG. 30A-FIG. 30D show that Sox9 suppression restricts AbSC and developmental reprogramming by scRNA-seq. FIG. 30A shows UMAP representation of scRNA-seq data from tdT+ cells isolated by FACS from Lgr5-tdT, Lgr5-Apc^{f/f}-tdT, and Lgr5-Apc^{f/f}-Sox9^{f/f}-tdT mice a month after tamoxifen induction colored by clusters and then separated by sample. Violin plot of normalized single cell Sox9 expression in each group. Pie charts indicating distribution of clusters in each sample. AbSC=aberrant stem cell-like, SC=stem cell, TA=transient amplifying, EP=enterocyte progenitor, E=enterocyte, EEC=enteroendocrine cell. FIG. 30B shows a violin plot of normalized expression of AbSC gene signature (top) and ISC gene signature (bottom) in each group. P-value calculated by Wilcoxon rank sum test with Bonferroni correction. FIG. 30C shows a violin plot of normalized expression of fetal-like intestines gene signature (left) and Ly6a Sca-1 (right) in each group. P-value calculated by Wilcoxon rank sum test with Bonferroni correction. FIG. 30D shows mRNA expression of Sox9, Krt20, and Ly6a Sca-1 in organoids derived from Lgr5-tdT, Lgr5-Apc^{f/f}-tdT, and Lgr5-Apc^{f/f}-Sox9^{f/f}-tdT mice expressed as a ratio of Ad^{GFP-Cre} to Ad^{GFP} treated (control). Data expressed as mean±S.D of three biological replicates. P-values calculated by two-sided Student's t-test.

FIG. 30E-FIG. 30I show that SOX9 KD impairs fetal reprogramming and induces differentiation in FAP adenoma organoids. FIG. 30E shows graphical representation (top) and phase contrast images (bottom) of differentiation phenotype in organoids. FIG. 30F shows phase contrast images depicting differentiation phenotype (folded) of organoids derived from normal adjacent mucosa and adenoma from a patent with FAP (top row); adenoma organoids expressing non-targeting control (NTC), or two distinct shRNAs against SOX9 (bottom row). Quantification of differentiating organoids in indicated cultures at day 2 and 3; Par=parental. FIG. 30G shows representative images of H&E, Alcian blue (AB) staining and SOX9, KRT20, and MUC2 IHC of NTC and SOX KD FAP adenoma. FIG. 30H shows normalized mRNA expression of SOX9, LGR5, AXIN2, KRT20, LY6E, TROP2 in indicated organoids: normal adjacent mucosa (N), adenoma-NTC (Ad) and adenoma-SOX9 KD (Ad-KD). FIG. 30I shows schematic summarizing AbSC transcriptional program and developmental reprogramming obstructing intestinal differentiation in CRC initiation and the ability of SOX9 suppression to reverse these effects.

FIG. 31A-FIG. 31E show elevated expression of Sox9 and reduced expression of differentiation genes in murine models of intestinal neoplasia. FIG. 31A shows a diagram showing a breeding scheme to achieve Lgr5-Apc^{f/f}-tdT mice for conditional Apc inactivation. FIG. 31B shows representative images showing gross anatomy of duodenal intestines from indicated mice. FIG. 31C shows a heat map of intestinal stem cell (red) and differentiation (blue) genes from previously published mouse models. RNA-seq data from the following three mice with and without dox are presented: shRNA against Renilla, Apc (shApc) alone or in combination with mutant K-ras allele. FIG. 31D shows high magnification representative images of Sox9 overexpression in dysplastic crypts of a AOM/DSS model. FIG. 31E shows high magnification representative images of Sox9 overexpression in carcinoma from a MNU model; scale bar=250 μM.

FIG. 32A-FIG. 32E show single cell transcriptional profiling of tdT+ cells from Lgr5-Apc^{f/f}-tdT mice Lgr5-Apc^{f/f}-tdT mice. FIG. 32A shows UMAP representation of experiment in FIG. 26A colored by ISC signature, which shows individual stem cell genes (Lgr5, Prom1, Olfm4, Ascl2), Wnt pathway gene Axin2, enterocyte signature (Haber, A. L., Biton, M., Rogel, N., Herbst, R. H., Shekhar, K., Smillie, C., Burgin, G., Delorey, T. M., Howitt, M. R., Katz, Y., et al. (2017). A single-cell survey of the small intestinal epithelium. Nature 551, 333-339), individual enterocyte genes (Krt20, Dpp4, Vil1), indicated Paneth cell gene signature, indicated Secretory cell gene signature, indicated enteroendocrine gene signature, indicated tuft cell gene signature, and indicated cell cycle gene signature. FIG. 32B shows representative images of intestinal lesions from Lgr5-Apc^{f/f}-tdT mice including H&E staining and tdT and Lysozyme (Lyz) IHC. Adjacent normal (Apc^{WT}) and adenoma (Apc^{KO}) regions as marked by tdT and labeled; scale bars=250 μM. FIG. 32C shows UMAP representation colored by Olfm4 expression (left) and a violin plot of Olfm4 expression in indicated samples (right). P-value calculated by Wilcoxon rank sum test with Bonferroni correction. FIG. 32D shows representative images of intestinal lesions from Lgr5-Apc^{f/f}-tdT showing tdT and Olfm4 IHC. Quantification of lesions that are positive (n=33) and negative (n=22) for Olfm4 expressed as a percentage of total tdT lesions; scale bars=250 μM. FIG. 32E shows transcription factor perturbation gene ontology analysis (EnrichR) of the top 100 genes upregulated in AbSC cluster.

FIG. 33A-FIG. 33O show that IFN signaling and fetal intestinal genes are upregulated in a AbSC program. FIG. 33A shows a volcano plot representing differentially expressed genes from the AbSC cluster. Genes associated with interferon signaling (red) are highlighted. FIG. 33B shows normalized expression of IFN-γ response gene signature in a UMAP plot. FIG. 33C shows MSigDB Hallmark significantly enriched gene-sets in the AbSC cluster (EnichR). FIG. 33D shows normalized expression of IFN-γ/fetal-like gene signature (Nusse, Y. M., Savage, A. K., Marangoni, P., Rosendahl-Huber, A. K. M., Landman, T. A., de Sauvage, F. J., Locksley, R. M., and Klein, O.D. (2018). Parasitic helminths induce fetal-like reversion in the intestinal stem cell niche. Nature 559, 109-113) on a UMAP plot. FIG. 33E shows normalized expression of fetal-like intestinal gene signature (Fernandez Vallone, V., Leprovots, M., Strollo, S., Vasile, G., Lefort, A., Libert, F., Vassart, G., and Garcia, M. I. (2016). Trop2 marks transient gastric fetal epithelium and adult regenerating cells after epithelial damage. Development 143, 1452-1463) on a UMAP plot. FIG. 33F shows a Venn diagram showing overlap genes among the three fetal-like intestinal gene-sets. FIG. 33G shows normalized expression of Ly6e on a UMAP plot. FIG. 33H shows normalized expression of Tacstd2/Trop2 on a UMAP plot. FIG. 33I shows chromatin accessibility at Ly6e genomic locus in tdT+ cells isolated by FACS from Lgr5-tdT and Lgr5-Apc^{f/f}-tdT mice by ATAC sequencing. FIG. 33J shows chromatin accessibility at Tacstd2/Trop2 genomic locus in tdT+ cells isolated by FACS from Lgr5-tdT and Lgr5-Apc^{f/f}-tdT mice by ATAC sequencing. FIG. 33K shows representative images of H&E staining, Ly6a/Sca-1, Lgr5 (eGFP), and tdT IHC from Lgr5-Apc^{f/f}-tdT mice; scale bars=100 μM. FIG. 33L shows representative images of H&E staining, Ly6a/Sca-1 and Olfm4 IHC from Lgr5-Apc^{f/f}-tdT mice; scale bars=100 μM. FIG. 33M shows percent Sca-1 positive cells in bone marrow preparations from Lgr5-tdT and Lgr5-Apc^{f/f}-tdT mice by FACS. FIG. 33N shows representative images of a single Sca-1+ and Sca-1− Lgr5-Apc^{f} f-tdT organoid growing over time at indicated time points. FIG. 33O shows schematic (left), quantification (middle), and representative images (right) of primary and secondary cultures of Sca-1+ and Sca-1− Lgr5-Apc^{f/f}-tdT organoids. Data expressed as mean±S.D (n=5); P-value calculated by two-sided Student's t-test.

FIG. 34A-FIG. 34H show genes and pathways in human FAP adenoma and adjacent normal tissue by scRNA-seq. FIG. 34A shows a UMAP representation of scRNA-seq of adenoma (blue) and paired normal (red) tissue from a patient with FAP. FIG. 34B shows a violin plot indicating normalized expression of enterocyte signature (Haber, A. L., Biton, M., Rogel, N., Herbst, R. H., Shekhar, K., Smillie, C., Burgin, G., Delorey, T. M., Howitt, M. R., Katz, Y., et al. (2017). A single-cell survey of the small intestinal epithelium. Nature 551, 333-33) in human adenoma and paired normal (left) and in different cell clusters (right); Ent=Enterocyte, Int=Intermediate, and Ab=aberrant stem cell-like. FIG. 34C shows a violin plot indicating normalized expression of differentiation markers MUC2 (left) and KRT20 (right) in different clusters. FIG. 34D shows normalized expression of SOX9 and KRT20 on a UMAP plot separated by sample. FIG. 34E shows a violin plot indicating normalized expression of ISC gene signatures from (Haber, A. L., Biton, M., Rogel, N., Herbst, R. H., Shekhar, K., Smillie, C., Burgin, G., Delorey, T. M., Howitt, M. R., Katz, Y., et al. (2017). A single-cell survey of the small intestinal epithelium. Nature 551, 333-339) and (Munoz, J., Stange, D. E., Schepers, A. G., van de Wetering, M., Koo, B. K., Itzkovitz, S., Volckmann, R., Kung, K. S., Koster, J., Radulescu, S., et al. (2012). The Lgr5 intestinal stem cell signature: robust expression of proposed quiescent ‘+4’ cell markers. EMBO J 31, 3079-3091) shown by sample and cluster type. FIG. 34F shows violin plot indicating normalized expression of AbSC gene signature by sample type. FIG. 34G shows violin plot indicating normalized expression of fetal-like gene signature (Mustata, R. C., Vasile, G., Fernandez-Vallone, V., Strollo, S., Lefort, A., Libert, F., Monteyne, D., Perez-Morga, D., Vassart, G., and Garcia, M. I. (2013). Identification of Lgr5-independent spheroid-generating progenitors of the mouse fetal intestinal epithelium. Cell reports 5, 421-432) by sample type. FIG. 34H shows normalized expression of TROP2/TACSTD2 (left) and LY6E on a UMAP plot.

FIG. 35A-FIG. 35I show that Apc^{KO} adenomas are dependent on Sox9. FIG. 35A shows schematic representation of mouse breeding to achieve conditional Lgr5-Apc^{f} f-Sox9^{f/f}-tdT mice. FIG. 35B shows Kaplan-Meier survival curve of Lgr5-Apc^{f/f}-tdT (n=5) and Lgr5-Apc^{f/f}-Sox9^{f/f}-tdT (n=9) mice using regular dose tamoxifen (Tam) for induction. Log-rank P-value=0.18. FIG. 35C shows representative images of H&E and AB staining and tdT, Sox9, eGFP (Lrg5), Axin2, Krt20, and Muc2 IHC from an Lgr5-Apc^{f/f}-Sox9^{f/f}-tdT mouse 17 days following tamoxifen induction. FIG. 35D shows organoids from Lgr5-Apc^{f/f}-Sox9^{f/f}-tdT mice were generated at experimental endpoint, treated with either AdGFP or Ad^{GFP-Cre} and subjected to a proliferation assay by CTG. Data expressed as mean±S.D of three biological replicates. FIG. 35E shows schematic (left) and analysis (right) of an in vivo experiment of Lgr5-Apc^{f/f}-tdT and Lgr5-Apc^{f/f}-Sox9^{f/f}-tdT mice in which organoid were generated 10 days following tamoxifen injection and then treated with Ad^{GFP-Cre} and subjected to a proliferation assay by CTG. Data expressed as mean±S.D of five biological replicates from three mice per genotype. FIG. 35F shows quantification of eGFP+ and tdT+ cells isolated by FACS from colonic tdT organoids infected with AdGFP or AdGFP-Cre.

FIG. 35G shows organoids derived from tdT, Apc^{f/f}-tdT, and Apc^{f/f}-Sox9^{f/f}-tdT mice were infected with LentiCre and percent tdT+ cells were quantified by FACS using the experimental approach in the schematic. FIG. 35H shows Apc^{f/f}-Sox9^{f/f}-tdT organoids were infected with AdGFP or Ad^{GFP-Cre} and subjected to semi-quantitative Sox9 recombination PCR at consecutive passages. FIG. 35I shows high magnification images of Ki67 IHC of tdT, Apc^{f/f}-tdT, and Apc^{f/f}-Sox9^{f/f}-tdT organoids infected with AdGFP or AdGFP-Cre, formalin fixed, and then processed for histopathology.

FIG. 36A-FIG. 36G show that Sox9 attenuation leads to reduction in pathways associated with AbSC by scRNA-seq. FIG. 36A shows a UMAP projection of scRNA data of tdT+ cells from Lgr5-tdT (green), Lgr5-Apc^{f/f}-tdT (blue), and Lgr5-Apc^{f/f}-Sox9^{f/f}-tdT (pink). FIG. 36B shows normalized expression of Sox9 on a UMAP plot separated by sample. FIG. 36C shows normalized expression of ISC, Enterocyte, Tuft, Goblet, Paneth, and Enteroendocrine (EEC) gene signatures. FIG. 36D shows a violin plot showing single cell expression of ISC gene signature (Haber, A. L., Biton, M., Rogel, N., Herbst, R. H., Shekhar, K., Smillie, C., Burgin, G., Delorey, T. M., Howitt, M. R., Katz, Y., et al. (2017). A single-cell survey of the small intestinal epithelium. Nature 551, 333-339) in samples. P-values calculated by Wilcoxon rank sum test with Bonferroni correction. FIG. 36E shows a violin plot showing single cell expression of Fetal-like gene signatures (Nusse, Y. M., Savage, A. K., Marangoni, P., Rosendahl-Huber, A. K. M., Landman, T. A., de Sauvage, F. J., Locksley, R. M., and Klein, O.D. (2018). Parasitic helminths induce fetal-like reversion in the intestinal stem cell niche. Nature 559, 109-113) and (Fernandez Vallone, V., Leprovots, M., Strollo, S., Vasile, G., Lefort, A., Libert, F., Vassart, G., and Garcia, M. I. (2016). Trop2 marks transient gastric fetal epithelium and adult regenerating cells after epithelial damage. Development 143, 1452-1463). P-values calculated by Wilcoxon rank sum test with Bonferroni correction. FIG. 36F shows violin plot showing single cell expression of Ly6e and Trop2/Tacstd2. P-values calculated by Wilcoxon rank sum test with Bonferroni correction. FIG. 36G shows a violin plot showing single cell expression of IFN-7 gene signature (Hallmark). P-value calculated by Wilcoxon rank sum test with Bonferroni correction.

FIG. 37A-FIG. 37B show SOX9 KD in FAP normal and adenoma organoids. FIG. 37A shows relative mRNA expression of SOX9 and KRT20 in FAP adenomas expressing non-targeting (NT)control, SOX9 shRNA #1, or SOX9 shRNA #5 by qRT-PCR; mean S.D. of three biological replicates. FIG. 37B shows quantification of percent undifferentiated and differentiated organoids in indicated cultures at day 3 (mean±S.D. of three biological replicates).

FIG. 38 shows that MRK demonstrates in vivo anti-tumor activity in Apc^{KO}-KrasG12D colon organoid xenograft model. The far left shows a schematic of in vivo experiment. The center shows percent tumor size change following MRK treatment. The far right shows a water-fall plot showing individual tumor response after 10 days of MRK treatment.

FIG. 39 shows a dual endogenous reporter system coupled with pooled CRISPR-Cas9 screen. This screen identifies genes associated with promoting differentiation and blocking the stem cell program in colorectal cancer cell lines. Rank sum of each gene in 3 technical replicates of the epigenetic CRISPR-Cas9 screen comparing the GFP positive to the mCherry positive sorted cell fractions of the HT-29^{SOX9-mKate2/KRT20-GFP} cell line. The y-axis shows the rank sum of each gRNA targeting each of the 88 genes in the epigenetic library shown on the x-axis. The rank sum is derived by summing the ordered rank of each sgRNA by fold change across the 3 technical replicates of the epigenetic CRISPR-Cas9 screen such that higher rank sum indicates depletion and lower rank sum indicates enrichment. Boxplots showing the distribution of sgRNA rank sums per gene are colored in green or red if there are at least 2 sgRNA targeting the same gene whose rank sums are above the enrichment threshold (green) or depletion threshold (red) respectively. Probability density plots showing the distribution of enriched (green), depleted (red), and all other gRNA (grey) rank sums (right panel).

For any figure showing a bar histogram, curve, or other data associated with a legend, the bars, curve, or other data presented from left to right for each indication correspond directly and in order to the boxes from top to bottom of the legend.

### DETAILED DESCRIPTION OF THE INVENTION

The present invention is based, at least in part, on the discovery that down-regulators of SOX9 signaling can treat colorectal cancer (CRC) and intestinal cancer. In particular, it is described herein that SRY-Box transcription factor 9 (SOX9) promotes CRC by endorsing a stem cell-like program that hinders intestinal differentiation. Human cell line dependency data indicated a potential requirement for SOX9 in CRC. Functional studies demonstrated that SOX9 suppression impairs primary tumor growth by inducing intestinal differentiation in human CRC cell lines and engineered neoplastic organoids. By binding to genome-wide enhancers, SOX9 directly activates genes associated with Paneth and stem cell activity, including PROM1. Additionally, a pentaspan transmembrane protein, PROM1 utilizes its first intracellular domain to block intestinal differentiation, at least in part through SOX9, reinforcing a PROM1-SOX9 positive feedback loop.

Accordingly, the present invention provides methods of treating one or more intestinal cancers (e.g., CRC) in a subject by administering to the subject an effective amount of an agent that modifies the copy number, the expression level, and/or the activity of one or more biomarkers listed in Table 1 or 2 or a fragment thereof. The present invention also provides methods of promoting differentiation of an intestinal cancer cell into a mature intestinal cell in a subject by administering to the subject an effective amount of an agent that modifies the copy number, the expression level, and/or the activity of one or more biomarkers listed in Table 1 or 2 or a fragment thereof. The present invention provides exemplary RNA interfering agents and small molecules that inhibit such regulators and can be used in a therapy and other methods described herein, such as agents that inhibit or promote the function and/or the ability of one or more biomarkers listed in Table 1 or 2. Similarly, methods of screening for modulators or agents of such regulators and methods of diagnosing, prognosing, and monitoring cancer involving such inhibitors and/or therapies are provided.

### I. Definitions

The articles “a” and “an” are used herein to refer to one or to more than one (i.e., to at least one) of the grammatical object of the article. By way of example, “an element” means one element or more than one element.

The term “activity” when used in connection with proteins or protein complexes means any physiological or biochemical activities displayed by or associated with a particular protein or protein complex including but not limited to activities exhibited in biological processes and cellular functions, ability to interact with or bind another molecule or a moiety thereof, binding affinity or specificity to certain molecules, in vitro or in vivo stability (e.g., protein degradation rate, or in the case of protein complexes ability to maintain the form of protein complex), antigenicity and immunogenicity, enzymatic activities, etc. Such activities may be detected or assayed by any of a variety of suitable methods as will be apparent to skilled artisans.

The term “altered amount” or “altered level” refers to increased or decreased copy number (e.g., germline and/or somatic) of a biomarker nucleic acid, e.g., increased or decreased expression level in a sample, as compared to the expression level or copy number of the biomarker nucleic acid in a control sample. The term “altered amount” of a biomarker also includes an increased or decreased protein level of a biomarker protein in a sample, as compared to the corresponding protein level in a normal, control sample. Furthermore, an altered amount of a biomarker protein may be determined by detecting posttranslational modification such as methylation status of the marker, which may affect the expression or activity of the biomarker protein.

The amount of a biomarker in a subject is “significantly” higher or lower than the normal amount of the biomarker, if the amount of the biomarker is greater or less, respectively, than the normal level by an amount greater than the standard error of the assay employed to assess amount, and preferably at least 10%, 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the amount of the biomarker in the subject can be considered “significantly” higher or lower than the normal amount if the amount is at least about two, and preferably at least about three, four, or five times, higher or lower, respectively, than the normal amount of the biomarker. Such “significance” can also be applied to any other measured parameter described herein, such as for expression, inhibition, cytotoxicity, cell growth, and the like.

The term “altered level of expression” of a biomarker refers to an expression level or copy number of the biomarker in a test sample, e.g., a sample derived from a patient suffering from cancer, that is greater or less than the standard error of the assay employed to assess expression or copy number, and is preferably at least twice, and more preferably three, four, five or ten or more times the expression level or copy number of the biomarker in a control sample (e.g., sample from a healthy subjects not having the associated disease) and preferably, the average expression level or copy number of the biomarker in several control samples. The altered level of expression is greater or less than the standard error of the assay employed to assess expression or copy number, and is preferably at least 10%, 20%, 30%, 40%, 50%, 60%, 70%, 80%, 90%, 100%, 150%, 200%, 300%, 350%, 400%, 500%, 600%, 700%, 800%, 900%, 1000% or more times the expression level or copy number of the biomarker in a control sample (e.g., sample from a healthy subjects not having the associated disease) and preferably, the average expression level or copy number of the biomarker in several control samples. In some embodiments, the level of the biomarker refers to the level of the biomarker itself, the level of a modified biomarker (e.g., phosphorylated biomarker), or to the level of a biomarker relative to another measured variable, such as a control (e.g., phosphorylated biomarker relative to an unphosphorylated biomarker).

The term “altered activity” of a biomarker refers to an activity of the biomarker which is increased or decreased in a disease state, e.g., in a sample from a subject having CRC and/or an intestinal cancer, as compared to the activity of the biomarker in a normal, control sample. Altered activity of the biomarker may be the result of, for example, altered expression of the biomarker, altered protein level of the biomarker, altered structure of the biomarker, or, e.g., an altered interaction with other proteins involved in the same or different pathway as the biomarker or altered interaction with transcriptional activators or inhibitors.

The term “altered structure” of a biomarker refers to the presence of mutations or allelic variants within a biomarker nucleic acid or protein, e.g., mutations which affect expression or activity of the biomarker nucleic acid or protein, as compared to the normal or wild-type gene or protein. For example, mutations include, but are not limited to substitutions, deletions, or addition mutations. Mutations may be present in the coding or non-coding region of the biomarker nucleic acid.

The term “administering” is intended to include modes and routes of administration which allow an agent to perform its intended function. Examples of routes of administration for treatment of a body which can be used include injection (subcutaneous, intravenous, parenterally, intraperitoneally, intrathecal, etc.), oral, inhalation, and transdermal routes. The injection can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. The agent also may be administered as a prodrug, which is converted to its active form in vivo.

Unless otherwise specified here within, the terms “antibody” and “antibodies” refers to antigen-binding portions adaptable to be expressed within cells as “intracellular antibodies.” (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publs. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

Antibodies may be polyclonal or monoclonal; xenogeneic, allogeneic, or syngeneic; or modified forms thereof (e.g. humanized, chimeric, etc.). Antibodies may also be fully human. Preferably, antibodies of the present invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized”, which is intended to include antibodies made by a non-human cell having variable and constant regions which have been altered to more closely resemble antibodies that would be made by a human cell. For example, by altering the nonhuman antibody amino acid sequence to incorporate amino acids found in human germline immunoglobulin sequences. The humanized antibodies of the present invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

The term “biomarker” refers to a measurable entity of the present invention that has been determined to be predictive of effects of combinatorial therapies comprising one or more inhibitors of one or more biomarkers listed in Tables 1-2, for example, one or more biomarkers listed in Tables 1-2, such as SOX9, PROM1, and/or KRT20. Biomarkers can include, without limitation, nucleic acids and proteins, including those shown in the Tables, the Examples, the Figures, and otherwise described herein. As described herein, any relevant characteristic of a biomarker can be used, such as the copy number, amount, activity, location, modification (e.g., phosphorylation), and the like.

TABLE 1
Representative biomarkers useful according to
methods encompassed by the present invention

| | Marker Properties |
|---|---|
| List of Biomarkers | (stem cell-like programs) |
| SOX9 (SRY-Box transcription factor 9) | Stem cell |
| YAP1 (Yes1-associated protein) | Intestine survival and |
| | proliferation |
| TAZ (tafazzin) | Intestine survival and |
| | proliferation |
| Lgr5 (leucine rich repeat containing G | Stem cell |
| protein-coupled receptor 5) | |
| Lrig1 (leucine rich repeats and | Stem cell |
| immunoglobulin like domains 1) | |
| PROM1 (Prominin-1; aka CD133) | Stem cell |
| Axin2 (Axin-related protein) | Wnt signaling |
| Ascl2 (achaete-scute family bHLH | Wnt signaling |
| transcription factor 2) | |
| TCF4 (transcription factor 4) | Wnt signaling |
| OLFM4 (olfactomedin 4) | Stem cell |
| SMOC2 (SPARC related modular | Stem cell |
| calcium binding 2) | |
| CDK6 (cyclin dependent kinase 6) | Stem cell |
| LYZ (lysozyme) | Paneth cell |
| DEFA5 (defensin alpha 5) | Paneth cell |
| DEFA6 (defensin alpha 6) | Paneth cell |
| TROP2 | Stem cell |
| LY6 (gene family) | Stem cell |

TABLE 2
Representative biomarkers useful according to
methods encompassed by the present invention

| | Marker Properties |
|---|---|
| List of Biomarkers | (pro-differentiation pathways) |
| Krt20 (Keratin-20) | Absorptive intestinal differentiation |
| DPP4 (dipeptidyl peptidase 4) | Absorptive intestinal differentiation |
| APOA4 (apolipoprotein A4) | Absorptive intestinal differentiation |
| ATOH1 (atonal bHLH transcription | Absorptive intestinal differentiation |
| factor 1) | |
| TFF2 (trefoil factor 2) | Absorptive intestinal differentiation |
| FCGBP (Fc fragment of IgG binding | Absorptive intestinal differentiation |
| protein) | |
| REG4 (regenerating family | Absorptive intestinal differentiation |
| member 4) | |
| AGR2 (anterior gradient 2) | Absorptive intestinal differentiation |
| MUC2 (mucin 2) | Secretory intestinal differentiation |
| CDX1 (caudal type homeobox 1) | Secretory intestinal differentiation |
| CDX2 (caudal type homeobox 2) | Secretory intestinal differentiation |
| SLC2A5 (solute carrier family 2 | Secretory intestinal differentiation |
| member 5) | |
| B3GALT5 (beta-1,3- | Secretory intestinal differentiation |
| galactosyltransferase 5) | |
| FUT3 (fucosyltransferase 3) | Secretory intestinal differentiation |
| FABP1 (fatty acid binding | Secretory intestinal differentiation |
| protein 1) | |
| FABP2 (fatty acid binding | Secretory intestinal differentiation |
| protein 2) | |
| CDH1 (Cadherin 1; aka E-cadherin) | General epithelial differentiation |
| BMP2 (Bone morphogenetic | BMP/TGF-β signaling |
| protein 2) | |
| TGF-β1 (Transforming growth | BMP/TGF-β signaling |
| factor-β1) | |

A “blocking” antibody or an antibody “antagonist” is one which inhibits or reduces at least one biological activity of the antigen(s) it binds. In certain embodiments, the blocking antibodies or antagonist antibodies or fragments thereof described herein substantially or completely inhibit a given biological activity of the antigen(s).

The term “body fluid” refers to fluids that are excreted or secreted from the body as well as fluids that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features. Unless otherwise stated, the terms include metaplasia. In some embodiments, such cells exhibit such characteristics in part or in full due to the expression and activity of signaling pathways regulated by one or more biomarkers listed in Tables 1-2. In some embodiments, the cancer cells described herein are not sensitive to at least one of immunotherapies. In some embodiments, the cancer cells are treatable with an agent capable of antagonizing regulators of the biomarkers described herein, such as inhibiting expression and/or function, as described herein.

Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., Merkel cell carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

As used herein, an “intestinal cancer” is either one which arises from the small intestinal or large intestinal cells that acquire properties of intestinal cells through an oncogenic process. Intestinal cancer can include, but is not limited to, CRC, small intestine cancer, adenocarcinoma, and/or carcinoid tumor.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present invention. In one embodiment, the control may comprise normal or noncancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control may comprise normal cells, cells from patients treated with combination chemotherapy, and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing standard of care therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods of the present invention are not limited to use of a specific cut-point in comparing the level of expression product in the test sample to the control.

The “copy number” of a biomarker nucleic acid refers to the number of DNA sequences in a cell (e.g., germline and/or somatic) encoding a particular gene product. Generally, for a given gene, a mammal has two copies of each gene. The copy number can be increased, however, by gene amplification or duplication, or reduced by deletion. For example, germline copy number changes include changes at one or more genomic loci, wherein said one or more genomic loci are not accounted for by the number of copies in the normal complement of germline copies in a control (e.g., the normal copy number in germline DNA for the same species as that from which the specific germline DNA and corresponding copy number were determined). Somatic copy number changes include changes at one or more genomic loci, wherein said one or more genomic loci are not accounted for by the number of copies in germline DNA of a control (e.g., copy number in germline DNA for the same subject as that from which the somatic DNA and corresponding copy number were determined).

The “normal” copy number (e.g., germline and/or somatic) of a biomarker nucleic acid or “normal” level of expression of a biomarker nucleic acid or protein is the activity/level of expression or copy number in a biological sample, e.g., a sample containing tissue, whole blood, serum, plasma, buccal scrape, saliva, cerebrospinal fluid, urine, stool, and bone marrow, from a subject, e.g., a human, not afflicted with an intestinal cancer or CRC, or from a corresponding non-affected tissue in the same afflicted subject.

The term “determining a suitable treatment regimen for the subject” is taken to mean the determination of a treatment regimen (i.e., a single therapy or a combination of different therapies that are used for the prevention and/or treatment of the cancer in the subject) for a subject that is started, modified and/or ended based or essentially based or at least partially based on the results of the analysis according to the present invention. One example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

The term “diagnosing cancer” includes the use of the methods, systems, and code of the present invention to determine the presence or absence of a cancer or subtype thereof in an individual. The term also includes methods, systems, and code for assessing the level of disease activity in an individual.

The term “down-regulate” includes the decrease, limitation, or blockage, of, for example a particular action, function, or interaction. In some embodiments, a signaling is “down-regulated” if at least one effect of the signaling is alleviated, terminated, slowed, or prevented. Similarly, a “down-regulator” of a signaling is an agent (e.g., a therapeutic agent) that down-regulates the signaling disclosed herein. The terms “promote” and “up-regulate” have the opposite meaning as compared to “down-regulate.”

A molecule is “fixed” or “affixed” to a substrate if it is covalently or non-covalently associated with the substrate such that the substrate can be rinsed with a fluid (e.g., standard saline citrate, pH 7.4) without a substantial fraction of the molecule dissociating from the substrate.

The term “expression signature” or “signature” refers to a group of one or more coordinately expressed biomarkers related to a measured phenotype. For example, the genes, proteins, metabolites, and the like making up this signature may be expressed in a specific cell lineage, stage of differentiation, or during a particular biological response. The biomarkers can reflect biological aspects of the tumors in which they are expressed, such as the cell of origin of the cancer, the nature of the non-malignant cells in the biopsy, and the oncogenic mechanisms responsible for the cancer. Expression data and gene expression levels can be stored on computer readable media, e.g., the computer readable medium used in conjunction with a microarray or chip reading device. Such expression data can be manipulated to generate expression signatures.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “immunotherapy” or “immunotherapies” refer to any treatment that uses certain parts of a subject's immune system to fight diseases such as cancer. The subject's own immune system is stimulated (or suppressed), with or without administration of one or more agent for that purpose. Immunotherapies that are designed to elicit or amplify an immune response are referred to as “activation immunotherapies.” Immunotherapies that are designed to reduce or suppress an immune response are referred to as “suppression immunotherapies.” Any agent believed to have an immune system effect on the genetically modified transplanted cancer cells can be assayed to determine whether the agent is an immunotherapy and the effect that a given genetic modification has on the modulation of immune response. In some embodiments, the immunotherapy is cancer cell-specific. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

The term “mode of administration” includes any approach of contacting a desired target (e.g., cells, a subject) with a desired agent (e.g., a therapeutic agent). The route of administration, as used herein, is a particular form of the mode of administration, and it specifically covers the routes by which agents are administered to a subject or by which biophysical agents are contacted with a biological material.

An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

The term “polyposis” refers to a condition characterized by the presence of internal polyps, such as polyps in the gastrointestinal tract. Polyposis can be a hereditary disease (e.g., familial adenomatous polyposis) which affects the colon. The polyps may be at risk for becoming malignant. As used herein, an “at risk” individual includes, but is not limited to, an individual afflicted with polyposis.

The term “pre-determined” biomarker amount and/or activity measurement(s) may be a biomarker amount and/or activity measurement(s) used to, by way of example only, evaluate a subject that may be selected for a particular treatment, evaluate a response to a treatment such as inhibitor(s) of the regulators of one or more biomarkers listed in Tables 1-2, or in combination with an immunotherapy, and/or evaluate the disease state. A pre-determined biomarker amount and/or activity measurement(s) may be determined in populations of patients with or without cancer. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements. In another embodiment, the amounts determined and/or compared in a method described herein are based on relative measurements, such as ratios (e.g., serum biomarker normalized to the expression of housekeeping or otherwise generally constant biomarker).

The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

The term “predictive” includes the use of a biomarker nucleic acid and/or protein status, e.g., over- or under-activity, emergence, expression, growth, remission, recurrence or resistance of tumors before, during or after therapy, for determining the likelihood of response of a cancer to inhibitor(s) of one or more biomarkers listed in Tables 1-2, or in combination with an immunotherapy (e.g., treatment with a combination of such inhibitor and an immunotherapy, such as an immune checkpoint inhibitor). Such predictive use of the biomarker may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression of a biomarker nucleic acid (e.g., by ISH, Northern Blot, or qPCR), increased or decreased biomarker protein (e.g., by IHC), or increased or decreased activity, e.g., in more than about 5%, 6%, 7%, 8%, 9%, 10%, 110, 12%, 13%, 14%, 15%, 20%, 25%, 30%, 40%, 50%, 60%, 70%, 80%, 90%, 95%, 100%, or more of assayed human cancers types or cancer samples; (2) its absolute or relatively modulated presence or absence in a biological sample, e.g., a sample containing tissue, whole blood, serum, plasma, buccal scrape, saliva, cerebrospinal fluid, urine, stool, or bone marrow, from a subject, e.g. a human, afflicted with cancer; (3) its absolute or relatively modulated presence or absence in clinical subset of patients with cancer (e.g., those responding to a particular inhibitor/immunotherapy combination therapy or those developing resistance thereto).

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “probe” refers to any molecule which is capable of selectively binding to a specifically intended target molecule, for example, a nucleotide transcript or protein encoded by or corresponding to a biomarker nucleic acid. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules. The term “prognosis” includes a prediction of the probable course and outcome of cancer or the likelihood of recovery from the disease. In some embodiments, the use of statistical algorithms provides a prognosis of cancer in an individual. For example, the prognosis can be surgery, development of a clinical subtype of cancer (e.g., solid tumors, such as esophageal cancer and gastric cancer), development of one or more clinical factors, or recovery from the disease.

The terms “response” or “responsiveness” refers to an anti-cancer response, e.g. in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

An “RNA interfering agent” as used herein, is defined as any agent which interferes with or inhibits expression of a target biomarker gene by RNA interference (RNAi). Such RNA interfering agents include, but are not limited to, nucleic acid molecules including RNA molecules which are homologous to the target biomarker gene of the present invention, or a fragment thereof, short interfering RNA (siRNA), and small molecules which interfere with or inhibit expression of a target biomarker nucleic acid by RNA interference (RNAi).

“RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn and Cullen (2002) J. Virol. 76:9225), thereby inhibiting expression of the target biomarker nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. “Short interfering RNA” (siRNA), also referred to herein as “small interfering RNA” is defined as an agent which functions to inhibit expression of a target biomarker nucleic acid, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA). In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501 incorporated by reference herein).

RNA interfering agents, e.g., siRNA molecules, may be administered to a patient having or at risk for having an intestinal cancer and/or a CRC, to inhibit expression of a biomarker gene which is overexpressed in the intestinal cancer and/or the CRC, and thereby treat, prevent, or inhibit the intestinal cancer and/or the CRC.

siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target biomarker nucleic acids. As used herein, “inhibition of target biomarker nucleic acid expression” or “inhibition of marker gene expression” includes any decrease in expression or protein activity or level of the target biomarker nucleic acid or protein encoded by the target biomarker nucleic acid. The decrease may be of at least 30%, 40%, 50%, 60%, 70%, 80%, 90%, 95% or 99% or more as compared to the expression of a target biomarker nucleic acid or the activity or level of the protein encoded by a target biomarker nucleic acid which has not been targeted by an RNA interfering agent.

The term “sample” used for detecting or determining the presence or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method of the present invention further comprises obtaining the sample from the individual prior to detecting or determining the presence or level of at least one marker in the sample.

The term “sensitize” means to alter cancer cells or tumor cells in a way that allows for more effective treatment of the associated cancer with a cancer therapy (e.g., anti-immune checkpoint, chemotherapeutic, and/or radiation therapy). In some embodiments, normal cells are not affected to an extent that causes the normal cells to be unduly injured by the therapies. An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res. 1982; 42: 2159-2164), cell death assays (Weisenthal L M, Shoemaker R H, Marsden J A, Dill P L, Baker J A, Moran E M, Cancer Res. 1984; 94: 161-173; Weisenthal L M, Lippman M E, Cancer Treat Rep 1985; 69: 615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993: 415-432; Weisenthal L M, Contrib Gynecol Obstet (1994) 19: 82-90). The sensitivity or resistance may also be measured in animal by measuring the tumor size reduction over a period of time, for example, 6 month for human and 4-6 weeks for mouse. A composition or a method sensitizes response to a therapeutic treatment if the increase in treatment sensitivity or the reduction in resistance is 25% or more, for example, 30%, 40%, 50%, 60%, 70%, 80%, or more, to 2-fold, 3-fold, 4-fold, 5-fold, 10-fold, 15-fold, 20-fold or more, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy can be equally applied to methods for sensitizing hyperproliferative or otherwise cancerous cells (e.g., resistant cells) to the cancer therapy.

The term “subject” refers to any healthy animal, mammal or human, or any animal, mammal or human afflicted with an intestinal cancer or a CRC. The term “subject” is interchangeable with “patient.”

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human.

The terms “therapeutically-effective amount” and “effective amount” as used herein means that amount of a compound, material, or composition comprising a compound encompassed by the present invention which is effective for producing some desired therapeutic effect in at least a sub-population of cells in an animal at a reasonable benefit/risk ratio applicable to any medical treatment. Toxicity and therapeutic efficacy of subject compounds can be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to administration of a suitable control agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to administration of a suitable control agent.

The term, “SOX9,” also known as SRY-box transcription factor 9, refers to a transcription factor that plays a key role in chondrocytes differentiation and skeletal development. The protein encoded by this gene recognizes the sequence CCTTGAG along with other members of the HMG-box class DNA-binding proteins SOX9 guides cell fate decisions during developmental and adult homeostasis in diverse tissue including cartilage (Wagner et al. (1994) Cell 79: 1111-1120; Akiyama et al. (2005) 102: 14665-14670), testis (Moreno-Mendoza et al. (2004) Biol Reprod 70: 114-122; Jakob and Lovell-Badge (2011) FEBS J 278: 1002-1009), skin (Kadaja et al. (2014) Genes Dev 28: 328-341; Adam et al. (2015), Nature 521: 366-370), and breast (Guo et al. (2012) Cell 148: 1015-1028). In the intestines, biallelic genetic inactivation of SOX9 led to impaired Paneth cell differentiation in genetically engineered mouse models (Bastide et al. (2007) The Journal of cell biology 178: 635-648; Mori-Akiyama et al. (2007) Gastroenterology 133: 539-546). However, the role of SOX9 in CRC remains unclear as there is evidence for oncogenic and tumor suppressor functions (Lu et al. (2008) Am J Clin Pathol 130: 897-904; Cancer Genome Atlas (2012) Nature 487: 330-337; Matheu et al. (2012) Cancer Res 72: 1301-1315; Carrasco-Garcia et al. (2016) Sci Rep 6: 32350; Prevostel et al. (2016) Oncotarget 7: 82228-82243; Prevostel and Blache (2017) Eur J Cancer 86: 150-157; Hiramatsu et al. (2019) Proc Natl Acad Sci USA 116: 1704-1713; Vasaikar et al. (2019) Cell 177: 1035-1049 e1019).

The term “SOX9” is intended to include fragments, variants (e.g., allelic variants) and derivatives thereof. The nucleic acid and amino acid sequences of a representative human SOX9 is available to the public at the GenBank database (Gene ID 6662) (NM_000346.4 and NP_000337.1). Nucleic acid and polypeptide sequences of SOX9 orthologs in organisms other than humans are well known and include, for example, mouse SOX9 (NM_011448.4 and NP_035578.3), rat SOX9 (NM_080403.1 and NP_536328.1), zebrafish SOX9 (NM_131643.1 and NP_571718.1), chimpanzee SOX9 (NM_001009029.1 and NP_001009029.1), and monkey SOX9 (NM_001032868.1 and NP_001028040.1).

The term “SOX9 activity” includes the ability of a SOX9 polypeptide (and its fragments, domains, and/or motifs thereof, discussed herein) to bind its substrates, and/or mediate its transcriptional activity.

The term “agents that decrease the copy number, the expression level, and/or the activity of SOX9” encompasses any natural or non-natural agent prepared, synthesized, manufactured, and/or purified by human that is capable of decreasing the expression level and/or activity of a SOX9 polypeptide (and its fragments, domains, and/or motifs thereof, discussed herein). In some embodiments, the agent may decrease the binding/interaction between SOX9 and other transcription factors or binding partners. In other embodiments, the agent may decrease the expression of a SOX9 polypeptide. In still another embodiment, such inhibitors may increase or promote the turnover rate, reduce or inhibit the expression and/or the stability (e.g., the half-life), and/or change the cellular localization of SOX9, resulting in at least a decrease in SOX9 levels and/or activity. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to SOX9 or also inhibit at least one of the binding partners.

The term, “YAP1,” also known as Yes1-associated protein, refers to a transcriptional regulator that can act both as a coactivator and a corepressor. YAP1 is a critical downstream regulatory target in the Hippo signaling pathway that plays a pivotal role in organ size control and tumor suppression by restricting proliferation and promoting apoptosis.

The term “YAP1” is intended to include fragments, variants (e.g., allelic variants) and derivatives thereof. The nucleic acid and amino acid sequences of a representative human YAP1 is available to the public at the GenBank database (Gene ID 10413). Human YAP1 variants include, but not limited to, the transcript variant 1 encoding isoform 1 (NM_001130145.3 and NP_001123617.1), the transcript variant 2 encoding isoform 2 (NM_006106.5 and NP_006097.2), the transcript variant 3 encoding isoform 3 (NM_001195044.2 and NP_001181973.1), the transcript variant 4 encoding isoform 4 (NM_001195045.2 and NP_001181974.1), the transcript variant 5 encoding isoform 5 (NM_001282098.2 and NP_001269027.1), the transcript variant 6 encoding isoform 6 (NM_001282097.2 and NP_001269026.1), the transcript variant 7 encoding isoform 7 (NM_001282099.2 and NP_001269028.1), the transcript variant 8 encoding isoform 8 (NM_001282100.2 and NP_001269029.1), and the transcript variant 9 encoding isoform 9 (NM_001282101.2 and NP_001269030.1).

Nucleic acid and polypeptide sequences of YAP1 orthologs in organisms other than humans are well known and include, for example, mouse YAP1 (NM_001171147.1 and NP_001164618.1), rat YAP1 (NM_001034002.2 and NP_001029174.2), zebrafish YAP1 (NM_001139480.1 and NP_001132952.1), chimpanzee YAP1 (XM_016921859.1 and XP_016777348.1), and monkey YAP1 (XM_015115542.2 and XP_014971028.1).

The term “YAP1 activity” includes the ability of a YAP1 polypeptide (and its fragments, domains, and/or motifs thereof, discussed herein) to bind its substrates, and/or mediate its activity and functions disclosed herein.

The term “agents that decrease the copy number, the expression level, and/or the activity of YAP1” encompasses any natural or non-natural agent prepared, synthesized, manufactured, and/or purified by human that is capable of decreasing the expression level and/or activity of a YAP1 polypeptide (and its fragments, domains, and/or motifs thereof, discussed herein). In some embodiments, the agent may decrease the binding/interaction between YAP1 and other transcription factors or binding partners. In other embodiments, the agent may decrease the expression of a YAP1 polypeptide. In still another embodiment, such inhibitors may increase or promote the turnover rate, reduce or inhibit the expression and/or the stability (e.g., the half-life), and/or change the cellular localization of YAP1, resulting in at least a decrease in YAP1 levels and/or activity. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to YAP1 or also inhibit at least one of the binding partners.

The term, “Taz,” also known as tafazzin, refers to a gene that encodes a mitochondrial phospholipid-lysophospholipid transacylase necessary for normal composition and content of cardiolipin. Mutations in Taz have been associated with a number of clinical disorders including Barth syndrome, dilated cardiomyopathy (DCM), hypertrophic DCM, endocardial fibroelastosis, and left ventricular noncompaction (LVNC). Multiple transcript variants encoding different isoforms have been described. For example, a long form and a short form of each of these isoforms is produced. The short form lacks a hydrophobic leader sequence and may exist as a cytoplasmic protein rather than being membrane-bound.

The term “Taz” is intended to include fragments, variants (e.g., allelic variants) and derivatives thereof. The nucleic acid and amino acid sequences of a representative human Taz is available to the public at the GenBank database (Gene ID 6901). Human Taz variants include, but not limited to, the transcript variant 1 encoding isoform 1 (NM_000116.5 and NP_000107.1), the transcript variant 2 encoding isoform 2 (NM_181311.4 and NP_851828.1), the transcript variant 3 encoding isoform 3 (NM_181312.4 and NP_851829.1), the transcript variant 4 encoding isoform 4 (NM_181313.4 and NP_851830.1), and the transcript variant 5 encoding isoform 5 (NM_001303465.2 and NP_001290394.1).

Nucleic acid and polypeptide sequences of Taz orthologs in organisms other than humans are well known and include, for example, mouse Taz (NM_001173547.2 and NP_001167018.1), rat Taz (NM_001025748.1 and NP_001020919.1), zebrafish Taz (NM_001001814.1 and NP_001001814.1), chimpanzee Taz (NM_001009011.1 and NP_001009011.1), and monkey Taz (NM_001032914.1 and NP_001028086.1).

The term “Taz activity” includes the ability of a Taz polypeptide (and its fragments, domains, and/or motifs thereof, discussed herein) to bind its substrates, and/or mediate its activity and functions disclosed herein.

The term “agents that decrease the copy number, the expression level, and/or the activity of Taz” encompasses any natural or non-natural agent prepared, synthesized, manufactured, and/or purified by human that is capable of decreasing the expression level and/or activity of a Taz polypeptide (and its fragments, domains, and/or motifs thereof, discussed herein). In some embodiments, the agent may decrease the binding/interaction between Taz and other transcription factors or binding partners. In other embodiments, the agent may decrease the expression of a Taz polypeptide. In still another embodiment, such inhibitors may increase or promote the turnover rate, reduce or inhibit the expression and/or the stability (e.g., the half-life), and/or change the cellular localization of Taz, resulting in at least a decrease in Taz levels and/or activity. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to Taz or also inhibit at least one of the binding partners.

The term, “Lgr5,” also known as leucine rich repeat containing G protein-coupled receptor 5, refers to a gene that encodes a leucine-rich repeat-containing receptor (LGR) and member of the G protein-coupled, 7-transmembrane receptor (GPCR) superfamily. The encoded protein is a receptor for R-spondins and is involved in the canonical Wnt signaling pathway. This protein plays a role in the formation and maintenance of adult intestinal stem cells during postembryonic development.

The term “Lgr5” is intended to include fragments, variants (e.g., allelic variants) and derivatives thereof. The nucleic acid and amino acid sequences of a representative human Lgr5 is available to the public at the GenBank database (Gene ID 8549). Human Lgr5 variants include, but not limited to, the transcript variant 1 encoding isoform 1 (NM_003667.4 and NP_003658.1), the transcript variant 2 encoding isoform 2 (NM_001277226.2 and NP_001264155.1), and the transcript variant 3 encoding isoform 3 (NM_001277227.2 and NP_001264156.1).

Nucleic acid and polypeptide sequences of Lgr5 orthologs in organisms other than humans are well known and include, for example, mouse Lgr5 (NM_010195.2 and NP_034325.2), rat Lgr5 (NM_001106784.1 and NP_001100254.1), chimpanzee Lgr5 (XM_003313861.4 and XP_003313909.1), and monkey Lgr5 (XM_001117502.4 and XP_001117502.1).

The term “Lgr5 activity” includes the ability of an Lgr5 polypeptide (and its fragments, domains, and/or motifs thereof, discussed herein) to bind its substrates, and/or mediate its activity and functions disclosed herein.

The term “agents that decrease the copy number, the expression level, and/or the activity of Lgr5” encompasses any natural or non-natural agent prepared, synthesized, manufactured, and/or purified by human that is capable of decreasing the expression level and/or activity of an Lgr5 polypeptide (and its fragments, domains, and/or motifs thereof, discussed herein). In some embodiments, the agent may decrease the binding/interaction between Lgr5 and other transcription factors or binding partners. In other embodiments, the agent may decrease the expression of an Lgr5 polypeptide. In still another embodiment, such inhibitors may increase or promote the turnover rate, reduce or inhibit the expression and/or the stability (e.g., the half-life), and/or change the cellular localization of Lgr5, resulting in at least a decrease in Lgr5 levels and/or activity. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to Lgr5 or also inhibit at least one of the binding partners.

The term, “Lrig1,” also known as leucine rich repeats and immunoglobulin like domains 1, refers to a Protein Coding gene. Diseases associated with Lrig1 include Hyaline Fibromatosis Syndrome and Verrucous Carcinoma. Among its related pathways are Signaling by GPCR and MET promotes cell motility. Lrig1 acts as a feedback negative regulator of signaling by receptor tyrosine kinases, through a mechanism that involves enhancement of receptor ubiquitination and accelerated intracellular degradation.

The term “Lrig1” is intended to include fragments, variants (e.g., allelic variants) and derivatives thereof. The nucleic acid and amino acid sequences of a representative human Lrig1 is available to the public at the GenBank database (Gene ID 26018). Human Lrig1 variants include, but not limited to, the transcript variant 1 encoding isoform 1 (NM_015541.3 and NP_056356.2), the transcript variant 2 encoding isoform 2 (NM_001377344.1 and NP_001364273.1), the transcript variant 3 encoding isoform 3 (NM_001377345.1 and NP_001364274.1), the transcript variant 4 encoding isoform 4 (NM_001377346.1 and NP_001364275.1), the transcript variant 5 encoding isoform 5 (NM_001377347.1 and NP_001364276.1), the transcript variant 6 encoding isoform 6 (NM_001377349.1 and NP_001364278.1), and the transcript variant 7 encoding isoform 7 (NM_001377348.1 and NP_001364277.1).

Nucleic acid and polypeptide sequences of Lrig1 orthologs in organisms other than humans are well known and include, for example, mouse Lrig1 (NM_008377.2 and NP_032403.2), rat Lrig1 (XM_232237.10 and XP_232237.6), zebrafish Lrig1 (XM_683725.9 and XP_688817.4), chimpanzee Lrig1 (XM_016941464.2 and XP_016796953.2), and monkey Lrig1 (XM_002802739.3 and XP_002802785.3).

The term “Lrig1 activity” includes the ability of an Lrig1 polypeptide (and its fragments, domains, and/or motifs thereof, discussed herein) to bind its substrates, and/or mediate its activity and functions disclosed herein.

The term “agents that decrease the copy number, the expression level, and/or the activity of Lrig1” encompasses any natural or non-natural agent prepared, synthesized, manufactured, and/or purified by human that is capable of decreasing the expression level and/or activity of an Lrig1 polypeptide (and its fragments, domains, and/or motifs thereof, discussed herein). In some embodiments, the agent may decrease the binding/interaction between Lrig1 and other transcription factors or binding partners. In other embodiments, the agent may decrease the expression of an Lrig1 polypeptide. In still another embodiment, such inhibitors may increase or promote the turnover rate, reduce or inhibit the expression and/or the stability (e.g., the half-life), and/or change the cellular localization of Lrig1, resulting in at least a decrease in Lrig1 levels and/or activity. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to Lrig1 or also inhibit at least one of the binding partners.

The term, “PROM1,” also known as prominin 1, refers to a gene that encodes a pentaspan transmembrane glycoprotein. The protein localizes to membrane protrusions and is often expressed on adult stem cells, where it is thought to function in maintaining stem cell properties by suppressing differentiation. Mutations in this gene have been shown to result in retinitis pigmentosa and Stargardt disease. Expression of this gene is also associated with several types of cancer. This gene is expressed from at least five alternative promoters that are expressed in a tissue-dependent manner.

The term “PROM1” is intended to include fragments, variants (e.g., allelic variants) and derivatives thereof. The nucleic acid and amino acid sequences of a representative human PROM1 is available to the public at the GenBank database (Gene ID 8842). Human PROM1 variants include, but not limited to, the transcript variant 1 encoding isoform 1 (NM_001145848.2 and NP_001139320.1), the transcript variant 2 encoding isoform 2 (NM_006017.3 and NP_006008.1), the transcript variant 3 encoding isoform 3 (NM_001145852.2 and NP_001139324.1), the transcript variant 4 encoding isoform 4 (NM_001145847.2 and NP_001139319.1), the transcript variant 5 encoding isoform 5 (NM_001371408.1 and NP_001358337.1), the transcript variant 6 encoding isoform 6 (NM_001371406.1 and NP_001358335.1), the transcript variant 7 encoding isoform 7 (NM_001145850.2 and NP_001139322.1), the transcript variant 8 encoding isoform 8 (NM_001371407.1 and NP_001358336.1), the transcript variant 9 encoding isoform 9 (NM_001145851.2 and NP_001139323.1), and the transcript variant 10 encoding isoform 10 (NM_001145849.2 and NP_001139321.1).

Nucleic acid and polypeptide sequences of PROM1 orthologs in organisms other than humans are well known and include, for example, mouse PROM1 (NM_008935.2 and NP_032961.2), rat PROM1 (NM_021751.2 and NP_068519.2), chimpanzee PROM1 (XM_024356037.1 and XP_024211805.1), and monkey PROM1 (XM_015138021.2 and XP_014993507.1).

The term “PROM1 activity” includes the ability of a PROM1 polypeptide (and its fragments, domains, and/or motifs thereof, discussed herein) to bind its substrates, and/or mediate its activity and functions disclosed herein.

The term “agents that decrease the copy number, the expression level, and/or the activity of PROM1” encompasses any natural or non-natural agent prepared, synthesized, manufactured, and/or purified by human that is capable of decreasing the expression level and/or activity of a PROM1 polypeptide (and its fragments, domains, and/or motifs thereof, discussed herein). In some embodiments, the agent may decrease the binding/interaction between PROM1 and other transcription factors or binding partners. In other embodiments, the agent may decrease the expression of a PROM1 polypeptide. In still another embodiment, such inhibitors may increase or promote the turnover rate, reduce or inhibit the expression and/or the stability (e.g., the half-life), and/or change the cellular localization of PROM1, resulting in at least a decrease in PROM1 levels and/or activity. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to PROM1 or also inhibit at least one of the binding partners

The term, “Axin2,” also known as Axin-related protein, refers to a protein coding gene. Axin2 presumably plays an important role in the regulation of the stability of beta-catenin in the Wnt signaling pathway. In mouse, conducting (a homolog of Axin2) organizes a multi-protein complex of APC (adenomatous polyposis of the colon), beta-catenin, glycogen synthase kinase 3-beta, and conducting, leading to the degradation of beta-catenin. The deregulation of beta-catenin is known to be involved in the genesis of a number of malignancies. The Axin2 gene has been mapped to 17q23-q24, a region that shows frequent loss of heterozygosity in breast cancer, neuroblastoma, and other tumors.

The term “Axin2” is intended to include fragments, variants (e.g., allelic variants) and derivatives thereof. The nucleic acid and amino acid sequences of a representative human Axin2 is available to the public at the GenBank database (Gene ID 8313). Human Axin2 variants include, but not limited to, the transcript variant 1 encoding isoform 1 (NM_004655.4 and NP_004646.3) and the transcript variant 2 encoding isoform 2 (NM_001363813.1 and NP_001350742.1).

Nucleic acid and polypeptide sequences of Axin2 orthologs in organisms other than humans are well known and include, for example, mouse Axin2 (NM_015732.4 and NP_056547.3), rat Axin2 (NM_024355.1 and NP_077331.1), zebrafish Axin2 (NM_131561.1 and NP_571636.1), and chimpanzee Axin2 (XM_016932766.2 and XP_016788255.2).

The term “Axin2 activity” includes the ability of an Axin2 polypeptide (and its fragments, domains, and/or motifs thereof, discussed herein) to bind its substrates, and/or mediate its activity and functions disclosed herein.

The term “agents that decrease the copy number, the expression level, and/or the activity of Axin2” encompasses any natural or non-natural agent prepared, synthesized, manufactured, and/or purified by human that is capable of decreasing the expression level and/or activity of an Axin2 polypeptide (and its fragments, domains, and/or motifs thereof, discussed herein). In some embodiments, the agent may decrease the binding/interaction between Axin2 and other transcription factors or binding partners. In other embodiments, the agent may decrease the expression of an Axin2 polypeptide. In still another embodiment, such inhibitors may increase or promote the turnover rate, reduce or inhibit the expression and/or the stability (e.g., the half-life), and/or change the cellular localization of Axin2, resulting in at least a decrease in Axin2 levels and/or activity. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to Axin2 or also inhibit at least one of the binding partners.

The term, “Ascl2,” also known as achaete-scute family bHLH transcription factor 2, refers to a member of the basic helix-loop-helix (BHLH) family of transcription factors. Ascl2 activates transcription by binding to the E box (5′-CANNTG-3′). Dimerization with other BHLH proteins is required for efficient DNA binding. Ascl2 is known to be involved in the determination of the neuronal precursors in the peripheral nervous system and the central nervous system.

The term “Ascl2” is intended to include fragments, variants (e.g., allelic variants) and derivatives thereof. The nucleic acid and amino acid sequences of a representative human Ascl2 is available to the public at the GenBank database (Gene ID 430) (NM_005170.3 and NP_005161.1).

Nucleic acid and polypeptide sequences of Ascl2 orthologs in organisms other than humans are well known and include, for example, mouse Ascl2 (NM_008554.3 and NP_032580.2), rat Ascl2 (NM_031503.2 and NP_113691.1), chimpanzee Ascl2 (XM_521719.6 and XP_521719.2), and monkey Ascl2 (XM_028833052.1 and XP_028688885.1).

The term “Ascl2 activity” includes the ability of an Ascl2 polypeptide (and its fragments, domains, and/or motifs thereof, discussed herein) to bind its substrates, and/or mediate its activity and functions disclosed herein.

The term “agents that decrease the copy number, the expression level, and/or the activity of Ascl2” encompasses any natural or non-natural agent prepared, synthesized, manufactured, and/or purified by human that is capable of decreasing the expression level and/or activity of an Ascl2 polypeptide (and its fragments, domains, and/or motifs thereof, discussed herein). In some embodiments, the agent may decrease the binding/interaction between Ascl2 and other transcription factors or binding partners. In other embodiments, the agent may decrease the expression of an Ascl2 polypeptide. In still another embodiment, such inhibitors may increase or promote the turnover rate, reduce or inhibit the expression and/or the stability (e.g., the half-life), and/or change the cellular localization of Ascl2, resulting in at least a decrease in Ascl2 levels and/or activity. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to Ascl2 or also inhibit at least one of the binding partners.

The term, “TCF4,” also known as transcription factor 4, refers to a gene that encodes transcription factor 4, a basic helix-loop-helix transcription factor. The encoded protein recognizes an Ephrussi-box (‘E-box’) binding site (‘CANNTG’)—a motif first identified in immunoglobulin enhancers. TCF4 is broadly expressed, and may play an important role in nervous system development. Defects in this gene are a cause of Pitt-Hopkins syndrome. In addition, an intronic CTG repeat normally numbering 10-37 repeat units can expand to >50 repeat units and cause Fuchs endothelial corneal dystrophy.

The term “TCF4” is intended to include fragments, variants (e.g., allelic variants) and derivatives thereof. The nucleic acid and amino acid sequences of a representative human TCF4 is available to the public at the GenBank database (Gene ID 6925). Human TCF4 variants include, but not limited to, the transcript variant 1 encoding isoform 1 (NM_001243226.3 and NP_001230155.2), the transcript variant 2 encoding isoform 2 (NM_001369584.1 and NP_001356513.1), the transcript variant 3 encoding isoform 3 (NM_001369576.1 and NP_001356505.1), the transcript variant 4 encoding isoform 4 (NM_001369577.1 and NP_001356506.1), and the transcript variant 5 encoding isoform 5 (NM_001369582.1 and NP_001356511.1).

Nucleic acid and polypeptide sequences of TCF4 orthologs in organisms other than humans are well known and include, for example, mouse TCF4 (XM_017317857.2 and XP_017173346.1), rat TCF4 (XM_039097159.1 and XP_038953087.1), monkey TCF4 (XM_015122035.2 and XP_014977521.1).

The term “TCF4 activity” includes the ability of a TCF4 polypeptide (and its fragments, domains, and/or motifs thereof, discussed herein) to bind its substrates, and/or mediate its activity and functions disclosed herein.

The term “agents that decrease the copy number, the expression level, and/or the activity of TCF4” encompasses any natural or non-natural agent prepared, synthesized, manufactured, and/or purified by human that is capable of decreasing the expression level and/or activity of a TCF4 polypeptide (and its fragments, domains, and/or motifs thereof, discussed herein). In some embodiments, the agent may decrease the binding/interaction between TCF4 and other transcription factors or binding partners. In other embodiments, the agent may decrease the expression of a TCF4 polypeptide. In still another embodiment, such inhibitors may increase or promote the turnover rate, reduce or inhibit the expression and/or the stability (e.g., the half-life), and/or change the cellular localization of TCF4, resulting in at least a decrease in TCF4 levels and/or activity. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to TCF4 or also inhibit at least one of the binding partners.

The term, “OLFM4,” also known as olfactomedin 4, refers to a gene that a member of the olfactomedin family. The encoded protein is an antiapoptotic factor that promotes tumor growth and is an extracellular matrix glycoprotein that facilitates cell adhesion. OLFM4 was originally cloned from human myeloblasts and found to be selectively expressed in inflamed colonic epithelium.

The term “OLFM4” is intended to include fragments, variants (e.g., allelic variants) and derivatives thereof. The nucleic acid and amino acid sequences of a representative human OLFM4 is available to the public at the GenBank database (Gene ID 10562) (NM_006418.5 and NP_006409.3).

Nucleic acid and polypeptide sequences of OLFM4 orthologs in organisms other than humans are well known and include, for example, mouse OLFM4 (NM_001351947.1 and NP_001338876.1), rat OLFM4 (NM_001106052.1 and NP_001099522.1), chimpanzee OLFM4 (XM_522764.4 and XP_522764.2), and monkey OLFM4 (XM_001085453.4 and XP_001085453.2).

The term “OLFM4 activity” includes the ability of an OLFM4 polypeptide (and its fragments, domains, and/or motifs thereof, discussed herein) to bind its substrates, and/or mediate its activity and functions disclosed herein.

The term “agents that decrease the copy number, the expression level, and/or the activity of OLFM4” encompasses any natural or non-natural agent prepared, synthesized, manufactured, and/or purified by human that is capable of decreasing the expression level and/or activity of an OLFM4 polypeptide (and its fragments, domains, and/or motifs thereof, discussed herein). In some embodiments, the agent may decrease the binding/interaction between OLFM4 and other transcription factors or binding partners. In other embodiments, the agent may decrease the expression of an OLFM4 polypeptide. In still another embodiment, such inhibitors may increase or promote the turnover rate, reduce or inhibit the expression and/or the stability (e.g., the half-life), and/or change the cellular localization of OLFM4, resulting in at least a decrease in OLFM4 levels and/or activity. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to OLFM4 or also inhibit at least one of the binding partners.

The term, “SMOC2,” also known as SPARC related modular calcium binding 2, refers to a gene that encodes a member of the SPARC family (secreted protein acidic and rich in cysteine/osteonectin/BM-40), which are highly expressed during embryogenesis and wound healing. The encoded protein is a matricellular protein which promotes matrix assembly and can stimulate endothelial cell proliferation and migration, as well as angiogenic activity. SMOC2 is associated with pulmonary function, this secretory gene product contains a Kazal domain, two thymoglobulin type-1 domains, and two EF-hand calcium-binding domains. The encoded protein may serve as a target for controlling angiogenesis in tumor growth and myocardial ischemia.

The term “SMOC2” is intended to include fragments, variants (e.g., allelic variants) and derivatives thereof. The nucleic acid and amino acid sequences of a representative human SMOC2 is available to the public at the GenBank database (Gene ID 664094). Human SMOC2 variants include, but not limited to, the transcript variant 1 encoding isoform 1 (NM_022138.3 and NP_071421.1) and the transcript variant 2 encoding isoform 2 (NM_001166412.2 and NP_001159884.1).

Nucleic acid and polypeptide sequences of SMOC2 orthologs in organisms other than humans are well known and include, for example, mouse SMOC2 (NM_022315.2 and NP_071710.2), rat SMOC2 (NM_001106215.2 and NP_001099685.1), zebrafish SMOC2 (XM_017358851.2 and XP_017214340.2), chimpanzee SMOC2 (XM_024357522.1 and XP_024213290.1), and monkey SMOC2 (XM_015137721.2 and XP_014993207.1).

The term “SMOC2 activity” includes the ability of a SMOC2 polypeptide (and its fragments, domains, and/or motifs thereof, discussed herein) to bind its substrates, and/or mediate its activity and functions disclosed herein.

The term “agents that decrease the copy number, the expression level, and/or the activity of SMOC2” encompasses any natural or non-natural agent prepared, synthesized, manufactured, and/or purified by human that is capable of decreasing the expression level and/or activity of a SMOC2 polypeptide (and its fragments, domains, and/or motifs thereof, discussed herein). In some embodiments, the agent may decrease the binding/interaction between SMOC2 and other transcription factors or binding partners. In other embodiments, the agent may decrease the expression of a SMOC2 polypeptide. In still another embodiment, such inhibitors may increase or promote the turnover rate, reduce or inhibit the expression and/or the stability (e.g., the half-life), and/or change the cellular localization of SMOC2, resulting in at least a decrease in SMOC2 levels and/or activity. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to SMOC2 or also inhibit at least one of the binding partners.

The term, “CDK6,” also known as cyclin dependent kinase 6, refers to a gene that encodes a member of the CMGC family of serine/threonine protein kinases. This kinase is a catalytic subunit of the protein kinase complex that is important for cell cycle G1 phase progression and G1/S transition. The activity of this kinase first appears in mid-G1 phase, which is controlled by the regulatory subunits including D-type cyclins and members of INK4 family of CDK inhibitors. CDK6, as well as CDK4, has been shown to phosphorylate, and thus regulate the activity of, tumor suppressor protein Rb. Altered expression of this gene has been observed in multiple human cancers. A mutation in this gene resulting in reduced cell proliferation, and impaired cell motility and polarity, and has been identified in patients with primary microcephaly.

The term “CDK6” is intended to include fragments, variants (e.g., allelic variants) and derivatives thereof. The nucleic acid and amino acid sequences of a representative human CDK6 is available to the public at the GenBank database (Gene ID 1021). Human CDK6 variants include, but not limited to, the transcript variant 1 encoding isoform 1 (NM_001145306.2 and NP_001138778.1) and the transcript variant 2 encoding isoform 2 (NM_001259.8 and NP_001250.1).

Nucleic acid and polypeptide sequences of CDK6 orthologs in organisms other than humans are well known and include, for example, mouse CDK6 (NM_009873.3 and NP_034003.1), rat CDK6 (NM_001191861.1 and NP_001178790.1), zebrafish CDK6 (NM_001144053.1 and NP_001137525.1), chimpanzee CDK6 (XM_003318579.4 and XP_003318627.1), and monkey CDK6 (NM_001261307.1 and NP_001248236.1).

The term “CDK6 activity” includes the ability of a CDK6 polypeptide (and its fragments, domains, and/or motifs thereof, discussed herein) to bind its substrates, and/or mediate its activity and functions disclosed herein.

The term “agents that decrease the copy number, the expression level, and/or the activity of CDK6” encompasses any natural or non-natural agent prepared, synthesized, manufactured, and/or purified by human that is capable of decreasing the expression level and/or activity of a CDK6 polypeptide (and its fragments, domains, and/or motifs thereof, discussed herein). In some embodiments, the agent may decrease the binding/interaction between CDK6 and other transcription factors or binding partners. In other embodiments, the agent may decrease the expression of a CDK6 polypeptide. In still another embodiment, such inhibitors may increase or promote the turnover rate, reduce or inhibit the expression and/or the stability (e.g., the half-life), and/or change the cellular localization of CDK6, resulting in at least a decrease in CDK6 levels and/or activity. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to CDK6 or also inhibit at least one of the binding partners.

The term, “LYZ” or “LYZ1” also known as lysozyme, refers to a gene that encodes human lysozyme, whose natural substrate is the bacterial cell wall peptidoglycan (cleaving the beta[1-4]glycosidic linkages between N-acetylmuramic acid and N-acetylglucosamine). Lysozyme is one of the antimicrobial agents found in human milk, and is also present in spleen, lung, kidney, white blood cells, plasma, saliva, and tears. The protein has antibacterial activity against a number of bacterial species. Missense mutations in this gene have been identified in heritable renal amyloidosis.

The term “LYZ” is intended to include fragments, variants (e.g., allelic variants) and derivatives thereof. The nucleic acid and amino acid sequences of a representative human LYZ is available to the public at the GenBank database (Gene ID 4069) (NM_000239.3 and NP_000230.1).

Nucleic acid and polypeptide sequences of LYZ orthologs in organisms other than humans are well known and include, for example, mouse LYZ (NM 013590.4 and NP_038618.1), chimpanzee LYZ (NM_001009073.1 and NP_001009073.1), and monkey LYZ (NM_001101733.1 and NP_001095203.1).

The term “LYZ activity” includes the ability of a LYZ polypeptide (and its fragments, domains, and/or motifs thereof, discussed herein) to bind its substrates, and/or mediate its activity and functions disclosed herein.

The term “agents that decrease the copy number, the expression level, and/or the activity of LYZ” encompasses any natural or non-natural agent prepared, synthesized, manufactured, and/or purified by human that is capable of decreasing the expression level and/or activity of a LYZ polypeptide (and its fragments, domains, and/or motifs thereof, discussed herein). In some embodiments, the agent may decrease the binding/interaction between LYZ and other transcription factors or binding partners. In other embodiments, the agent may decrease the expression of a LYZ polypeptide. In still another embodiment, such inhibitors may increase or promote the turnover rate, reduce or inhibit the expression and/or the stability (e.g., the half-life), and/or change the cellular localization of LYZ, resulting in at least a decrease in LYZ levels and/or activity. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to LYZ or also inhibit at least one of the binding partners.

The term, “DEFA5,” also known as defensin alpha 5, refers to a family of antimicrobial and cytotoxic peptides thought to be involved in host defense. These are abundant in the granules of neutrophils and also found in the epithelia of mucosal surfaces such as those of the intestine, respiratory tract, urinary tract, and vagina. Members of the defensin family are highly similar in protein sequence and distinguished by a conserved cysteine motif. Several of the alpha defensin genes appear to be clustered on chromosome 8. The protein encoded by this gene, defensin, alpha 5, is highly expressed in the secretory granules of Paneth cells of the ileum.

The term “DEFA5” is intended to include fragments, variants (e.g., allelic variants) and derivatives thereof. The nucleic acid and amino acid sequences of a representative human DEFA5 is available to the public at the GenBank database (Gene ID 1670) (NM_021010.3).

The term “DEFA5 activity” includes the ability of a DEFA5 polypeptide (and its fragments, domains, and/or motifs thereof, discussed herein) to bind its substrates, and/or mediate its activity and functions disclosed herein.

The term “agents that decrease the copy number, the expression level, and/or the activity of DEFA5” encompasses any natural or non-natural agent prepared, synthesized, manufactured, and/or purified by human that is capable of decreasing the expression level and/or activity of a DEFA5 polypeptide (and its fragments, domains, and/or motifs thereof, discussed herein). In some embodiments, the agent may decrease the binding/interaction between DEFA5 and other transcription factors or binding partners. In other embodiments, the agent may decrease the expression of a DEFA5 polypeptide. In still another embodiment, such inhibitors may increase or promote the turnover rate, reduce or inhibit the expression and/or the stability (e.g., the half-life), and/or change the cellular localization of DEFA5, resulting in at least a decrease in DEFA5 levels and/or activity. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to DEFA5 or also inhibit at least one of the binding partners.

The term, “DEFA6,” also known as defensin alpha 6, refers to a family of antimicrobial and cytotoxic peptides thought to be involved in host defense. These are abundant in the granules of neutrophils and also found in the epithelia of mucosal surfaces such as those of the intestine, respiratory tract, urinary tract, and vagina. Members of the defensin family are highly similar in protein sequence and distinguished by a conserved cysteine motif Several alpha defensin genes appear to be clustered on chromosome 8. The protein encoded by this gene, defensin, alpha 6, is highly expressed in the secretory granules of Paneth cells of the small intestine, and likely plays a role in host defense of human bowel.

The term “DEFA6” is intended to include fragments, variants (e.g., allelic variants) and derivatives thereof. The nucleic acid and amino acid sequences of a representative human DEFA6 is available to the public at the GenBank database (Gene ID 1671) (NM_001926.4).

Nucleic acid and polypeptide sequences of DEFA6 orthologs in organisms other than humans are well known and include, for example, mouse DEFA6 (NM_001177523.1 and NP_001170994.1), chimpanzee DEFA6 (NM_001033907.1), and monkey DEFA6 (XM_001098733.4).

The term “DEFA6 activity” includes the ability of a DEFA6 polypeptide (and its fragments, domains, and/or motifs thereof, discussed herein) to bind its substrates, and/or mediate its activity and functions disclosed herein.

The term “agents that decrease the copy number, the expression level, and/or the activity of DEFA6” encompasses any natural or non-natural agent prepared, synthesized, manufactured, and/or purified by human that is capable of decreasing the expression level and/or activity of a DEFA6 polypeptide (and its fragments, domains, and/or motifs thereof, discussed herein). In some embodiments, the agent may decrease the binding/interaction between DEFA6 and other transcription factors or binding partners. In other embodiments, the agent may decrease the expression of a DEFA6 polypeptide. In still another embodiment, such inhibitors may increase or promote the turnover rate, reduce or inhibit the expression and/or the stability (e.g., the half-life), and/or change the cellular localization of DEFA6, resulting in at least a decrease in DEFA6 levels and/or activity. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to DEFA6 or also inhibit at least one of the binding partners.

The term “TROP2” refers to tumor-associated calcium signal transducer 2, and is a protein in humans encoded by the TACSTD2 gene. The term is intended to include fragments, variants (e.g., allelic variants) and derivatives thereof. The nucleic acid and amino acid sequences of a representative human TROP2 is available to the public at the GenBank database (Gene ID 4070) (NC_000001.11).

Nucleic acid and polypeptide sequences of TROP2 orthologs in organisms other than humans are well known and include, for example, mouse TROP2 (NC_000070.7 and NC_000072.7), and rat TROP2 (NC_051339.1).

The term “TROP2 activity” includes the ability of a TROP2 polypeptide (and its fragments, domains, and/or motifs thereof, discussed herein) to bind its substrates, and/or mediate its activity and functions disclosed herein.

The term “agents that decrease the copy number, the expression level, and/or the activity of TROP2” encompasses any natural or non-natural agent prepared, synthesized, manufactured, and/or purified by human that is capable of decreasing the expression level and/or activity of a TROP2 polypeptide (and its fragments, domains, and/or motifs thereof, discussed herein). In some embodiments, the agent may decrease the binding/interaction between TROP2 and other transcription factors or binding partners. In other embodiments, the agent may decrease the expression of a TROP2 polypeptide. In still another embodiment, such inhibitors may increase or promote the turnover rate, reduce or inhibit the expression and/or the stability (e.g., the half-life), and/or change the cellular localization of TROP2, resulting in at least a decrease in TROP2 levels and/or activity. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to TROP2 or also inhibit at least one of the binding partners.

The term “LY6” refers to lymphocyte antigen 6 complex, and also includes all members of the lymphostromal cell membrane Ly6 superfamily. It is also intended to include fragments, variants (e.g., allelic variants) and derivatives thereof. The nucleic acid and amino acid sequences of a representative LY6 is available to the public at the GenBank database (Gene ID 17062).

Nucleic acid and polypeptide sequences of LY6 protein family in humans are well known and include, for example, those encoded by LY6D (NM_003695.3), LY6E (NM_002346.3 and NM_001127213.2), LY6H (NM_001130478.2), LY6K (NM_017527.4), PSCA (NR_033343.2), LYPD2 (NM_205545.3), SLURP1 (NM_020427.3), GML (NM_002066.3), GPIHBP1 (NM_178172.6), and LYNX1 (NM_001356370.1). An exemplary member of the lymphostromal cell membrane Ly6 superfamily is LYE6 (Gene ID 4061). As used herein, “LY6” includes LYE6.

The term “LY6 activity” includes the ability of a LY6 polypeptide (and its fragments, domains, and/or motifs thereof, discussed herein) to bind its substrates, and/or mediate its activity and functions disclosed herein.

The term “agents that decrease the copy number, the expression level, and/or the activity of LY6” encompasses any natural or non-natural agent prepared, synthesized, manufactured, and/or purified by human that is capable of decreasing the expression level and/or activity of a LY6 polypeptide (and its fragments, domains, and/or motifs thereof, discussed herein). In some embodiments, the agent may decrease the binding/interaction between LY6 and other transcription factors or binding partners. In other embodiments, the agent may decrease the expression of a LY6 polypeptide. In still another embodiment, such inhibitors may increase or promote the turnover rate, reduce or inhibit the expression and/or the stability (e.g., the half-life), and/or change the cellular localization of LY6, resulting in at least a decrease in LY6 levels and/or activity. Such inhibitors may be any molecule, including but not limited to small molecule compounds, antibodies or intrabodies, RNA interfering (RNAi) agents (including at least siRNAs, shRNAs, microRNAs (miRNAs), piwi, and other well-known agents). Such inhibitors may be specific to LY6 or also inhibit at least one of the binding partners.

The term, “Krt20,” also known as keratin 20, refers to a gene that encodes a member of the keratin family. The keratins are intermediate filament proteins responsible for the structural integrity of epithelial cells and are subdivided into cytokeratins and hair keratins. The type I cytokeratins consist of acidic proteins which are arranged in pairs of heterotypic keratin chains. This cytokeratin is a major cellular protein of mature enterocytes and goblet cells and is specifically expressed in the gastric and intestinal mucosa.

The term “Krt20” is intended to include fragments, variants (e.g., allelic variants) and derivatives thereof. The nucleic acid and amino acid sequences of a representative human Krt20 is available to the public at the GenBank database (Gene ID 54474) (NM_019010.3 and NP_061883.1).

Nucleic acid and polypeptide sequences of Krt20 orthologs in organisms other than humans are well known and include, for example, mouse Krt20 (NM_023256.2 and NP_075745.1), rat Krt20 (NM_173128.1 and NP_775151.1), chimpanzee Krt20 (XM_001168931.5 and XP_001168931.1), and monkey Krt20 (XM_028836479.1 and XP_028692312.1).

The term “Krt20 activity” includes the ability of a Krt20 polypeptide (and its fragments, domains, and/or motifs thereof, discussed herein) to bind its substrates, and/or mediate its activity and functions disclosed herein.

The term “agents that increase the copy number, the expression level, and/or the activity of Krt20” encompasses any natural or non-natural agent prepared, synthesized, manufactured, and/or purified by human that is capable of increasing the expression level and/or activity of a Krt20 polypeptide (and its fragments, domains, and/or motifs thereof, discussed herein). In some embodiments, the agent may increase the binding/interaction between Krt20 and other transcription factors or binding partners. In other embodiments, the agent may increase the expression of a Krt20 polypeptide. In still another embodiment, such enhancers or modulators may increase or promote the turnover rate, enhance or modulate the expression and/or the stability (e.g., the half-life), and/or change the cellular localization of Krt20, resulting in at least an increase in Krt20 levels and/or activity. In some embodiments, the agent that may decrease the copy number, the expression level, and/or the activity of biomarkers listed in Table 1, may increase the copy number, the expression level, and/or the activity of Krt20. Such enhancers or modulators may be any molecule, including but not limited to small molecule compounds, antibodies or intrabodies, RNA interfering (RNAi) agents (including at least siRNAs, shRNAs, microRNAs (miRNAs), piwi, and other well-known agents). Such enhancers or modulators may be specific to Krt20 or also enhance at least one of the binding partners.

The term, “DPP4,” also known as dipeptidyl peptidase 4, refers to a gene that encodes dipeptidyl peptidase 4, which is identical to adenosine deaminase complexing protein-2, and to the T-cell activation antigen CD26. It is an intrinsic type II transmembrane glycoprotein and a serine exopeptidase that cleaves X-proline dipeptides from the N-terminus of polypeptides. Dipeptidyl peptidase 4 is highly involved in glucose and insulin metabolism, as well as in immune regulation.

The term “DPP4” is intended to include fragments, variants (e.g., allelic variants) and derivatives thereof. The nucleic acid and amino acid sequences of a representative human DPP4 is available to the public at the GenBank database (Gene ID 6901). Human DPP4 variants include, but not limited to, the transcript variant 1 encoding isoform 1 (NM_000116.5 and NP_000107.1), the transcript variant 2 encoding isoform 2 (NM_181311.4 and NP_851828.1), the transcript variant 3 encoding isoform 3 (NM_181312.4 and NP_851829.1), the transcript variant 4 encoding isoform 4 (NM_181313.4 and NP_851830.1), and the transcript variant 5 encoding isoform 5 (NM_001303465.2 and NP_001290394.1).

Nucleic acid and polypeptide sequences of DPP4 orthologs in organisms other than humans are well known and include, for example, mouse DPP4 (NM_001173547.2 and NP_001167018.1), rat DPP4 (NM_001025748.1 and NP_001020919.1), zebrafish DPP4 (NM_001001814.1 and NP_001001814.1), chimpanzee DPP4 (NM_001009011.1 and NP_001009011.1), and monkey DPP4 (NM_001032914.1 and NP_001028086.1).

The term “DPP4 activity” includes the ability of a DPP4 polypeptide (and its fragments, domains, and/or motifs thereof, discussed herein) to bind its substrates, and/or mediate its activity and functions disclosed herein.

The term “agents that increase the copy number, the expression level, and/or the activity of DPP4” encompasses any natural or non-natural agent prepared, synthesized, manufactured, and/or purified by human that is capable of increasing the expression level and/or activity of a DPP4 polypeptide (and its fragments, domains, and/or motifs thereof, discussed herein). In some embodiments, the agent may increase the binding/interaction between DPP4 and other transcription factors or binding partners. In other embodiments, the agent may increase the expression of a DPP4 polypeptide. In still another embodiment, such enhancers or modulators may increase or promote the turnover rate, enhance or modulate the expression and/or the stability (e.g., the half-life), and/or change the cellular localization of DPP4, resulting in at least an increase in DPP4 levels and/or activity. In some embodiments, the agent that may decrease the copy number, the expression level, and/or the activity of biomarkers listed in Table 1, may increase the copy number, the expression level, and/or the activity of DPP4. Such enhancers or modulators may be any molecule, including but not limited to small molecule compounds, antibodies or intrabodies, RNA interfering (RNAi) agents (including at least siRNAs, shRNAs, microRNAs (miRNAs), piwi, and other well-known agents). Such enhancers or modulators may be specific to DPP4 or also enhance at least one of the binding partners.

The term, “APOA4,” also known as apolipoprotein A4, refers to a gene that contains 3 exons separated by two introns. A sequence polymorphism has been identified in the 3′UTR of the third exon. The primary translation product is a 396-residue preprotein which after proteolytic processing is secreted its primary site of synthesis, the intestine, in association with chylomicron particles. Although its precise function is not known, apo A-IV is a potent activator of lecithin-cholesterol acyltransferase in vitro.

The term “APOA4” is intended to include fragments, variants (e.g., allelic variants) and derivatives thereof. The nucleic acid and amino acid sequences of a representative human APOA4 is available to the public at the GenBank database (Gene ID 337) (NM_000482.4 and NP_000473.2).

Nucleic acid and polypeptide sequences of APOA4 orthologs in organisms other than humans are well known and include, for example, mouse APOA4 (NM 007468.2 and NP_031494.2), rat APOA4 (NM_012737.2 and NP_036869.2), chimpanzee APOA4 (XM_522192.6 and XP_522192.2), and monkey APOA4 (XM_028834005.1 and XP_028689838.1).

The term “APOA4 activity” includes the ability of a APOA4 polypeptide (and its fragments, domains, and/or motifs thereof, discussed herein) to bind its substrates, and/or mediate its activity and functions disclosed herein.

The term “agents that increase the copy number, the expression level, and/or the activity of APOA4” encompasses any natural or non-natural agent prepared, synthesized, manufactured, and/or purified by human that is capable of increasing the expression level and/or activity of a APOA4 polypeptide (and its fragments, domains, and/or motifs thereof, discussed herein). In some embodiments, the agent may increase the binding/interaction between APOA4 and other transcription factors or binding partners. In other embodiments, the agent may increase the expression of a APOA4 polypeptide. In still another embodiment, such enhancers or modulators may increase or promote the turnover rate, enhance or modulate the expression and/or the stability (e.g., the half-life), and/or change the cellular localization of APOA4, resulting in at least an increase in APOA4 levels and/or activity. In some embodiments, the agent that may decrease the copy number, the expression level, and/or the activity of biomarkers listed in Table 1, may increase the copy number, the expression level, and/or the activity of APOA4. Such enhancers or modulators may be any molecule, including but not limited to small molecule compounds, antibodies or intrabodies, RNA interfering (RNAi) agents (including at least siRNAs, shRNAs, microRNAs (miRNAs), piwi, and other well-known agents). Such enhancers or modulators may be specific to APOA4 or also enhance at least one of the binding partners.

The term, “ATOH1,” also known as atonal bHLH transcription factor 1, refers to a Protein Coding gene. Diseases associated with ATOH1 include Serous Labyrinthitis and Goblet Cell Carcinoid. Gene Ontology (GO) annotations related to this gene include DNA-binding transcription factor activity and RNA polymerase II proximal promoter sequence-specific DNA binding. ATOH1 is an important paralog of NEUROD2.

The term “ATOH1” is intended to include fragments, variants (e.g., allelic variants) and derivatives thereof. The nucleic acid and amino acid sequences of a representative human ATOH1 is available to the public at the GenBank database (Gene ID 474) (NM_005172.2 and NP_005163.1).

Nucleic acid and polypeptide sequences of ATOH1 orthologs in organisms other than humans are well known and include, for example, mouse ATOH1 (NM_007500.5 and NP_031526.1), rat ATOH1 (NM_001109238.1 and NP_001102708.1), zebrafish ATOH1 (NM_131091.2 and NP_571166.2), chimpanzee ATOH1 (NM_001012432.1 and NP_001012434.1), and monkey ATOH1 (XM_001102247.4 and XP_001102247.1).

The term “ATOH1 activity” includes the ability of an ATOH1 polypeptide (and its fragments, domains, and/or motifs thereof, discussed herein) to bind its substrates, and/or mediate its activity and functions disclosed herein.

The term “agents that increase the copy number, the expression level, and/or the activity of ATOH1” encompasses any natural or non-natural agent prepared, synthesized, manufactured, and/or purified by human that is capable of increasing the expression level and/or activity of an ATOH1 polypeptide (and its fragments, domains, and/or motifs thereof, discussed herein). In some embodiments, the agent may increase the binding/interaction between ATOH1 and other transcription factors or binding partners. In other embodiments, the agent may increase the expression of an ATOH1 polypeptide. In still another embodiment, such enhancers or modulators may increase or promote the turnover rate, enhance or modulate the expression and/or the stability (e.g., the half-life), and/or change the cellular localization of ATOH1, resulting in at least an increase in ATOH1 levels and/or activity. In some embodiments, the agent that may decrease the copy number, the expression level, and/or the activity of biomarkers listed in Table 1, may increase the copy number, the expression level, and/or the activity of ATOH1. Such enhancers or modulators may be any molecule, including but not limited to small molecule compounds, antibodies or intrabodies, RNA interfering (RNAi) agents (including at least siRNAs, shRNAs, microRNAs (miRNAs), piwi, and other well-known agents). Such enhancers or modulators may be specific to ATOH1 or also enhance at least one of the binding partners.

The term, “TFF2,” also known as trefoil factor 2, refers to a protein coding gene. Members of the trefoil family are characterized by having at least one copy of the trefoil motif, a 40-amino acid domain that contains three conserved disulfides. They are stable secretory proteins expressed in gastrointestinal mucosa. Their functions are not defined, but they may protect the mucosa from insults, stabilize the mucus layer and affect healing of the epithelium. The encoded protein inhibits gastric acid secretion. This gene and two other related trefoil family member genes are found in a cluster on chromosome 21.

The term “TFF2” is intended to include fragments, variants (e.g., allelic variants) and derivatives thereof. The nucleic acid and amino acid sequences of a representative human TFF2 is available to the public at the GenBank database (Gene ID 7032) (NM_005423.5 and NP_005414.1).

Nucleic acid and polypeptide sequences of TFF2 orthologs in organisms other than humans are well known and include, for example, mouse TFF2 (NM_009363.3 and NP_033389.2), rat TFF2 (NM_053844.2 and NP_446296.2), chimpanzee TFF2 (XM_525480.4 and XP_525480.2), and monkey TFF2 (XM_015132798.2 and XP_014988284.2).

The term “TFF2 activity” includes the ability of a TFF2 polypeptide (and its fragments, domains, and/or motifs thereof, discussed herein) to bind its substrates, and/or mediate its activity and functions disclosed herein.

The term “agents that increase the copy number, the expression level, and/or the activity of TFF2” encompasses any natural or non-natural agent prepared, synthesized, manufactured, and/or purified by human that is capable of increasing the expression level and/or activity of a TFF2 polypeptide (and its fragments, domains, and/or motifs thereof, discussed herein). In some embodiments, the agent may increase the binding/interaction between TFF2 and other transcription factors or binding partners. In other embodiments, the agent may increase the expression of a TFF2 polypeptide. In still another embodiment, such enhancers or modulators may increase or promote the turnover rate, enhance or modulate the expression and/or the stability (e.g., the half-life), and/or change the cellular localization of TFF2, resulting in at least an increase in TFF2 levels and/or activity. In some embodiments, the agent that may decrease the copy number, the expression level, and/or the activity of biomarkers listed in Table 1, may increase the copy number, the expression level, and/or the activity of TFF2. Such enhancers or modulators may be any molecule, including but not limited to small molecule compounds, antibodies or intrabodies, RNA interfering (RNAi) agents (including at least siRNAs, shRNAs, microRNAs (miRNAs), piwi, and other well-known agents). Such enhancers or modulators may be specific to TFF2 or also enhance at least one of the binding partners.

The term, “FCGBP,” also known as Fc fragment of IgG binding protein, refers to a coding gene that may be involved in the maintenance of the mucosal structure as a gel-like component of the mucosa.

The term “FCGBP” is intended to include fragments, variants (e.g., allelic variants) and derivatives thereof. The nucleic acid and amino acid sequences of a representative human FCGBP is available to the public at the GenBank database (Gene ID 8857) (NM_003890.2 and NP_003881.2).

Nucleic acid and polypeptide sequences of FCGBP orthologs in organisms other than humans are well known and include, for example, mouse FCGBP (XM_021166163.1 and XP_021021822.1), chimpanzee FCGBP (XM_024351358.1 and XP_024207126.1), and monkey FCGBP (XM_028838934.1 and XP_028694767.1).

The term “FCGBP activity” includes the ability of a FCGBP polypeptide (and its fragments, domains, and/or motifs thereof, discussed herein) to bind its substrates, and/or mediate its activity and functions disclosed herein.

The term “agents that increase the copy number, the expression level, and/or the activity of FCGBP” encompasses any natural or non-natural agent prepared, synthesized, manufactured, and/or purified by human that is capable of increasing the expression level and/or activity of a FCGBP polypeptide (and its fragments, domains, and/or motifs thereof, discussed herein). In some embodiments, the agent may increase the binding/interaction between FCGBP and other transcription factors or binding partners. In other embodiments, the agent may increase the expression of a FCGBP polypeptide. In still another embodiment, such enhancers or modulators may increase or promote the turnover rate, enhance or modulate the expression and/or the stability (e.g., the half-life), and/or change the cellular localization of FCGBP, resulting in at least an increase in FCGBP levels and/or activity. In some embodiments, the agent that may decrease the copy number, the expression level, and/or the activity of biomarkers listed in Table 1, may increase the copy number, the expression level, and/or the activity of FCGBP. Such enhancers or modulators may be any molecule, including but not limited to small molecule compounds, antibodies or intrabodies, RNA interfering (RNAi) agents (including at least siRNAs, shRNAs, microRNAs (miRNAs), piwi, and other well-known agents). Such enhancers or modulators may be specific to FCGBP or also enhance at least one of the binding partners.

The term, “REG4,” also known as regenerating family member 4, refers to a protein coding gene. Diseases associated with REG4 include ulcerative colitis and urachus cancer. Among its related pathways are gastric cancer and adhesion. Gene ontology (GO) annotations related to this gene include calcium ion binding and heparin binding. An important paralog of this gene is REG1B.

The term “REG4” is intended to include fragments, variants (e.g., allelic variants) and derivatives thereof. The nucleic acid and amino acid sequences of a representative human REG4 is available to the public at the GenBank database (Gene ID 83998). Human REG4 variants include, but not limited to, the transcript variant 1 encoding isoform 1 (NM_032044.4 and NP_114433.1), the transcript variant 2 encoding isoform 2 (NM_001159352.2 and NP_001152824.1), and the transcript variant 3 encoding isoform 3 (NM_001159353.2 and NP_001152825.1).

Nucleic acid and polypeptide sequences of REG4 orthologs in organisms other than humans are well known and include, for example, mouse REG4 (NM_026328.2 and NP_080604.2), rat REG4 (NM_001004096.1 and NP_001004096.1), chimpanzee REG4 (XM_016924613.1 and XP_016780102.1), and monkey REG4 (XM_001084012.4 and XP_001084012.1).

The term “REG4 activity” includes the ability of a REG4 polypeptide (and its fragments, domains, and/or motifs thereof, discussed herein) to bind its substrates, and/or mediate its activity and functions disclosed herein.

The term “agents that increase the copy number, the expression level, and/or the activity of REG4” encompasses any natural or non-natural agent prepared, synthesized, manufactured, and/or purified by human that is capable of increasing the expression level and/or activity of a REG4 polypeptide (and its fragments, domains, and/or motifs thereof, discussed herein). In some embodiments, the agent may increase the binding/interaction between REG4 and other transcription factors or binding partners. In other embodiments, the agent may increase the expression of a REG4 polypeptide. In still another embodiment, such enhancers or modulators may increase or promote the turnover rate, enhance or modulate the expression and/or the stability (e.g., the half-life), and/or change the cellular localization of REG4, resulting in at least an increase in REG4 levels and/or activity. In some embodiments, the agent that may decrease the copy number, the expression level, and/or the activity of biomarkers listed in Table 1, may increase the copy number, the expression level, and/or the activity of REG4. Such enhancers or modulators may be any molecule, including but not limited to small molecule compounds, antibodies or intrabodies, RNA interfering (RNAi) agents (including at least siRNAs, shRNAs, microRNAs (miRNAs), piwi, and other well-known agents). Such enhancers or modulators may be specific to REG4 or also enhance at least one of the binding partners.

The term, “AGR2,” also known as anterior gradient 2, protein disulphide isomerase family member, refers to a gene that encodes a member of the disulfide isomerase (PDI) family of endoplasmic reticulum (ER) proteins that catalyze protein folding and thiol-disulfide interchange reactions. The encoded protein has an N-terminal ER-signal sequence, a catalytically active thioredoxin domain, and a C-terminal ER-retention sequence. This protein plays a role in cell migration, cellular transformation and metastasis and is as a p53 inhibitor. As an ER-localized molecular chaperone, it plays a role in the folding, trafficking, and assembly of cysteine-rich transmembrane receptors and the cysteine-rich intestinal gylcoprotein mucin. This gene has been implicated in inflammatory bowel disease and cancer progression.

The term “AGR2” is intended to include fragments, variants (e.g., allelic variants) and derivatives thereof. The nucleic acid and amino acid sequences of a representative human AGR2 is available to the public at the GenBank database (Gene ID 10551) (NM_006408.4 and NP_006399.1).

Nucleic acid and polypeptide sequences of AGR2 orthologs in organisms other than humans are well known and include, for example, mouse AGR2 (NM_011783.2 and NP_035913.1), rat AGR2 (NM_001106725.1 and NP_001100195.1), zebrafish AGR2 (NM_001012481.2 and NP_001012499.1), chimpanzee AGR2 (XM_003318332.4 and XP_003318380.1), and monkey AGR2 (NM_001194304.1 and NP_001181233.1).

The term “AGR2 activity” includes the ability of an AGR2 polypeptide (and its fragments, domains, and/or motifs thereof, discussed herein) to bind its substrates, and/or mediate its activity and functions disclosed herein.

The term “agents that increase the copy number, the expression level, and/or the activity of AGR2” encompasses any natural or non-natural agent prepared, synthesized, manufactured, and/or purified by human that is capable of increasing the expression level and/or activity of an AGR2 polypeptide (and its fragments, domains, and/or motifs thereof, discussed herein). In some embodiments, the agent may increase the binding/interaction between AGR2 and other transcription factors or binding partners. In other embodiments, the agent may increase the expression of an AGR2 polypeptide. In still another embodiment, such enhancers or modulators may increase or promote the turnover rate, enhance or modulate the expression and/or the stability (e.g., the half-life), and/or change the cellular localization of AGR2, resulting in at least an increase in AGR2 levels and/or activity. In some embodiments, the agent that may decrease the copy number, the expression level, and/or the activity of biomarkers listed in Table 1, may increase the copy number, the expression level, and/or the activity of AGR-2. Such enhancers or modulators may be any molecule, including but not limited to small molecule compounds, antibodies or intrabodies, RNA interfering (RNAi) agents (including at least siRNAs, shRNAs, microRNAs (miRNAs), piwi, and other well-known agents). Such enhancers or modulators may be specific to AGR2 or also enhance at least one of the binding partners

The term, “MLUC2,” also known as mucin 2, refers to a gene that encodes a member of the mucin protein family. Mucins are high molecular weight glycoproteins produced by many epithelial tissues. The protein encoded by this gene is secreted and forms an insoluble mucous barrier that protects the gut lumen. The protein polymerizes into a gel of which 80% is composed of oligosaccharide side chains by weight. The protein features a central domain containing tandem repeats rich in threonine and proline that varies between 50 and 115 copies in different individuals. Downregulation of this gene has been observed in patients with Crohn disease and ulcerative colitis.

The term “MUTC2” is intended to include fragments, variants (e.g., allelic variants) and derivatives thereof. The nucleic acid and amino acid sequences of a representative human MUC2 is available to the public at the GenBank database (Gene ID 4583) (NM_002457.4 and NP_002448.4).

Nucleic acid and polypeptide sequences of MUC2 orthologs in organisms other than humans are well known and include, for example, mouse MUC2 (NM_023566.4 and NP_076055.4), rat MUC2 (XM_039101270.1 and XP_038957198.1), and monkey MUC2 (XM_028832923.1 and XP_028688756.1).

The term “MUC2 activity” includes the ability of a MUC2 polypeptide (and its fragments, domains, and/or motifs thereof, discussed herein) to bind its substrates, and/or mediate its activity and functions disclosed herein.

The term “agents that increase the copy number, the expression level, and/or the activity of MUC2” encompasses any natural or non-natural agent prepared, synthesized, manufactured, and/or purified by human that is capable of increasing the expression level and/or activity of a MUC2 polypeptide (and its fragments, domains, and/or motifs thereof, discussed herein). In some embodiments, the agent may increase the binding/interaction between MUC2 and other transcription factors or binding partners. In other embodiments, the agent may increase the expression of a MUC2 polypeptide. In still another embodiment, such enhancers or modulators may increase or promote the turnover rate, enhance or modulate the expression and/or the stability (e.g., the half-life), and/or change the cellular localization of MUC2, resulting in at least an increase in MUC2 levels and/or activity. In some embodiments, the agent that may decrease the copy number, the expression level, and/or the activity of biomarkers listed in Table 1, may increase the copy number, the expression level, and/or the activity of MUC2. Such enhancers or modulators may be any molecule, including but not limited to small molecule compounds, antibodies or intrabodies, RNA interfering (RNAi) agents (including at least siRNAs, shRNAs, microRNAs (miRNAs), piwi, and other well-known agents). Such enhancers or modulators may be specific to MUC2 or also enhance at least one of the binding partners.

The term, “CDX1,” also known as caudal type homeobox 1, refers to a gene that is a member of the caudal-related homeobox transcription factor gene family. The encoded DNA-binding protein regulates intestine-specific gene expression and enterocyte differentiation. It has been shown to induce expression of the intestinal alkaline phosphatase gene, and inhibit beta-catenin/T-cell factor transcriptional activity.

The term “CDX” is intended to include fragments, variants (e.g., allelic variants) and derivatives thereof. The nucleic acid and amino acid sequences of a representative human CDX1 is available to the public at the GenBank database (Gene ID 1044) (NM_001804.3 and NP_001795.2).

Nucleic acid and polypeptide sequences of CDX1 orthologs in organisms other than humans are well known and include, for example, mouse CDX1 (NM_009880.3 and NP_034010.3), rat CDX1 (XM_006254816.3 and XP_006254878.3), zebrafish CDX1 (NM_001098762.1 and NP_001092232.1), chimpanzee CDX1 (XM_016954032.2 and XP_016809521.2), and monkey CDX1 (XM_001108012.4 and XP_001108012.1).

The term “CDX1 activity” includes the ability of a CDX1 polypeptide (and its fragments, domains, and/or motifs thereof, discussed herein) to bind its substrates, and/or mediate its activity and functions disclosed herein.

The term “agents that increase the copy number, the expression level, and/or the activity of CDX1” encompasses any natural or non-natural agent prepared, synthesized, manufactured, and/or purified by human that is capable of increasing the expression level and/or activity of a CDX1 polypeptide (and its fragments, domains, and/or motifs thereof, discussed herein). In some embodiments, the agent may increase the binding/interaction between CDX1 and other transcription factors or binding partners. In other embodiments, the agent may increase the expression of a CDX1 polypeptide. In still another embodiment, such enhancers or modulators may increase or promote the turnover rate, enhance or modulate the expression and/or the stability (e.g., the half-life), and/or change the cellular localization of CDX1, resulting in at least an increase in CDX1 levels and/or activity. In some embodiments, the agent that may decrease the copy number, the expression level, and/or the activity of biomarkers listed in Table 1, may increase the copy number, the expression level, and/or the activity of CDX. Such enhancers or modulators may be any molecule, including but not limited to small molecule compounds, antibodies or intrabodies, RNA interfering (RNAi) agents (including at least siRNAs, shRNAs, microRNAs (miRNAs), piwi, and other well-known agents). Such enhancers or modulators may be specific to CDX1 or also enhance at least one of the binding partners.

The term, “CDX2,” also known as caudal type homeobox 2, refers to a gene that is a member of the caudal-related homeobox transcription factor gene family. The encoded protein is a major regulator of intestine-specific genes involved in cell growth and differentiation. This protein also plays a role in early embryonic development of the intestinal tract. Aberrant expression of this gene is associated with intestinal inflammation and tumorigenesis.

The term “CDX2” is intended to include fragments, variants (e.g., allelic variants) and derivatives thereof. The nucleic acid and amino acid sequences of a representative human CDX2 is available to the public at the GenBank database (Gene ID 1045). Human CDX2 variants include, but not limited to, the transcript variant 1 encoding isoform 1 (NM_001265.6 and NP_001256.4) and the transcript variant 2 encoding isoform 2 (NM_001354700.2 and NP_001341629.1).

Nucleic acid and polypeptide sequences of CDX2 orthologs in organisms other than humans are well known and include, for example, mouse CDX2 (NM_007673.3 and NP_031699.2), rat CDX2 (NM_023963.2 and NP_076453.1), and monkey CDX2 (XM_001096874.4 and XP_001096874.1).

The term “CDX2 activity” includes the ability of a CDX2 polypeptide (and its fragments, domains, and/or motifs thereof, discussed herein) to bind its substrates, and/or mediate its activity and functions disclosed herein.

The term “agents that increase the copy number, the expression level, and/or the activity of CDX2” encompasses any natural or non-natural agent prepared, synthesized, manufactured, and/or purified by human that is capable of increasing the expression level and/or activity of a CDX2 polypeptide (and its fragments, domains, and/or motifs thereof, discussed herein). In some embodiments, the agent may increase the binding/interaction between CDX2 and other transcription factors or binding partners. In other embodiments, the agent may increase the expression of a CDX2 polypeptide. In still another embodiment, such enhancers or modulators may increase or promote the turnover rate, enhance or modulate the expression and/or the stability (e.g., the half-life), and/or change the cellular localization of CDX2, resulting in at least an increase in CDX2 levels and/or activity. In some embodiments, the agent that may decrease the copy number, the expression level, and/or the activity of biomarkers listed in Table 1, may increase the copy number, the expression level, and/or the activity of CDX2. Such enhancers or modulators may be any molecule, including but not limited to small molecule compounds, antibodies or intrabodies, RNA interfering (RNAi) agents (including at least siRNAs, shRNAs, microRNAs (miRNAs), piwi, and other well-known agents). Such enhancers or modulators may be specific to CDX2 or also enhance at least one of the binding partners.

The term, “SLC2A5,” also known as solute carrier family 2 member 5, refers to a gene that encodes a fructose transporter responsible for fructose uptake by the small intestine. The encoded protein also is necessary for the increase in blood pressure due to high dietary fructose consumption.

The term “SLC2A5” is intended to include fragments, variants (e.g., allelic variants) and derivatives thereof. The nucleic acid and amino acid sequences of a representative human SLC2A5 is available to the public at the GenBank database (Gene ID 6518). Human SLC2A5 variants include, but not limited to, the transcript variant 1 encoding isoform 1 (NM_001328619.2 and NP_001315550.1), the transcript variant 2 encoding isoform 2 (NM_001328620.2 and NP_001315549.1), the transcript variant 3 encoding isoform 3 (NM_003039.3 and NP_003030.1), the transcript variant 4 encoding isoform 4 (NM_001328620.2 and NP_001315549.1), and the transcript variant 5 encoding isoform 5 (NM_001135585.2 and NP_001129057.1).

Nucleic acid and polypeptide sequences of SLC2A5 orthologs in organisms other than humans are well known and include, for example, mouse SLC2A5 (NM_019741.3 and NP_062715.2), rat SLC2A5 (NM_031741.1 and NP_113929.1), zebrafish SLC2A5 (NM_001001814.1 and NP_001001814.1), chimpanzee SLC2A5 (NM_001246591.1 and NP_001233520.1), and monkey SLC2A5 (XM_015146614.2 and XP_015002100.1).

The term “SLC2A5 activity” includes the ability of a SLC2A5 polypeptide (and its fragments, domains, and/or motifs thereof, discussed herein) to bind its substrates, and/or mediate its activity and functions disclosed herein.

The term “agents that increase the copy number, the expression level, and/or the activity of SLC2A5” encompasses any natural or non-natural agent prepared, synthesized, manufactured, and/or purified by human that is capable of increasing the expression level and/or activity of a SLC2A5 polypeptide (and its fragments, domains, and/or motifs thereof, discussed herein). In some embodiments, the agent may increase the binding/interaction between SLC2A5 and other transcription factors or binding partners. In other embodiments, the agent may increase the expression of a SLC2A5 polypeptide. In still another embodiment, such enhancers or modulators may increase or promote the turnover rate, enhance or modulate the expression and/or the stability (e.g., the half-life), and/or change the cellular localization of SLC2A5, resulting in at least an increase in SLC2A5 levels and/or activity. In some embodiments, the agent that may decrease the copy number, the expression level, and/or the activity of biomarkers listed in Table 1, may increase the copy number, the expression level, and/or the activity of SLC2A5. Such enhancers or modulators may be any molecule, including but not limited to small molecule compounds, antibodies or intrabodies, RNA interfering (RNAi) agents (including at least siRNAs, shRNAs, microRNAs (miRNAs), piwi, and other well-known agents). Such enhancers or modulators may be specific to SLC2A5 or also enhance at least one of the binding partners.

The term, “B3GALT5,” also known as beta-1,3-galactosyltransferase 5, refers to a gene that encodes a member of a family of membrane-bound glycoproteins. The encoded protein may synthesize type 1 Lewis antigens, which are elevated in gastrointestinal and pancreatic cancers. Alternatively spliced transcript variants using multiple alternate promoters have been observed for this gene.

The term “B3GALT5” is intended to include fragments, variants (e.g., allelic variants) and derivatives thereof. The nucleic acid and amino acid sequences of a representative human B3GALT5 is available to the public at the GenBank database (Gene ID 10317). Human B3GALT5 variants include, but not limited to, the transcript variant 1 encoding isoform 1 (NM_001356336.2 and NP_001343265.1), the transcript variant 2 encoding isoform 2 (NM_001278650.2 and NP_001265579.1), the transcript variant 3 encoding isoform 3 (NM_001356338.2 and NP_001343267.1), the transcript variant 4 encoding isoform 4 (NM_001356339.2 and NP_001343268.1), the transcript variant 5 encoding isoform 5 (NM_033171.3 and NP_149361.1), the transcript variant 6 encoding isoform 1 (NM_033172.3 and NP_149362.2), the transcript variant 7 encoding isoform 2 (NM_006057.3 and NP_006048.1), and the transcript variant 8 encoding isoform 3 (NM_033170.3 and NP_149360.1).

Nucleic acid and polypeptide sequences of B3GALT5 orthologs in organisms other than humans are well known and include, for example, mouse B3GALT5 (NM_001122993.1 and NP_001116465.1), rat B3GALT5 (NM_001105887.2 and NP_001099357.1), chimpanzee B3GALT5 (XM_016938553.2 and XP_016794042.1), and monkey B3GALT5 (XM_001108171.4 and XP_001108171.2).

The term “B3GALT5 activity” includes the ability of a B3GALT5 polypeptide (and its fragments, domains, and/or motifs thereof, discussed herein) to bind its substrates, and/or mediate its activity and functions disclosed herein.

The term “agents that increase the copy number, the expression level, and/or the activity of B3GALT5” encompasses any natural or non-natural agent prepared, synthesized, manufactured, and/or purified by human that is capable of increasing the expression level and/or activity of a B3GALT5 polypeptide (and its fragments, domains, and/or motifs thereof, discussed herein). In some embodiments, the agent may increase the binding/interaction between B3GALT5 and other transcription factors or binding partners. In other embodiments, the agent may increase the expression of a B3GALT5 polypeptide. In still another embodiment, such enhancers or modulators may increase or promote the turnover rate, enhance or modulate the expression and/or the stability (e.g., the half-life), and/or change the cellular localization of B3GALT5, resulting in at least an increase in B3GALT5 levels and/or activity. In some embodiments, the agent that may decrease the copy number, the expression level, and/or the activity of biomarkers listed in Table 1, may increase the copy number, the expression level, and/or the activity of B3GALT5. Such enhancers or modulators may be any molecule, including but not limited to small molecule compounds, antibodies or intrabodies, RNA interfering (RNAi) agents (including at least siRNAs, shRNAs, microRNAs (miRNAs), piwi, and other well-known agents). Such enhancers or modulators may be specific to B3GALT5 or also enhance at least one of the binding partners.

The term, “FUT3,” also known as fucosyltransferase 3, refers to a gene that is a member of the fucosyltransferase family, which catalyzes the addition of fucose to precursor polysaccharides in the last step of Lewis antigen biosynthesis. FUT3 encodes an enzyme with alpha(1,3)-fucosyltransferase and alpha(1,4)-fucosyltransferase activities.

Mutations in this gene are responsible for the majority of Lewis antigen-negative phenotypes. Differences in the expression of this gene are associated with host susceptibility to viral infection.

The term “FUT3” is intended to include fragments, variants (e.g., allelic variants) and derivatives thereof. The nucleic acid and amino acid sequences of a representative human FUT3 is available to the public at the GenBank database (Gene ID 2525). Human FUT3 variants include, but not limited to, the transcript variant 1 encoding isoform 1 (NM_001382749.1 and NP_001369678.1), the transcript variant 2 encoding isoform 2 (NM_001382750.1 and NP_001369679.1), the transcript variant 3 encoding isoform 3 (NM_001382748.1 and NP_001369677.1), the transcript variant 4 encoding isoform 4 (NM_001097641.3 and NP_001091110.3), the transcript variant 5 encoding isoform 5 (NM_001097640.3 and NP_001091109.3), the transcript variant 6 encoding isoform 6 (NM_001374740.1 and NP_001361669.1), the transcript variant 7 encoding isoform 7 (NM_001097639.3 and NP_001091108.3), the transcript variant 8 encoding isoform 8 (NM_001382744.1 and NP_001369673.1), the transcript variant 9 encoding isoform 9 (NM_001382747.1 and NP_001369676.1), the transcript variant 10 encoding isoform 10 (NM_001382746.1 and NP_001369675.1), the transcript variant 11 encoding isoform 11 (NM_001382745.1 and NP_001369674.1), and the transcript variant 12 encoding isoform 12 (NM_000149.4 and NP_000140.1).

Nucleic acid and polypeptide sequences of FUT3 orthologs in organisms other than humans are well known and include, for example, chimpanzee FUT3 (NM_001009149.1 and NP_001009149.1), and monkey FUT3 (XM_003846215.3 and XP_003846263.3).

The term “FUT3 activity” includes the ability of a FUT3 polypeptide (and its fragments, domains, and/or motifs thereof, discussed herein) to bind its substrates, and/or mediate its activity and functions disclosed herein.

The term “agents that increase the copy number, the expression level, and/or the activity of FUT3” encompasses any natural or non-natural agent prepared, synthesized, manufactured, and/or purified by human that is capable of increasing the expression level and/or activity of a FUT3 polypeptide (and its fragments, domains, and/or motifs thereof, discussed herein). In some embodiments, the agent may increase the binding/interaction between FUT3 and other transcription factors or binding partners. In other embodiments, the agent may increase the expression of a FUT3 polypeptide. In still another embodiment, such enhancers or modulators may increase or promote the turnover rate, enhance or modulate the expression and/or the stability (e.g., the half-life), and/or change the cellular localization of FUT3, resulting in at least an increase in FUT3 levels and/or activity. In some embodiments, the agent that may decrease the copy number, the expression level, and/or the activity of biomarkers listed in Table 1, may increase the copy number, the expression level, and/or the activity of FUT3. Such enhancers or modulators may be any molecule, including but not limited to small molecule compounds, antibodies or intrabodies, RNA interfering (RNAi) agents (including at least siRNAs, shRNAs, microRNAs (miRNAs), piwi, and other well-known agents). Such enhancers or modulators may be specific to FUT3 or also enhance at least one of the binding partners.

The term, “FABP1,” also known as fatty acid binding protein 1, refers to a gene that encodes the fatty acid binding protein found in liver. Fatty acid binding proteins are a family of small, highly conserved, cytoplasmic proteins that bind long-chain fatty acids and other hydrophobic ligands. FABP1 protein and FABP6 (the ileal fatty acid binding protein) are also able to bind bile acids. FABPs roles may include fatty acid uptake, transport, and metabolism.

The term “FABP1” is intended to include fragments, variants (e.g., allelic variants) and derivatives thereof. The nucleic acid and amino acid sequences of a representative human FABP1 is available to the public at the GenBank database (Gene ID 2168) (NM_001443.3 and NP_001434.1).

Nucleic acid and polypeptide sequences of FABP1 orthologs in organisms other than humans are well known and include, for example, mouse FABP1 (NM_017399.5 and NP_059095.1), rat FABP1 (NM_012556.2 and NP_036688.1), zebrafish FABP1 (NM_001044712.1 and NP_001038177.1), chimpanzee FABP1 (XM_001140263.3 and XP_001140263.1), and monkey FABP1 (XM_015112816.2 and XP_014968302.1). The term “FABP1 activity” includes the ability of a FABP1 polypeptide (and its fragments, domains, and/or motifs thereof, discussed herein) to bind its substrates, and/or mediate its activity and functions disclosed herein.

The term “agents that increase the copy number, the expression level, and/or the activity of FABP1” encompasses any natural or non-natural agent prepared, synthesized, manufactured, and/or purified by human that is capable of increasing the expression level and/or activity of a FABP1 polypeptide (and its fragments, domains, and/or motifs thereof, discussed herein). In some embodiments, the agent may increase the binding/interaction between FABP1 and other transcription factors or binding partners. In other embodiments, the agent may increase the expression of a FABP1 polypeptide. In still another embodiment, such enhancers or modulators may increase or promote the turnover rate, enhance or modulate the expression and/or the stability (e.g., the half-life), and/or change the cellular localization of FABP1, resulting in at least an increase in FABP1 levels and/or activity. In some embodiments, the agent that may decrease the copy number, the expression level, and/or the activity of biomarkers listed in Table 1, may increase the copy number, the expression level, and/or the activity of FABP1. Such enhancers or modulators may be any molecule, including but not limited to small molecule compounds, antibodies or intrabodies, RNA interfering (RNAi) agents (including at least siRNAs, shRNAs, microRNAs (miRNAs), piwi, and other well-known agents). Such enhancers or modulators may be specific to FABP1 or also enhance at least one of the binding partners.

The term, “FABP2,” also known as fatty acid binding protein 2, refers to a gene that encodes an intracellular fatty acid-binding protein that participates in the uptake, intracellular metabolism, and transport of long-chain fatty acids. The encoded protein is also involved in the modulation of cell growth and proliferation. FABP2 protein binds saturated long-chain fatty acids with high affinity, and may act as a lipid sensor to maintain energy homeostasis.

The term “FABP2” is intended to include fragments, variants (e.g., allelic variants) and derivatives thereof. The nucleic acid and amino acid sequences of a representative human FABP2 is available to the public at the GenBank database (Gene ID 2169) (NM_000134.4 and NP_000125.2).

Nucleic acid and polypeptide sequences of FABP2 orthologs in organisms other than humans are well known and include, for example, mouse FABP2 (NM_007980.3 and NP_032006.1), rat FABP2 (NM_013068.1 and NP_037200.1), zebrafish FABP2 (NM_131431.1 and NP_571506.1), chimpanzee FABP2 (XM_016952137.1 and XP_016807626.1), and monkey FABP2 (XM_015139143.2 and XP_014994629.2).

The term “FABP2 activity” includes the ability of a FABP2 polypeptide (and its fragments, domains, and/or motifs thereof, discussed herein) to bind its substrates, and/or mediate its activity and functions disclosed herein.

The term “agents that increase the copy number, the expression level, and/or the activity of FABP2” encompasses any natural or non-natural agent prepared, synthesized, manufactured, and/or purified by human that is capable of increasing the expression level and/or activity of a FABP2 polypeptide (and its fragments, domains, and/or motifs thereof, discussed herein). In some embodiments, the agent may increase the binding/interaction between FABP2 and other transcription factors or binding partners. In other embodiments, the agent may increase the expression of a FABP2 polypeptide. In still another embodiment, such enhancers or modulators may increase or promote the turnover rate, enhance or modulate the expression and/or the stability (e.g., the half-life), and/or change the cellular localization of FABP2, resulting in at least an increase in FABP2 levels and/or activity. In some embodiments, the agent that may decrease the copy number, the expression level, and/or the activity of biomarkers listed in Table 1, may increase the copy number, the expression level, and/or the activity of FABP2. Such enhancers or modulators may be any molecule, including but not limited to small molecule compounds, antibodies or intrabodies, RNA interfering (RNAi) agents (including at least siRNAs, shRNAs, microRNAs (miRNAs), piwi, and other well-known agents). Such enhancers or modulators may be specific to FABP2 or also enhance at least one of the binding partners.

The term, “CDH1” or “E-cadherin”, also known as cadherin 1, refers to a gene that encodes a classical cadherin of the cadherin superfamily. Alternative splicing results in multiple transcript variants, at least one of which encodes a preproprotein that is proteolytically processed to generate the mature glycoprotein. This calcium-dependent cell-cell adhesion protein is comprised of five extracellular cadherin repeats, a transmembrane region and a highly conserved cytoplasmic tail. Mutations in this gene are correlated with gastric, breast, colorectal, thyroid and ovarian cancer. Loss of function of this gene is thought to contribute to cancer progression by increasing proliferation, invasion, and/or metastasis. The ectodomain of this protein mediates bacterial adhesion to mammalian cells and the cytoplasmic domain is required for internalization.

The term “CDH1” is intended to include fragments, variants (e.g., allelic variants) and derivatives thereof. The nucleic acid and amino acid sequences of a representative human CDH1 is available to the public at the GenBank database (Gene ID 999). Human CDH1 variants include, but not limited to, the transcript variant 1 encoding isoform 1 (NM_004360.5 and NP_004351.1), the transcript variant 2 encoding isoform 2 (NM_001317184.2 and NP_001304113.1), the transcript variant 3 encoding isoform 3 (NM_001317185.2 and NP_001304114.1), and the transcript variant 4 encoding isoform 4 (NM_001317186.2 and NP_001304115.1).

Nucleic acid and polypeptide sequences of CDH1 orthologs in organisms other than humans are well known and include, for example, mouse CDH1 (NM_009864.3 and NP_033994.1), rat CDH1 (NM_031334.1 and NP_112624.1), chimpanzee CDH1l (XM_001168150.4 and XP_001168150.1), and monkey CDH1 (XM_015126485.2 and XP_014981971.2).

The term “CDH1 activity” includes the ability of a CDH1 polypeptide (and its fragments, domains, and/or motifs thereof, discussed herein) to bind its substrates, and/or mediate its activity and functions disclosed herein.

The term “agents that increase the copy number, the expression level, and/or the activity of CDH1” encompasses any natural or non-natural agent prepared, synthesized, manufactured, and/or purified by human that is capable of increasing the expression level and/or activity of a CDH1 polypeptide (and its fragments, domains, and/or motifs thereof, discussed herein). In some embodiments, the agent may increase the binding/interaction between CDH1 and other transcription factors or binding partners. In other embodiments, the agent may increase the expression of a CDH1 polypeptide. In still another embodiment, such enhancers or modulators may increase or promote the turnover rate, enhance or modulate the expression and/or the stability (e.g., the half-life), and/or change the cellular localization of CDH1, resulting in at least an increase in CDH1 levels and/or activity. In some embodiments, the agent that may decrease the copy number, the expression level, and/or the activity of biomarkers listed in Table 1, may increase the copy number, the expression level, and/or the activity of CDH1. Such enhancers or modulators may be any molecule, including but not limited to small molecule compounds, antibodies or intrabodies, RNA interfering (RNAi) agents (including at least siRNAs, shRNAs, microRNAs (miRNAs), piwi, and other well-known agents). Such enhancers or modulators may be specific to CDH1 or also enhance at least one of the binding partners.

The term, “BMP2,” also known as bone morphogenetic protein 2, refers to a gene that encodes a secreted ligand of the TGF-beta (transforming growth factor-beta) superfamily of proteins. Ligands of this family bind various TGF-beta receptors leading to recruitment and activation of SMAD family transcription factors that regulate gene expression. The encoded preproprotein is proteolytically processed to generate each subunit of the disulfide-linked homodimer, which plays a role in bone and cartilage development. Duplication of a regulatory region downstream of this gene causes a form of brachydactyly characterized by a malformed index finger and second toe in human patients.

The term “BMP2” is intended to include fragments, variants (e.g., allelic variants) and derivatives thereof. The nucleic acid and amino acid sequences of a representative human BMP2 is available to the public at the GenBank database (Gene ID 650) (NM_001200.4 and NP_001191.1).

Nucleic acid and polypeptide sequences of BMP2 orthologs in organisms other than humans are well known and include, for example, mouse BMP2 (NM_007553.3 and NP_031579.2), rat BMP2 (NM_017178.2 and NP_058874.2), zebrafish BMP2 (NM_131359.1 and NP_571434.1), chimpanzee BMP2 (XM_514508.3 and XP_514508.2), and monkey BMP2 (XM_001115987.3 and XP_001115987.1).

The term “BMP2 activity” includes the ability of a BMP2 polypeptide (and its fragments, domains, and/or motifs thereof, discussed herein) to bind its substrates, and/or mediate its activity and functions disclosed herein.

The term “agents that increase the copy number, the expression level, and/or the activity of BMP2” encompasses any natural or non-natural agent prepared, synthesized, manufactured, and/or purified by human that is capable of increasing the expression level and/or activity of a BMP2 polypeptide (and its fragments, domains, and/or motifs thereof, discussed herein). In some embodiments, the agent may increase the binding/interaction between BMP2 and other transcription factors or binding partners. In other embodiments, the agent may increase the expression of a BMP2 polypeptide. In still another embodiment, such enhancers or modulators may increase or promote the turnover rate, enhance or modulate the expression and/or the stability (e.g., the half-life), and/or change the cellular localization of BMP2, resulting in at least an increase in BMP2 levels and/or activity. In some embodiments, the agent that may decrease the copy number, the expression level, and/or the activity of biomarkers listed in Table 1, may increase the copy number, the expression level, and/or the activity of BMP2. Such enhancers or modulators may be any molecule, including but not limited to small molecule compounds, antibodies or intrabodies, RNA interfering (RNAi) agents (including at least siRNAs, shRNAs, microRNAs (miRNAs), piwi, and other well-known agents). Such enhancers or modulators may be specific to BMP2 or also enhance at least one of the binding partners.

The term, “TGF-β1”, also known as transforming growth factor beta 1, refers to a gene that encodes a secreted ligand of the TGF-beta (transforming growth factor-beta) superfamily of proteins. Ligands of this family bind various TGF-beta receptors leading to recruitment and activation of SMAD family transcription factors that regulate gene expression. The encoded preproprotein is proteolytically processed to generate a latency-associated peptide (LAP) and a mature peptide, and is found in either a latent form composed of a mature peptide homodimer, a LAP homodimer, and a latent TGF-beta binding protein, or in an active form consisting solely of the mature peptide homodimer. The mature peptide may also form heterodimers with other TGFB family members. This encoded protein regulates cell proliferation, differentiation and growth, and can modulate expression and activation of other growth factors including interferon gamma and tumor necrosis factor alpha. TGF-β1 is frequently upregulated in tumor cells, and mutations in this gene result in Camurati-Engelmann disease.

The term “TGF-β1” is intended to include fragments, variants (e.g., allelic variants) and derivatives thereof. The nucleic acid and amino acid sequences of a representative human TGF-β1 is available to the public at the GenBank database (Gene ID 7040) (NM_000660.7 and NP_000651.3).

Nucleic acid and polypeptide sequences of TGF-β1 orthologs in organisms other than humans are well known and include, for example, mouse TGF-β1 (NM_011577.2 and NP_035707.1), rat TGF-β1 (NM_021578.2 and NP_067589.1), chimpanzee TGF-β1 (XM_016936045.2 and XP_016791534.1), and monkey TGF-β1 (XM_028839781.1 and XP_028695614.1).

The term “TGF-β1 activity” includes the ability of a TGF-β1 polypeptide (and its fragments, domains, and/or motifs thereof, discussed herein) to bind its substrates, and/or mediate its activity and functions disclosed herein.

The term “agents that increase the copy number, the expression level, and/or the activity of TGF-β1” encompasses any natural or non-natural agent prepared, synthesized, manufactured, and/or purified by human that is capable of increasing the expression level and/or activity of a TGF-β1 polypeptide (and its fragments, domains, and/or motifs thereof, discussed herein). In some embodiments, the agent may increase the binding/interaction between TGF-β1 and other transcription factors or binding partners. In other embodiments, the agent may increase the expression of a TGF-β1 polypeptide. In still another embodiment, such enhancers or modulators may increase or promote the turnover rate, enhance or modulate the expression and/or the stability (e.g., the half-life), and/or change the cellular localization of TGF-β 1, resulting in at least an increase in TGF-β1 levels and/or activity. In some embodiments, the agent that may decrease the copy number, the expression level, and/or the activity of biomarkers listed in Table 1, may increase the copy number, the expression level, and/or the activity of TGF-β 1. Such enhancers or modulators may be any molecule, including but not limited to small molecule compounds, antibodies or intrabodies, RNA interfering (RNAi) agents (including at least siRNAs, shRNAs, microRNAs (miRNAs), piwi, and other well-known agents). Such enhancers or modulators may be specific to TGF-β1 or also enhance at least one of the binding partners.

### II. Subjects

In one embodiment, the subject for whom predicted likelihood of efficacy of an inhibitor of one or more biomarkers listed in Tables 1-2 is determined, is a mammal (e.g., mouse, rat, primate, non-human mammal, domestic animal, such as a dog, cat, cow, horse, and the like), and is preferably a human. In another embodiment, the subject is an animal model of cancer. For example, the animal model can be an orthotopic xenograft animal model of a human-derived cancer.

In another embodiment of the methods of the present invention, the subject has not undergone treatment, such as chemotherapy, radiation therapy, targeted therapy, and/or immunotherapies. In still another embodiment, the subject has undergone treatment, such as chemotherapy, radiation therapy, targeted therapy, and/or immunotherapies.

In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient. The methods of the present invention can be used to determine the responsiveness to inhibitor(s) of one or more biomarkers listed in Tables 1-2, or in combination with immunotherapy of many different cancers in subjects such as those described herein.

### III. Sample Collection, Preparation and Separation

In some embodiments, biomarker amount and/or activity measurement(s) in a sample derived from a subject is compared to a predetermined control (standard) sample. The sample from the subject is typically from a diseased tissue, such as cancer cells or tissues. The control sample can be from the same subject or from a different subject. The control sample is typically a normal, non-diseased sample. However, in some embodiments, such as for staging of disease or for evaluating the efficacy of treatment, the control sample can be from a diseased tissue. The control sample can be a combination of samples from several different subjects.

In some embodiments, the biomarker amount and/or activity measurement(s) from a subject is compared to a pre-determined level. This pre-determined level is typically obtained from normal samples. As described herein, a “pre-determined” biomarker amount and/or activity measurement(s) may be a biomarker amount and/or activity measurement(s) used to, by way of example only, evaluate a subject that may be selected for treatment (e.g., based on the number of genomic mutations and/or the number of genomic mutations causing non-functional proteins for DNA repair genes), evaluate a response to an inhibitor of one or more biomarkers listed in Tables 1-2, and/or evaluate a response to an inhibitor of one or more biomarkers listed in Tables 1-2, and a treatment with one or more additional anti-cancer therapies. A pre-determined biomarker amount and/or activity measurement(s) may be determined in populations of patients with or without cancer. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually.

In one embodiment, the amounts determined and/or compared in a method described herein are based on absolute measurements. In another embodiment, the amounts determined and/or compared in a method described herein are based on relative measurements, such as ratios (e.g., biomarker copy numbers, level, and/or activity before a treatment vs. after a treatment, such biomarker measurements relative to a spiked or man-made control, such biomarker measurements relative to the expression of a housekeeping gene, and the like). For example, the relative analysis can be based on the ratio of pre-treatment biomarker measurement as compared to post-treatment biomarker measurement. Pre-treatment biomarker measurement can be made at any time prior to initiation of anti-cancer therapy. Post-treatment biomarker measurement can be made at any time after initiation of anticancer therapy. In some embodiments, post-treatment biomarker measurements are made 1, 2, 3, 4, 5, 6, 7, 8, 9, 10, 11, 12, 13, 14, 15, 16, 17, 18, 19, 20 weeks or more after initiation of anticancer therapy, and even longer toward indefinitely for continued monitoring. Treatment can comprise anti-cancer therapy, such as a therapeutic regimen comprising one or more inhibitors of one or more biomarkers listed in Tables 1-2, and alone or in combination with other immunotherapy combination treatment.

The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

In some embodiments of the present invention the change of biomarker amount and/or activity measurement(s) from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cutoff values apply equally when the measurement is based on relative changes, such as based on the ratio of pre-treatment biomarker measurement as compared to posttreatment biomarker measurement. Biological samples can be collected from a variety of sources from a patient including a body fluid sample, cell sample, or a tissue sample comprising nucleic acids and/or proteins. “Body fluids” refer to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In a preferred embodiment, the subject and/or control sample is selected from the group consisting of cells, cell lines, histological slides, paraffin embedded tissues, biopsies, whole blood, nipple aspirate, serum, plasma, buccal scrape, saliva, cerebrospinal fluid, urine, stool, and bone marrow. In one embodiment, the sample is serum, plasma, or urine. In another embodiment, the sample is serum.

The samples can be collected from individuals repeatedly over a longitudinal period of time (e.g., once or more on the order of days, weeks, months, annually, biannually, etc.). Obtaining numerous samples from an individual over a period of time can be used to verify results from earlier detections and/or to identify an alteration in biological pattern as a result of, for example, disease progression, drug treatment, etc. For example, subject samples can be taken and monitored every month, every two months, or combinations of one, two, or three month intervals according to the present invention. In addition, the biomarker amount and/or activity measurements of the subject obtained over time can be conveniently compared with each other, as well as with those of normal controls during the monitoring period, thereby providing the subject's own values, as an internal, or personal, control for long-term monitoring.

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids. The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g., aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation is a method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermeable membrane in a process in which ions are transported through semi-permeable membranes from one solution to another under the influence of a potential gradient. Since the membranes used in electrodialysis may have the ability to selectively transport ions having positive or negative charge, reject ions of the opposite charge, or to allow species to migrate through a semipermeable membrane based on size and charge, it renders electrodialysis useful for concentration, removal, or separation of electrolytes.

Separation and purification in the present invention may include any procedure known in the art, such as capillary electrophoresis (e.g., in capillary or on-chip) or chromatography (e.g., in capillary, column or on a chip). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray. Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CIEF) allows weakly-ionizable amphoteric molecules, to be separated by electrophoresis in a pH gradient. CEC is a hybrid technique between traditional high performance liquid chromatography (HPLC) and CE.

Separation and purification techniques used in the present invention include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC), etc.

### IV. Biomarker Nucleic Acids and Polypeptides

One aspect of the present invention pertains to the use of isolated nucleic acid molecules that correspond to biomarker nucleic acids that encode a biomarker polypeptide or a portion of such a polypeptide. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences that naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

A biomarker nucleic acid molecule of the present invention can be isolated using standard molecular biology techniques and the sequence information in the database records described herein. Using all or a portion of such nucleic acid sequences, nucleic acid molecules of the present invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N Y, 1989).

A nucleic acid molecule of the present invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid molecule of the present invention can be prepared by standard synthetic techniques, e.g., using an automated DNA synthesizer.

Moreover, a nucleic acid molecule of the present invention can comprise only a portion of a nucleic acid sequence, wherein the full length nucleic acid sequence comprises a marker of the present invention or which encodes a polypeptide corresponding to a marker of the present invention. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a biomarker nucleic acid sequence. Probes based on the sequence of a biomarker nucleic acid molecule can be used to detect transcripts or genomic sequences corresponding to one or more markers of the present invention. The probe comprises a label group attached thereto, e.g., a radioisotope, a fluorescent compound, an enzyme, or an enzyme co-factor.

A biomarker nucleic acid molecules that differ, due to degeneracy of the genetic code, from the nucleotide sequence of nucleic acid molecules encoding a protein which corresponds to the biomarker, and thus encode the same protein, are also contemplated.

In addition, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

The term “allele,” which is used interchangeably herein with “allelic variant,” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. For example, biomarker alleles can differ from each other in a single nucleotide, or several nucleotides, and can include substitutions, deletions, and insertions of nucleotides. An allele of a gene can also be a form of a gene containing one or more mutations.

The term “allelic variant of a polymorphic region of gene” or “allelic variant”, used interchangeably herein, refers to an alternative form of a gene having one of several possible nucleotide sequences found in that region of the gene in the population. As used herein, allelic variant is meant to encompass functional allelic variants, non-functional allelic variants, SNPs, mutations and polymorphisms.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site. SNP's may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing, or it may have no effect on the function of the protein.

As used herein, the terms “gene” and “recombinant gene” refer to nucleic acid molecules comprising an open reading frame encoding a polypeptide corresponding to a marker of the present invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the present invention.

In another embodiment, a biomarker nucleic acid molecule is at least 7, 15, 20, 25, 30, 40, 60, 80, 100, 150, 200, 250, 300, 350, 400, 450, 550, 650, 700, 800, 900, 1000, 1100, 1200, 1300, 1400, 1500, 1600, 1700, 1800, 1900, 2000, 2200, 2400, 2600, 2800, 3000, 3500, 4000, 4500, or more nucleotides in length and hybridizes under stringent conditions to a nucleic acid molecule corresponding to a marker of the present invention or to a nucleic acid molecule encoding a protein corresponding to a marker of the present invention. As used herein, the term “hybridizes under stringent conditions” is intended to describe conditions for hybridization and washing under which nucleotide sequences at least 60% (65%, 70%, 75%, 80%, preferably 85%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

In addition to naturally-occurring allelic variants of a nucleic acid molecule of the present invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “nonessential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the present invention pertains to nucleic acid molecules encoding a polypeptide of the present invention that contain changes in amino acid residues that are not essential for activity. Such polypeptides differ in amino acid sequence from the naturally-occurring proteins which correspond to the markers of the present invention, yet retain biological activity. In one embodiment, a biomarker protein has an amino acid sequence that is at least about 40% identical, 50%, 60%, 70%, 75%, 80%, 83%, 85%, 87.5%, 90%, 91%, 92%, 93%, 94%, 95%, 96%, 97%, 98%, 99% or identical to the amino acid sequence of a biomarker protein described herein.

An isolated nucleic acid molecule encoding a variant protein can be created by introducing one or more nucleotide substitutions, additions or deletions into the nucleotide sequence of nucleic acids of the present invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

In some embodiments, the present invention further contemplates the use of anti-biomarker antisense nucleic acid molecules, i.e., molecules which are complementary to a sense nucleic acid of the present invention, e.g., complementary to the coding strand of a double-stranded cDNA molecule corresponding to a marker of the present invention or complementary to an mRNA sequence corresponding to a marker of the present invention. Accordingly, an antisense nucleic acid molecule of the present invention can hydrogen bond to (i.e., anneal with) a sense nucleic acid of the present invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the present invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the present invention are typically administered to a subject or generated in situ such that they hybridize with or bind to cellular mRNA and/or genomic DNA encoding a polypeptide corresponding to a selected marker of the present invention to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the present invention includes direct injection at a tissue site or infusion of the antisense nucleic acid into a blood- or bone marrow-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the present invention can be an α-anomeric nucleic acid molecule. An α-anomeric nucleic acid molecule forms specific double-stranded hybrids with complementary RNA in which, contrary to the usual α-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The present invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a polypeptide corresponding to a marker of the present invention can be designed based upon the nucleotide sequence of a cDNA corresponding to the marker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the present invention can be used to select a catalytic RNA having a specific ribonuclease activity from a pool of RNA molecules (see, e.g., Bartel and Szostak, 1993, Science 261:1411-1418).

The present invention also encompasses nucleic acid molecules which form triple helical structures. For example, expression of a biomarker protein can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acid molecules (see Hyrup et al. (1996) Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) supra; Perry-O'Keefe et al. (1996) Proc. Natl. Acad. Sci. U.S.A. 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. U.S.A. 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. U.S.A. 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

Another aspect of the present invention pertains to the use of biomarker proteins and biologically active portions thereof. In one embodiment, the native polypeptide corresponding to a marker can be isolated from cells or tissue sources by an appropriate purification scheme using standard protein purification techniques. In another embodiment, polypeptides corresponding to a marker of the present invention are produced by recombinant DNA techniques. Alternative to recombinant expression, a polypeptide corresponding to a marker of the present invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly, such preparations of the protein have less than about 30%, 20%, 10%, 5% (by dry weight) of chemical precursors or compounds other than the polypeptide of interest.

Biologically active portions of a biomarker polypeptide include polypeptides comprising amino acid sequences sufficiently identical to or derived from a biomarker protein amino acid sequence described herein, but which includes fewer amino acids than the full length protein, and exhibit at least one activity of the corresponding full-length protein. Typically, biologically active portions comprise a domain or motif with at least one activity of the corresponding protein. A biologically active portion of a protein of the present invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the present invention.

Preferred polypeptides have an amino acid sequence of a biomarker protein encoded by a nucleic acid molecule described herein. Other useful proteins are substantially identical (e.g., at least about 40%, preferably 50%, 60%, 70%, 75%, 80%, 83%, 85%, 88%, 90%, 91%, 92%, 93%, 94%, 95%, 96%, 97%, 98%, or 99%) to one of these sequences and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. U.S.A. 87:2264-2268, modified as in Karlin and Altschul (1993) Proc. Natl. Acad. Sci. U.S.A. 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the present invention. BLAST protein searches can be performed with the XBLAST program, score=50, wordlength=3 to obtain amino acid sequences homologous to a protein molecules of the present invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See the World Wide Web at ncbi.nlm.nih.gov. Another preferred, non-limiting example of a mathematical algorithm utilized for the comparison of sequences is the algorithm of Myers and Miller, (1988) Comput Appl Biosci, 4:11-7. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. U.S.A. 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted. The present invention also provides chimeric or fusion proteins corresponding to a biomarker protein. As used herein, a “chimeric protein” or “fusion protein” comprises all or part (preferably a biologically active part) of a polypeptide corresponding to a marker of the present invention operably linked to a heterologous polypeptide (i.e., a polypeptide other than the polypeptide corresponding to the marker). Within the fusion protein, the term “operably linked” is intended to indicate that the polypeptide of the present invention and the heterologous polypeptide are fused in-frame to each other. The heterologous polypeptide can be fused to the amino-terminus or the carboxyl-terminus of the polypeptide of the present invention.

One useful fusion protein is a GST fusion protein in which a polypeptide corresponding to a marker of the present invention is fused to the carboxyl terminus of GST sequences. Such fusion proteins can facilitate the purification of a recombinant polypeptide of the present invention.

In another embodiment, the fusion protein contains a heterologous signal sequence, immunoglobulin fusion protein, toxin, or other useful protein sequence. Chimeric and fusion proteins of the present invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the present invention can be cloned into such an expression vector such that the fusion moiety is linked in-frame to the polypeptide encompassed by the present invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the present invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the biomarker polypeptides described herein. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a biomarker protein that function as either agonists (mimetics) or as antagonists can be identified by screening combinatorial libraries of mutants, e.g., truncation mutants, of the protein of the present invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the present invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of fragments of the coding sequence of a polypeptide corresponding to a marker of the present invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the present invention (Arkin and Yourvan, 1992, Proc. Natl. Acad. Sci. U.S.A. 89:7811-7815; Delgrave et al., 1993, Protein Engineering 6(3):327-331). An isolated polypeptide or a fragment thereof (or a nucleic acid encoding such a polypeptide) corresponding to one or more biomarkers encompassed by the present invention, including the biomarkers listed in Tables 1-2, or fragments thereof, can be used as an immunogen to generate antibodies that bind to said immunogen, using standard techniques for polyclonal and monoclonal antibody preparation according to well-known methods in the art. An antigenic peptide comprises at least 8 amino acid residues and encompasses an epitope present in the respective full length molecule such that an antibody raised against the peptide forms a specific immune complex with the respective full length molecule. Preferably, the antigenic peptide comprises at least 10 amino acid residues. In one embodiment such epitopes can be specific for a given polypeptide molecule from one species, such as mouse or human (i.e., an antigenic peptide that spans a region of the polypeptide molecule that is not conserved across species is used as immunogen; such non conserved residues can be determined using an alignment such as that provided herein).

In one embodiment, an antibody, especially an intrabody, binds substantially specifically to one or more biomarkers listed in Tables 1-2, and inhibits or enhances its biological function. In another embodiment, an antibody, especially an intrabody, binds substantially specifically to the binding partner(s) of one or more biomarkers listed in Tables 1-2, such as substrates of such one or more biomarkers described herein, and inhibits or enhances its biological function, such as by interrupting its interaction to one or more biomarkers listed in Tables 1-2.

For example, a polypeptide immunogen typically is used to prepare antibodies by immunizing a suitable subject (e.g., rabbit, goat, mouse or other mammal) with the immunogen. A preferred animal is a mouse deficient in the desired target antigen. This results in a wider spectrum of antibody recognition possibilities as antibodies reactive to common mouse and human epitopes are not removed by tolerance mechanisms. An appropriate immunogenic preparation can contain, for example, a recombinantly expressed or chemically synthesized molecule or fragment thereof to which the immune response is to be generated. The preparation can further include an adjuvant, such as Freund's complete or incomplete adjuvant, or similar immunostimulatory agent. Immunization of a suitable subject with an immunogenic preparation induces a polyclonal antibody response to the antigenic peptide contained therein.

Polyclonal antibodies can be prepared as described above by immunizing a suitable subject with a polypeptide immunogen. The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well-known techniques, such as protein A chromatography, to obtain the IgG fraction. At an appropriate time after immunization, e.g., when the antibody titers are highest, antibody-producing cells can be obtained from the subject and used to prepare monoclonal antibodies by standard techniques, such as the hybridoma technique (originally described by Kohler and Milstein (1975) Nature 256:495-497) (see also Brown et al. (1981) J. Immunol. 127:539-46; Brown et al. (1980) J Biol. Chem. 255:4980-83; Yeh et al. (1976) Proc. Natl. Acad. Sci. 76:2927-31; Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well-known (see generally Kenneth, R. H. in Monoclonal Antibodies: A New Dimension In Biological Analyses, Plenum Publishing Corp., New York, New York (1980); Lerner, E. A. (1981) Yale J. Biol. Med 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically. In some embodiments, the immunization is performed in a cell or animal host that has a knockout of a target antigen of interest (e.g., does not produce the antigen prior to immunization).

Any of the many well-known protocols used for fusing lymphocytes and immortalized cell lines can be applied for the purpose of generating a monoclonal antibody against one or more biomarkers encompassed by the present invention, including the biomarkers listed in Tables 1-2, or a fragment thereof (see, e.g., Galfre, G. et al. (1977) Nature 266:55052; Gefter et al. (1977) supra; Lerner (1981) supra; Kenneth (1980) supra). Moreover, the ordinary skilled worker will appreciate that there are many variations of such methods which also would be useful.

As an alternative to preparing monoclonal antibody-secreting hybridomas, a monoclonal specific for one of the above described polypeptides can be identified and isolated by screening a recombinant combinatorial immunoglobulin library (e.g., an antibody phage display library) with the appropriate polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening an antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) (NY) 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. U.S.A. 89:3576-3580; Garrard et al. (1991) (NY) 9:1373-1377; Hoogenboom et al. (1991) Nucleic Acids Res. 19:4133-4137; Barbas et al. (1991) Proc. Natl. Acad. Sci. U.S.A. 88:7978-7982; and McCafferty et al. (1990) Nature 348:552-554.

Since it is well-known in the art that antibody heavy and light chain CDR3 domains play a particularly important role in the binding specificity/affinity of an antibody for an antigen, the recombinant monoclonal antibodies of the present invention prepared as set forth above preferably comprise the heavy and light chain CDR3s of variable regions of the antibodies described herein and well-known in the art. Similarly, the antibodies can further comprise the CDR2s of variable regions of said antibodies. The antibodies can further comprise the CDR1s of variable regions of said antibodies. In other embodiments, the antibodies can comprise any combinations of the CDRs.

The CDR1, 2, and/or 3 regions of the engineered antibodies described above can comprise the exact amino acid sequence(s) as those of variable regions of the present invention described herein. However, the ordinarily skilled artisan will appreciate that some deviation from the exact CDR sequences may be possible while still retaining the ability of the antibody, especially an intrabody, to bind a desired target, such as one or more biomarkers listed in Tables 1-2, and/or a binding partner thereof, either alone or in combination with an immunotherapy, such as the one or more biomarkers, the binding partners/substrates of such biomarkers, or an immunotherapy effectively (e.g., conservative sequence modifications). Accordingly, in another embodiment, the engineered antibody may be composed of one or more CDRs that are, for example, 50%, 60%, 70%, 80%, 85%, 90%, 91%, 92%, 93%, 94%, 95%, 96%, 97%, 98%, 99%, or 99.5% identical to one or more CDRs of the present invention described herein or otherwise publicly available.

For example, the structural features of non-human or human antibodies (e.g., a rat anti-mouse/anti-human antibody) can be used to create structurally related human antibodies, especially intrabodies, that retain at least one functional property of the antibodies of the present invention, such as binding to one or more biomarkers listed in Tables 1-2, the binding partners/substrates of such one or more biomarkers. Another functional property includes inhibiting binding of the original known, non-human or human antibodies in a competition ELISA assay.

Antibodies, immunoglobulins, and polypeptides encompassed by the present invention can be used in an isolated (e.g., purified) form or contained in a vector, such as a membrane or lipid vesicle (e.g. a liposome). Moreover, amino acid sequence modification(s) of the antibodies described herein are contemplated. For example, it may be desirable to improve the binding affinity and/or other biological properties of the antibody. It is known that when a humanized antibody is produced by simply grafting only CDRs in VH and VL of an antibody derived from a non-human animal in FRs of the VH and VL of a human antibody, the antigen binding activity is reduced in comparison with that of the original antibody derived from a non-human animal. It is considered that several amino acid residues of the VH and VL of the non-human antibody, not only in CDRs but also in FRs, are directly or indirectly associated with the antigen binding activity. Hence, substitution of these amino acid residues with different amino acid residues derived from FRs of the VH and VL of the human antibody would reduce binding activity and can be corrected by replacing the amino acids with amino acid residues of the original antibody derived from a non-human animal.

Similarly, modifications and changes may be made in the structure of the antibodies described herein, and in the DNA sequences encoding them, and still obtain a functional molecule that encodes an antibody and polypeptide with desirable characteristics. For example, antibody glycosylation patterns can be modulated to, for example, increase stability. By “altering” is meant deleting one or more carbohydrate moieties found in the antibody, and/or adding one or more glycosylation sites that are not present in the antibody. Glycosylation of antibodies is typically N-linked. “N-linked” refers to the attachment of the carbohydrate moiety to the side chain of an asparagine residue. The tripeptide sequences asparagine-X-serine and asparagines-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the antibody is conveniently accomplished by altering the amino acid sequence such that it contains one or more of the above-described tripeptide sequences (for N-linked glycosylation sites). Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. For example, such methods are described in WO87/05330.

Similarly, removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Sojahr et al. (1987) and by Edge et al. (1981). Enzymatic cleavage of carbohydrate moieties on antibodies can be achieved by the use of a variety of endo-and exo-glycosidases as described by Thotakura et al. (1987).

Other modifications can involve the formation of immunoconjugates. For example, in one type of covalent modification, antibodies or proteins are covalently linked to one of a variety of non proteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Conjugation of antibodies or other proteins of the present invention with heterologous agents can be made using a variety of bifunctional protein coupling agents including but not limited to N-succinimidyl (2-pyridyldithio) propionate (SPDP), succinimidyl (N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6diisocyanate), and bis-active fluorine compounds (such as 1,5-difluoro-2,4-dinitrobenzene). For example, carbon labeled 1-isothiocyanatobenzyl methyldiethylene triaminepentaacetic acid (MX-DTPA) is an exemplary chelating agent for conjugation of radionucleotide to the antibody (WO 94/11026).

In another aspect, the present invention features antibodies conjugated to a therapeutic moiety, such as a cytotoxin, a drug, and/or a radioisotope. When conjugated to a cytotoxin, these antibody conjugates are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). An antibody of the present invention can be conjugated to a radioisotope, e.g., radioactive iodine, to generate cytotoxic radiopharmaceuticals for treating a related disorder, such as a cancer.

Conjugated antibodies, in addition to therapeutic utility, can be useful for diagnostically or prognostically to monitor polypeptide levels in tissue as part of a clinical testing procedure, e.g., to determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include a flag tag, a myc tag, an hemagglutinin (HA) tag, streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin (PE); an example of a luminescent material includes luminol; examples of bioluminescent materials include luciferase, luciferin, and aequorin, and examples of suitable radioactive material include ¹²⁵I, ¹³¹I, ³⁵S, or ³H. As used herein, the term “labeled”, with regard to the antibody, is intended to encompass direct labeling of the antibody by coupling (i.e., physically linking) a detectable substance, such as a radioactive agent or a fluorophore (e.g. fluorescein isothiocyanate (FITC) or phycoerythrin (PE) or indocyanine (Cy5)) to the antibody, as well as indirect labeling of the antibody by reactivity with a detectable substance.

The antibody conjugates of the present invention can be used to modify a given biological response. The therapeutic moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, Pseudomonas exotoxin, or diphtheria toxin; a protein such as tumor necrosis factor or interferon-.gamma.; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other cytokines or growth factors.

In one embodiment, an antibody for use in the instant invention is a bispecific or multispecific antibody. A bispecific antibody has binding sites for two different antigens within a single antibody polypeptide. Antigen binding may be simultaneous or sequential. Triomas and hybrid hybridomas are two examples of cell lines that can secrete bispecific antibodies. Examples of bispecific antibodies produced by a hybrid hybridoma or a trioma are disclosed in U.S. Pat. No. 4,474,893. Bispecific antibodies have been constructed by chemical means (Staerz et al. (1985) Nature 314:628, and Perez et al. (1985) Nature 316:354) and hybridoma technology (Staerz and Bevan (1986) Proc. Natl. Acad. Sci. U.S.A., 83:1453, and Staerz and Bevan (1986) Immunol. Today 7:241). Bispecific antibodies are also described in U.S. Pat. No. 5,959,084. Fragments of bispecific antibodies are described in U.S. Pat. No. 5,798,229.

Bispecific agents can also be generated by making heterohybridomas by fusing hybridomas or other cells making different antibodies, followed by identification of clones producing and co-assembling both antibodies. They can also be generated by chemical or genetic conjugation of complete immunoglobulin chains or portions thereof such as Fab and Fv sequences. The antibody component can bind to a polypeptide or a fragment thereof of one or more biomarkers encompassed by the present invention, including one or more biomarkers listed in Tables 1-2, or a fragment thereof. In one embodiment, the bispecific antibody could specifically bind to both a polypeptide or a fragment thereof and its natural binding partner(s) or a fragment(s) thereof. Techniques for modulating antibodies, such as humanization, conjugation, recombinant techniques, and the like are well-known in the art.

In another aspect of this invention, peptides or peptide mimetics can be used to modulate expression (e.g., increase expression or decrease expression) and/or activity (e.g., agonize or antagonize) of one or more biomarkers encompassed by the present invention, including one or more biomarkers listed in Tables 1-2, or a fragment(s) thereof. In one embodiment, variants of one or more biomarkers listed in Tables 1-2 that function as a modulating agent for the respective full length protein, can be identified by screening combinatorial libraries of mutants, e.g., truncation mutants, for antagonist activity. In one embodiment, a variegated library of variants is generated by combinatorial mutagenesis at the nucleic acid level and is encoded by a variegated gene library. A variegated library of variants can be produced, for instance, by enzymatically ligating a mixture of synthetic oligonucleotides into gene sequences such that a degenerate set of potential polypeptide sequences is expressible as individual polypeptides containing the set of polypeptide sequences therein. There are a variety of methods which can be used to produce libraries of polypeptide variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential polypeptide sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a polypeptide coding sequence can be used to generate a variegated population of polypeptide fragments for screening and subsequent selection of variants of a given polypeptide. In one embodiment, a library of coding sequence fragments can be generated by treating a double stranded PCR fragment of a polypeptide coding sequence with a nuclease under conditions wherein nicking occurs only about once per polypeptide, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of interest (Arkin and Youvan (1992) Proc. Natl. Acad. Sci. U.S.A. 89:7811-7815; Delagrave et al. (1993) Protein Eng. 6(3):327-331). In one embodiment, cell based assays can be exploited to analyze a variegated polypeptide library. For example, a library of expression vectors can be transfected into a cell line which ordinarily synthesizes one or more biomarkers encompassed by the present invention, including one or more biomarkers listed in Tables 1-2, or a fragment thereof. The transfected cells are then cultured such that the full length polypeptide and a particular mutant polypeptide are produced and the effect of expression of the mutant on the full length polypeptide activity in cell supernatants can be detected, e.g., by any of a number of functional assays. Plasmid DNA can then be recovered from the cells which score for inhibition, or alternatively, potentiation of full length polypeptide activity, and the individual clones further characterized.

Systematic substitution of one or more amino acids of a polypeptide amino acid sequence with a D-amino acid of the same type (e.g., D-lysine in place of L-lysine) can be used to generate more stable peptides. In addition, constrained peptides comprising a polypeptide amino acid sequence of interest or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide. The amino acid sequences described herein will enable those of skill in the art to produce polypeptides corresponding peptide sequences and sequence variants thereof. Such polypeptides can be produced in prokaryotic or eukaryotic host cells by expression of polynucleotides encoding the peptide sequence, frequently as part of a larger polypeptide. Alternatively, such peptides can be synthesized by chemical methods. Methods for expression of heterologous proteins in recombinant hosts, chemical synthesis of polypeptides, and in vitro translation are well-known in the art and are described further in Maniatis et al. Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif; Merrifield, J. (1969) J. Am. Chem. Soc. 91:501; Chaiken I. M. (1981) CRC Crit. Rev. Biochem. 11: 255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference).

Peptides can be produced, typically by direct chemical synthesis. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments encompassed by the present invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others. Peptides described herein can be used therapeutically to treat disease, e.g., by altering costimulation in a patient.

Peptidomimetics (Fauchere (1986) Adv. Drug Res. 15:29; Veber and Freidinger (1985) TINS p. 392; and Evans et al. (1987) J. Med Chem. 30:1229, which are incorporated herein by reference) are usually developed with the aid of computerized molecular modeling. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biological or pharmacological activity), but have one or more peptide linkages optionally replaced by a linkage selected from the group consisting of: —CH2NH—, —CH2S—, —CH2-CH2-, —CH═CH— (cis and trans), —COCH2-, —CH(OH)CH2-, and —CH2SO—, by methods known in the art and further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2-); Spatola, A. F. et al. (1986) Life Sci. 38:1243-1249 (—CH2-S); Hann, M. M. (1982) J. Chem. Soc. Perkin Trans. L 307-314 (—CH—CH—, cis and trans); Almquist, R. G. et al. (190) J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982)(—CH(OH)CH2-); Holladay, M. W. et al. (1983) Tetrahedron Lett. (1983) 24:4401-4404 (—C(OH)CH2-); and Hruby, V. J. (1982) Life Sci. (1982) 31:189-199 (—CH2-S—); each of which is incorporated herein by reference. A particularly preferred non-peptide linkage is —CH2NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macropolypeptides(s) to which the peptidomimetic binds to produce the therapeutic effect. Derivatization (e.g., labeling) of peptidomimetics should not substantially interfere with the desired biological or pharmacological activity of the peptidomimetic.

Also encompassed by the present invention are small molecules which can modulate (either enhance or inhibit) interactions, e.g., between biomarkers described herein or listed in Tables 1-2 and their natural binding partners. The small molecules of the present invention can be obtained using any of the numerous approaches in combinatorial library methods known in the art, including: spatially addressable parallel solid phase or solution phase libraries; synthetic library methods requiring deconvolution; the ‘one-bead one-compound’ library method; and synthetic library methods using affinity chromatography selection. (Lam, K. S. (1997) Anticancer Drug Des. 12:145).

Examples of methods for the synthesis of molecular libraries can be found in the art, for example in: DeWitt et al. (1993) Proc. Natl. Acad Sci. U.S.A. 90:6909; Erb et al. (1994) Proc. Natl. Acad Sci. U.S.A. 91:11422; Zuckermann et al. (1994) J. Med Chem. 37:2678; Cho et al. (1993) Science 261:1303; Carrell et al. (1994) Angew. Chem. Int. Ed Engl. 33:2059; Carell et al. (1994) Angew. Chem. Int. Ed Engl. 33:2061; and in Gallop et al. (1994) J. Med Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc. Natl. Acad Sci. U.S.A. 89:1865-1869) or on phage (Scott and Smith (1990) Science 249:386-390); (Devlin (1990) Science 249:404-406); (Cwirla et al. (1990) Proc. Natl. Acad Sci. U.S.A. 87:6378-6382); (Felici (1991) J. Mol. Biol. 222:301-310); (Ladner supra.). Compounds can be screened in cell based or non-cell based assays. Compounds can be screened in pools (e.g. multiple compounds in each testing sample) or as individual compounds.

Chimeric or fusion proteins can be prepared for the inhibitor(s) of one or more biomarkers listed in Tables 1-2, and/or agents for the immunotherapies described herein, such as inhibitors to the biomarkers encompassed by the present invention, including the biomarkers listed in Tables 1-2, or fragments thereof. As used herein, a “chimeric protein” or “fusion protein” comprises one or more biomarkers encompassed by the present invention, including one or more biomarkers listed in Tables 1-2, or a fragment thereof, operatively linked to another polypeptide having an amino acid sequence corresponding to a protein which is not substantially homologous to the respective biomarker. In a preferred embodiment, the fusion protein comprises at least one biologically active portion of one or more biomarkers encompassed by the present invention, including one or more biomarkers listed in Tables 1-2, or fragments thereof. Within the fusion protein, the term “operatively linked” is intended to indicate that the biomarker sequences and the non-biomarker sequences are fused in-frame to each other in such a way as to preserve functions exhibited when expressed independently of the fusion. The “another” sequences can be fused to the N-terminus or C-terminus of the biomarker sequences, respectively.

Such a fusion protein can be produced by recombinant expression of a nucleotide sequence encoding the first peptide and a nucleotide sequence encoding the second peptide. The second peptide may optionally correspond to a moiety that alters the solubility, affinity, stability or valency of the first peptide, for example, an immunoglobulin constant region. In another preferred embodiment, the first peptide consists of a portion of a biologically active molecule (e.g. the extracellular portion of the polypeptide or the ligand binding portion). The second peptide can include an immunoglobulin constant region, for example, a human Cγ1 domain or Cγ 4 domain (e.g., the hinge, CH2 and CH3 regions of human IgCγ1, or human IgCγ4, see e.g., Capon et al. U.S. Pat. Nos. 5,116,964; 5,580,756; 5,844,095 and the like, incorporated herein by reference). Such constant regions may retain regions which mediate effector function (e.g. Fc receptor binding) or may be altered to reduce effector function. A resulting fusion protein may have altered solubility, binding affinity, stability and/or valency (i.e., the number of binding sites available per polypeptide) as compared to the independently expressed first peptide, and may increase the efficiency of protein purification. Fusion proteins and peptides produced by recombinant techniques can be secreted and isolated from a mixture of cells and medium containing the protein or peptide. Alternatively, the protein or peptide can be retained cytoplasmically and the cells harvested, lysed and the protein isolated. A cell culture typically includes host cells, media and other byproducts. Suitable media for cell culture are well-known in the art. Protein and peptides can be isolated from cell culture media, host cells, or both using techniques known in the art for purifying proteins and peptides. Techniques for transfecting host cells and purifying proteins and peptides are known in the art.

Preferably, a fusion protein encompassed by the present invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992).

The fusion proteins encompassed by the present invention can be used as immunogens to produce antibodies in a subject. Such antibodies may be used to purify the respective natural polypeptides from which the fusion proteins were generated, or in screening assays to identify polypeptides which inhibit the interactions between one or more biomarkers polypeptide or a fragment thereof and its natural binding partner(s) or a fragment(s) thereof.

Also provided herein are compositions comprising one or more nucleic acids comprising or capable of expressing at least 1, 2, 3, 4, 5, 10, 20 or more small nucleic acids or antisense oligonucleotides or derivatives thereof, wherein said small nucleic acids or antisense oligonucleotides or derivatives thereof in a cell specifically hybridize (e.g., bind) under cellular conditions, with cellular nucleic acids (e.g., small non-coding RNAS such as miRNAs, pre-miRNAs, pri-miRNAs, miRNA*, anti-miRNA, a miRNA binding site, a variant and/or functional variant thereof, cellular mRNAs or a fragments thereof). In one embodiment, expression of the small nucleic acids or antisense oligonucleotides or derivatives thereof in a cell can inhibit expression or biological activity of cellular nucleic acids and/or proteins, e.g., by inhibiting transcription, translation and/or small nucleic acid processing of, for example, one or more biomarkers encompassed by the present invention, including one or more biomarkers listed in Tables 1-2, or fragment(s) thereof. In one embodiment, the small nucleic acids or antisense oligonucleotides or derivatives thereof are small RNAs (e.g., microRNAs) or complements of small RNAs. In another embodiment, the small nucleic acids or antisense oligonucleotides or derivatives thereof can be single or double stranded and are at least six nucleotides in length and are less than about 1000, 900, 800, 700, 600, 500, 400, 300, 200, 100, 50, 40, 30, 25, 24, 23, 22, 21, 20, 19, 18, 17, 16, 15, or 10 nucleotides in length. In another embodiment, a composition may comprise a library of nucleic acids comprising or capable of expressing small nucleic acids or antisense oligonucleotides or derivatives thereof, or pools of said small nucleic acids or antisense oligonucleotides or derivatives thereof. A pool of nucleic acids may comprise about 25, 5-10, 10-20, 10-30 or more nucleic acids comprising or capable of expressing small nucleic acids or antisense oligonucleotides or derivatives thereof. In one embodiment, binding may be by conventional base pair complementarity, or, for example, in the case of binding to DNA duplexes, through specific interactions in the major groove of the double helix. In general, “antisense” refers to the range of techniques generally employed in the art, and includes any process that relies on specific binding to oligonucleotide sequences.

It is well-known in the art that modifications can be made to the sequence of a miRNA or a pre-miRNA without disrupting miRNA activity. As used herein, the term “functional variant” of a miRNA sequence refers to an oligonucleotide sequence that varies from the natural miRNA sequence, but retains one or more functional characteristics of the miRNA (e.g. cancer cell proliferation inhibition, induction of cancer cell apoptosis, enhancement of cancer cell susceptibility to chemotherapeutic agents, specific miRNA target inhibition). In some embodiments, a functional variant of a miRNA sequence retains all of the functional characteristics of the miRNA. In certain embodiments, a functional variant of a miRNA has a nucleobase sequence that is a least about 60%, 65%, 70%, 75%, 80%, 85%, 90%, 91%, 92%, 93%, 94%, 95%, 96%, 97%, 98% or 99% identical to the miRNA or precursor thereof over a region of about 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases, or that the functional variant hybridizes to the complement of the miRNA or precursor thereof under stringent hybridization conditions. Accordingly, in certain embodiments the nucleobase sequence of a functional variant is capable of hybridizing to one or more target sequences of the miRNA.

miRNAs and their corresponding stem-loop sequences described herein may be found in miRBase, an online searchable database of miRNA sequences and annotation, found on the world wide web at microrna.sanger.ac.uk. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence.

In some embodiments, miRNA sequences encompassed by the present invention may be associated with a second RNA sequence that may be located on the same RNA molecule or on a separate RNA molecule as the miRNA sequence. In such cases, the miRNA sequence may be referred to as the active strand, while the second RNA sequence, which is at least partially complementary to the miRNA sequence, may be referred to as the complementary strand. The active and complementary strands are hybridized to create a double-stranded RNA that is similar to a naturally occurring miRNA precursor. The activity of a miRNA may be optimized by maximizing uptake of the active strand and minimizing uptake of the complementary strand by the miRNA protein complex that regulates gene translation. This can be done through modification and/or design of the complementary strand.

In some embodiments, the complementary strand is modified so that a chemical group other than a phosphate or hydroxyl at its 5′ terminus. The presence of the 5′ modification apparently eliminates uptake of the complementary strand and subsequently favors uptake of the active strand by the miRNA protein complex. The 5′ modification can be any of a variety of molecules known in the art, including NH₂, NHCOCH₃, and biotin.

In another embodiment, the uptake of the complementary strand by the miRNA pathway is reduced by incorporating nucleotides with sugar modifications in the first 2-6 nucleotides of the complementary strand. It should be noted that such sugar modifications can be combined with the 5′ terminal modifications described above to further enhance miRNA activities.

In some embodiments, the complementary strand is designed so that nucleotides in the 3′ end of the complementary strand are not complementary to the active strand. This results in double-strand hybrid RNAs that are stable at the 3′ end of the active strand but relatively unstable at the 5′ end of the active strand. This difference in stability enhances the uptake of the active strand by the miRNA pathway, while reducing uptake of the complementary strand, thereby enhancing miRNA activity.

Small nucleic acid and/or antisense constructs of the methods and compositions presented herein can be delivered, for example, as an expression plasmid which, when transcribed in the cell, produces RNA which is complementary to at least a unique portion of cellular nucleic acids (e.g., small RNAs, mRNA, and/or genomic DNA). Alternatively, the small nucleic acid molecules can produce RNA which encodes mRNA, miRNA, pre-miRNA, pri-miRNA, miRNA*, anti-miRNA, or a miRNA binding site, or a variant thereof. For example, selection of plasmids suitable for expressing the miRNAs, methods for inserting nucleic acid sequences into the plasmid, and methods of delivering the recombinant plasmid to the cells of interest are within the skill in the art. See, for example, Zeng et al. (2002) Mol. Cell 9:1327-1333; Tuschl (2002), Nat. Biotechnol. 20:446-448; Brummelkamp et al. (2002) Science 296:550-553; Miyagishi et al. (2002) Nat. Biotechnol. 20:497-500; Paddison et al. (2002) Genes Dev. 16:948-958; Lee et al. (2002) Nat. Biotechnol. 20:500-505; and Paul et al. (2002) Nat. Biotechnol. 20:505-508, the entire disclosures of which are herein incorporated by reference.

Alternatively, small nucleic acids and/or antisense constructs are oligonucleotide probes that are generated ex vivo and which, when introduced into the cell, results in hybridization with cellular nucleic acids. Such oligonucleotide probes are preferably modified oligonucleotides that are resistant to endogenous nucleases, e.g., exonucleases and/or endonucleases, and are therefore stable in vivo. Exemplary nucleic acid molecules for use as small nucleic acids and/or antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668.

Antisense approaches may involve the design of oligonucleotides (either DNA or RNA) that are complementary to cellular nucleic acids (e.g., complementary to biomarkers listed in Tables 1-2). Absolute complementarity is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a nucleic acid (e.g., RNA) it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well (Wagner (1994) Nature 372:333). Therefore, oligonucleotides complementary to either the 5′ or 3′ untranslated, non-coding regions of genes could be used in an antisense approach to inhibit translation of endogenous mRNAs. Oligonucleotides complementary to the 5′ untranslated region of the mRNA may include the complement of the AUG start codon. Antisense oligonucleotides complementary to mRNA coding regions are less efficient inhibitors of translation but could also be used in accordance with the methods and compositions presented herein. Whether designed to hybridize to the 5′, 3′ or coding region of cellular mRNAs, small nucleic acids and/or antisense nucleic acids should be at least six nucleotides in length, and can be less than about 1000, 900, 800, 700, 600, 500, 400, 300, 200, 100, 50, 40, 30, 25, 24, 23, 22, 21, 20, 19, 18, 17, 16, 15, or 10 nucleotides in length.

Regardless of the choice of target sequence, it is preferred that in vitro studies are first performed to quantitate the ability of the antisense oligonucleotide to inhibit gene expression. In one embodiment, these studies utilize controls that distinguish between antisense gene inhibition and nonspecific biological effects of oligonucleotides. In another embodiment, these studies compare levels of the target nucleic acid or protein with that of an internal control nucleic acid or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

Small nucleic acids and/or antisense oligonucleotides can be DNA or RNA or chimeric mixtures or derivatives or modified versions thereof, single-stranded or double-stranded. Small nucleic acids and/or antisense oligonucleotides can be modified at the base moiety, sugar moiety, or phosphate backbone, for example, to improve stability of the molecule, hybridization, etc., and may include other appended groups such as peptides (e.g., for targeting host cell receptors), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. U.S.A. 84:648-652; PCT Publication No. WO88/09810) or the blood-brain barrier (see, e.g., PCT Publication No. WO89/10134), hybridization-triggered cleavage agents. (See, e.g., Krol et al. (1988) BioTech. 6:958-976) or intercalating agents. (See, e.g., Zon (1988) Pharm. Res. 5:539549). To this end, small nucleic acids and/or antisense oligonucleotides may be conjugated to another molecule, e.g., a peptide, hybridization triggered cross-linking agent, transport agent, hybridization-triggered cleavage agent, etc.

Small nucleic acids and/or antisense oligonucleotides may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Small nucleic acids and/or antisense oligonucleotides may also comprise at least one modified sugar moiety selected from the group including but not limited to arabinose, 2-fluoroarabinose, xylulose, and hexose.

In certain embodiments, a compound comprises an oligonucleotide (e.g., a miRNA or miRNA encoding oligonucleotide) conjugated to one or more moieties which enhance the activity, cellular distribution or cellular uptake of the resulting oligonucleotide. In certain such embodiments, the moiety is a cholesterol moiety (e.g., antagomirs) or a lipid moiety or liposome conjugate. Additional moieties for conjugation include carbohydrates, phospholipids, biotin, phenazine, folate, phenanthridine, anthraquinone, acridine, fluoresceins, rhodamines, coumarins, and dyes. In certain embodiments, a conjugate group is attached directly to the oligonucleotide. In certain embodiments, a conjugate group is attached to the oligonucleotide by a linking moiety selected from amino, hydroxyl, carboxylic acid, thiol, unsaturations (e.g., double or triple bonds), 8-amino-3,6-dioxaoctanoic acid (ADO), succinimidyl 4-(N-maleimidomethyl) cyclohexane-1-carboxylate (SMCC), 6-aminohexanoic acid (AHEX or AHA), substituted C1-C10 alkyl, substituted or unsubstituted C2-C10 alkenyl, and substituted or unsubstituted C2-C10 alkynyl. In certain such embodiments, a substituent group is selected from hydroxyl, amino, alkoxy, carboxy, benzyl, phenyl, nitro, thiol, thioalkoxy, halogen, alkyl, aryl, alkenyl and alkynyl. In certain such embodiments, the compound comprises the oligonucleotide having one or more stabilizing groups that are attached to one or both termini of the oligonucleotide to enhance properties such as, for example, nuclease stability. Included in stabilizing groups are cap structures. These terminal modifications protect the oligonucleotide from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps.

Suitable cap structures include a 4′,5′-methylene nucleotide, a 1-(beta-D-erythrofuranosyl) nucleotide, a 4′-thio nucleotide, a carbocyclic nucleotide, a 1,5-anhydrohexitol nucleotide, an L-nucleotide, an alpha-nucleotide, a modified base nucleotide, a phosphorodithioate linkage, a threo-pentofuranosyl nucleotide, an acyclic 3′,4′-seco nucleotide, an acyclic 3,4-dihydroxybutyl nucleotide, an acyclic 3,5-dihydroxypentyl nucleotide, a 3′-3′-inverted nucleotide moiety, a 3′-3′-inverted abasic moiety, a 3′-2′-inverted nucleotide moiety, a 3′-2′-inverted abasic moiety, a 1,4-butanediol phosphate, a 3′-phosphoramidate, a hexylphosphate, an aminohexyl phosphate, a 3′-phosphate, a 3′-phosphorothioate, a phosphorodithioate, a bridging methylphosphonate moiety, and a non-bridging methylphosphonate moiety 5′-amino-alkyl phosphate, a 1,3-diamino-2-propyl phosphate, 3-aminopropyl phosphate, a 6-aminohexyl phosphate, a 1,2-aminododecyl phosphate, a hydroxypropyl phosphate, a 5′-5′-inverted nucleotide moiety, a 5′-5′-inverted abasic moiety, a 5′-phosphoramidate, a 5′-phosphorothioate, a 5′-amino, a bridging and/or non-bridging 5′-phosphoramidate, a phosphorothioate, and a 5′-mercapto moiety.

Small nucleic acids and/or antisense oligonucleotides can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, small nucleic acids and/or antisense oligonucleotides comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof. In a further embodiment, small nucleic acids and/or antisense oligonucleotides are α-anomeric oligonucleotides. An α-anomeric oligonucleotide forms specific double-stranded hybrids with complementary RNA in which, contrary to the usual b-units, the strands run parallel to each other (Gautier et al. (1987) Nucl. Acids Res. 15:6625-6641). The oligonucleotide is a 2′-O-methylribonucleotide (Inoue et al. (1987) Nucl. Acids Res. 15:61316148), or a chimeric RNA-DNA analogue (Inoue et al. (1987) FEBS Lett. 215:327-330).

Small nucleic acids and/or antisense oligonucleotides of the methods and compositions presented herein may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988) Nucl. Acids Res. 16:3209, methylphosphonate oligonucleotides can be prepared by use of controlled pore glass polymer supports (Sarin et al. (1988) Proc. Natl. Acad. Sci. U.S.A. 85:7448-7451), etc. For example, an isolated miRNA can be chemically synthesized or recombinantly produced using methods known in the art. In some instances, miRNA are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., U.S.A.), Pierce Chemical (part of Perbio Science, Rockford, Ill., U.S.A.), Glen Research (Sterling, Va., U.S.A.), ChemGenes (Ashland, Mass., U.S.A.), Cruachem (Glasgow, UK), and Exiqon (Vedbaek, Denmark).

Small nucleic acids and/or antisense oligonucleotides can be delivered to cells in vivo. A number of methods have been developed for delivering small nucleic acids and/or antisense oligonucleotides DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

In one embodiment, small nucleic acids and/or antisense oligonucleotides may comprise or be generated from double stranded small interfering RNAs (siRNAs), in which sequences fully complementary to cellular nucleic acids (e.g. mRNAs) sequences mediate degradation or in which sequences incompletely complementary to cellular nucleic acids (e.g., mRNAs) mediate translational repression when expressed within cells, or piwiRNAs. In another embodiment, double stranded siRNAs can be processed into single stranded antisense RNAs that bind single stranded cellular RNAs (e.g., microRNAs) and inhibit their expression. RNA interference (RNAi) is the process of sequence-specific, post-transcriptional gene silencing in animals and plants, initiated by double-stranded RNA (dsRNA) that is homologous in sequence to the silenced gene. in vivo, long dsRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (293) and HeLa cells (Elbashir et al. (2001) Nature 411:494-498). Accordingly, translation of a gene in a cell can be inhibited by contacting the cell with short double stranded RNAs having a length of about 15 to 30 nucleotides or of about 18 to 21 nucleotides or of about 19 to 21 nucleotides. Alternatively, a vector encoding for such siRNAs or short hairpin RNAs (shRNAs) that are metabolized into siRNAs can be introduced into a target cell (see, e.g., McManus et al. (2002) RNA 8:842; Xia et al. (2002) Nat. Biotechnol. 20:1006; and Brummelkamp et al. (2002) Science 296:550). Vectors that can be used are commercially available, e.g., from OligoEngine under the name pSuper RNAi System™.

Ribozyme molecules designed to catalytically cleave cellular mRNA transcripts can also be used to prevent translation of cellular mRNAs and expression of cellular polypeptides, or both (See, e.g., PCT International Publication WO90/11364, published Oct. 4, 1990; Sarver et al. (1990) Science 247:1222-1225 and U.S. Pat. No. 5,093,246). While ribozymes that cleave mRNA at site specific recognition sequences can be used to destroy cellular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well-known in the art and is described more fully in Haseloff and Gerlach (1988) Nature 334:585-591. The ribozyme may be engineered so that the cleavage recognition site is located near the 5′ end of cellular mRNAs; i.e., to increase efficiency and minimize the intracellular accumulation of non-functional mRNA transcripts.

The ribozymes of the methods presented herein also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug et al. (1984) Science 224:574-578; Zaug et al. (1986) Science 231:470-475; Zaug et al. (1986) Nature 324:429-433; WO 88/04300; and Been et al. (1986) Cell 47:207-216). The Cech-type ribozymes have an eight base pair active site which hybridizes to a target RNA sequence whereafter cleavage of the target RNA takes place. The methods and compositions presented herein encompasses those Cech-type ribozymes which target eight base-pair active site sequences that are present in cellular genes.

As in the antisense approach, the ribozymes can be composed of modified oligonucleotides (e.g., for improved stability, targeting, etc.). A preferred method of delivery involves using a DNA construct “encoding” the ribozyme under the control of a strong constitutive pol III or pol II promoter, so that transfected cells will produce sufficient quantities of the ribozyme to destroy endogenous cellular messages and inhibit translation. Because ribozymes unlike antisense molecules, are catalytic, a lower intracellular concentration is required for efficiency.

Nucleic acid molecules to be used in triple helix formation for the inhibition of transcription of cellular genes are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex. Small nucleic acids (e.g., miRNAs, pre-miRNAs, pri-miRNAs, miRNA*, anti-miRNA, or a miRNA binding site, or a variant thereof), antisense oligonucleotides, ribozymes, and triple helix molecules of the methods and compositions presented herein may be prepared by any method known in the art for the synthesis of DNA and RNA molecules. These include techniques for chemically synthesizing oligodeoxyribonucleotides and oligoribonucleotides well-known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone. One of skill in the art will readily understand that polypeptides, small nucleic acids, and antisense oligonucleotides can be further linked to another peptide or polypeptide (e.g., a heterologous peptide), e.g., that serves as a means of protein detection. Non-limiting examples of label peptide or polypeptide moieties useful for detection in the invention include, without limitation, suitable enzymes such as horseradish peroxidase, alkaline phosphatase, beta-galactosidase, or acetylcholinesterase; epitope tags, such as FLAG, MYC, HA, or HIS tags; fluorophores such as green fluorescent protein; dyes; radioisotopes; digoxygenin; biotin; antibodies; polymers; as well as others known in the art, for example, in Principles of Fluorescence Spectroscopy, Joseph R. Lakowicz (Editor), Plenum Pub Corp, 2nd edition (July 1999).

The modulatory agents described herein (e.g., antibodies, small molecules, peptides, fusion proteins, or small nucleic acids) can be incorporated into pharmaceutical compositions and administered to a subject in vivo. The compositions may contain a single such molecule or agent or any combination of agents described herein. “Single active agents” described herein can be combined with other pharmacologically active compounds (“second active agents”) known in the art according to the methods and compositions provided herein.

The production and use of biomarker nucleic acid and/or biomarker polypeptide molecules described herein can be facilitated by using standard recombinant techniques. In some embodiments, such techniques use vectors, preferably expression vectors, containing a nucleic acid encoding a biomarker polypeptide or a portion of such a polypeptide. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the present invention is intended to include such other forms of expression vectors, such as viral vectors (e.g., replication defective retroviruses, adenoviruses and adeno-associated viruses), which serve equivalent functions.

The recombinant expression vectors of the present invention comprise a nucleic acid of the present invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, CA (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the present invention can be introduced into host cells to thereby produce proteins or peptides, including fusion proteins or peptides, encoded by nucleic acids as described herein.

The recombinant expression vectors for use in the present invention can be designed for expression of a polypeptide corresponding to a marker of the present invention in prokaryotic (e.g., E. coli) or eukaryotic cells (e.g., insect cells {using baculovirus expression vectors}, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, MA) and pRIT5 (Pharmacia, Piscataway, NJ) which fuse glutathione S-transferase (GST), maltose E binding protein, or protein A, respectively, to the target recombinant protein.

Examples of suitable inducible non-fusion E. coli expression vectors include pTrc (Amann et al., 1988, Gene 69:301-315) and pET 11d (Studier et al., p. 60-89, In Gene Expression Technology: Methods in Enzymology vol. 185, Academic Press, San Diego, C A, 1991). Target biomarker nucleic acid expression from the pTrc vector relies on host RNA polymerase transcription from a hybrid trp-lac fusion promoter. Target biomarker nucleic acid expression from the pET 11d vector relies on transcription from a T7 gn10-lac fusion promoter mediated by a co-expressed viral RNA polymerase (T7 gn1). This viral polymerase is supplied by host strains BL21 (DE3) or HMS174 (DE3) from a resident prophage harboring a T7 gn1 gene under the transcriptional control of the lacUV 5 promoter.

One strategy to maximize recombinant protein expression in E. coli is to express the protein in a host bacterium with an impaired capacity to proteolytically cleave the recombinant protein (Gottesman, p. 119-128, In Gene Expression Technology: Methods in Enzymology vol. 185, Academic Press, San Diego, C A, 1990. Another strategy is to alter the nucleic acid sequence of the nucleic acid to be inserted into an expression vector so that the individual codons for each amino acid are those preferentially utilized in E. coli (Wada et al., 1992, Nucleic Acids Res. 20:2111-2118). Such alteration of nucleic acid sequences of the present invention can be carried out by standard DNA synthesis techniques.

In another embodiment, the expression vector is a yeast expression vector. Examples of vectors for expression in yeast S. cerevisiae include pYepSec1 (Baldari et al., 1987, EMBO J 6:229-234), pMFa (Kurjan and Herskowitz, 1982, Cell 30:933-943), pJRY88 (Schultz et al., 1987, Gene 54:113-123), pYES2 (Invitrogen Corporation, San Diego, CA), and pPicZ (Invitrogen Corp, San Diego, CA).

Alternatively, the expression vector is a baculovirus expression vector. Baculovirus vectors available for expression of proteins in cultured insect cells (e.g., Sf 9 cells) include the pAc series (Smith et al., 1983, Mol. Cell Biol. 3:2156-2165) and the pVL series (Lucklow and Summers, 1989, Virology 170:31-39).

In yet another embodiment, a nucleic acid of the present invention is expressed in mammalian cells using a mammalian expression vector. Examples of mammalian expression vectors include pCDM8 (Seed, 1987, Nature 329:840) and pMT2PC (Kaufman et al., 1987, EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra. In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J 8:729-733) and immunoglobulins (Banerji et al., 1983, Cell 33:729-740; Queen and Baltimore, 1983, Cell 33:741-748), neuron-specific promoters (e.g., the neurofilament promoter; Byrne and Ruddle, 1989, Proc. Natl. Acad. Sci. U.S.A. 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the α-fetoprotein promoter (Camper and Tilghman, 1989, Genes Dev. 3:537-546).

The present invention further provides a recombinant expression vector comprising a DNA molecule cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the present invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes (see Weintraub et al., 1986, Trends in Genetics, Vol. 1(1)).

Another aspect of the present invention pertains to host cells into which a recombinant expression vector of the present invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

### V. Analyzing Biomarker Nucleic Acids and Polypeptides

Biomarker nucleic acids and/or biomarker polypeptides can be analyzed according to the methods described herein and techniques known to the skilled artisan to identify such genetic or expression alterations useful for the present invention including, but not limited to, 1) an alteration in the level of a biomarker transcript or polypeptide, 2) a deletion or addition of one or more nucleotides from a biomarker gene, 4) a substitution of one or more nucleotides of a biomarker gene, 5) aberrant modification of a biomarker gene, such as an expression regulatory region, and the like.

a. Methods for Detection of Copy Number

Methods of evaluating the copy number of a biomarker nucleic acid are well-known to those of skill in the art. The presence or absence of chromosomal gain or loss can be evaluated simply by a determination of copy number of the regions or markers identified herein.

In one embodiment, a biological sample is tested for the presence of copy number changes in genomic loci containing the genomic marker. A copy number of at least 3, 4, 5, 6, 7, 8, 9, or 10 is predictive of poorer outcome of inhibitor(s) of one or more biomarkers listed in Tables 1-2, or in combination with immunotherapy.

Methods of evaluating the copy number of a biomarker locus include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In one embodiment, evaluating the biomarker gene copy number in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal RNA (e.g., a non-amplified portion of the same or related cell, tissue, organ, etc.) provides an estimate of the relative copy number of the target nucleic acid. Alternatively, other methods well-known in the art to detect RNA can be used, such that higher or lower expression relative to an appropriate control (e.g., a non-amplified portion of the same or related cell tissue, organ, etc.) provides an estimate of the relative copy number of the target nucleic acid. An alternative means for determining genomic copy number is in situ hybridization (e.g., Angerer (1987) Meth. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application. In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained. The probes are typically labeled, e.g., with radioisotopes or fluorescent reporters. In one embodiment, probes are sufficiently long so as to specifically hybridize with the target nucleic acid(s) under stringent conditions. Probes generally range in length from about 200 bases to about 1000 bases. In some applications it is necessary to block the hybridization capacity of repetitive sequences. Thus, in some embodiments, tRNA, human genomic DNA, or Cot-I DNA is used to block non-specific hybridization.

An alternative means for determining genomic copy number is comparative genomic hybridization. In general, genomic DNA is isolated from normal reference cells, as well as from test cells (e.g., tumor cells) and amplified, if necessary. The two nucleic acids are differentially labeled and then hybridized in situ to metaphase chromosomes of a reference cell. The repetitive sequences in both the reference and test DNAs are either removed or their hybridization capacity is reduced by some means, for example by prehybridization with appropriate blocking nucleic acids and/or including such blocking nucleic acid sequences for said repetitive sequences during said hybridization. The bound, labeled DNA sequences are then rendered in a visualizable form, if necessary. Chromosomal regions in the test cells which are at increased or decreased copy number can be identified by detecting regions where the ratio of signal from the two DNAs is altered. For example, those regions that have decreased in copy number in the test cells will show relatively lower signal from the test DNA than the reference compared to other regions of the genome.

Regions that have been increased in copy number in the test cells will show relatively higher signal from the test DNA. Where there are chromosomal deletions or multiplications, differences in the ratio of the signals from the two labels will be detected and the ratio will provide a measure of the copy number. In another embodiment of CGH, array CGH (aCGH), the immobilized chromosome element is replaced with a collection of solid support bound target nucleic acids on an array, allowing for a large or complete percentage of the genome to be represented in the collection of solid support bound targets. Target nucleic acids may comprise cDNAs, genomic DNAs, oligonucleotides (e.g., to detect single nucleotide polymorphisms) and the like. Array-based CGH may also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays. Methods of preparing immobilized chromosomes or arrays and performing comparative genomic hybridization are well-known in the art (see, e.g., U.S. Pat. Nos. 6,335,167; 6,197,501; 5,830,645; and 5,665,549 and Albertson (1984) EMBO J 3: 1227-1234; Pinkel (1988) Proc. Natl. Acad. Sci. U.S.A. 85: 9138-9142; EPO Pub. No. 430,402; Methods in Molecular Biology, Vol. 33: In situ Hybridization Protocols, Choo, ed., Humana Press, Totowa, N.J. (1994), etc.) In another embodiment, the hybridization protocol of Pinkel, et al. (1998) Nature Genetics 20: 207211, or of Kallioniemi (1992) Proc. Natl Acad Sci U.S.A. 89:5321-5325 (1992) is used.

In still another embodiment, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

Methods of “quantitative” amplification are well-known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the present invention. In fluorogenic quantitative PCR, quantitation is based on amount of fluorescence signals, e.g., TaqMan and SYBR green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. U.S.A. 86: 1173), self-sustained sequence replication (Guatelli, et al. (1990) Proc. Nat. Acad. Sci. U.S.A. 87: 1874), dot PCR, and linker adapter PCR, etc.

Loss of heterozygosity (LOH) and major copy proportion (MCP) mapping (Wang, Z. C., et al. (2004) Cancer Res 64(1):64-71; Seymour, A. B., et al. (1994) Cancer Res 54, 2761-4; Hahn, S. A., et al. (1995) Cancer Res 55, 4670-5; Kimura, M., et al. (1996) Genes Chromosomes Cancer 17, 88-93; Li et al., (2008) MBC Bioinform. 9, 204-219) may also be used to identify regions of amplification or deletion.

b. Methods for Detection of Biomarker Nucleic Acid Expression

Biomarker expression may be assessed by any of a wide variety of well-known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In preferred embodiments, activity of a particular gene is characterized by a measure of gene transcript (e.g. mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In another embodiment, detecting or determining expression levels of a biomarker and functionally similar homologs thereof, including a fragment or genetic alteration thereof (e.g., in regulatory or promoter regions thereof) comprises detecting or determining RNA levels for the marker of interest. In one embodiment, one or more cells from the subject to be tested are obtained and RNA is isolated from the cells. In a preferred embodiment, a sample of breast tissue cells is obtained from the subject.

In one embodiment, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated (see, e.g., Bonner et al. (1997) Science 278: 1481; Emmert-Buck et al. (1996) Science 274:998; Fend et al. (1999) Am. J. Path. 154: 61 and Murakami et al. (2000) Kidney Int. 58:1346). For example, Murakami et al., supra, describe isolation of a cell from a previously immunostained tissue section.

It is also possible to obtain cells from a subject and culture the cells in vitro, such as to obtain a larger population of cells from which RNA can be extracted. Methods for establishing cultures of non-transformed cells, i.e., primary cell cultures, are known in the art.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the tissue or cells obtained from a subject is snap frozen as soon as possible.

RNA can be extracted from the tissue sample by a variety of methods, e.g., the guanidium thiocyanate lysis followed by CsCl centrifugation (Chirgwin et al., 1979, Biochemistry 18:5294-5299). RNA from single cells can be obtained as described in methods for preparing cDNA libraries from single cells, such as those described in Dulac, C. (1998) Curr. Top. Dev. Biol. 36, 245 and Jena et al. (1996) J. Immunol. Methods 190:199. Care to avoid RNA degradation must be taken, e.g., by inclusion of RNAsin. The RNA sample can then be enriched in particular species. In one embodiment, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, NY).

In a preferred embodiment, the RNA population is enriched in marker sequences. Enrichment can be undertaken, e.g., by primer-specific cDNA synthesis, or multiple rounds of linear amplification based on cDNA synthesis and template-directed in vitro transcription (see, e.g., Wang et al. (1989) Proc. Natl. Acad. Sci. U.S.A. 86: 9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). Real time PCR may also be used.

Other known amplification methods which can be utilized herein include but are not limited to the so-called “NASBA” or “3SR” technique described in PNAS U.S.A. 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. U.S.A., 87, 1874 (1990)); and transcription amplification (see, e.g., Kwoh et al., Proc. Natl. Acad. Sci. U.S.A. 86, 1173 (1989)).

Many techniques are known in the state of the art for determining absolute and relative levels of gene expression, commonly used techniques suitable for use in the present invention include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Labeled nucleic acids of a test sample obtained from a subject may be hybridized to a solid surface comprising biomarker DNA. Positive hybridization signal is obtained with the sample containing biomarker transcripts. Methods of preparing DNA arrays and their use are well-known in the art (see, e.g., U.S. Pat. Nos: 6,618,6796; 6,379,897; 6,664,377; 6,451,536; 548,257; U.S. 20030157485 and Schena et al. (1995) Science 20, 467-470; Gerhold et al. (1999) Trends In Biochem. Sci. 24, 168-173; and Lennon et al. (2000) Drug Discovery Today 5, 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescently-labeled cDNA probes are generated. The microarrays capable of hybridizing to marker cDNA are then probed with the labeled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the marker. As herein used, the term “stringent conditions” means hybridization will occur only if there is at least 95% identity in nucleotide sequences. In another embodiment, hybridization under “stringent conditions” occurs when there is at least 97% identity between the sequences.

The form of labeling of the probes may be any that is appropriate, such as the use of radioisotopes, for example, ³²P and ¹⁵S. Labeling with radioisotopes may be achieved, whether the probe is synthesized chemically or biologically, by the use of suitably labeled bases.

In one embodiment, the biological sample contains polypeptide molecules from the test subject. Alternatively, the biological sample can contain mRNA molecules from the test subject or genomic DNA molecules from the test subject.

In another embodiment, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

c. Methods for Detection of Biomarker Protein Expression

The activity or level of a biomarker protein can be detected and/or quantified by detecting or quantifying the expressed polypeptide. The polypeptide can be detected and quantified by any of a number of means well-known to those of skill in the art. Aberrant levels of polypeptide expression of the polypeptides encoded by a biomarker nucleic acid and functionally similar homologs thereof, including a fragment or genetic alteration thereof (e.g., in regulatory or promoter regions thereof) are associated with the likelihood of response of a cancer to inhibitor(s) of one or more biomarkers listed in Tables 1-2, or in combination with immunotherapy. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

For example, ELISA and RIA procedures may be conducted such that a desired biomarker protein standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker protein antibody is allowed to react with the system, and radioactivity or the enzyme assayed (ELISA-sandwich assay). Other conventional methods may also be employed as suitable.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

In one embodiment, a method for measuring biomarker protein levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein.

Enzymatic and radiolabeling of biomarker protein and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect biomarker protein according to a practitioner's preference based upon the present disclosure. One such technique is Western blotting (Towbin et al., Proc. Nat. Acad. Sci. 76:4350 (1979)), wherein a suitably treated sample is run on an SDS-PAGE gel before being transferred to a solid support, such as a nitrocellulose filter. Anti-biomarker protein antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabeling. The assay is scored visually, using microscopy.

Anti-biomarker protein antibodies, such as intrabodies, may also be used for imaging purposes, for example, to detect the presence of biomarker protein in cells and tissues of a subject. Suitable labels include radioisotopes, iodine (¹²⁵I, ¹²¹I), carbon (¹⁴C), sulphur (³⁵S), tritium (³H), indium (¹¹²In), and technetium (⁹⁹mTc), fluorescent labels, such as fluorescein and rhodamine, and biotin.

For in vivo imaging purposes, antibodies are not detectable, as such, from outside the body, and so must be labeled, or otherwise modified, to permit detection. Markers for this purpose may be any that do not substantially interfere with the antibody binding, but which allow external detection. Suitable markers may include those that may be detected by X-radiography, NMR or MRI. For X-radiographic techniques, suitable markers include any radioisotope that emits detectable radiation but that is not overtly harmful to the subject, such as barium or cesium, for example. Suitable markers for NMR and MRI generally include those with a detectable characteristic spin, such as deuterium, which may be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99. The labeled antibody or antibody fragment will then preferentially accumulate at the location of cells which contain biomarker protein. The labeled antibody or antibody fragment can then be detected using known techniques.

Antibodies that may be used to detect biomarker protein include any antibody, whether natural or synthetic, full length or a fragment thereof, monoclonal or polyclonal, that binds sufficiently strongly and specifically to the biomarker protein to be detected. An antibody may have a K_{d} of at most about 10⁻⁶M, 10⁻⁷M, 10⁻⁸M, 10⁻⁹M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the biomarker protein relative to other proteins, such as related proteins. Antibodies are commercially available or may be prepared according to methods known in the art.

Antibodies and derivatives thereof that may be used encompass polyclonal or monoclonal antibodies, chimeric, human, humanized, primatized (CDR-grafted), veneered or single-chain antibodies as well as functional fragments, i.e., biomarker protein binding fragments, of antibodies. For example, antibody fragments capable of binding to a biomarker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′) 2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., 10: 14551460 (1992), regarding primatized antibody, and Ladner et al., U.S. Pat. No. 4,946,778 and Bird, R. E. et al., Science, 242: 423-426 (1988)) regarding single-chain antibodies. Antibodies produced from a library, e.g., phage display library, may also be used.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

d. Methods for Detection of Biomarker Structural Alterations

The following illustrative methods can be used to identify the presence of a structural alteration in a biomarker nucleic acid and/or biomarker polypeptide molecule in order to, for example, identify the one or more biomarkers listed in Tables 1-2, or other biomarkers used in the immunotherapies described herein that are overexpressed, overfunctional, and the like.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. U.S.A. 91:360-364), the latter of which can be particularly useful for detecting point mutations in a biomarker nucleic acid such as a biomarker gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a biomarker gene under conditions such that hybridization and amplification of the biomarker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self-sustained sequence replication (Guatelli, J. C. et al. (1990) Proc. Natl. Acad. Sci. U.S.A. 87:1874-1878), transcriptional amplification system (Kwoh, D. Y. et al. (1989) Proc. Natl. Acad. Sci. U.S.A. 86:1173-1177), Q-Beta Replicase (Lizardi, P. M. et al. (1988) Bio-Technology 6:1197), or any other nucleic acid amplification method, followed by the detection of the amplified molecules using techniques well-known to those of skill in the art. These detection schemes are especially useful for the detection of nucleic acid molecules if such molecules are present in very low numbers.

In an alternative embodiment, mutations in a biomarker nucleic acid from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in biomarker nucleic acid can be identified by hybridizing a sample and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, biomarker genetic mutations can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential, overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene. Such biomarker genetic mutations can be identified in a variety of contexts, including, for example, germline and somatic mutations.

In yet another embodiment, any of a variety of sequencing reactions known in the art can be used to directly sequence a biomarker gene and detect mutations by comparing the sequence of the sample biomarker with the corresponding wild-type (control) sequence. Examples of sequencing reactions include those based on techniques developed by Maxam and Gilbert (1977) Proc. Natl. Acad. Sci. U.S.A. 74:560 or Sanger (1977) Proc. Natl. Acad Sci. U.S.A. 74:5463. It is also contemplated that any of a variety of automated sequencing procedures can be utilized when performing the diagnostic assays (Naeve (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a biomarker gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type biomarker sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. U.S.A. 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in biomarker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a biomarker sequence, e.g., a wild-type biomarker treated with a DNA mismatch repair enzyme, and the cleavage products, if any, can be detected from electrophoresis protocols or the like (e.g., U.S. Pat. No. 5,459,039.)

In other embodiments, alterations in electrophoretic mobility can be used to identify mutations in biomarker genes. For example, single strand conformation polymorphism (SSCP) may be used to detect differences in electrophoretic mobility between mutant and wild type nucleic acids (Orita et al. (1989) Proc Natl. Acad. Sci U.S.A. 86:2766; see also Cotton (1993) Mutat. Res. 285:125-144 and Hayashi (1992) Genet. Anal. Tech. Appl. 9:73-79). Single-stranded DNA fragments of sample and control biomarker nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Nat/. Acad. Sci. U.S.A. 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci U.S.A. 88:189). In such cases, ligation will occur only if there is a perfect match at the 3′ end of the 5′ sequence making it possible to detect the presence of a known mutation at a specific site by looking for the presence or absence of amplification.

### VI. Anti-Cancer Therapies

The efficacy of inhibitors of one or more biomarkers listed in Tables 1-2 is predicted according to biomarker amount and/or activity associated with a cancer in a subject according to the methods described herein. In one embodiment, such inhibitor in combination with one or more additional anti-cancer therapies, such as immunotherapy can be administered, particularly if a subject has first been indicated as being a likely responder to inhibitor and immunotherapy combination treatment. In another embodiment, such inhibitor can be avoided once a subject is indicated as not being a likely responder to inhibitor and immunotherapy combination treatment and an alternative treatment regimen, such as targeted and/or untargeted anti-cancer therapies can be administered.

Combination therapies are also contemplated and can comprise, for example, one or more chemotherapeutic agents and radiation, one or more chemotherapeutic agents and immunotherapy, or one or more chemotherapeutic agents, radiation and chemotherapy, each combination of which can be with anti-immune checkpoint therapy. In addition, any representative embodiment of an agent to modulate a particular target can be adapted to any other target described herein by the ordinarily skilled artisan.

Similarly, agents which directly block the interaction between one or more biomarkers listed in Tables 1-2, and the binding partners and/or substrates of such one or more biomarkers, and the like, can remove the inhibition to such one or more biomarkers-regulated signaling and its downstream immune responses, such as increasing sensitivity to interferon signaling.

Alternatively, agents that indirectly block the interaction between such one or more biomarkers and its binding partners/substrates can remove the inhibition to such one or more biomarkers-regulated signaling and its downstream immune responses. For example, a truncated or dominant negative form of such one or more biomarkers, such as biomarker fragments without functional activity, by binding to a substrate of such one or more biomarkers and indirectly reducing the effective concentration of such substrate available to bind to the one or more biomarkers in cell. Exemplary agents include monospecific or bispecific blocking antibodies, especially intrabodies, against the one or more biomarkers and/or their substrate(s) that block the interaction between the one or more biomarkers and their substrate(s); a non-active form of such one or more biomarkers and/or their substrate(s) (e.g., a dominant negative polypeptide), small molecules or peptides that block the interaction between such one or more biomarkers and their substrate(s) or the activity of such one or more biomarkers; and a non-activating form of a natural biomarker and/or its substrate(s).

Immunotherapies that are designed to elicit or amplify an immune response are referred to as “activation immunotherapies.” Immunotherapies that are designed to reduce or suppress an immune response are referred to as “suppression immunotherapies.” In some embodiments, the immunotherapy is cancer cell-specific. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In one embodiment, immunotherapy comprises adoptive cell-based immunotherapies. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, patient-specific neoantigen vaccines, and the like.

In another embodiment, immunotherapy comprises non-cell-based immunotherapies. In one embodiment, compositions comprising antigens with or without vaccine-enhancing adjuvants are used. Such compositions exist in many well-known forms, such as peptide compositions, oncolytic viruses, recombinant antigen comprising fusion proteins, and the like. In still another embodiment, immunomodulatory interleukins, such as IL-2, IL-6, IL-7, IL-12, IL-17, IL-23, and the like, as well as modulators thereof (e.g., blocking antibodies or more potent or longer lasting forms) are used. In yet another embodiment, immunomodulatory cytokines, such as interferons, G-CSF, imiquimod, TNFalpha, and the like, as well as modulators thereof (e.g., blocking antibodies or more potent or longer lasting forms) are used. In another embodiment, immunomodulatory chemokines, such as CCL3, CCL26, and CXCL7, and the like, as well as modulators thereof (e.g., blocking antibodies or more potent or longer lasting forms) are used. In another embodiment, immunomodulatory molecules targeting immunosuppression, such as STAT3 signaling modulators, NFkappaB signaling modulators, and immune checkpoint modulators, are used.

In still another embodiment, immunomodulatory drugs, such as immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-xB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), I3C(indole-3-carbinol)/DIM(di-indolmethane) (13C/DIM), Bay 11-7082, luteolin, cell permeable peptide SN-50, IKBa.-super repressor overexpression, NFKB decoy oligodeoxynucleotide (ODN), or a derivative or analog of any thereof, are used. In yet another embodiment, immunomodulatory antibodies or protein are used. For example, antibodies that bind to CD40, Toll-like receptor (TLR), OX40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 an antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL12 inhibitor, an IL23 inhibitor, ustekinumab, and the like. Nutritional supplements that enhance immune responses, such as vitamin A, vitamin E, vitamin C, and the like, are well-known in the art (see, for example, U.S. Pat. Nos. 4,981,844 and 5,230,902 and PCT Publ. No. WO 2004/004483) can be used in the methods described herein.

Similarly, other agents can be used with in combination with inhibitors of one or more biomarkers listed in Tables 1-2, with or without immunotherapies to stimulate an immune response to thereby treat a condition that would benefit therefrom. For example, chemotherapy, radiation, epigenetic modifiers (e.g., histone deacetylase (HDAC) modifiers, methylation modifiers, phosphorylation modifiers, and the like), targeted therapy, and the like are well-known in the art.

The term “untargeted therapy” refers to administration of agents that do not selectively interact with a chosen biomolecule yet treat cancer. Representative examples of untargeted therapies include, without limitation, chemotherapy, gene therapy, and radiation therapy. In one embodiment, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of 0-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97110(14)). Poly(ADP-ribose) polymerase 1 (PARP1) is a key molecule in the repair of DNA single-strand breaks (SSBs) (de Murcia J. et al. 1997. Proc Natl Acad Sci U.S.A. 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913917; Farmer H, et al. (2005) Nature 434:917-921). The foregoing examples of chemotherapeutic agents are illustrative, and are not intended to be limiting.

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, surgical intervention can occur to physically remove cancerous cells and/or tissues. In still another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In yet another embodiment, hyperthermia, a procedure in which body tissue is exposed to high temperatures (up to 106° F.) is used. Heat may help shrink tumors by damaging cells or depriving them of substances they need to live. Hyperthermia therapy can be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area may be heated externally with high-frequency waves aimed at a tumor from a device outside the body. To achieve internal heating, one of several types of sterile probes may be used, including thin, heated wires or hollow tubes filled with warm water; implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It can be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, can cause discomfort or even significant local pain in about half the patients treated. It can also cause blisters, which generally heal rapidly.

In still another embodiment, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT can be directed through a fiber-optic (a very thin glass strand). The fiber-optic is placed close to the cancer to deliver the proper amount of light. The fiber-optic can be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and can include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early non-small cell lung cancer in patients for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In yet another embodiment, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and may be used to cut through steel or to shape diamonds. Lasers also can be used for very precise surgical work, such as repairing a damaged retina in the eye or cutting through tissue (in place of a scalpel). Although there are several different kinds of lasers, only three kinds have gained wide use in medicine: Carbon dioxide (CO₂) laser—This type of laser can remove thin layers from the skin's surface without penetrating the deeper layers. This technique is particularly useful in treating tumors that have not spread deep into the skin and certain precancerous conditions. As an alternative to traditional scalpel surgery, the CO₂ laser is also able to cut the skin. The laser is used in this way to remove skin cancers. Neodymium:yttrium-aluminum-garnet (Nd:YAG) laser—Light from this laser can penetrate deeper into tissue than light from the other types of lasers, and it can cause blood to clot quickly. It can be carried through optical fibers to less accessible parts of the body. This type of laser is sometimes used to treat throat cancers. Argon laser—This laser can pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time may be needed because the precision of the laser allows for a smaller incision. Healing time is often shortened; since laser heat seals blood vessels, there is less bleeding, swelling, or scarring. Laser surgery may be less complicated. For example, with fiber optics, laser light can be directed to parts of the body without making a large incision. More procedures may be done on an outpatient basis. Lasers can be used in two ways to treat cancer: by shrinking or destroying a tumor with heat, or by activating a chemical—known as a photosensitizing agent—that destroys cancer cells. In PDT, a photosensitizing agent is retained in cancer cells and can be stimulated by light to cause a reaction that kills cancer cells. CO₂ and Nd:YAG lasers are used to shrink or destroy tumors. They may be used with endoscopes, tubes that allow physicians to see into certain areas of the body, such as the bladder. The light from some lasers can be transmitted through a flexible endoscope fitted with fiber optics. This allows physicians to see and work in parts of the body that could not otherwise be reached except by surgery and therefore allows very precise aiming of the laser beam. Lasers also may be used with low-power microscopes, giving the doctor a clear view of the site being treated. Used with other instruments, laser systems can produce a cutting area as small as 200 microns in diameter-less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers may be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat may help shrink tumors by damaging cells or depriving them of substances they need to live. In this treatment, lasers are directed to interstitial areas (areas between organs) in the body. The laser light then raises the temperature of the tumor, which damages or destroys cancer cells.

The duration and/or dose of treatment with therapies may vary according to the particular therapeutic agent or combination thereof. An appropriate treatment time for a particular cancer therapeutic agent will be appreciated by the skilled artisan. The present invention contemplates the continued assessment of optimal treatment schedules for each cancer therapeutic agent, where the phenotype of the cancer of the subject as determined by the methods of the present invention is a factor in determining optimal treatment doses and schedules.

Any means for the introduction of a polynucleotide into mammals, human or non-human, or cells thereof may be adapted to the practice of this invention for the delivery of the various constructs of the present invention into the intended recipient. In one embodiment of the present invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al., Am J Respir Cell Mol Biol 10:24-29, 1994; Tsan et al., Am J Physiol 268; Alton et al., Nat Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below).

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

The nucleic acids encoding a protein or nucleic acid of interest may be in a plasmid or viral vector, or other vector as is known in the art. Such vectors are well-known and any can be selected for a particular application. In one embodiment of the present invention, the gene delivery vehicle comprises a promoter and a demethylase coding sequence. Preferred promoters are tissue-specific promoters and promoters which are activated by cellular proliferation, such as the thymidine kinase and thymidylate synthase promoters. Other preferred promoters include promoters which are activatable by infection with a virus, such as the α- and β-interferon promoters, and promoters which are activatable by a hormone, such as estrogen. Other promoters which can be used include the Moloney virus LTR, the CMV promoter, and the mouse albumin promoter. A promoter may be constitutive or inducible.

In another embodiment, naked polynucleotide molecules are used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al., Hum. Gene. Ther. 3:147-154, 1992. Other vehicles which can optionally be used include DNA-ligand (Wu et al., J Biol. Chem. 264:16985-16987, 1989), lipid-DNA combinations (Felgner et al., Proc. Natl. Acad. Sci. U.S.A. 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. U.S.A. 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805). Other viral vector systems that can be used to deliver a polynucleotide of the present invention have been derived from herpes virus, e.g., Herpes Simplex Virus (U.S. Pat. No. 5,631,236 by Woo et al., issued May 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth, Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Preferred viruses include an alphavirus, a poxivirus, an arena virus, a vaccinia virus, a polio virus, and the like. They offer several attractive features for various mammalian cells (Friedmann (1989) Science, 244:1275-1281; Ridgeway, 1988, supra; Baichwal and Sugden, 1986, supra; Coupar et al., 1988; Horwich et al. (1990) J. Virol., 64:642-650).

In other embodiments, target DNA in the genome can be manipulated using well-known methods in the art. For example, the target DNA in the genome can be manipulated by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis.

In other embodiments, recombinant biomarker polypeptides, and fragments thereof, can be administered to subjects. In some embodiments, fusion proteins can be constructed and administered which have enhanced biological properties. In addition, the biomarker polypeptides, and fragment thereof, can be modified according to well-known pharmacological methods in the art (e.g., pegylation, glycosylation, oligomerization, etc.) in order to further enhance desirable biological activities, such as increased bioavailability and decreased proteolytic degradation.

### VII. Clinical Efficacy

Clinical efficacy can be measured by any method known in the art. For example, the response to a therapy, such as inhibitors of one or more biomarkers listed in Tables 1-2 relates to any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al., J. Clin. Oncol. (2007) 25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular anti-immune checkpoint therapeutic regimen is at least 25%, 30%, 35%, 40%, 45%, 50%, 55%, 60%, 65%, 70%, 75%, 80%, 85%, or more.

Additional criteria for evaluating the response to immunotherapies, such as anti-immune checkpoint therapies, are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

### VIII. Further Uses and Methods of the Present Invention

The compositions described herein can be used in a variety of diagnostic, prognostic, and therapeutic applications. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

a. Screening Methods

One aspect of the present invention relates to screening assays, including non-cell based assays and xenograft animal model assays. In one embodiment, the assays provide a method for identifying whether a cancer is likely to respond to inhibitors of one or more biomarkers listed in Tables 1-2, such as in a human by using a xenograft animal model assay, and/or whether an agent can inhibit the growth of or kill a cancer cell that is unlikely to respond to inhibitors of one or more biomarkers listed in Tables 1-2.

In one embodiment, the present invention relates to assays for screening test agents which bind to, or modulate the biological activity of, at least one biomarker described herein (e.g., in the tables, figures, examples, or otherwise in the specification). In one embodiment, a method for identifying such an agent entails determining the ability of the agent to modulate, e.g. inhibit, the at least one biomarker described herein.

In one embodiment, an assay is a cell-free or cell-based assay, comprising contacting at least one biomarker described herein, with a test agent, and determining the ability of the test agent to modulate (e.g., inhibit) the enzymatic activity of the biomarker, such as by measuring direct binding of substrates or by measuring indirect parameters as described below.

For example, in a direct binding assay, biomarker protein (or their respective target polypeptides or molecules) can be coupled with a radioisotope or enzymatic label such that binding can be determined by detecting the labeled protein or molecule in a complex. For example, the targets can be labeled with ¹²⁵I, ³⁵S, ¹⁴C, or ³H, either directly or indirectly, and the radioisotope detected by direct counting of radioemission or by scintillation counting. Alternatively, the targets can be enzymatically labeled with, for example, horseradish peroxidase, alkaline phosphatase, or luciferase, and the enzymatic label detected by determination of conversion of an appropriate substrate to product. Determining the interaction between biomarker and substrate can also be accomplished using standard binding or enzymatic analysis assays. In one or more embodiments of the above described assay methods, it may be desirable to immobilize polypeptides or molecules to facilitate separation of complexed from uncomplexed forms of one or both of the proteins or molecules, as well as to accommodate automation of the assay.

Binding of a test agent to a target can be accomplished in any vessel suitable for containing the reactants. Non-limiting examples of such vessels include microtiter plates, test tubes, and micro-centrifuge tubes. Immobilized forms of the antibodies described herein can also include antibodies bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

In an alternative embodiment, determining the ability of the agent to modulate the interaction between the biomarker and a substrate or a biomarker and its natural binding partner can be accomplished by determining the ability of the test agent to modulate the activity of a polypeptide or other product that functions downstream or upstream of its position within the signaling pathway (e.g., feedback loops). Such feedback loops are well-known in the art (see, for example, Chen and Guillemin (2009) Int. J. Tryptophan Res. 2:1-19).

The present invention further pertains to novel agents identified by the above-described screening assays. Accordingly, it is within the scope of this invention to further use an agent identified as described herein, such as in an appropriate animal model. For example, an agent identified as described herein can be used in an animal model to determine the efficacy, toxicity, or side effects of treatment with such an agent. Alternatively, an antibody identified as described herein can be used in an animal model to determine the mechanism of action of such an agent.

b. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining the amount and/or activity level of a biomarker described herein in the context of a biological sample (e.g., blood, serum, cells, or tissue) to thereby determine whether an individual afflicted with a cancer is likely to respond to inhibitors of one or more biomarkers listed in Tables 1-2 such as in a cancer. Such assays can be used for prognostic or predictive purpose alone, or can be coupled with a therapeutic intervention to thereby prophylactically treat an individual prior to the onset or after recurrence of a disorder characterized by or associated with biomarker polypeptide, nucleic acid expression or activity. The skilled artisan will appreciate that any method can use one or more (e.g., combinations) of biomarkers described herein, such as those in the tables, figures, examples, and otherwise described in the specification.

Another aspect of the present invention pertains to monitoring the influence of agents (e.g., drugs, compounds, and small nucleic acid-based molecules) on the expression or activity of a biomarker described herein. These and other agents are described in further detail in the following sections.

The skilled artisan will also appreciated that, in certain embodiments, the methods of the present invention implement a computer program and computer system. For example, a computer program can be used to perform the algorithms described herein. A computer system can also store and manipulate data generated by the methods of the present invention which comprises a plurality of biomarker signal changes/profiles which can be used by a computer system in implementing the methods of this invention. In certain embodiments, a computer system receives biomarker expression data; (ii) stores the data; and (iii) compares the data in any number of ways described herein (e.g., analysis relative to appropriate controls) to determine the state of informative biomarkers from cancerous or pre-cancerous tissue. In other embodiments, a computer system (i) compares the determined expression biomarker level to a threshold value; and (ii) outputs an indication of whether said biomarker level is significantly modulated (e.g., above or below) the threshold value, or a phenotype based on said indication.

In certain embodiments, such computer systems are also considered part of the present invention. Numerous types of computer systems can be used to implement the analytic methods of this invention according to knowledge possessed by a skilled artisan in the bioinformatics and/or computer arts. Several software components can be loaded into memory during operation of such a computer system. The software components can comprise both software components that are standard in the art and components that are special to the present invention (e.g., dCHIP software described in Lin et al. (2004) Bioinformatics 20, 1233-1240; radial basis machine learning algorithms (RBM) known in the art).

The methods of the present invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.).

In certain embodiments, the computer comprises a database for storage of biomarker data. Such stored profiles can be accessed and used to perform comparisons of interest at a later point in time. For example, biomarker expression profiles of a sample derived from the noncancerous tissue of a subject and/or profiles generated from population-based distributions of informative loci of interest in relevant populations of the same species can be stored and later compared to that of a sample derived from the cancerous tissue of the subject or tissue suspected of being cancerous of the subject.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

c. Diagnostic Assays

The present invention provides, in part, methods, systems, and code for accurately classifying whether a biological sample is associated with a cancer that is likely to respond to modulators of one or more biomarkers listed in Tables 1-2. In some embodiments, the present invention is useful for classifying a sample (e.g., from a subject) as associated with or at risk for responding to or not responding to modulators of one or more biomarkers listed in Tables 1-2 using a statistical algorithm and/or empirical data (e.g., the amount or activity of a biomarker described herein, such as in the tables, figures, examples, and otherwise described in the specification).

An exemplary method for detecting the amount or activity of a biomarker described herein, and thus useful for classifying whether a sample is likely or unlikely to respond to modulators of one or more biomarkers listed in Tables 1-2 involves obtaining a biological sample from a test subject and contacting the biological sample with an agent, such as a protein-binding agent like an antibody or antigen-binding fragment thereof, or a nucleic acid-binding agent like an oligonucleotide, capable of detecting the amount or activity of the biomarker in the biological sample. In some embodiments, at least one antibody or antigen-binding fragment thereof is used, wherein two, three, four, five, six, seven, eight, nine, ten, or more such antibodies or antibody fragments can be used in combination (e.g., in sandwich ELISAs) or in serial. In certain instances, the statistical algorithm is a single learning statistical classifier system. For example, a single learning statistical classifier system can be used to classify a sample as a based upon a prediction or probability value and the presence or level of the biomarker. The use of a single learning statistical classifier system typically classifies the sample as, for example, a likely immunotherapy responder or progressor sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the sample classification results to a clinician, e.g., an oncologist.

In another embodiment, the diagnosis of a subject is followed by administering to the individual a therapeutically effective amount of a defined treatment based upon the diagnosis.

In one embodiment, the methods further involve obtaining a control biological sample (e.g., biological sample from a subject who does not have a cancer or whose cancer is susceptible to modulators of one or more biomarkers listed in Tables 1-2), a biological sample from the subject during remission, or a biological sample from the subject during treatment for developing a cancer progressing despite modulators of one or more biomarkers listed in Tables 1-2.

d. Prognostic Assays

The diagnostic methods described herein can furthermore be utilized to identify subjects having or at risk of developing a cancer that is likely or unlikely to be responsive to modulators of one or more biomarkers listed in Tables 1-2. The assays described herein, such as the preceding diagnostic assays or the following assays, can be utilized to identify a subject having or at risk of developing a disorder associated with a misregulation of the amount or activity of at least one biomarker described herein, such as in cancer. Alternatively, the prognostic assays can be utilized to identify a subject having or at risk for developing a disorder associated with a misregulation of the at least one biomarker described herein, such as in cancer. Furthermore, the prognostic assays described herein can be used to determine whether a subject can be administered an agent (e.g., an agonist, antagonist, peptidomimetic, polypeptide, peptide, nucleic acid, small molecule, or other drug candidate) to treat a disease or disorder associated with the aberrant biomarker expression or activity.

e. Treatment Methods

The therapeutic compositions described herein, such as modulators of one or more biomarkers listed in Tables 1-2 can be used in a variety of in vitro and in vivo therapeutic applications using the formulations and/or combinations described herein. In one embodiment, the therapeutic agents can be used to treat cancers determined to be responsive thereto. For example, single or multiple agents that inhibit or enhance one or more biomarkers listed in Tables 1-2 can be used to treat cancers in subjects identified as likely responders thereto.

Modulatory methods of the present invention involve contacting a cell, such as an immune cell with an agent that inhibits or blocks the expression and/or activity of such one or more biomarkers. Exemplary agents useful in such methods are described above. Such agents can be administered in vitro or ex vivo (e.g., by contacting the cell with the agent) or, alternatively, in vivo (e.g., by administering the agent to a subject). As such, the present invention provides methods useful for treating an individual afflicted with a condition that would benefit from an increased immune response, such as an infection or a cancer like colorectal cancer.

Immune responses can also be enhanced in an infected patient through an ex vivo approach, for instance, by removing immune cells from the patient, contacting immune cells in vitro with an agent described herein and reintroducing the in vitro stimulated immune cells into the patient.

In certain instances, it may be desirable to further administer other agents that upregulate immune responses, for example, forms of other B7 family members that transduce signals via costimulatory receptors, in order to further augment the immune response. Such additional agents and therapies are described further below. Agents that upregulate an immune response can be used prophylactically in vaccines against various polypeptides (e.g., polypeptides derived from pathogens). Immunity against a pathogen (e.g., a virus) can be induced by vaccinating with a viral protein along with an agent that upregulates an immune response, in an appropriate adjuvant.

In another embodiment, upregulation or enhancement of an immune response function, as described herein, is useful in the induction of tumor immunity.

In another embodiment, the immune response can be stimulated by the methods described herein, such that preexisting tolerance, clonal deletion, and/or exhaustion (e.g., T cell exhaustion) is overcome. For example, immune responses against antigens to which a subject cannot mount a significant immune response, e.g., to an autologous antigen, such as a tumor specific antigens can be induced by administering appropriate agents described herein that upregulate the immune response. In one embodiment, an autologous antigen, such as a tumor-specific antigen, can be coadministered. In another embodiment, the subject agents can be used as adjuvants to boost responses to foreign antigens in the process of active immunization.

In one embodiment, immune cells are obtained from a subject and cultured ex vivo in the presence of an agent as described herein, to expand the population of immune cells and/or to enhance immune cell activation. In a further embodiment the immune cells are then administered to a subject. Immune cells can be stimulated in vitro by, for example, providing to the immune cells a primary activation signal and a costimulatory signal, as is known in the art. Various agents can also be used to costimulate proliferation of immune cells. In one embodiment immune cells are cultured ex vivo according to the method described in PCT Application No. WO 94/29436. The costimulatory polypeptide can be soluble, attached to a cell membrane, or attached to a solid surface, such as a bead.

### IX. Administration of Agents

Administration of a therapeutically active amount of the therapeutic composition of the present invention is defined as an amount effective, at dosages and for periods of time necessary, to achieve the desired result. For example, a therapeutically active amount of an agent may vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

Inhibiting or enhancing expression and/or activity of one or more biomarkers listed in Tables 1-2, alone or in combination with an immunotherapy, can be accomplished by combination therapy with the modulatory agents described herein. Combination therapy describes a therapy in which one or more biomarkers are inhibited or blocked with an immunotherapy simultaneously. This may be achieved by administration of the modulatory agent described herein with the immunotherapy simultaneously (e.g., in a combination dosage form or by simultaneous administration of single agents) or by administration of single inhibitory agent for such one or more biomarkers and the immunotherapy, according to a schedule that results in effective amounts of each modulatory agent present in the patient at the same time.

The therapeutic agents described herein can be administered in a convenient manner such as by injection (subcutaneous, intravenous, etc.), oral administration, inhalation, transdermal application, or rectal administration. Depending on the route of administration, the active compound can be coated in a material to protect the compound from the action of enzymes, acids and other natural conditions which may inactivate the compound. For example, for administration of agents, by other than parenteral administration, it may be desirable to coat the agent with, or co-administer the agent with, a material to prevent its inactivation.

An agent can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that modulates (e.g., inhibits) biomarker expression and/or activity, or expression and/or activity of the complex encompassed by the present invention. These salts can be prepared in situ during the final isolation and purification of the therapeutic agents, or by separately reacting a purified therapeutic agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66:1-19).

In other cases, the agents useful in the methods of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates (e.g., inhibits) biomarker expression and/or activity, or expression and/or activity of the complex. These salts can likewise be prepared in situ during the final isolation and purification of the therapeutic agents, or by separately reacting the purified therapeutic agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra). Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well-known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent that modulates (e.g., inhibits) biomarker expression and/or activity, with the carrier and, optionally, one or more accessory ingredients. In general, the formulations are prepared by uniformly and intimately bringing into association a therapeutic agent with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a therapeutic agent as an active ingredient. A compound may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well-known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more therapeutic agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate. Dosage forms for the topical or transdermal administration of an agent that modulates (e.g., inhibits) biomarker expression and/or activity include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a therapeutic agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., inhibits) biomarker expression and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agent that modulates (e.g., inhibits or enhances) biomarker expression and/or activity, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a therapeutic agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel. Ophthalmic formulations, eye ointments, powders, solutions and the like, are also contemplated as being within the scope of this invention.

Pharmaceutical compositions of this invention suitable for parenteral administration comprise one or more therapeutic agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates (e.g., inhibits) biomarker expression and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the therapeutic agents of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

The nucleic acid molecules of the present invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. U.S.A. 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

In one embodiment, an agent encompassed by the present invention is an antibody. As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment may increase or decrease over the course of a particular treatment. Changes in dosage may result from the results of diagnostic assays.

### EXAMPLES

### Example 1: Materials and Methods for Examples 2-7

a. Animal Studies

All procedures involving mice and experimental protocols were approved by Institutional Animal Care and Use Committee (IACUC) of Dana-Farber Cancer Institute (11-009). For tumor xenografts, 1.5×10⁶ Apc^{KO}Kras^{G12D} colon organoid cells or 1×10⁶ HT-29 CRC cells were injected into flanks of athymic Ncr-nu/nu mice. Tumor measurements were made by caliper and tumor volumes were calculated using the formula: volume=length×width²×0.5. At the end-point of experiments, tumors were harvested, fixed in 10% formalin overnight, and paraffin-embedded for histological analysis. Fresh tissue was also collected for RNA isolation, protein collection and flash-frozen for long-term storage.

b. Histopathology

Paraffin-embedded intestines, organoids or xenograft tumors were serially sectioned and mounted on a slide. Sections were subjected to hematoxylin and eosin (H&E), Alcian blue-Periodic Acid Schiff (AB-PAS), as well as immunostaining, using standard procedures. For morphological analysis, sections were serially dehydrated in xylene and ethanol, stained with H&E for histological assessment or AB-PAS to identify goblet cells and mucus.

c. Immunofluorescence and Immunohistochemistry

For immunostaining, antigen retrieval was performed using a sodium citrate buffer. Slides were permeabilized using a 0.2% Triton X100 for 30 minutes at room temperature and blocked with donkey serum for 1 hour. The primary antibodies used for immunohistochemistry were rabbit anti-Sox9 (1:600, CST, #82630), rabbit anti-Mucin2 (1:200, Santa Cruz, sc-15334), and rabbit anti-Ki67 (1:1,000, CST, #12202). Binding of primary antibody was detected with 3,39-diamino-benzidine-tetrahydrochloride-dihydrate and counterstained with hematoxylin. For immunofluorescence studies, staining was performed with an anti-Keratin20 antibody (1:400, CST, #13063). The primary antibody was recognized using donkey-anti-rabbit Alexa Fluor 488 antibody (1:1,000, Life Technologies). For double-labeling studies, anti-E-Cadherin antibody (1:200, BD, 610181) was detected with anti-mouse Alexa-488 (1:1,000, Life Technologies), and anti-Vimentin (1:100, CST, #5741) was detected with anti-rabbit Alexa-555 (1:1,000, Life Technologies). Hoechst 33342 (Invitrogen) was incubated at room temperature for 10 min to stain the nuclei. Slides were counterstained with 4′,6-diamidino-2-phenylindole (DAPI) and mounted using the Prolong Gold anti-fade mounting media (Invitrogen). No staining was detected in slides incubated without primary antibody.

d. Intestinal Organoid Culture

Colonic glands were isolated, treated with EDTA, and then resuspended in 30-50 ul of Matrigel (BD Bioscience) and plated in 24-well plates. Wnt/R-spondin-deprived medium, DMEM/F12 with HEPES (Sigma-Aldrich) containing 20% FBS, 1% penicillin/streptomycin and 50 ng/ml recombinant mouse EGF (Life Technologies), was used for culturing Apc^{KO} colon organoids. For the first 2-3 days after seeding, the media was also supplemented with 10 μM ROCK inhibitor Y-27632 (Sigma Aldrich) and 10 μM SB431542 (Sigma Aldrich), an inhibitor for the transforming growth factor (TGF)-β type I receptor to avoid anoikis. For passage, colon organoids were dispersed by trypsin-EDTA and transferred to fresh Matrigel. Passage was performed every 3-4 days with a 1:3-1:5 split ratio.

e. Cell Culture, Lentivirus Packing, and Transduction

All cell lines were maintained at 37° C. with 5% C02. The human colorectal cancer cell lines were obtained from the CCLE core facility and used at early passage for the experiments. HEK293T, HT-115, HT-29, and COLO-205 cells were maintained in DMEM medium supplemented with 10% FBS and 1% penicillin/streptomycin. LS513, LS123, SW1463, and LS180 cells were cultured in RPMI 1640 containing 10% FBS and 1% penicillin/streptomycin. CL-40 cells were cultured in DMEM/F12 (1:1) supplemented with 20% FBS and 1% penicillin/streptomycin.

To generate lentiviruses, expression vectors were co-transfected into HEK293T cells with the lentiviral packaging constructs psPAX2 and pMD2.G (VSV-G) in a 1:1:1 ratio using X-tremeGENE 9 DNA Transfection Reagent (Roche) according to the manufacturer's instructions. Cell culture media was changed the following day and lentiviral supernatant was harvested 48 h and 72 h later and filtered through a 0.45 m filter (Millipore). Lentiviruses were aliquoted and stored at −80° C. until use.

To transduce colonic organoids, spheroids in one well (24-well plate) were trypsinized and a one-fourth to one-eighth volume of cell suspension was used for each infection. Cells were resuspended in 500 μl lentiviral supernatant with 8 μg/mL polybrene and 10 μM Y-27632, centrifuged at 600 g 32° C. 1 hours, and incubated for 6 hours in cell culture incubator. The infected cells were suspended in 30-50 ul of Matrigel and cultured with Wnt/R-spondin-deprived medium containing 10 μM Y-27632 and 10 μM SB431542.

To perform lentiviral infection, the CRC cells were plated in a 6-cm dishes and infected with 0.5-1 mL virus in media containing 8 mg/mL polybrene overnight.

f. Cell Proliferation Assays

Cell viability was quantified by measuring cellular ATP content using the CellTiterGlo Cell Viability assay (Promega) according to the manufacturer's instructions. All experiments were performed in triplicate in 96-well plates. Area measurements and quantification of low-attachment colonies was measured using ImageJ software.

g. Generation of Stable Cell Lines

All genetically manipulated colon organoid lines were generated using the protocol described previously (Shalem et al. (2014) Science 343: 84-87). In brief, sgRNAs targeting SOX9 were designed, and cloned into Lenti-CRISPR v2 (Sanjana et al. (2014) Nature methods 11: 783-784). shRNAs against SOX9/PROM1 were cloned into PLKO.1, TET-PLKO and TET-Cellecta vectors. To generate SOX9 KO or KD and PROM1 KD stable cells, CRC cells were selected with 1.5 μg/ml puromycin and colon organoids were selected with 3 μg/ml puromycin at 24 hours post-infection. To generate stable cells constitutively expressing SOX9 or PROM1, PLX304 vectors containing indicated cDNA were constructed and 15 μg/ml blasticidin selection was started 24 hours after lentiviral infection. For V-tagged inducible expression of SOX9, PLIX43 vectors were used and 15 g/ml blasticidin selection was started 24 hours after infection. For complete oligos and cloning, please reference Table 3.

TABLE 3
CAS9/CRISPR and shRNA cloning

| CRISPR | | | | | | | |
|---|---|---|---|---|---|---|---|
| CLONING | | | | | | | |
| NG | | | | | | | |
| V2 | | | | | | | |
| vector | | | Sequence | | | Location | |
| sgRNA | | | GAGGCTAAGCGTCGCAA | | | Distal U6 promoter | |
| Control | | | | | | | |
| hSOX9- | | CACCG | GCAGCACAAGAAGGACCACC | | | exon 2 | |
| sg2F | | | | | | | |
| hSOX9- | | AAAC | GGTGGTCCTTCTTGTGCTGC | C | | | |
| sg2R | | | | | | | |
| hSOX9- | | CACCG | GCTCGGACACCGAGAACACG | | | exon 1 | |
| sg4F | | | | | | | |
| hSOX9- | | AAAC | CGTGTTCTCGGTGTCCGAGC | C | | | |
| sg4R | | | | | | | |
| hSOX9- | | CACCG | CATGAAGATGACCGACGAGC | | | exon 1 | |
| sg5F | | | | | | | |
| hSOX9- | | AAAC | GCTCGTCGGTCATCTTCATG | C | | | |
| sg5R | | | | | | | |
| hSOX9- | | CACCG | GGCACCCCCGCAGCAGCCAC | | | exon 3 | |
| sg9F | | | | | | | |
| hSOX9- | | AAAC | GTGGCTGCTGCGGGGGTGCC | C | | | |
| sg9R | | | | | | | |
| SOX9 | | | | | | | |
| shRNA | | | | | | | |
| cloning | | | | | | | |
| PLKO. | | | | | | | |
| 1 and | | Restriction | | | | | |
| TET- | | enzyme | | | | | |
| PLKO | | site | | | | | |
| human | | AGE1/ECOR1 | sense | | | antisense | |
| shRNA# | top | CCGG | GCATCCTTCAATTICTGTATA | | CTCGAG | TATACAGAAATTGAAG | TTTT |
| 1 | | | | | | GATGC | T |
| 3'UTR | bottom | AATT | AAAAAGCATOCTTCAATTTCT | | CTCGAG | TATACAGAAATTGAAG | |
| | | | GTATA | | | GATGC | |
| shRNA# | top | CCGG | ACTTCTGAACGAGAGCGAGA | | CTCGAG | TTCTCGCTCTCGTTCAG | TTT |
| 2 | | | A | | | AAGT | TT |
| coding | bottom | AATT | AAAAAACTTCTGAACGAGAG | | CTCGAG | TTCTCGCTCTCGTTCAG | |
| region | | | CGAGAA | | | AAGT | |
| shRNA# | top | CCGG | ACCTTCGATGTCAACGAGTTT | | CTCGAG | AAACTCGTTGACATCG | TTTT |
| 4 | | | | | | AAGGT | T |
| coding | bottom | AATT | AAAAAACCTTCGATGTCAAC | | CTCGAG | AAACTCGTTGACATCG | |
| region | | | GAGTTT | | | AAGGT | |
| shRNA# | top | CCGG | CTCCACCTTCACCTACATGAA | | CTCGAG | TTCATGTAGGTGAAGG | TTTT |
| 5 | | | | | | TGGAG | T |
| coding | bottom | AATT | AAAAACTCCACCTTCACCTAC | | CTCGAG | TTCATGTAGGTGAAGG | |
| region | | | ATGAA | | | TGGAG | |
| mouse | | | | | | | |
| shRNA# | top | CCGG | CGTCAATGAGTTTGACCAAT | | CTCGAG | TATTGGTCAAACTCATT | TTTT |
| 2 | | | A | | | GACG | T |
| coding | bottom | AATT | AAAAACGTCAATGAGTTIGA | | CTCGAG | TATTGGTCAAACTCATT | |
| region | | | CCAATA | | | GACG | |
| shRNA# | top | CCGG | CTCCACCITCACITACATGAA | | CTCGAG | TTCATGTAAGTGAAGG | |
| 5 | | | | | | TGGAG | |
| coding | bottom | AATT | AAAAACTCCACCTTCACTTAC | | CTCGAG | TTCATGTAAGTGAAGG | TTTT |
| region | | | ATGAA | | | TGGAG | T |
| TET- | | Restriction | | | | | |
| Cellecta | | enzyme | | | | | |
| | | site | | | | | |
| mouse | | BbsI | sense | | | antisense | |
| shRNA# | top | ACCGG | CGTCAATGAGTTTGACCAAT | | GTTAATATTCA | TATTGGTCAAACTCATT | TTTT |
| 2 | | | A | | TAGC | GACG | |
| coding | bottom | CGAAAA | CGTCAATGAGTTTGACCAAT | | GCTATGAATAT | TATTGGTCAAACTCATT | C |
| region | | AA | A | | TAAC | GACG | |
| shRNA# | top | ACCGG | CTCCACCTTCACTTACATGAA | | GTTAATATTCA | TTCATGTAAGTGAAGG | TTTT |
| 5 | | | | | TAGC | TGGAG | |
| coding | bottom | CGAAAA | CTCCACCTTCACTTACATGAA | | GCTATGAATAT | TTCATGTAAGTGAAGG | C |
| region | | AA | | | TAAC | TGGAG | |
| PROM1 | | | | | | | |
| shRNA | | | | | | | |
| cloning | | | | | | | |
| | | Restriction | | | | | |
| TET- | | enzyme | | | | | |
| Cellecta | | site | | | | | |
| human | | BbsI | sense | | | antisense | |
| shRNA# | top | ACCGG | CCCAACATCATCCCTGTTCTT | | GTTAATATTCA | AAGAACAGGGATGATG | TTTT |
| 1 | | | | | TAGC | TTGGG | |
| coding | bottom | CGAAAA | CCCAACATCATCCCTGTTCTT | | GCTATGAATAT | AAGAACAGGGATGATG | C |
| region | | AA | | | TAAC | TTGGG | |
| shRNA# | top | ACCGG | GCGTCTTCCTATTCAGGATAT | | GTTAATATTCA | ATATCCTGAATAGGAA | TTTT |
| 2 | | | | | TAGC | GACGC | |
| coding | bottom | CGAAAA | GCGTCTTCCTATTCAGGATAT | | GCTATGAATAT | ATATCCTGAATAGGAA | C |
| region | | AA | | | TAAC | GACGC | |
| shRNA# | top | ACCGG | GTGTACAGTAAACGGTGTAT | | GTTAATATTCA | TATACACCGTTTACTGT | TTTT |
| 4 | | | A | | TAGC | ACAC | |
| 3'UTR | bottom | CGAAAA | GTGTACAGTAAACGGTGTAT | | GCTATGAATAT | TATACACCGTTTACTGT | C |
| | | AA | A | | TAAC | ACAC | |
| shRNA# | top | ACCGG | TGTGGTACAGCCGCGTGATTT | | GTTAATATTCA | AAATCACGCGGCTGTA | TTTT |
| 5 | | | | | TAGC | CCACA | |
| coding | bottom | CGAAAA | TGTGGTACAGCCGCGTGATTT | | GCTATGAATAT | AAATCACGCGGCTGTA | C |
| region | | AA | | | TAAC | CCACA | |
| NTC | top | ACCGG | CAACAAGATGAAGAGCACCA | | GTTAATATTCA | TTGGTGCTCTTCATCTT | TTTT |
| | | | A | | TAGC | GTTG | |
| | bottom | CGAAAA | CAACAAGATGAAGAGCACCA | | GCTATGAATAT | TTGGTGCTCTTCATCTT | C |
| | | AA | A | | TAAC | GTTG | |

h. RNA Isolation and qPCR

Total RNA was isolated using the RNeasy Mini Kit (Qiagen, Germantown, MD, USA) and cDNA was synthesized using the iScript™ Reverse Transcription Supermix for RT-qPCR (Bio-Rad) according to the manufacturer's instructions. Gene-specific primers for SYBR Green real-time PCR were either obtained from previously published sequences or designed by PrimerBLAST (available on the World Wide Web at ncbi.nlm.nih.gov/tools/primer-blast/) and synthesized by Integrated DNA Technologies or ETON biosciences. Real-time PCR was performed and analyzed using CFX96 Real-Time PCR Detection System (Bio-Rad Laboratories, Inc., Hercules, CA) and using Power SYBR Green PCR Master Mix (Thermo Fisher Scientific) according to the manufacturer's instructions. Relative mRNA expression was determined by normalizing to GAPDH expression, which served as an internal control. See Table 4 for primers used for qPCR.

i. Immunoblot, Antibodies, and Inhibitors

Immunoblot analysis was performed as previously described (Wong et al. (2018) Nature medicine 24: 968-977). Briefly, cells were lysed in RIPA buffer supplemented with a protease inhibitor cocktail (Roche). Whole cell extracts were resolved by SDS-PAGE, transferred to PVDF membranes, and probed with indicated primary antibodies. Bound antibodies were detected with horseradish peroxidase (HRP)-conjugated secondary antibodies and chemiluminescent HRP substrate.

The following primary antibodies were used for western blotting (all from Cell Signaling Technologies, Beverly, MA, USA, unless otherwise indicated): anti-SOX9 (#82630, 1:1,000), anti-TAZ (#8418S, 1:1,000), anti-Vinculin (#13901, 1:1,000), anti-E-cadherin (#3195, 1:1,000), anti-Vimentin (#5741, 1:1,000), anti-GAPDH (#2118, 1:1,000), anti-KRT20 (1:1,000, #13063), anti-Krt20 (1:1,000, ab97511, Abcam), anti-Axin2 (1:1,000, #5863), anti-PROM1 (1:1,000, #64326), anti-Prom1 (1:1,000, ab19898, Abcam), anti-P-AKT (1:1,000, #9271), anti-AKT (1:1,000, #9272), anti-P-S6 (1:1,000, #4858), anti-β-Actin (A5441, 1:1,000, Sigma), and anti-V5 (R960-25, 1:2,500, Thermo Fisher)

The following agents were used: recombinant WNT3A as a Wnt activator and ICG-001 as a Wnt pathway inhibitor, all of which were employed at indicated concentrations and durations.

TABLE 4
RT-PCT primers

| | | Sequence | |
|---|---|---|---|
| Species | Gene | forward | reverse |
| Human | SOX9 #1 | GTACCCGCACTTGCACAAC | TCTCGCTCTCGTTCAGAAGTC |
| Human | SOX9 #2 | AGACAGCCCCCTATCGACTTC | TGCTGCTTGGACATCCACAC |
| Human | SOX9 #3 | AAACATGACCTATCCAAGCGCATTA | TTAGCACACTGATCACACGATTCTC |
| Human | GAPDH | TGTTGCCATCAATGACCCCTT | CTCCACGACGTACTCAGCG |
| Human | CDH1 | GCCGCTGGCGTCTGTAGGAA | TGACCACCGCTCTCCTCCGA |
| Human | MUC2 | CGACTACTACAACCCTCCGC | GGGAGGAGTTGGTACACACG |
| Human | CDX2 | AGACCAACAACCCAAACAGC | CCCGAACAGGGACTTGTTTA |
| Human | KRT20 | TGGCCTACACAAGCATCTGG | TAACTGGCTGCTGTAACGGG |
| Human | CD44 | GACACATATTGTTTCAATGCTTCAGC | GATGCCAAGATGATCAGCCATTCTGGAAT |
| Human | BMI1 | TGGAGAAGGAATGGTCCACTTC | GTGAGGAAACTGTGGATGAGGA |
| Human | SNAIL | GCACATCCGAAGCCACAC | GGAGAAGGTCCGAGCACAC |
| Human | PROM1 | AACCTACAGCATATTCTTCA | AACGAACAGCATTTCTCTCTCAAGA |
| Human | | | |
| mouse | Sox9 #1 | CCACGGAACAGACTCACATC | CCCTCTCGCTTCAGATCAAC |
| mouse | Sox9 #2 | CAAGCGGAGGCCGAAGA | CAGCTTGCACGTCGGTTT |
| mouse | | | |
| mouse | Gapdh | TCCCACTCTTCCACCTTCGATGC | GGGTCTGGGATGGAAATTGTGAGG |
| | primer #1 | | |
| mouse | Gapdh | CCCATGTTTGTGATGGGTGT | GTGATGGCATGGACTGTGGT |
| | primer #2 | | |
| mouse | Lrig1 | TAGAGGAGTGGCATCTGGAG | CCATGCGCTAAGGATTAAAA |
| mouse | Lgr5 | GACGCTGGGTTATTTCAAGTTCAA | CAGCCAGCTACCAAATAGGTGCTC |
| mouse | Axin2 | CTCCCCACCTTGAATGAAGA | TGGCTGGTGCAAAGACATAG |
| mouse | Ascl2 | GCCTACTCGTCGGAGGAA | CCAACTGGAAAAGTCAAGCA |
| mouse | Prom1 | GATCAGGCCAACAACTATGG | CCAGGAGTGTTATGGAATGC |
| mouse | Krt20 | CGCTCCCTGGAGCAATCCAA | CAATTCGCAGGACGCACTGG |

j. Chromatin Immunoprecipitation and DNA Sequencing (ChIP-Seq)

HT115 cells were washed with PBS and crosslinked with 1% formaldehyde for 10 minutes for H3K27ac immunoprecipitation or crosslinked with two agents starting with 2 mM DSG (Pierce) for 45 minutes at room temperature followed by 1 mL of 1% formaldehyde for 10 minutes for the V5. Cross-linked cell lines were quenched with 0.125 M glycine for 5 minutes at room temperature. Cross-linked material was resuspended in 1% SDS (50 mM Tris-HCl pH8, 10 mM EDTA) and sonicated for 5 minutes with a Covaris E220 instrument (5% duty cycle, 140 Peak Incident Power, 200 Cycles per burst, 1 mL AFA Fiber milliTUBEs). Soluble chromatin (5 μg) was immunoprecipitated with 10 μg of H3K27ac (Diagenode C15410196 antibody) or 40 ug chromatin with 10 ug V5 Tag Antibody (Invitrogen Cat #R96025 Lot #1949337). ChIP-seq libraries were constructed using Accel-NGS 2S DNA library kit from Swift Biosciences. Fragments of the desired size were enriched using AMPure XP beads (Beckman Coulter). 36-bp paired-end reads were sequenced on a Nextseq instrument (Illumina).

k. ChIP-Seq Analysis

The ChiLin pipeline 2.0.0 (Qin et al. (2016) BMC Bioinformatics 17: 404) was used for quality control and pre-processing of the data. A Burrows-Wheeler Aligner (BWA Version: 0.7.17-r1188) was used as a read mapping tool, and Model-based Analysis of ChIP-Seq (MACS2) (Zhang et al. (2008) Genome biology 9: R137)(v2.1.0.20140616) as a peak caller using default parameters. CEAS analysis is used to annotate resulting peaks with genome features. Differential analysis of peaks was determined by DESeq (Anders S et al. (2010) Genome Biology 11: 10). BETA (Wang et al. (2013) Nat Protoc 8: 2502-2515) was used to integrate ChIP-seq of transcription factors or chromatin regulators with differential gene expression data to infer direct target genes. Super-enhancers were called by ROSE (Whyte et al. (2013) Cell 153: 307-319) in H3K27ac ChIP-seq data. Cistrome toolkit was used to probe which factors might regulate the user-defined genes. Genomic Regions Enrichment of Annotations Tool (GREAT) (McLean et al. (2010) Nat Biotechnol 28: 495-501) was used to annotate peaks with their biological functions. Conservation plots were obtained with the Conservation Plot (version 1.0.0) tool available in Cistrome.

l. ChIP-Seq Data Visualization

Normalized profiles corresponding to read coverage per 1 million reads were used for heatmaps and for visualization using the integrative genomics viewer (IGV) (Thorvaldsdottir et al. (2013) Brief Bioinform 14: 178-192). Wiggle tracks were visualized using the integrative genomics viewer. Heat maps were prepared using deepTools (version 2.5.4) (Ramirez et al. 2014).

### m Reporter Assay

A 537-bp region in intron 1 of PROM1 (hg19, ch4:16,068,931-16,069,504, negative strand) was identified as a putative SOX9-regulated enhancer based on V5-SOX9 and H3K27Ac CHIP data. PCR-amplified products or gene blocks (gBlocks, IDT) consisting of the putative PROM1 enhancer, a minimal promoter, eGFP, and PGK-hygromycin resistance cassette was cloned into the lentiviral LV 1-5 backbone vector using Gibson modular assembly platform (Akama-Garren et al. (2016) Sci Rep 6: 16836). Briefly, PROM1 enhancer was generated as a gBlock by adding the following primer sequencing to the 5′ and 3′ end of the 537-bp region, respectively: 5′→3′ Forward (site 1), GATCAGTGTGAGGGAGTGTAAAGCTGGTTT and 5′→3′ Reverse, CTAACTCGAACGCTAGCTGTGCGATCGTTT (site 2); the minimal promoter was PCR amplified from the 7TFP WT CDH1 reporter plasmid using the following primer sequences: 5′→3′ Forward (includes site 2), CTAACTCGAACGCTAGCTGTGCGATCGTTTAGTCAGTTCAGACTCCAGCCC and 5′→3′ Reverse, AGGCCTCGGGATTcctaggAACAGCGGTTTGTGGCTTTACCAACAGTACCGGAA (includes site 3); eGFP was generated by adding the following primer sequences to the 5′ and 3′ end, respectively: 5′→3′ Forward (site 3), AAACCGCTGTTcctaggAATCCCGAGGCCT and 5′→3′ Reverse, GACCCGACATTagcgctACAGCTTAAGCGG (site 4); and PGK-hygro was PCR amplified from plenti-PGK-hygro-gateway destination vector using the following primers: 5′→3′ Forward (includes site 4), GACCCGACATTagcgctACAGCTTAAGCGGACTAGTGATCTCTCGAGGTTAACGAA T and 5′→3′ Reverse, AGAGTAATTCAACCCCAAACAACAACGTTTTCAGCTCTTGTTCGGTCGGCA

(includes site 5). PCR products were extracted from agarose gels after electrophoresis using the QIAquick Gel Extraction kit (Qiagen) according to the manufacturer's protocol. PCR products and gBlocks were then adjusted to 57 nM with Tris EDTA buffer (pH=8.0). 5 μL of PCR products and gBlocks (5.7×10⁻² pmol each) were added to 15 μl of isothermal master mix, incubated at 50° C. for 20 min, and transformed into competent bacteria. Isothermal master mix was prepared by combining 320 μL of 5× isothermal assembly reaction buffer, 1.2 μL of T5 exonuclease (NEB), 20 μL of Phusion polymerase (NEB), 160 μL of Taq ligase (NEB) and 700 μL of water. 5× isothermal assembly reaction buffer was prepared by combining 3 ML of 1M Tris-HCL (pH=7.5), 300 uL of 1M MgCl2, 600 μl of 10 mM dNTPs, 300 μL of 1M DTT, 1.5 g of PEG-8000, 20 mg of NAD and water to a total of 6 mL. The resultant reporter vector was transiently transfected into HEK293T cells and either treated with Wnt3a and/or concomitantly transfected with indicated expression plasmids.

n. Genetic Dependencies

To evaluate genetic dependencies, the genetic dependency combined RNAi dataset and CRISPR Avana dataset at DepMap portal (available on the World Wide Web at depmap.org) (19Q4 data release), which is a public catalog of essential genes and dependencies for cell line proliferation as determined by genome-wide RNAi (three publicly available dataset: Achilles, Novartis and Marcotte) and CRISPR screening, were interrogated. Higher negative values of combined RNAi or CRISPR dependency score represent higher dependency for that gene.

o. Statistical Analysis and Reproducibility

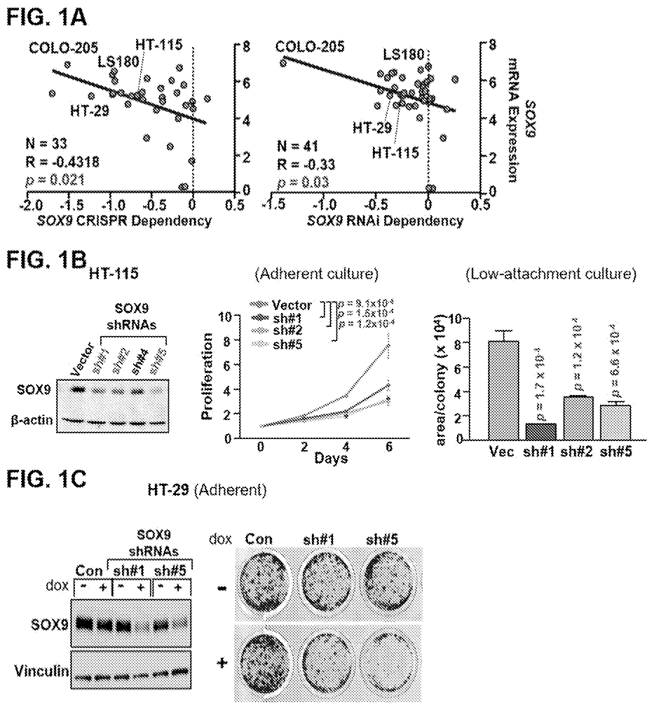

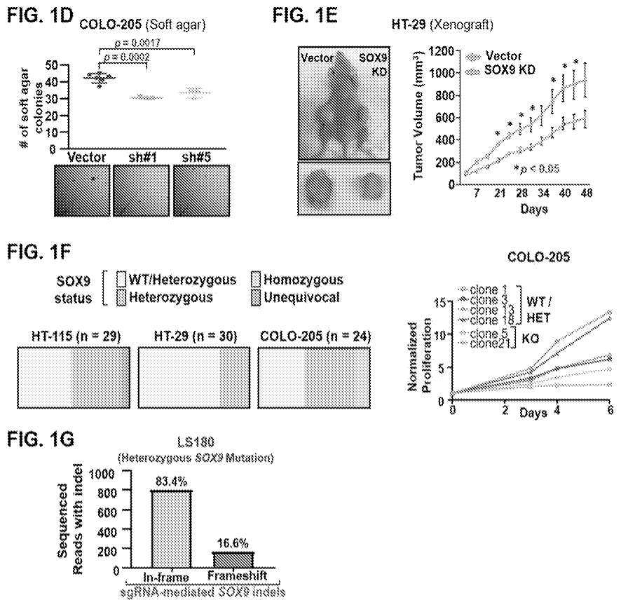

Experiments were performed in triplicate. Data are represented as mean±s.d unless indicated otherwise. For each experiment, either independent biological or technical replicates are as noted in the FIG. 1 legends and were repeated with similar results. Statistical analysis was performed using Microsoft Office or Prism 7.0 (GraphPad) statistical tools. Pairwise comparisons between groups (that is, experimental versus control) were performed using an unpaired two-tailed Student's t-test or Kruskal-Wallis test as appropriate unless otherwise indicated. For all experiments, the variance between comparison groups was found to be equivalent. Animals were excluded from analysis if they were euthanized due to health reasons unrelated to tumor volume endpoint.

### Example 2: Human CRC is Dependent on SOX9

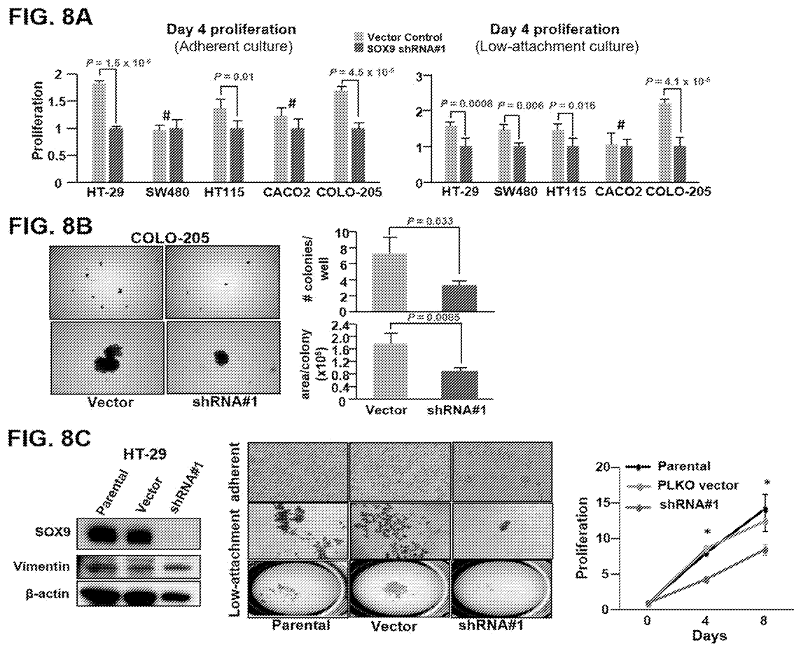

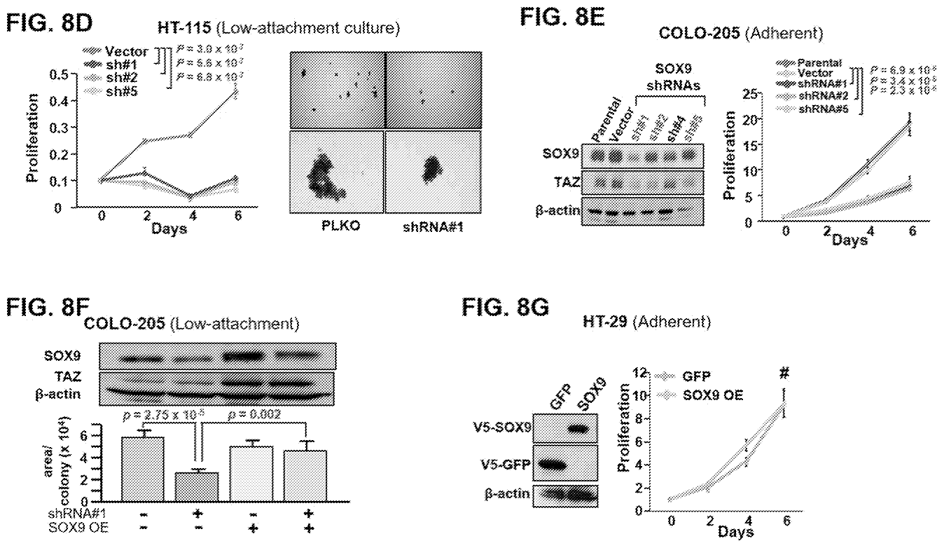

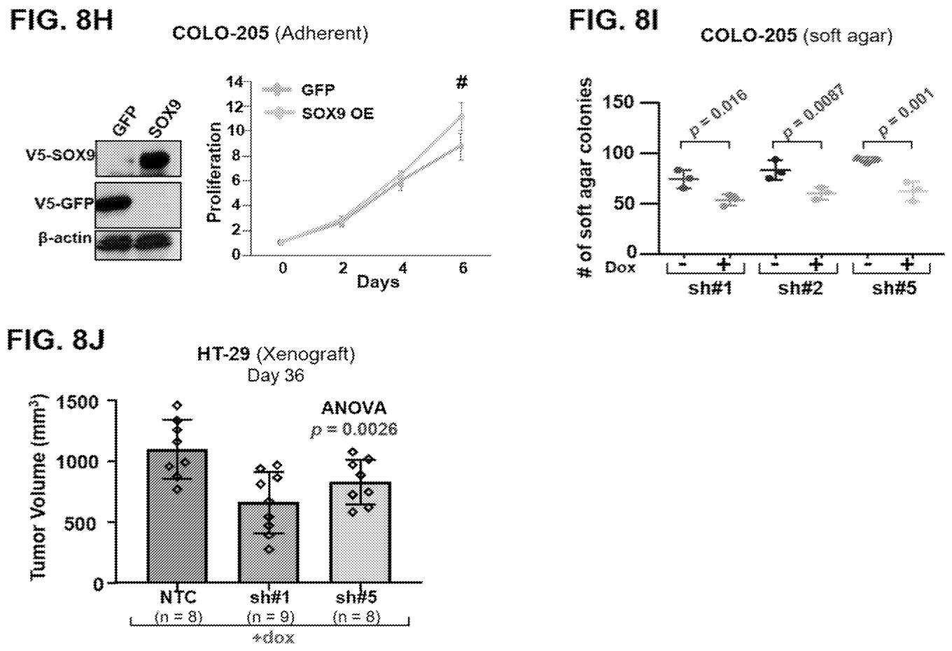

To investigate whether SOX9 is required for proliferation in human CRC, large genome-scale functional cell line datasets were analyzed (Ghandi et al. (2019) Nature 569: 503-508). SOX9 knockdown (KD) or knockout (KO) using RNA interference (n=41) and clustered regularly interspaced short palindromic repeats (CRISPR)/Cas9 (n=33), respectively, led to proliferation defects in CRC cell lines (FIG. 1A); cell lines with higher SOX9 expression were more dependent. To confirm these results, SOX9 were suppressed using stable shRNA expression and observed proliferation defects in several CRC cell lines surveyed during adherent or ultra-low attachment culture (FIG. 8A-C). Focusing on CRC cell lines most sensitive to SOX9 depletion, constitutive or inducible expression of multiple SOX9 shRNAs impaired proliferation of HT-115, HT-29, and COLO-205 CRC cells (FIG. 1B-C; FIG. 8D-F). Consistent with these phenotypes, SOX9 KD was associated with reduced expression of Hippo/Yes-associated protein (YAP) downstream target TAZ (FIG. 8E), which is a marker of survival and proliferation in the intestines (Moroishi et al. (2015b) Nat Rev Cancer 15: 73-79; Taniguchi et al. (2015) Nature 519: 57-62) and driver of CRC tumorigenesis (Moroishi et al. (2015a) Genes Dev 29: 1271-1284). Re-expression of SOX9 cDNA rescued ultra-low attachment colony-forming defects and restored TAZ expression in COLO-205 cells stably expressing a 3′UTR-targeting SOX9 shRNA (FIG. 8F). Overexpression of SOX9 alone did not improve adherent growth or ultra-low attachment colony formation in CRC cells, presumably due to high endogenous SOX9 expression levels in CRC (FIG. 8F-H). Constitutive or inducible SOX9 KD partially reduced soft-agar colony formation in vitro (FIG. 1D; FIG. 8I) and impaired primary tumor growth in vivo (FIG. 1E; FIG. 8J).

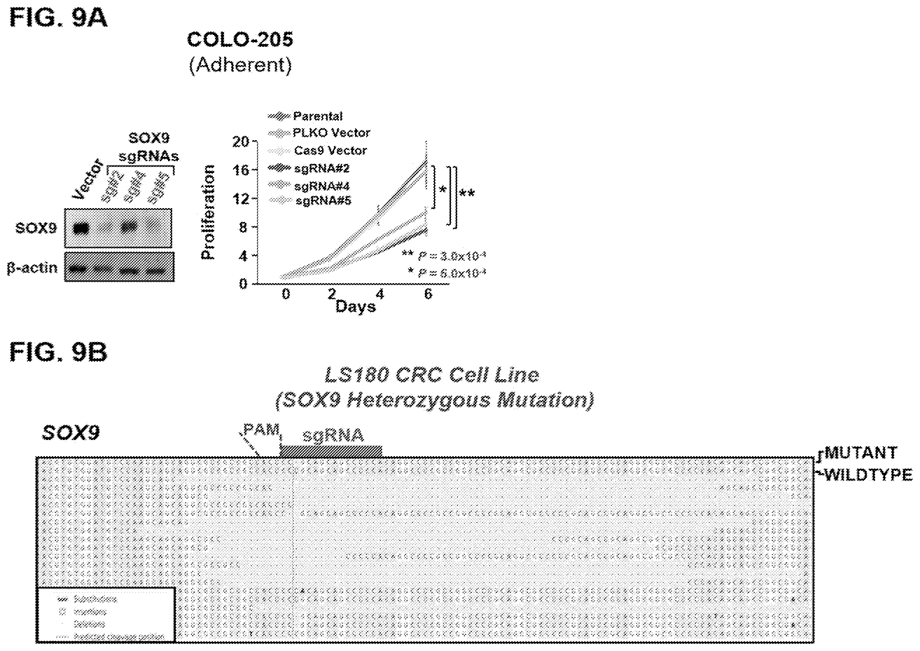

Next, SOX9 in CRC cell lines using CRISPR/Cas9 were attempted to eliminate. Among the CRC cell lines tested, COLO-205 was the only one that appeared to tolerate SOX9 KO, which resulted in compromised proliferation relative to multiple controls (FIG. 9A). To further investigate this result, single cell clones from three cell lines stably expressing an inactivating SOX9 sgRNA and Cas9 were analyzed. Consistent with the previous results, HT-115 and HT-29 clones did not demonstrate homozygous deletions whereas a minority of COLO-205 clones harbored biallelic SOX9 inactivation (FIG. 1F). As anticipated, COLO-205 clones with biallelic SOX9 deletion grew poorly relatively to isogenic controls (FIG. 1F). Furthermore, compared to parental controls, COLO-205 clones with biallelic SOX9 KO were unable to form primary tumor xenografts in vivo (0/10 vs 10/10).

SOX9 is mutated in approximately 10% of CRC (Cancer Genome Atlas (2012) Nature 487: 330-337; Liu et al. (2018) Cancer Cell 33: 721-735 e728). While the functional significance of these mutations is unknown, the majority have been heterozygous alterations (Duronio et al., unpublished). It was investigated whether CRC with heterozygous SOX9 mutations are dependent on the remaining wildtype (WT) SOX9 allele. Genomic analysis of LS180, a CRC cell line harboring a heterozygous SOX9 mutation, stably expressing an inactivating SOX9 sgRNA and Cas9 demonstrated that the majority of indels are in-frame (FIG. 1G, FIG. 9B), likely preserving the function of WT SOX9. Among the frameshift mutations that could be verified, the majority were found in the mutant SOX9 allele. Collectively, these results suggest that the majority of human CRC is dependent on SOX9.

### Example 3: SOX9 Blocks Intestinal Differentiation in Human CRC

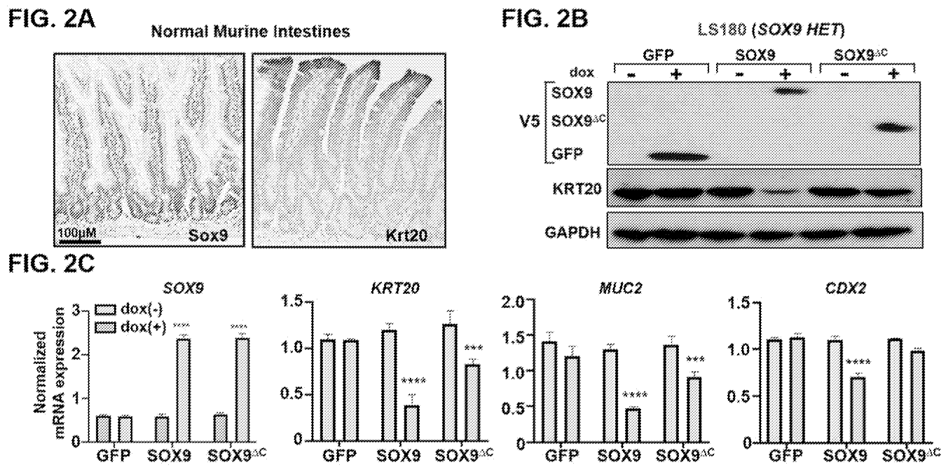

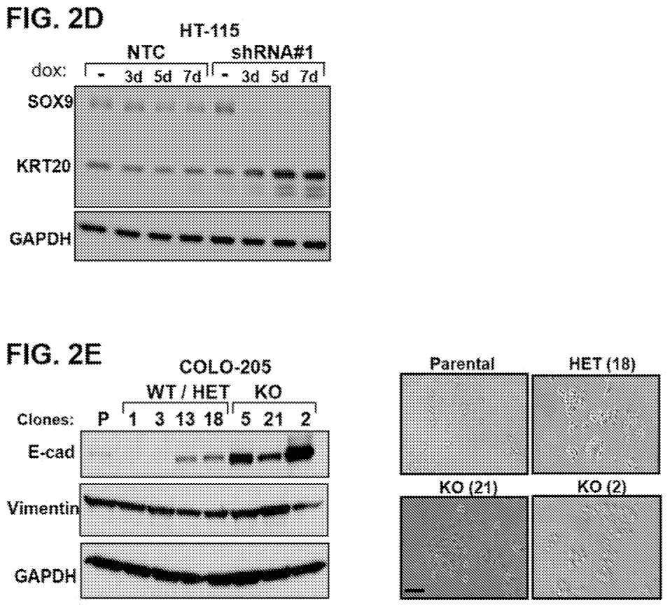

Blocking differentiation is a key mechanism by which cancers survive, persist, and grow, especially in regenerative organs such as the intestines. Known for its regulation of cell fate decisions in many tissues, SOX9 dependency in human CRC may relate to an ability to regulate intestinal differentiation. Keratin-20 (Krt20) is a tissue-specific marker of intestinal differentiation (Dow et al. (2015) Cell 161: 1539-1552) as shown by its villus-restricted expression in the normal mouse intestines (FIG. 2A). In a mutually exclusive pattern, Sox9 is expressed in the crypt of murine intestines (FIG. 2A), suggesting that Sox9 may restrict intestinal differentiation.

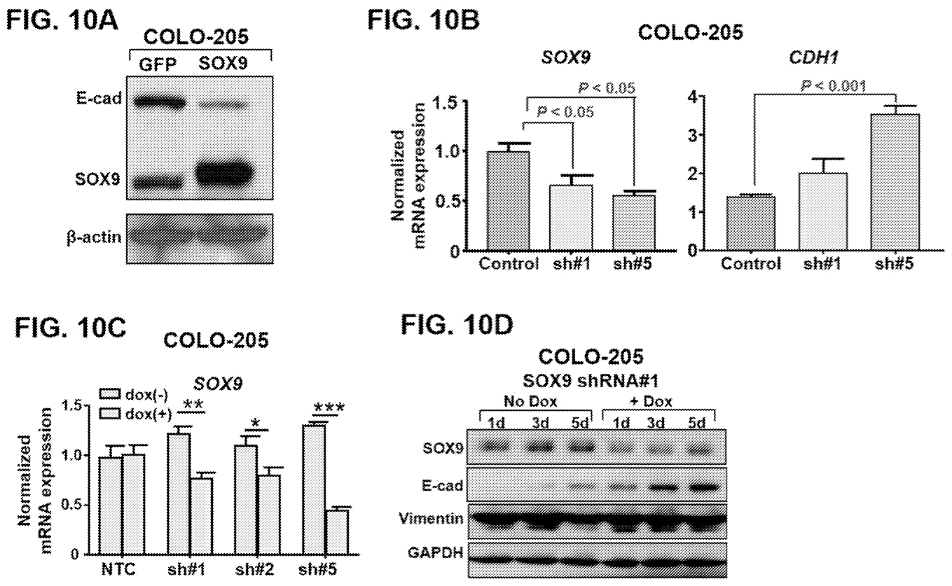

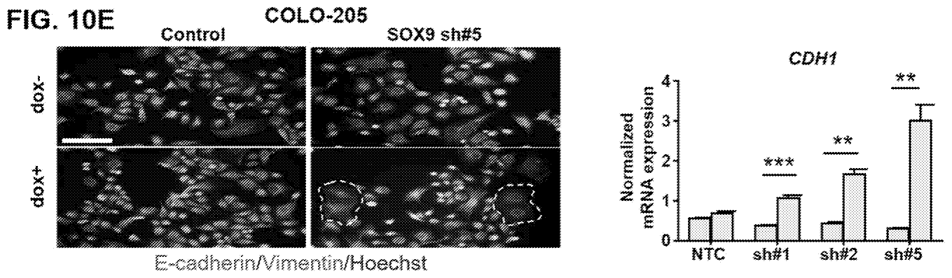

To investigate this hypothesis, SOX9 expression was manipulated in human CRC cell lines. LS180 CRC cells were chosen for most overexpression experiments given low endogenous expression of SOX9 due to the endogenous heterozygous mutation. Inducible overexpression of SOX9 reduced expression of absorptive and secretory intestinal differentiation markers KR T20, MUC2, and CDX2 in LS180 CRC cells, whereas overexpression of a truncated, transcriptionally inactive SOX9 (SOX9^{ΔC}) had diminished to no effect (FIGS. 2B,C). Constitutive SOX9 overexpression also led to diminished expression of general epithelial differentiation marker E-cadherin in COLO-205, a CRC cell line without KRT20 expression (FIG. 10A). These data suggest that SOX9 negatively regulates intestinal differentiation in human CRC.

By contrast, inducible shRNA-mediated SOX9 KD promoted intestinal differentiation in HT-115 CRC cells as indicated by elevated KRT20 expression (FIG. 2D). Suppression of SOX9 under constitutive or inducible conditions in COLO-205 cells also led to elevated expression of CDH1/E-cadherin in a manner that correlated to the strength of shRNA-mediated KD (FIGS. 10B-D). Inducible SOX9 KD led to junctional localization of E-cadherin by immunofluorescence (FIG. 10E). Notably, the few COLO-205 clones that tolerated biallelic SOX9 KO displayed a pronounced “cobblestone” morphology that corresponded to markedly elevated E-cadherin expression (FIG. 2E), phenocopying SOX9 KID. These data indicate that SOX9 disruption promotes intestinal differentiation and likely explains the resultant impairment in human CRC growth.

### Example 4: SOX9 KD Impairs Proliferation and Induces Differentiation in Neoplastic Mouse Organoids

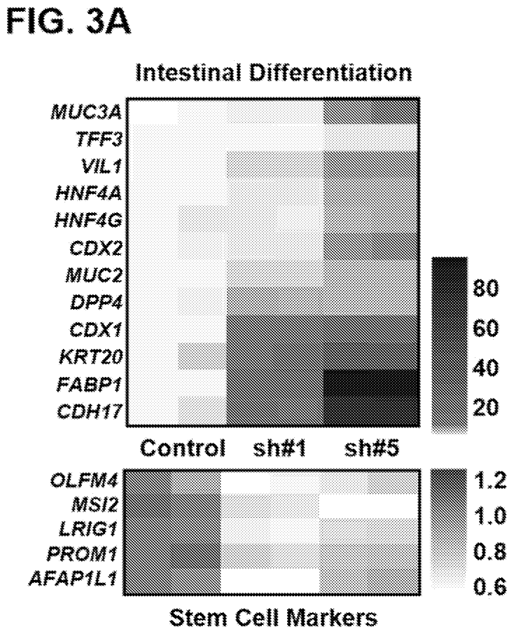

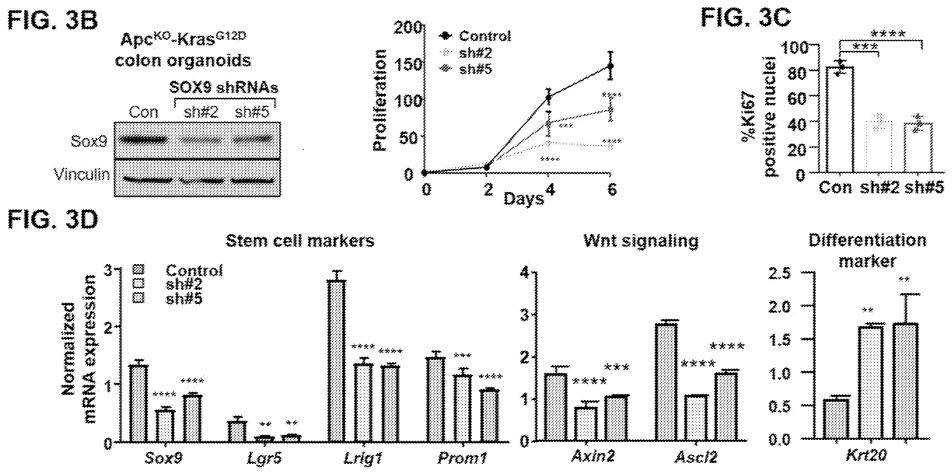

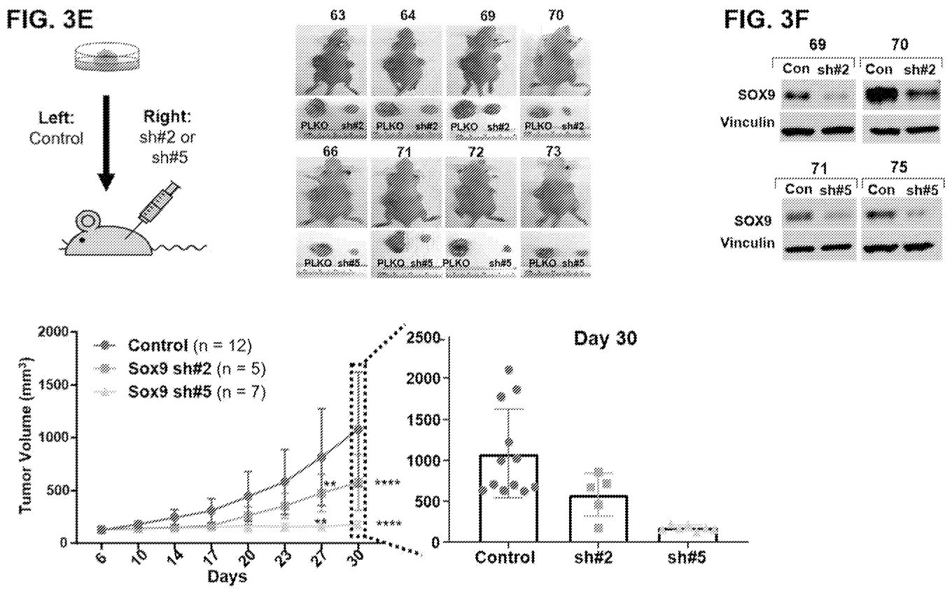

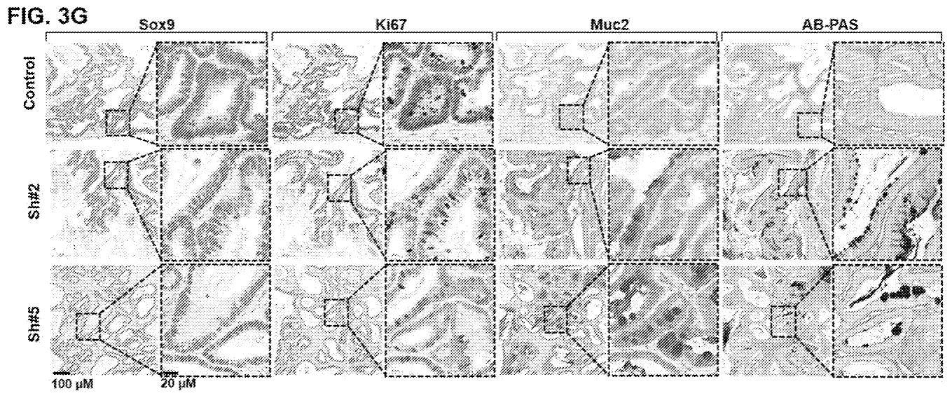

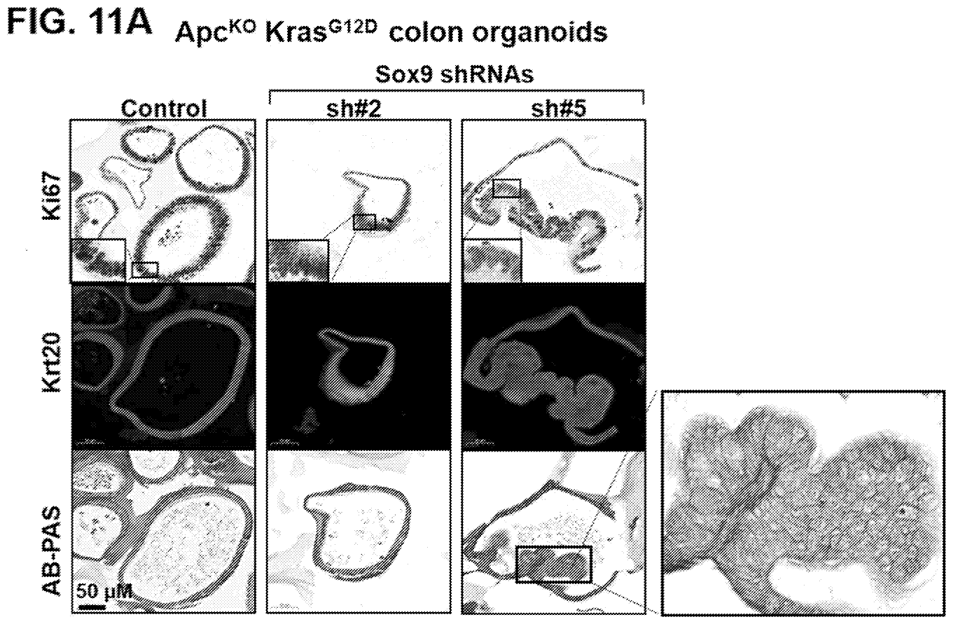

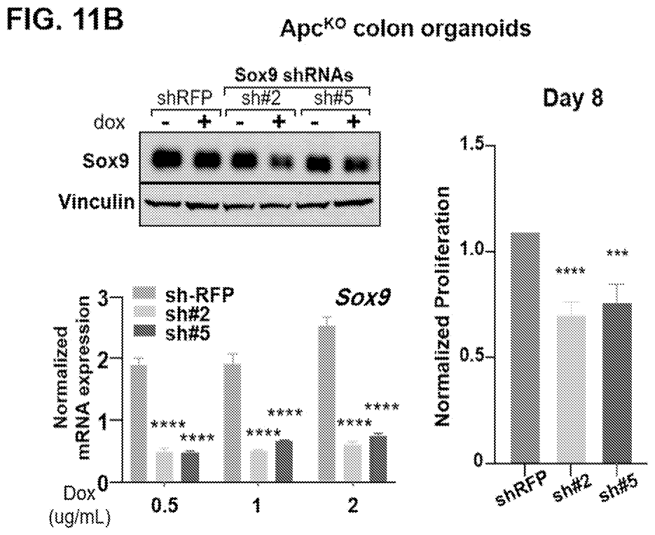

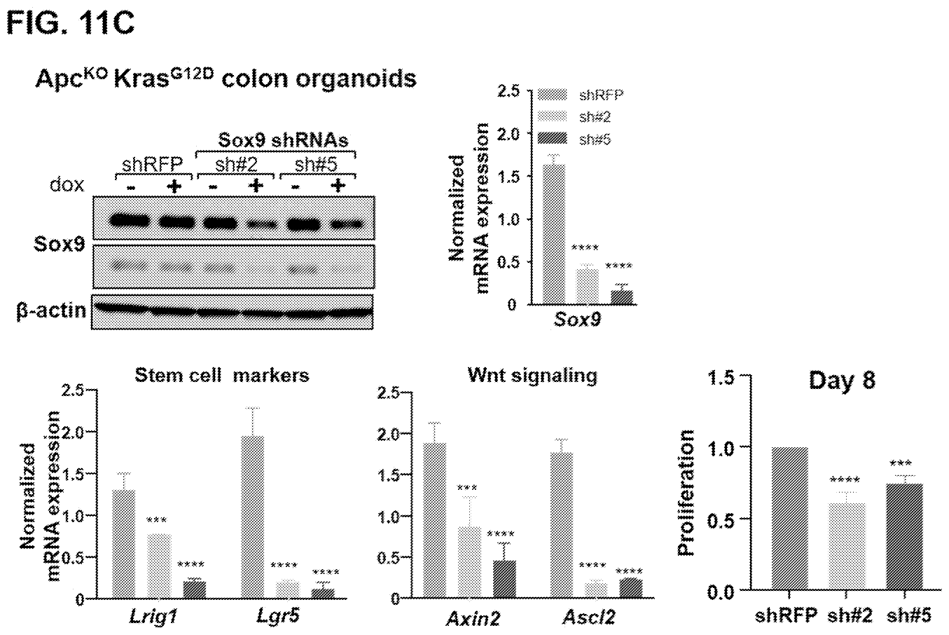

Organoids capture intestinal stem cell behavior and differentiation dynamics (Sato et al. (2009) Nature 459: 262-265), serving as a well-suited platform to investigate SOX9 function in CRC. It was examined whether SOX9 KD promotes intestinal differentiation in normal human colon organoids. Gene expression profiling by RNA-seg revealed that SOX9 suppression induces a broad intestinal differentiation program and reduced expression of a subset of stem cell markers (FIG. 3A). Apc^{KO}Kras^{G12D} neoplastic organoids derived from the colon of genetically-engineered mice were then utilized to investigate the impact of Sox9 KD on proliferation and differentiation. Constitutive Sox9 KD reduced growth by 2 to 3-fold in Apc^{KO}Kras^{G12D} colon organoids as indicated by in vitro culture assays and Ki67 immunohistochemistry (IHC) after fixation (FIG. 3B-C; FIG. 11A). The growth defects were associated with induction of intestinal differentiation as shown by alcian blue staining, which indicates presence of mucin-producing cells, and Krt20 immunofluorescence on fixed organoids (FIG. 11A). Sox9 KD reduced mRNA expression of stem cell markers and Wnt signaling components, while it induced Krt20 expression (FIG. 3D). These results were validated with constitutive Sox9 KD in Apc^{KO} colon organoids and inducible Sox9 KD in Apc^{KO}Kras^{G12D} colon organoids (FIG. 11B-C).

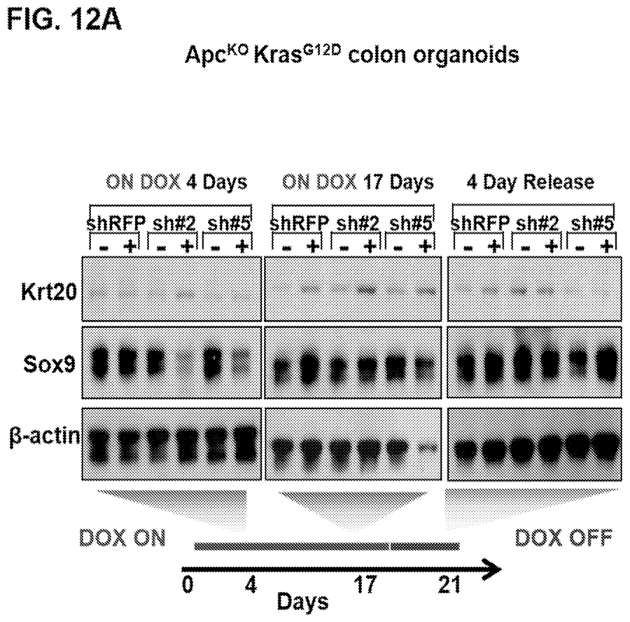

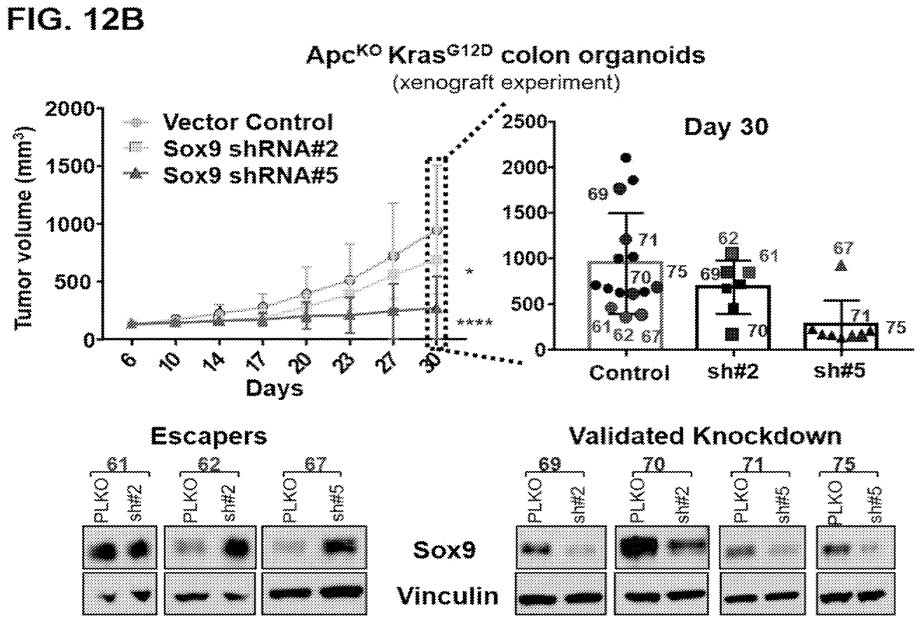

Using the doxycycline-inducible shRNA system, the kinetics and reversibility of intestinal differentiation in response to Sox9 KID were assessed by Krt20 expression in Apc^{KO} Kras^{G12D} colon organoids. A modest induction of intestinal differentiation was observed between 4 and 17 days of doxycycline treatment. A stronger differentiation induction was potentially counteracted by Wnt3A/R-spondin/Noggin (WRN) media, which is rich in stem cell growth factors, and less pronounced Sox9 suppression at day 17, perhaps due to the outgrowth of organoids that escaped shRNA-mediated KD. Following 4 days of doxycycline withdrawal, restoration of Sox9 and baseline Krt20 expression equivalent to levels found in control organoids (FIG. 12A) were observed. These findings suggest that intestinal differentiation induced by SOX9 KD is reversible.

Next, the ability of Sox9 KD to impact Apc^{KO} Kras^{G12D} colon organoids growth and differentiation in vivo were evaluated using primary tumor xenograft assays. Compared to controls injected into the contralateral flanks of nude mice, Sox9 KD organoids displayed significantly impaired primary tumor growth (FIG. 3E). Histopathological analysis confirmed reduced proliferative capacity by Ki67 IHC and demonstrated a clear induction of intestinal differentiation by Muc2 IHC and alcian blue staining in Sox9 KD xenografts (FIG. 3G). Proteins from tumors were collected at the experimental endpoint to validate Sox9 suppression. Although the majority of Sox9 KD tumors demonstrated reduced Sox9 expression (FIG. 3F), there were a subset of outliers that escaped shRNA-mediated Sox9 KD and demonstrated increased xenograft growth (FIG. 12B). These results demonstrate that Sox9 KD leads to intestinal differentiation and decreased tumor growth in neoplastic murine organoid models.

### Example 5: SOX9 Activates a Stem and Paneth Cell Transcriptional Program by Binding Genome-Wide Enhancers

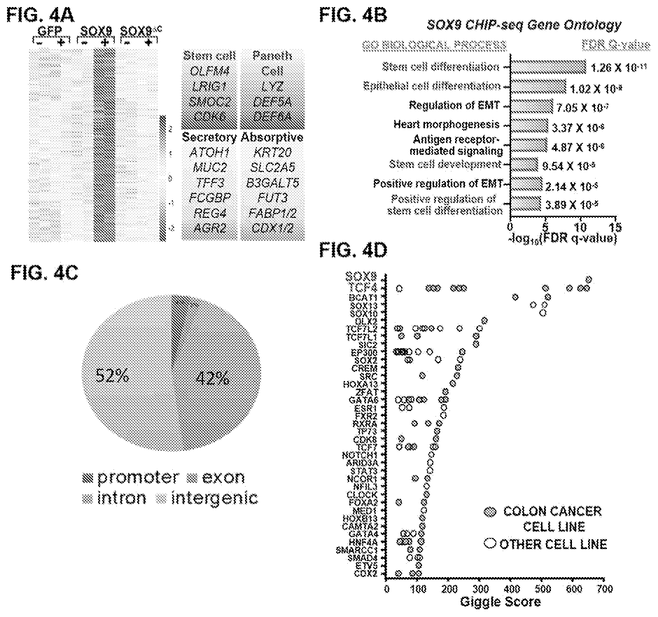

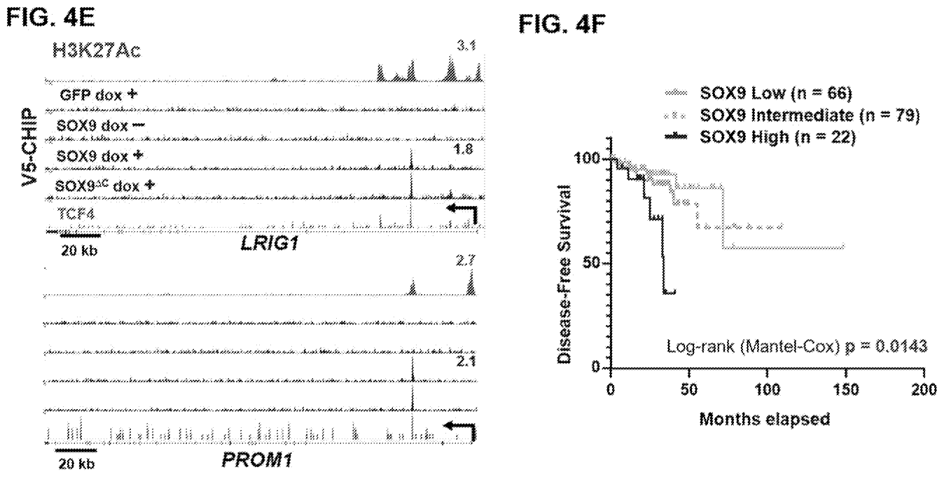

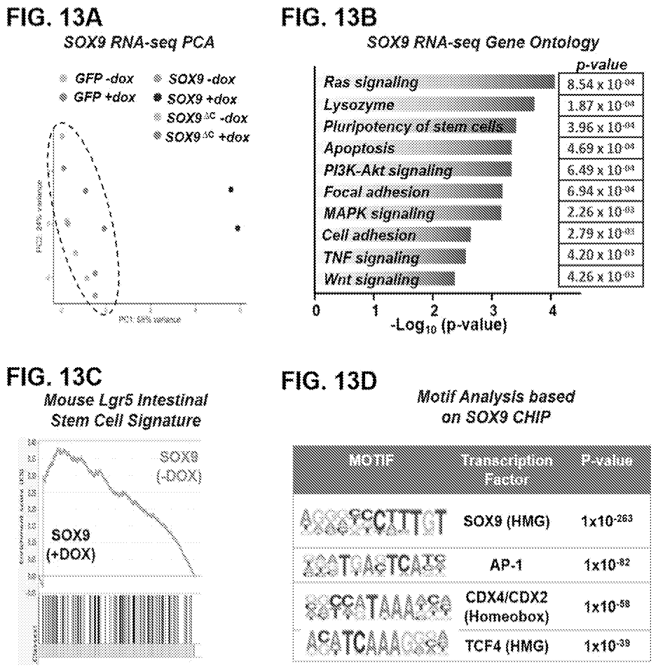

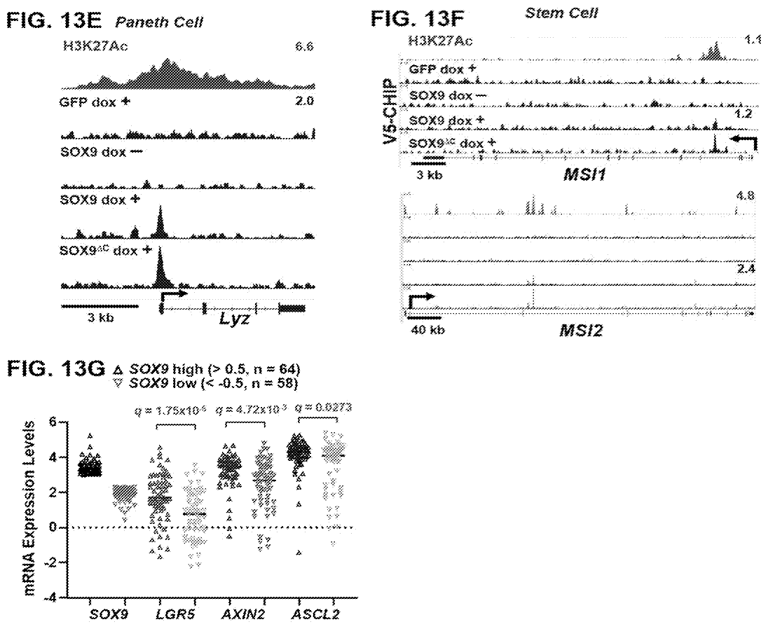

To determine the mechanism by which SOX9 blocks differentiation in human CRC, gene expression profiling of LS180 CRC cells engineered to conditionally overexpress GFP control, WT SOX9, or SOX9^{ΔC} by RNA-seq (FIG. 4A) was performed. Principle component analysis of top 500 differentially expressed genes indicated that SOX9 induces a distinct transcriptional state (FIG. 13A). Gene ontology analysis demonstrated that Ras signaling, Lysozyme and Pluripotency are the top three gene-sets upregulated upon inducible SOX9 expression (FIG. 13B), consistent with individual gene expression (FIG. 4A). Furthermore, genes upregulated by SOX9 overexpression were enriched for an Lgr5 intestinal stem cell signature (Munoz et al. (2012) EMBO J 31: 3079-3091) by GSEA (FIG. 13C). By contrast, differentiation genes associated with both absorptive and secretory cell lineages were among significantly downregulated genes following SOX9 overexpression (FIG. 4A). These data indicate that SOX9 may block differentiation by promoting a stem cell-like and Paneth cell transcriptional program.

To investigate the direct transcriptional program mediated by SOX9 in CRC, genome-wide V5 and H3K27Ac chromatin immunoprecipitation were performed followed by DNA sequencing (CHIP-seq) in CRC cells conditionally expressing V5-tagged GFP, WT SOX9 and SOX9^{ΔC}. Indeed, genes directly bound and activated by SOX9 were significantly enriched in gene-sets associated with stem cell activity (FIG. 4B). 94% of SOX9 binding occurred at intergenic and intronic regions of the genome (FIG. 4C) and over a fifth of genome-wide regions occupied by SOX9 are typical enhancers (˜374 sites). Superenhancers are composed of a high density of individual enhancers and often regulate tissue-specific genes involved in cellular identity (Heintzman et al. (2009) Nature 459: 108-112; Visel et al. (2009) Nature 457: 854-858). It was observed that SOX9 binding sites are more prevalent in super enhancers than typical enhancers. Of the H3K27Ac-marked super enhancers, 26% (33/128) are bound by SOX9, which is greater than the 15% (374/2455) of typical enhancers occupied by SOX9. These results are consistent with a potential role for SOX9 as a transcriptional regulator of stem cell-like activity by engaging enhancers.

Next, it was investigated whether other transcription factors bind to regions occupied by SOX9, reasoning that these factors may co-regulate stem cell-like behavior. Motif analysis inferred that binding sites for CDX2, an intestinal lineage transcription factor (James et al. (1994) The Journal of biological chemistry 269: 15229-15237; Suh et al. (1994) Mol Cell Biol 14: 7340-7351), and TCF712/TCF4, an essential Wnt signaling co-factor (Korinek et al. (1997) Science 275: 1784-1787; Morin et al. (1997) Science 275: 1787-1790, Korinek et al. (1998) Nat Genet 19: 379-383), are enriched at regions bound by SOX9 (FIG. 13D). Supporting this result, publicly-available genome-wide transcription factor binding data indicated a strong overlap among TCF4 and SOX9-bound regions, with most of these studies utilizing CRC cell lines (FIG. 4D). Consistent with the gene ontology analysis, SOX9 occupied intronic enhancers of several stem cell genes and the promoter of Paneth cell gene Lyz (FIG. 4E; FIG. 13E-F). LRIG1 and PROM1 are two stem cell genes co-occupied by SOX9 and TCF4 at intronic enhancers (FIG. 4E). These results suggest that the Wnt effector TCF4 may collaborate with SOX9 at specific enhancers to regulate a stem cell-like program in CRC.

To determine whether these relationships are found in tumors of patients with CRC, TCGA transcriptional profiles from samples with high and low expression of SOX9 were investigated. Several genes associated with stem cell activity displayed elevated expression in CRC samples with high SOX9 expression (FIG. 13G). Furthermore, high SOX9 expression portended a poor disease-free survival compared to intermediate and low expression (FIG. 4F). These data indicate that SOX9 mediates a stem cell-like transcriptional program by binding to genome-wide enhancers in CRC, which likely drives aggressive cancer biology.

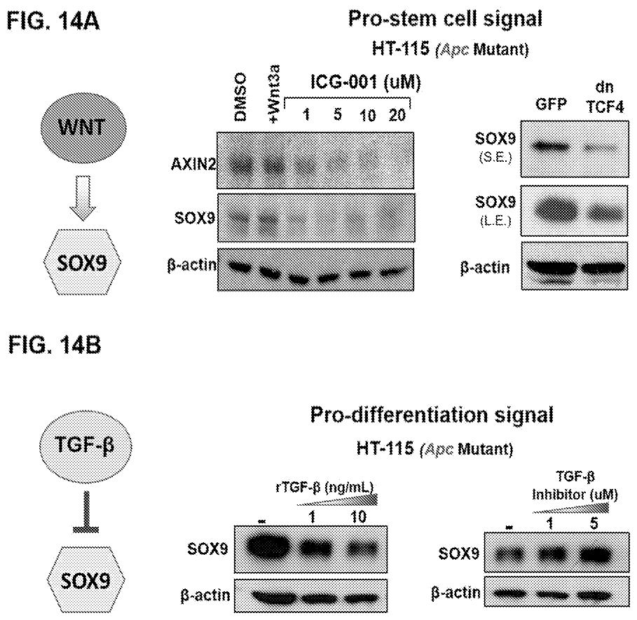

To examine whether SOX9 is regulated by stem cell and differentiation cues, the impact of the WNT and BMP/TGF-β signaling pathways on SOX9 expression, respectively, was evaluated. WNT signaling represses intestinal differentiation by activating stem cell transcriptional programs in the enteric crypt whereas BMP/TGF-β activity promotes post-mitotic differentiation in the villus (Sancho et al. (2004) Annu Rev Cell Dev Biol 20: 695-723). Consistent with literature (Blache et al. (2004) The Journal of cell biology 166: 37-47; Feng et al., (2013) Am J Pathol 183: 493-503), disrupting the WNT pathway using the chemical inhibitor ICG-001 or dominant-negative form of TCF4 (dnTCF4) reduced SOX9 expression in CRC cells (FIG. 14A), confirming that SOX9 is positively regulated by the WNT pathway. By contrast, TGF-β signaling negatively regulated SOX9 expression: recombinant TGF-β treatment reduced SOX9 expression whereas exposure to a TGF-β inhibitor increased SOX9 expression in CRC cells (FIG. 14B). These results position SOX9 as a central effector that can integrate pro-stem cell cues mediated by WNT activation and pro-differentiation signals from TGF-β signaling.

### Example 6: SOX9 Directly Activates PROM1 Via a WNT-Responsive Intronic Enhancer

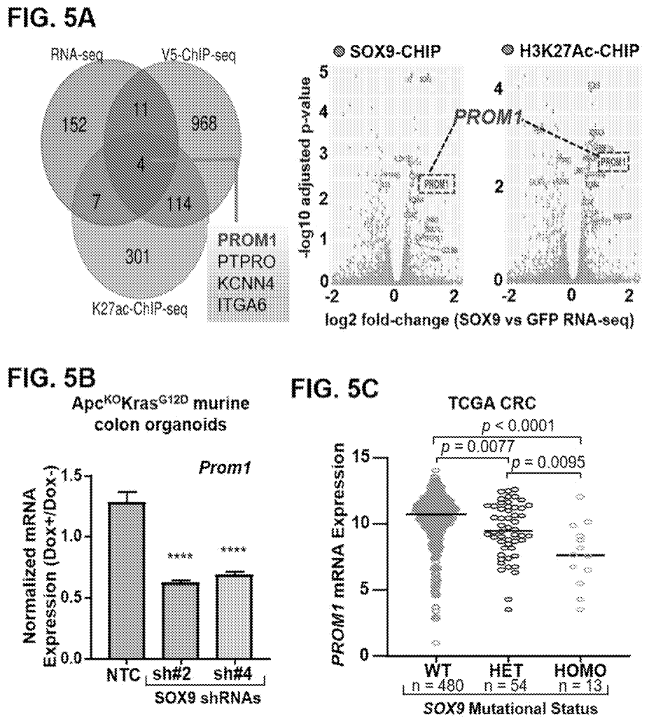

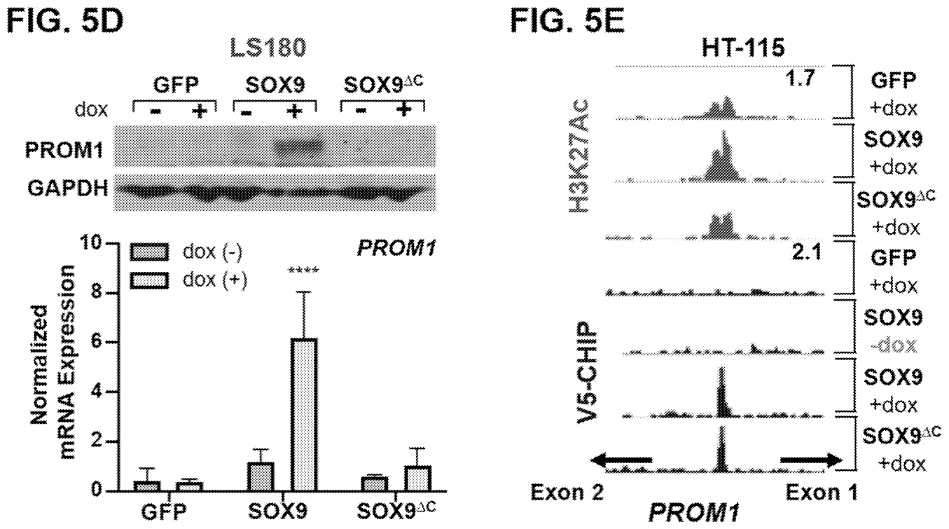

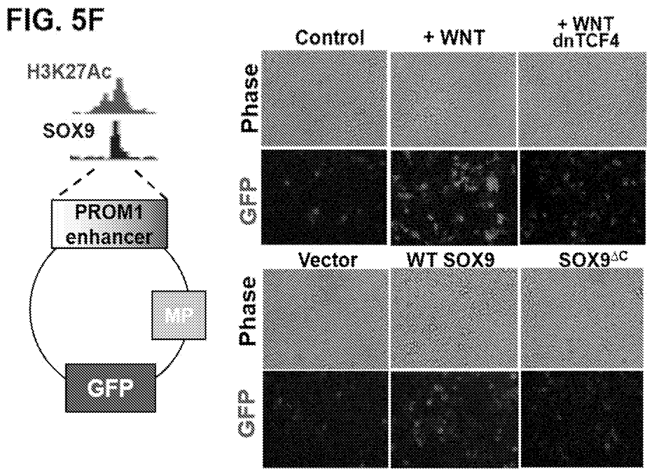

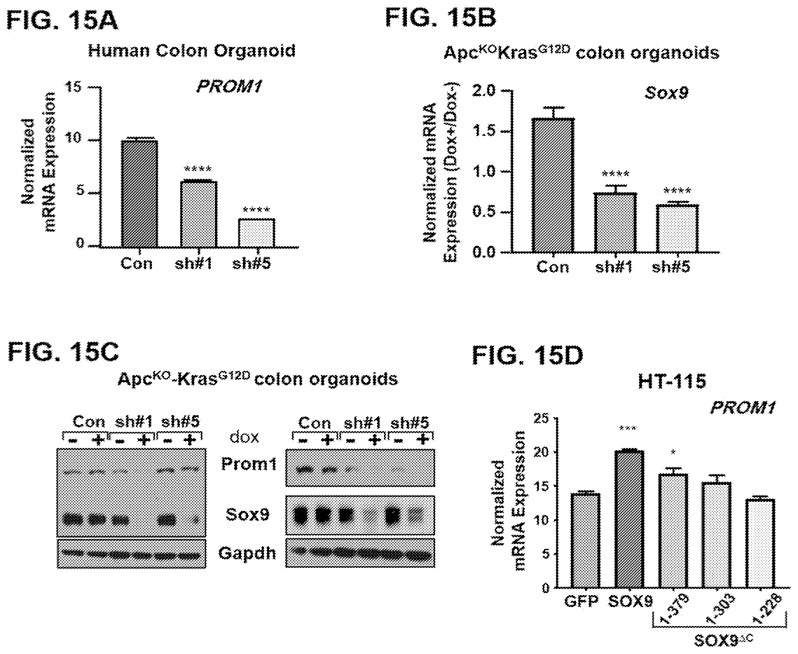

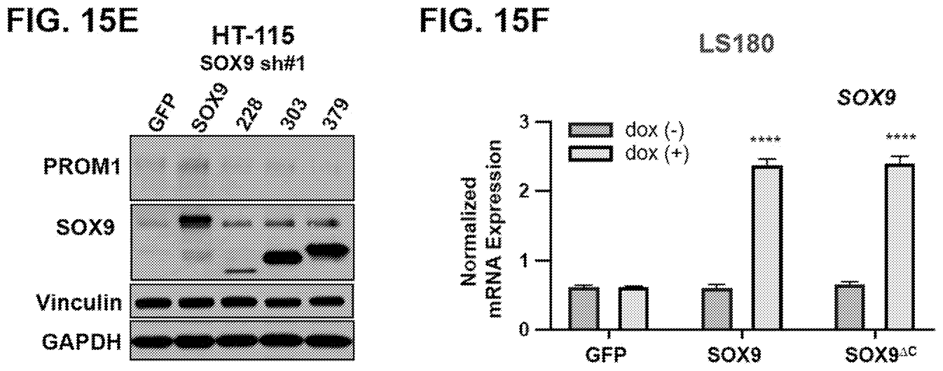

To identify members of the SOX9-mediated stem cell-like program in CRC, an integrative analysis of RNA-seq, V5-SOX9 CHIP-seq, and H3K27Ac CHIP-seq data were performed. Among the genes implicated by all three data sets (FIG. 5A), PROM1 (Prominin-1; also known as CD133) was the most attractive candidate based on: (1) its association with stem cell and crypt biology in intestinal development (Snippert et al. (2009) Gastroenterology 136: 2187-2194 e2181), and (2) its elevated expression in poor prognosis human CRCs (Espersen et al. (2015) Clinical colorectal cancer 14: 63-71; Spelt et al. (2018) Anticancer Res 38: 313-320). Therefore, it was examined whether PROM1 is broadly activated by SOX9 in relevant experimental systems. SOX9 KD reduced PROM1 expression in normal human organoids and neoplastic murine organoids (FIG. 5B; FIG. 15A-C). Moreover, PROM1 expression corresponded to the genomic status of SOX9 in human CRC tumors (FIG. 5C). SOX9 overexpression led to a modest but consistent increase in mRNA and protein expression of PROM1 in HT-115 CRC cells; proportional to the severity of truncation, overexpression of various SOX9^{ΔC} proteins had little to no impact on PROM1 activation (FIGS. 15D and E). Inducible overexpression of WT SOX9 but not SOX9^{ΔC} led to a 6-fold induction in PROM1 mRNA levels in LS180 CRC cells, which corresponded to protein levels by immunoblot (FIG. 5D; FIG. 15F).

To investigate the precise mechanism of PROM1 activation, V5-SOX9 and H3K27Ac CHIP-seq data were analyzed. Although WT SOX9 and SOX9^{ΔC} both bound to an enhancer in intron 1 of PROM1, only WT SOX9 led to greater H3K27Ac deposition at the locus (FIG. 5E), suggesting that PROM1 is positively regulated by SOX9 at this intronic enhancer. To further study this regulatory element, a 573-bp sequence encompassing the PROM1 intronic enhancer was cloned into a GFP reporter construct using Gibson methodology (please see methods for details). Activation of the WNT pathway using recombinant WNT3A stimulated reporter activity by 3-fold, whereas genetic inhibition using dnTCF4 blocked WNT3A-mediated reporter induction (FIG. 5F, top panel). Moreover, WT SOX9 but not SOX9^{ΔC} stimulated reporter activity by 2-fold (FIG. 5F, bottom panel). Together, these data indicate that SOX9 directly activates PROM1 using a Wnt/TCF4-responsive intronic enhancer.

### Example 7: A PROM1-SOX9 Positive Feedback Loop Blocks Differentiation in CRC

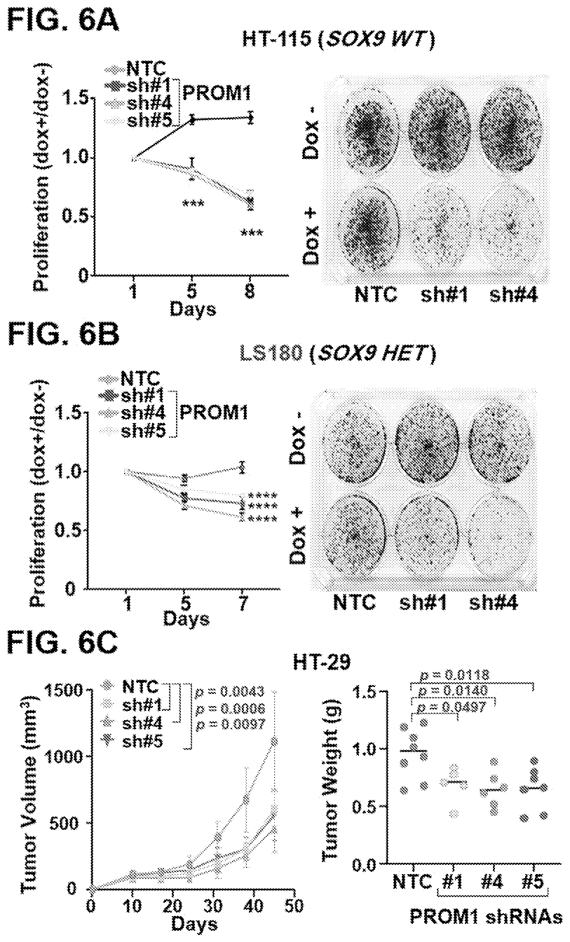

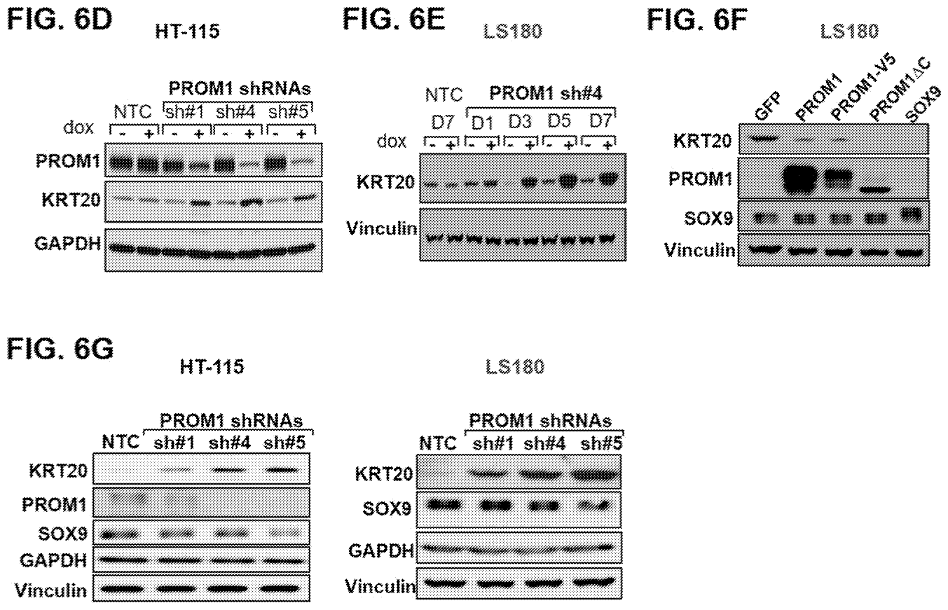

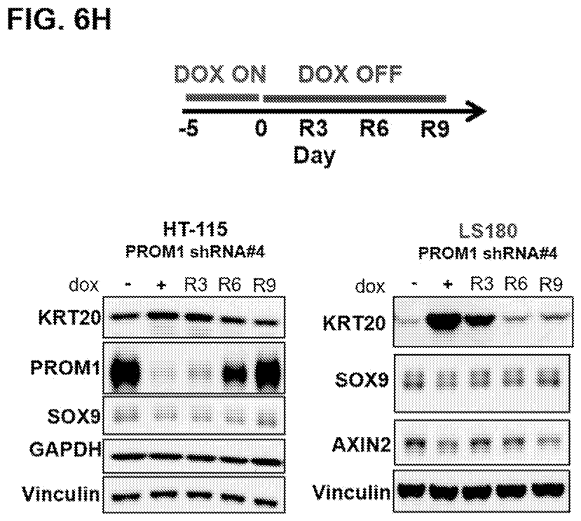

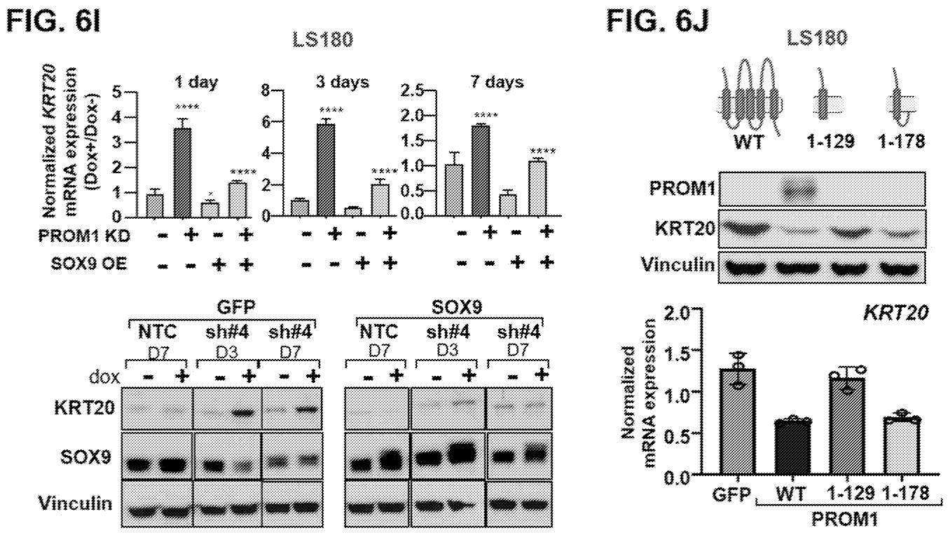

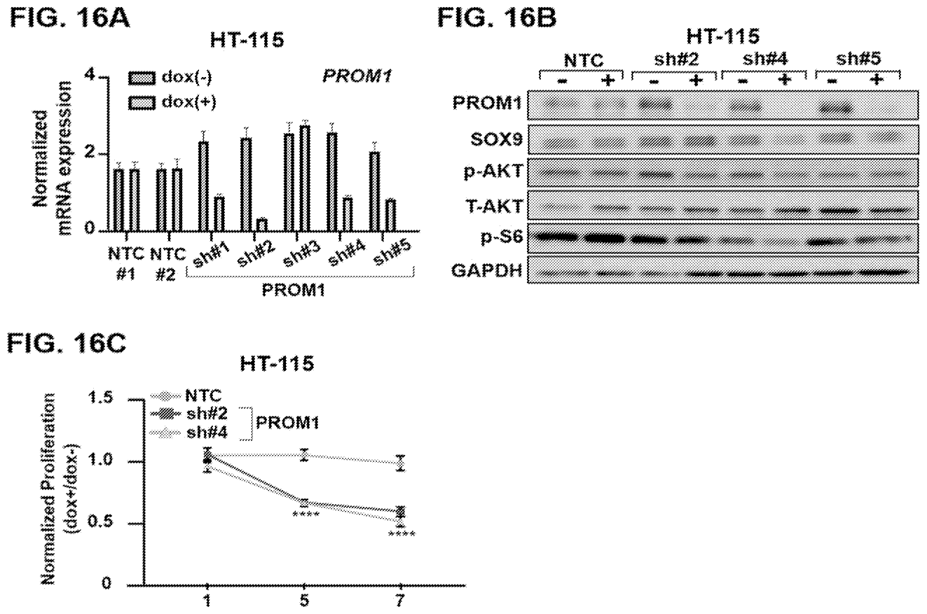

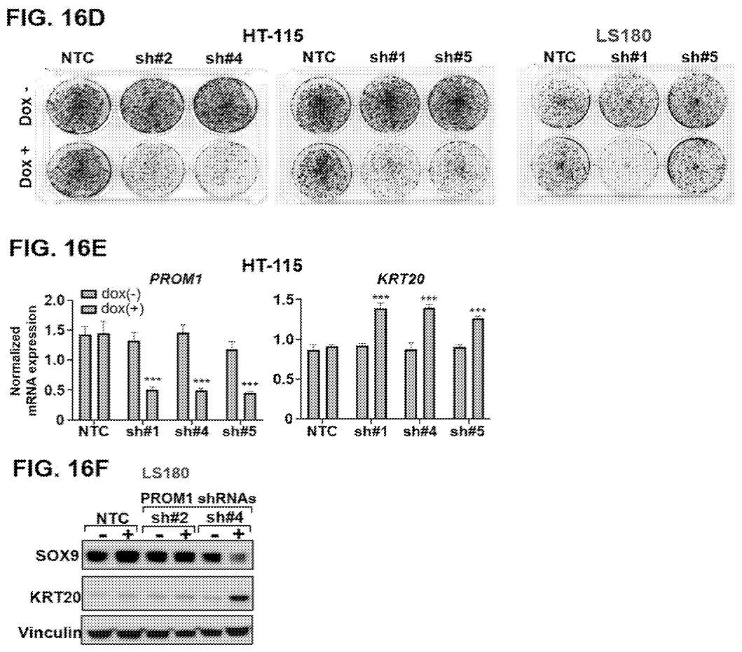

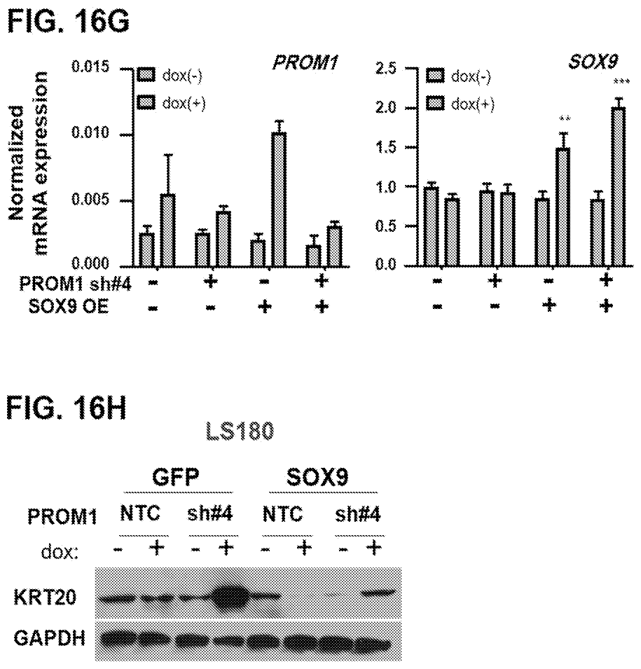

PROM1 is a marker for stem cell activity (Hou et al. (2011) Mol Biol Rep 38: 997-1004) and tumorigenic capacity (Zhu et al. (2009) Nature 457: 603-607; Elsaba et al. (2010) PLoS One 5: e10714; Arena et al. (2011) Anticancer Res 31: 4273-4275) in the intestines. However, whether PROM1 is functionally required for human CRC has not been fully addressed. To investigate whether PROM1 is required for CRC growth, PROM1 using multiple shRNAs in CRC was conditionally suppressed (FIG. 16A-B). Phenocopying SOX9 suppression, PROM1 KD led to proliferation defects in HT-115 and LS180 cells in vitro and impaired primary tumor growth of HT-29 in vivo (FIG. 6A-C; FIG. 16C-D). Inducible PROM1 KD also stimulated intestinal differentiation in HT-115 and LS180 cells as shown by KRT20 induction (FIGS. 6D-E; FIG. 16E). By contrast, overexpression of PROM1 blocked intestinal differentiation in LS180 cells, albeit to a slightly lesser extent than SOX9 (FIG. 6F). These data suggest that, like SOX9, PROM1 blocks differentiation in CRC, perhaps by activating stem cell signaling.

PROM1 is a pentaspan heavily glycosylated apical transmembrane protein. Next, it was investigated whether a molecular mediator connects its membrane localization to its ability to block intestinal differentiation. As a result, constitutive and inducible PROM1 suppression potently reduced SOX9 expression (FIG. 6G-H), suggesting that PROM1 may also function upstream and potentially block differentiation through SOX9 activity. Consistent with this hypothesis, the extent of SOX9 repression by individual PROM1 shRNAs correlated with the magnitude of intestinal differentiation induction as shown by KRT20 expression in HT-115 and LS180 cells (FIG. 6G). A time-course of PROM1 expression restoration following doxycycline withdrawal also showed a gradual return of SOX9 levels that corresponded to KRT20 repression (FIG. 6H). In fact, although PROM1 shRNA #2 could partially suppress PROM1 expression (FIG. 16B), it was not strong enough to reduce SOX9 expression, which led to an inability to induce intestinal differentiation (FIG. 16F), further supporting a requirement for SOX9 suppression in PROM1 KD mediated intestinal differentiation. To definitively test whether SOX9 functions downstream of PROM1, SOX9 in PROM1 KD CRC cell lines were overexpressed and investigated whether it can rescue induction of intestinal differentiation. SOX9 overexpression effectively blocked induction of intestinal differentiation mediated by PROM1 KD (FIG. 6I; FIG. 16H). These data indicate that PROM1 obstructs intestinal differentiation through SOX9, establishing a positive feedback loop.

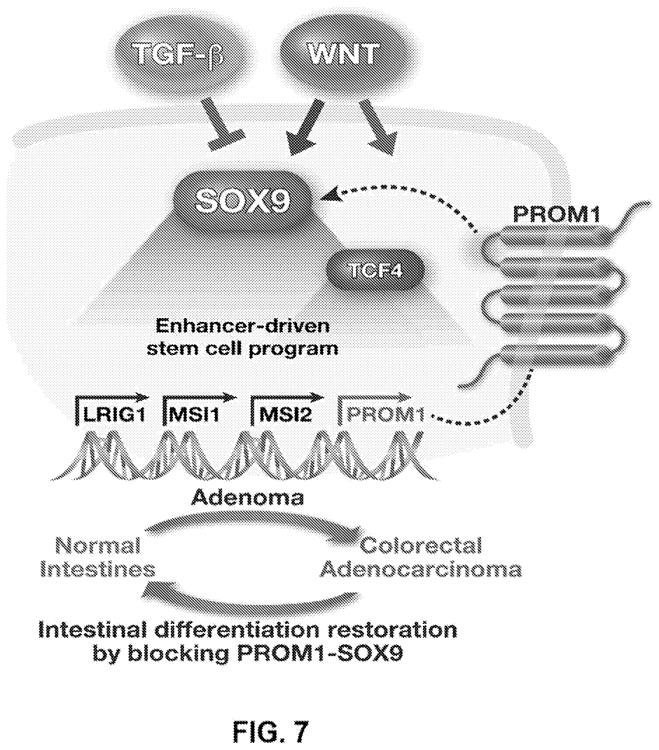

PROM1 has three intracellular domains: the first intracellular loop engages HDAC6 to stabilize β-catenin (Mak et al. (2012) Cell reports 2: 951-963) whereas the 59-amino acid c-terminal domain participates in PI3K/AKT signaling (Wei et al. (2013) Proc Natl Acad Sci USA 110: 6829-6834; Shimozato et al. (2015) Oncogene 34: 1949-1960). Consistent with its regulation of 0-catenin, inducible PROM1 KD reduced levels of canonical WNT-target AXIN2 (FIG. 6H). Since the WNT pathway positively regulates SOX9 activity (FIG. 14B) (Blache et al. (2004) The Journal of cell biology 166: 37-47), it was suspected that PROM1 regulates SOX9 and stem cell activity through its ability to stabilize β-catenin via its first intracellular domain. Consistent with this model, overexpression of a truncated PROM1 protein without its c-terminal intracellular domain (PROM1ΔC) retained the ability to block intestinal differentiation as indicated by KRT20 expression, suggesting that the c-terminal domain is expendable for blocking differentiation (FIG. 6F). By contrast, overexpression of a more severe PROM1 truncated protein lacking the first intracellular domain (PROM1¹⁻¹²⁹) lost most of its ability to suppress intestinal differentiation (FIG. 6J). Notably, overexpression of a truncated PROM1 protein that preserves the first intracellular domain (PROM1¹⁻¹⁷⁸) regained the ability to suppress intestinal differentiation (FIG. 6J). These findings support a reinforcing feedback loop whereby PROM1 and SOX9 positively regulate each other to activate a stem cell-like transcriptional program that counteracts intestinal differentiation in CRC (FIG. 7).

Aberrant activation of stem cell-like programs and their ability to block proper differentiation are central to the pathogenesis of CRC. As described herein, it was shown that SOX9 mediates a stem cell-like transcriptional program by engaging genome-wide enhancers, effectively prohibiting intestinal differentiation in CRC. Hyperactive stem cell signaling portends a poor prognosis and disease relapse in CRC (Merlos-Suarez et al. (2011) Cell Stem Cell 8: 511-524). SOX9 is also overexpressed in human CRC as shown by a recent proteogenomic investigation using patient samples (Vasaikar et al., (2019) Cell 177: 1035-1049 e1019).

Although there is evidence that SOX9 can act as a transcriptional repressor, blocking the expression of differentiation genes CDX2 and MUC2 (Blache et al. (2004) The Journal of cell biology 166: 37-47), data disclosed herein indicate that SOX9 behaves as a transcriptional activator, binding to enhancers that regulate genes associated with stem cell function, likely in coordination with the Wnt/TCF4 pathway. SOX9 regulates Paneth cell differentiation as biallelic genetic inactivation led to a reduction in the number of intestinal Paneth cells in genetically engineered mouse models (Bastide et al. (2007) The Journal of cell biology 178: 635-648; Mori-Akiyama et al. (2007) Gastroenterology 133: 539-546). Consistent with this result, it was found that SOX9 strongly activates a Paneth cell transcriptional program and directly binds to a super enhancer spanning LYZ. Paneth cells indirectly support the stem cell niche (Sato et al. (2011) Nature 469: 415-418) as well as directly engender self-renewal capacity and progenitor functions (Yu et al. (2018) Cell Stem Cell 23: 46-59 e45). Using several murine and human models, the role of SOX9 was extended to one that is also capable of directly stimulating stem cell-like activity in a cell-autonomous fashion. SOX9 may therefore regulate a crypt-restricted transcription program without distinguishing a stem cell or Paneth cell fate, which is likely discerned through distinct transcriptional programs (van Es et al. (2012) Nature cell biology 14: 1099-1104). Suppressing SOX9 reliably induced absorptive and secretory intestinal differentiation in human CRC and neoplastic murine organoids. The extent to which intestinal differentiation was induced by SOX9 KD was variable, with stronger phenotypes observed in normal human colon organoids and Apc^{KO}-Kras^{G12D} murine organoids compared to human CRC cell lines, which carry several pathogenic mutations. These data indicate that advanced cancers with a greater burden of defects that prohibit differentiation may be more difficult to reprogram, and therefore treat, than early lesions such as well-differentiated adenomas.

Wnt and TGF-β are powerful developmental pathways that orchestrate intestinal stem cell and differentiation cues, respectively (Sancho et al. (2004) Annu Rev Cell Dev Biol 20: 695-723). In CRC, oncogenes tend to support stem cell programs whereas tumor suppressor pathways endorse differentiated cell fates. For example, the TGF-β-SMAD4 pathway, known to be a tumor suppressor in gastrointestinal tissue (Hahn et al. (1996) Science 271: 350-353; Thiagalingam et al. (1996) Nat Genet 13: 343-346), ensures a differentiated enterocyte cell identify by engaging a reinforcing positive feedback circuit involving HNF4α- and HNF4γ activity (Chen et al. (2019a) Development 146; Chen et al. (2019b) Nat Genet 51: 777-785). As such, the evolution of CRC can be viewed as a progressive deviation from proper cellular differentiation and serial acquisition of aberrant stem cell-like behavior. Reprograming CRC to obey differentiation cues and regulate stem cell signaling is therefore a promising therapeutic approach. Expressing functional APC can restore Wnt pathway regulation, promote cellular differentiation and inhibit cancer functions (Groden et al. (1995) Cancer Res 55: 1531-1539; Faux et al. (2004) J Cell Sci 117: 427-439; Dow et al. (2015) Cell 161: 1539-1552). Furthermore, inhibiting Lgr5+ stem cells can impair CRC tumor growth and metastasis (de Sousa e Melo et al. (2017) Nature 543: 676-680; Shimokawa et al. (2017) Science 343: 84-87). Translating this concept into effective therapy, however, has proven difficult as Wnt pathway inhibitors have not advanced in preclinical and clinical testing. By defining critical mediators of the stem cell-like program co-opted by CRC, new therapeutic strategies can be designed to restore cellular differentiation. Drug compounds that inhibit stem cell-like activity mediated by PROM1 and SOX9 have the potential to restore intestinal differentiation without evoking toxicities associated with disrupting the pleotropic effects of Wnt signaling.

Like SOX9, the apical transmembrane protein PROM1/CD133 is expressed on Lgr5+ stem cells and transit amplifying cells in the intestines (Snippert et al. (2009) Gastroenterology 136: 2187-2194 e2181). As disclosed herein, PROM1 activation can block intestinal differentiation via SOX9, which in turn activates PROM1 transcription, reinforcing a positive feedback circuit. A deeper look into how PROM1 outer membrane modifications (i.e., glycosylation), membrane-bound and intracellular partners, and downstream pathway mediators impact signal transduction could yield greater insight into this regulation. Once activated, SOX9 transcriptionally regulates a stem cell-like program, however the precise chromatin-level mechanisms remain unknown. Since the c-terminus of SOX9 is required for transcriptional activation, it will be useful to characterize cofactors that physically bind to this domain. It is possible that SOX9 recruits proteins involved in chromatin remodeling and architecture to regulate genes associated with stem cell function, some of which may be targets of existing therapies.

As disclosed herein, it was shown that human CRC is dependent on an enhancer-driven stem cell-like program supported by a PROM1-SOX9 positive feedback loop. Blocking the PROM1-SOX9 axis restores intestinal differentiation and impairs CRC growth, providing rationale for the development of therapeutic agents that disrupt this pathway.

### Example 8: Materials and Methods for Examples 9-13

a. FASTQ Demultiplexing and Read Count

Abundance of sgRNA reads was determined using an adaptation of the pyahocorasick module, which employs the Aho-Crasick string-search algorithm to demultiplex FASTQ files and tabulates the gRNA read counts for each fraction. The pyahocorasick module leverages the computational speed of the compiled language C under the hood with linear time complexity for multi-string searching and the readability of Python to be readily adapted as a versatile demultiplexing and read count tool.

b. Normalization of sgRNA Representation

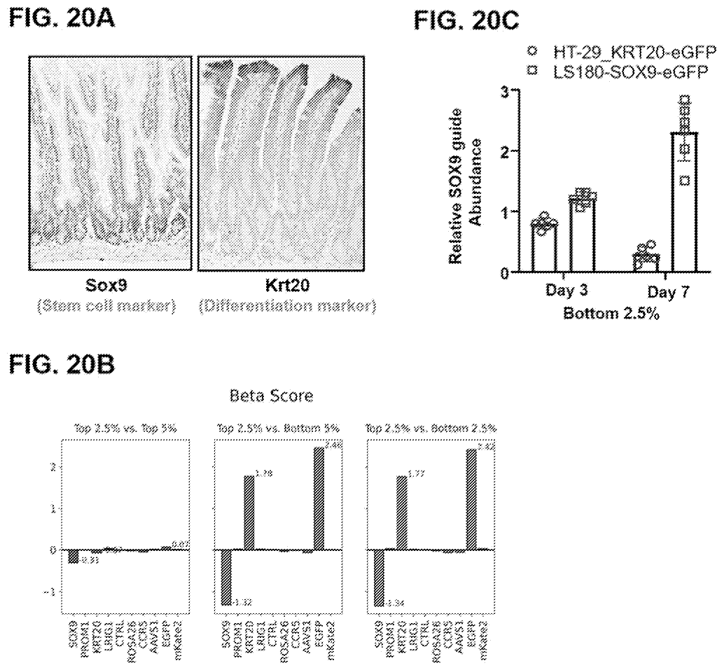

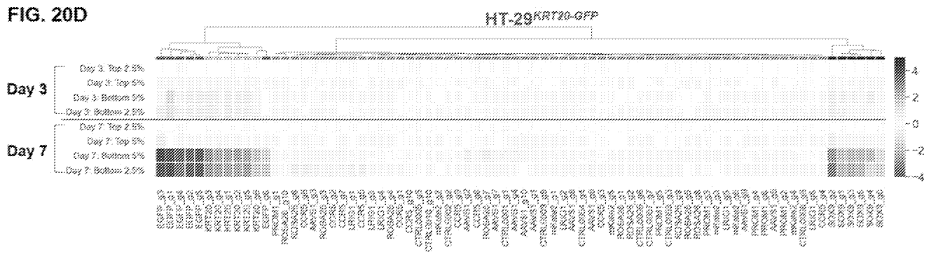

The total count normalization assumes that the total expression of gRNA reads is the same under the different experimental conditions. The total count normalization step divides the read count for each gRNA by the total number of read counts in the sample. After dividing by total sample size, the output normalized read counts represent the proportion of each gRNA reads relative to the total sample size (FIG. 20A-D).

Library pool normalization considers the uneven gRNA representation within the library and assumes that within the pool each gRNA read should have similar read counts on average in each experimental condition. The library pool normalization step divides the read count for each gRNA in every sample by the mean of the read counts for each gRNA from 3 independent library pool control populations, effectively controlling for technical variability. After dividing by the mean of the library pool controls, the output normalized read counts control for the natural variation in library preparation (FIG. 20A-D).

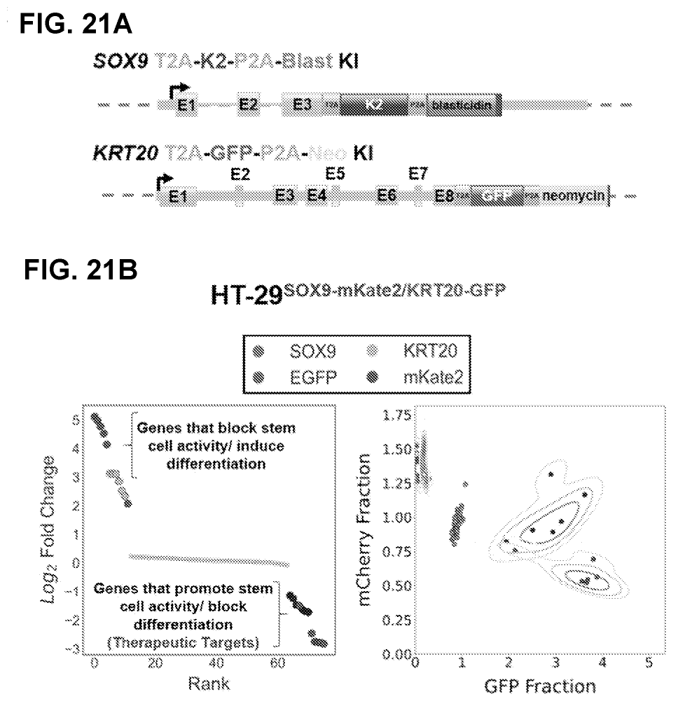

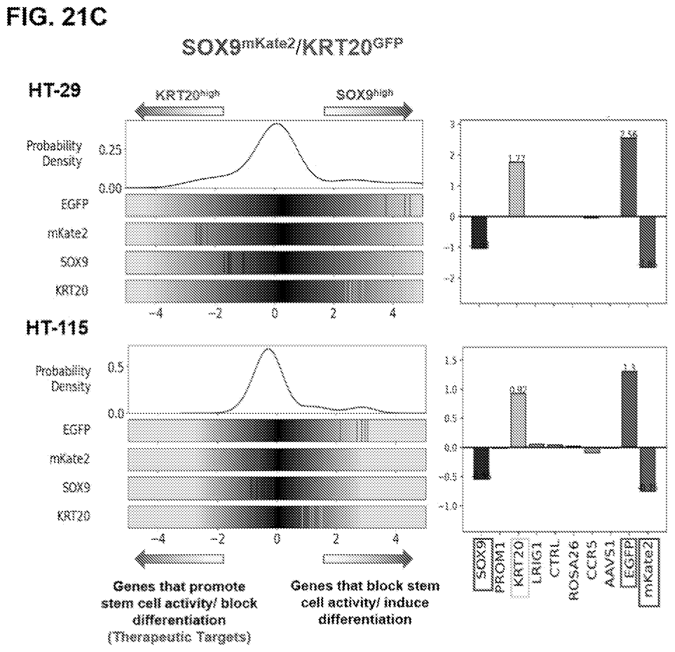

Population normalization assumes that between each cell line, each gRNA should have similar read counts on average and represents the basic gRNA distribution at population level before sorting. The population normalization step divides the read count for each gRNA in each fraction separated by fluorescent intensity by the total read count for each gRNA across all fractions of the population. After dividing by the population read count, the output normalized read counts control for the variation due to different cell lines (FIG. 21C).

Fraction to fraction comparison resulting the gRNA enrichment in one fraction compare to the other (FIG. 21D). Based on these results and observations it was concluded that the total read normalization is the most critical factor during the normalization steps and influence the results most significantly.

### c. 5-Way Comparison

Four fractions for each sample were obtained using flow sorting based on fluorescent intensity of green fluorescent protein and red fluorescent protein. For cell lines with knock-in of the dual reporter system, the A fraction represents cells within the population which exhibit relatively high green fluorescent intensity and the B fraction represents cells within the population which exhibit relatively high red fluorescent intensity. The C fraction and D fraction both represent the relatively low green and red fluorescent intensity and each serve as a unique negative control for knock-in of fluorescent proteins. Between the four fractions, five pairwise comparisons between fractions can be made to evaluate relative differential expression of normalized gRNA read counts under different experimental conditions within one cell line: A vs. B, A vs. C, A vs. D, B vs. C, and B vs. D.

d. Scatter Plot of Normalized Read Counts

The scatter plot visualizes gRNA representation (log 2 scale) of normalized read counts between two unique fractions. Five scatter plots are shown for the 5 comparisons of interest between the 4 experimental fractions. The black line indicates the line of null effect, where normalized read counts of gRNA from two fractions plotted along this line are not differentially expressed. Relative gRNA read count enrichment and depletion are plotted further away from the black line. The different gRNA designed to target the SOX9 (blue), KRT20 (orange), EGFP (green), and mKate2 (red) genes are color coded.

e. Heat Map of Normalized Read Counts

The heatmap visualizes the gRNA representation (log₂ scale) of normalized read counts within one sample at one time point. For Sample 1 to Sample 10, two replicates were conducted for each of the four fractions, with the exception of Sample 2 which does not have a replicate. Log₂ transformed normalized read counts are visualized in red to represent enrichment and blue to represent depletion. GRNA on the bottom are clustered using a hierarchical clustering method. The different gRNA designed to target the SOX9 (blue), KRT20 (orange), EGFP (green), and mKate2 (red) genes are color coded.

f. Volcano Plot

Volcano plot plots the negative, log₁₀ transformation of the p value from Welch's t-test between two fractions for each gRNA on the y-axis and the log₂ transform of the fold change of normalized read counts between two fractions for each gRNA on the x-axis. The different gRNA designed to target the SOX9 (blue), KRT20 (orange), EGFP (green), and mKate2 (red) genes are color coded.

g. Log 2-Fold Change Cumulative Density Plots

The gaussian kernel density estimation plot plots the log 2 transform of the fold change of normalized read counts between two fractions. The kernel density estimation plot is derived by estimating the distance of each gRNA at a discrete location along the probability density distribution. The integral of the curve approximates to one. The estimation is higher where gRNA are more closely grouped due to the higher probability of observing a point at that location.

h. MaGeCK Beta Score

The Model-based Analysis of Genome-wide CRISPR/Cas9 Knockout (MaGeCK) method was used to identify gRNA hits from the CRISPR screen that were differentially expressed. The MaGeCK test function was used with default parameters for the Maximum Expectation-Maximization (EM) algorithm, including median ratio normalization between samples for each gRNA read to produce the beta score. Beta scores represent the extent of selection for each treatment compared to the initial gRNA abundance in a control. Positive beta scores reflect positive selection while negative beta scores reflect negative selection of the gene.

i. Cell Culture, Lentivirus Packing, and Transduction

All cell lines were maintained at 37° C. with 5% C02. The human colorectal cancer cell lines were obtained from the CCLE core facility and used at early passage for the experiments. HT-115 and HT-29 cells were maintained in DMEM medium supplemented with 10% FBS and 1% penicillin/streptomycin. LS180 cells were cultured in RPMI 1640 containing 10% FBS and 1% penicillin/streptomycin.

To generate lentiviruses, expression vectors were co-transfected into HEK293T cells with the lentiviral packaging constructs psPAX2 and pMD2.G (VSV-G) in a 1:1:1 ratio using X-tremeGENE 9 DNA Transfection Reagent (Roche) according to the manufacturer's instructions. Cell culture media was changed the following day and lentiviral supernatant was harvested 48 h and 72 h later and filtered through a 0.45 m filter (Millipore). Lentiviruses were aliquoted and stored at −80° C. until use.

To perform lentiviral infection, the CRC cells were plated in a 6-cm dishes and infected with 0.5-1 mL virus in media containing 8 mg/mL polybrene overnight.

j. Statistical Analysis and Reproducibility

Experiments were performed in triplicate. Data are represented as mean±s.d unless indicated otherwise. For each experiment, either independent biological or technical replicates are as noted in the Figure legends and were repeated with similar results. Statistical analysis was performed using Microsoft Office or Prism 7.0 (GraphPad) statistical tools. Pairwise comparisons between groups (that is, experimental versus control) were performed using an unpaired two-tailed Student's t-test or Kruskal-Wallis test as appropriate unless otherwise indicated. For all experiments, the variance between comparison groups was found to be equivalent. Animals were excluded from analysis if they were euthanized due to health reasons unrelated to tumor volume endpoint.

### Example 9: Development of an Endogenous Intestinal Stem Cell and Differentiation Reporter System in Colorectal Cancer

Genomic alterations that encourage stem cell activity and hinder proper maturation are central to the development of colorectal cancer (CRC). To identify molecular mediators that regulate stem cell and differentiation pathways in CRC, an endogenous stem cell and differentiation reporter system were developed by genome-editing human CRC cell lines. The endogenous stem cell reporter utilizes the SOX9 locus to faithfully readout stem cell activity, whereas the endogenous differentiation reporter takes advantage of the KRT20 locus. After genetic and biological validation of single endogenous reporters in CRISPR screening formats, a dual reporter system that simultaneously monitors stem cell and differentiation activity was engineered in the same CRC cell line, improving the signal to noise discrimination. Of translational importance, the platform as disclosed herein was utilize to identify therapeutic agents that block stem cell activity and promote differentiation in CRC. By performing a focused small molecular inhibitor screen, two epigenetic inhibitors that reduce viability of CRC cells were discovered by inducing intestinal differentiation. These results disclosed herein highlight the utility of a biologically designed endogenous reporter system to uncover novel therapeutic agents for the treatment of cancer.

Disrupting the balance between stem cell and differentiation programs is one of the defining properties of CRC, which remains the third most common and second most deadly malignancy worldwide (Siegel et al. (2018) CA: a cancer journal for clinicians 68: 7-30). The near-universal initiating event in sporadic CRC involves genomic alterations that activate Wnt signaling, most often through loss-of-function APC mutations (Kinzler and Vogelstein (1996) Cell 87: 159-170; Marmol et al. (2017) Int J Mol Sci 18). Aberrant Wnt activation shifts the crypt-villus homeostasis in favor of stem cell activity, which leads to CRC initiation. As these early colonic lesions evolve, mitogenic pathway mutations ensue (e.g., KRAS, BRAF) alongside alterations in the pro-differentiation pathways of transforming growth factor β (TGF-β) and bone morphogenetic protein (BMP) (Takeda et al. (2019) Proc Natl Acad Sci USA 116: 15635-15644). As such, genomic alterations that hinder intestinal differentiation, either by activating stem cell-like programs or inactivating pro-differentiation pathways, are central to CRC development. Despite the intellectual clarity of this observation, it is yet to uncover critical regulators of aberrantly active stem cell-like programs or convert this understanding into efficacious treatment for CRC.

Unbiased genetic screens have emerged as a powerful tool to identify cancer dependencies. While most of these screens use cell viability and proliferation as readouts, there are examples for biologically defined outputs that better define the mechanism of action underlying potential therapeutic targets. A functional genetic viral-based, CRISPR-based pooled screen in primary mouse Treg cells was recently designed to discover regulatory programs involved in the promotion or disruption of Foxp3 expression, a master regulator of the Treg cell fate (Cortez et al., 2019). While expression levels of cell surface markers such as Foxp3 can readily be measured and adapted for functional screens using fluorochrome-conjugated antibodies, intracellular and nuclear protein expression is more difficult to determine. To overcome this hurdle, a combination of CRISPR/Cas9 and site-directed homology recombination has been utilized to fluorescently tag or report endogenous proteins and/or gene transcriptional activity, respectively (Stewart-Ornstein and Lahav (2016) Cell reports 14: 1800-1811). Therefore, a development of a novel screening platform to identify genetic perturbations and therapeutic agents that block stem cell activity and induce intestinal differentiation in CRC is much needed.

### Example 10: Development of an Endogenous Stem Cell Reporter by Genome Editing SOX9 Locus

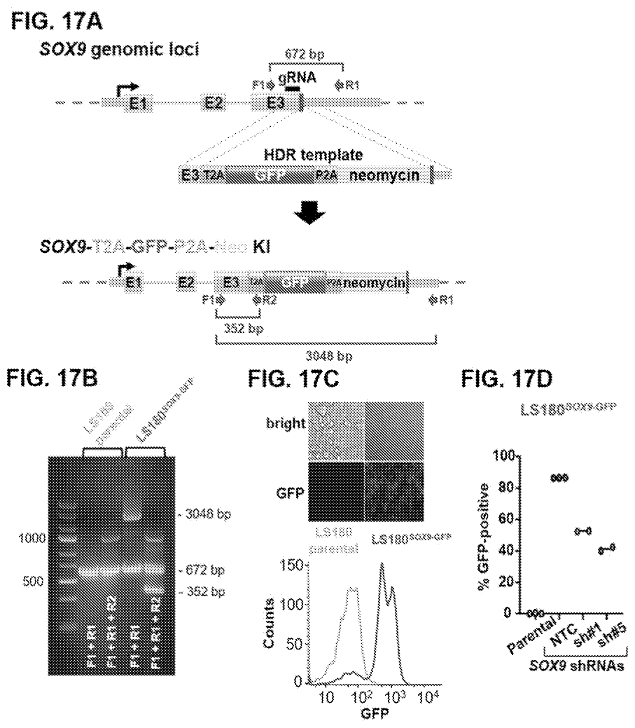

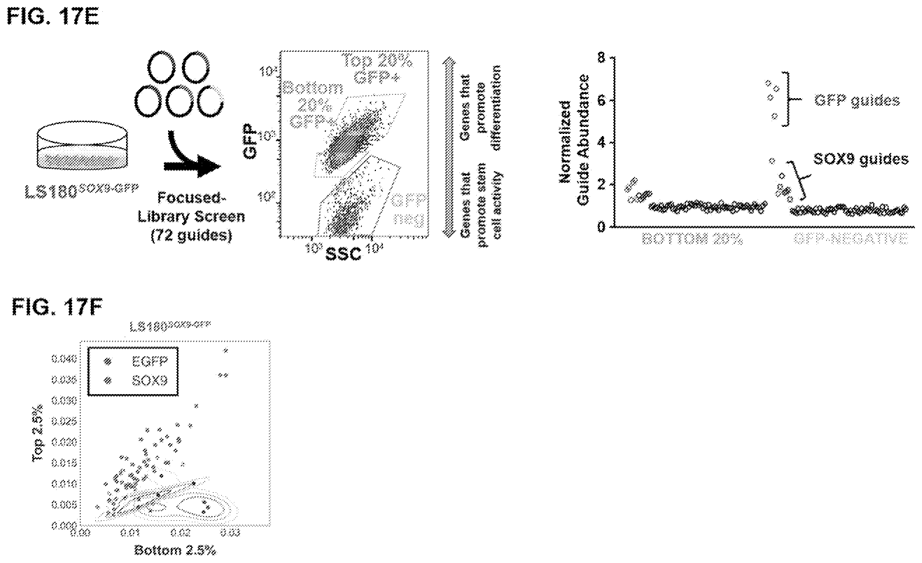

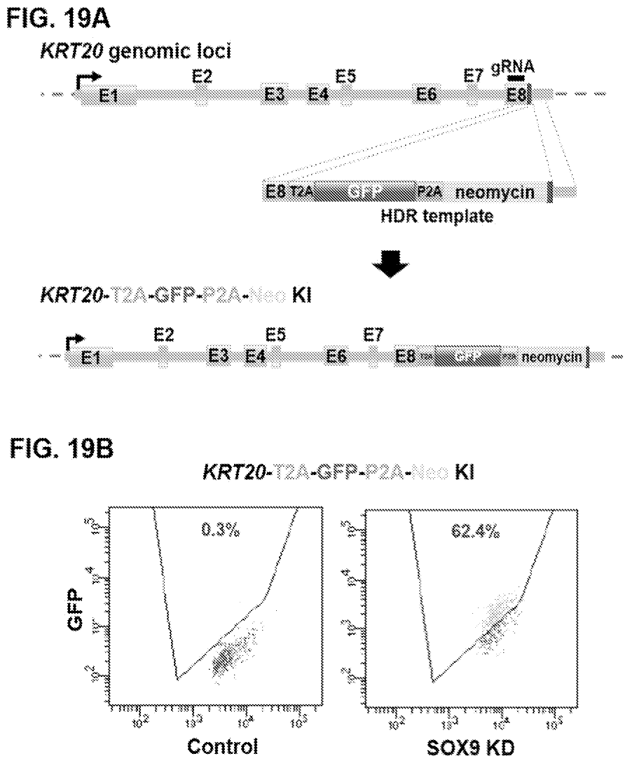

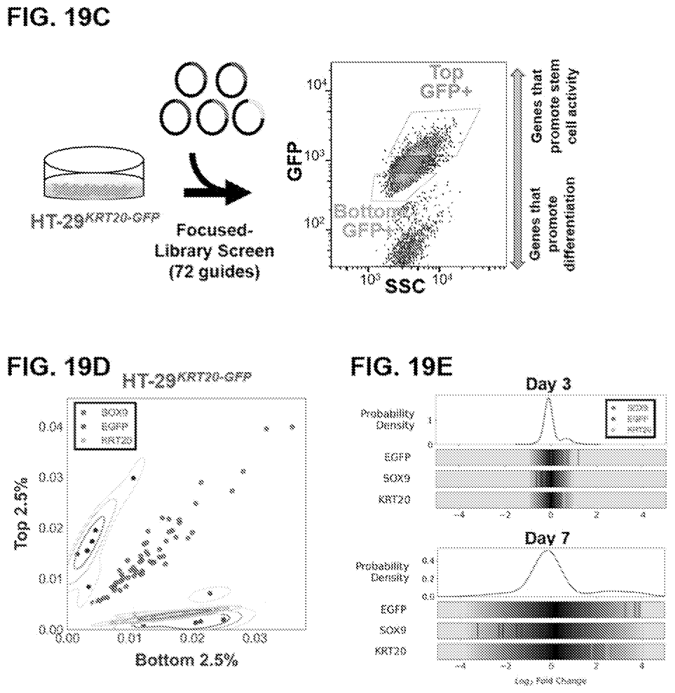

As disclosed herein, SOX9 functionally blocks differentiation by activating a stem cell-like transcriptional program in human CRC. SOX9 suppression induces differentiation and impairs CRC growth. Of translational importance, these results implicate a dependency of CRC on specific stem cell programs and inspire therapeutic approaches directed at promoting intestinal differentiation. Based on these findings, it appears that genetic perturbations and small molecules that disrupt stem cell signaling and induce intestinal differentiation will have therapeutic value in the treatment of CRC. To this end, an endogenous knock-in reporter system was designed and engineered by introducing a fluorescent probe into genomic loci that readout stem cell (i.e., SOX9) and intestinal differentiation (i.e., KRT20) activity in CRC cells. To establish an endogenous stem cell activity reporter, a cassette containing GFP and neomycin antibiotic resistance in-frame were knocked-in at the end of the SOX9 coding region of a CRC cell line using the combination of CRISPR/Cas9 technology and template-based homologous recombination (Stewart-Ornstein and Lahav (2016) Cell reports 14: 1800-1811) (FIG. 17A). sgRNAs were designed and tested to target the last exon of SOX9 in closest proximity to the stop codon. Cas9 pre-preloaded in vitro with the best performing sgRNA (see methods for T7 assay) was electroporated into LS180 CRC cells for precise and efficient genome editing. A double stranded break (DSB) followed by homology directed repair (HDR) facilitated integration of the GFP fluorescent reporter cassette. Following recovery from electroporation, CRC cells were propagated in media containing neomycin to select populations with in-frame integration. Accurate genomic integration was confirmed using site-specific PCR with primers against genomic locus and cassette (FIG. 17B). Compared to the parental CRC cell line, the engineered GFP knock-in stem cell reporter line demonstrated high GFP levels due to elevated SOX9 expression in CRC (FIGS. 19C and 20A). Since SOX9 drives a stem cell-like transcriptional program, GFP levels in the engineered reporter line will reflect stem cell activity. Notably, ˜87% of the LS180 expressed high GFP levels, which likely reflects the proportion CRC cells with stem cell-like transcriptional activity, whereas the remaining 13% of cells displayed low GFP, and therefore low endogenous SOX9 expression, potentially reflecting the smaller differentiating fraction of CRC cells. To validate whether GFP faithfully reflects SOX9, two SOX9 shRNAs were stably expressed in the LS180^{SOX9-GFP} reporter line, which led to a >50% reduction of GFP levels (FIGS. 19D and 20B-C). These studies established an endogenous reporter CRC cell line that reflects stem cell-like activity through GFP expression.

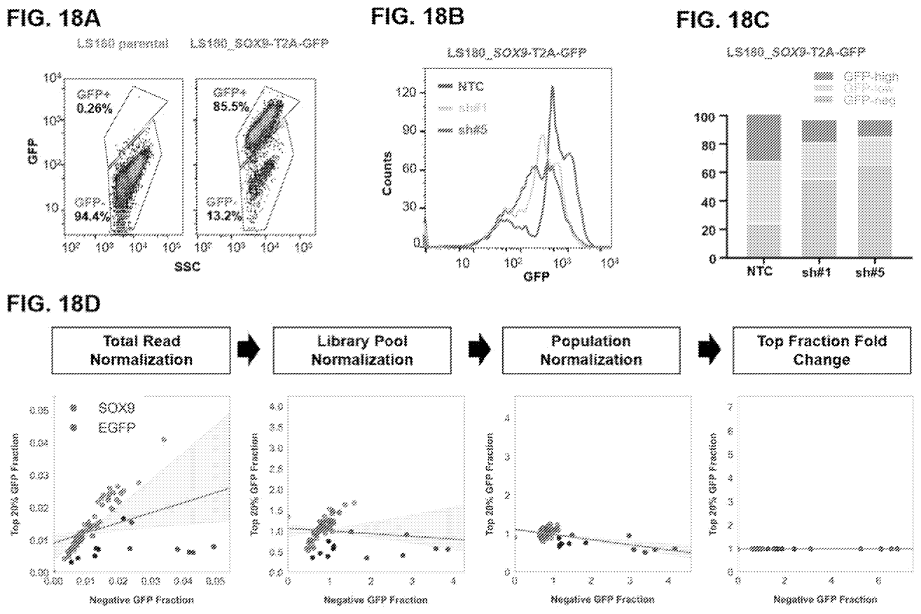

Next, a focused library CRISPR/Cas9 screen (76 sgRNAs targeting 9 genes, Table 5) were performed using the LS180_{SOX9-GFP} reporter to test its functionality in a pooled genetic perturbation format. After introducing a pool of 6 sgRNAs targeting GFP, 6 sgRNAs targeting SOX9, and 58 sgRNAs targeting 8 control genes, the cells were sorted after three days based on GFP expression (FIG. 17E). Genomic DNA was extracted and sequenced to determine sgRNA representation in the top 20%, bottom 20%, and GFP negative fractions. To optimize the signal to noise ratio, a series of normalization steps were performed (FIG. 18D). The aim of normalization is to produce comparable read counts that reflect the true differences in gRNA abundance among samples and different fractions. The normalization steps control for natural variation in cell lines and library preparation, enabling accurate comparisons between representative normalized read counts across different conditions. Three different levels of normalization were applied to the data: 1) total count, 2) library pool and 3) population level. For gRNA enrichment/depletion interpretation, fraction to fraction comparison was performed. It was demonstrated how these normalization strategies impact gRNA distribution, representation, and visualization (FIG. 20D).

TABLE 5
List of sgRNAs targeting 9 genes

| | |
|---|---|
| SOX9_g1 | ACTTCCGCGACGTGGACAT |
| SOX9_g2 | TCACCGACTTCCTCCGCCG |
| SOX9_g3 | CCATGTCCGAGGACTCCGC |
| SOX9_g4 | CTCACCCGAGTGCTCGCCG |
| SOX9_g5 | AAGGCTGACCTGAAGCGAG |
| SOX9_g6 | GTCGGTCATCTTCATGAAG |
| PROM1_g1 | GTAGTAAAAAATCACGATG |
| PROM1_g2 | TGTGAACCTTACACGAGCA |
| PROM1_g3 | TCACCTGCTACGACAGTCG |
| PROM1_g4 | GTCGTTGCTGTAACAAATG |
| PROM1_g5 | CACGCCACACAGTAAGCCC |
| PROM1_g6 | GTTGCTGTAACAAATGTGG |
| KRT20_g1 | ACTATGGGAGCGATCTCAC |
| KRT20_g2 | AGAGGAATACGTCTAACAG |
| KRT20_g3 | TAGTCGCGACCAGCCCTCG |
| KRT20_g4 | CAGCACCCCCATAAACGCT |
| KRT20_g5 | GGCAACACTGTCAATGTGG |
| KRT20_g6 | TTCTCCTTCCAGAAGGCGG |
| LRIG1_g1 | ATGTGAAAATAGATGACGC |
| LRIG1_g2 | GTGGTTGGTGATGACACAT |
| LRIG1_g3 | CCTTTATAGGCGGTCCGTG |
| LRIG1_g4 | TCCTGCCAATTAGCCACGG |
| LRIG1_g5 | ATGGTGAAGATGCCTACCG |
| LRIG1_g6 | GGTAGCTTGGAACATCTGG |
| CTRL0001_g1 | TAGCGAACGTGTCCGGCGT |
| CTRL0002_g2 | ACCGGAACGATCTCGCGTA |
| CTRL0003_g3 | GCAGTCGTTCGGTTGATAT |
| CTRL0004_g4 | CTTGAGCACATACGCGAAT |
| CTRL0005_g5 | TGGTAGAATAACGTATTAC |
| CTRL0006_g6 | TCATACATGGATAAGGCTA |
| CTRL0007_g7 | ATACACGAAGCATCACTAG |
| CTRL0008_g8 | AACGTTGGCACTACTTCAC |
| CTRL0009_g9 | ATCCATGTAATGCGTTCGA |
| CTRL0010_g10 | TCGTGAAGTGCATTCGATC |
| ROSA26_g1 | GGCCTCAACTGCAAGAGGA |
| ROSA26_g2 | CGCCGGCCTCAACTGCAAG |
| ROSA26_g3 | AATCCTAGGGAGCGGGTAA |
| ROSA26_g4 | AAAGGACGCGTTCCGCACA |
| ROSA26_g5 | CTTCTAAAAAATGCAGCTG |
| ROSA26_g6 | GCCGGGGGAAGTATTAGAG |
| ROSA26_g7 | GTGAGAGTAGTACCTGGTT |
| ROSA26_g8 | TTAGAAATGCTCAACTGGC |
| ROSA26_g9 | TATCTTCACAAAGCTGCTG |
| ROSA26_g10 | TCAGCTTTCCCCTAGAGAA |
| CCR5_g1 | ATATAGACAGTATAAAAGT |
| CCR5_g2 | AACAGGCCAAGCAGCTGAG |
| CCR5_g3 | ATCTCAGAAGCTAACTAAC |
| CCR5_g4 | ACTCCCTGCACCTTAGACT |
| CCR5_g5 | GGCCGGGTGCCCAAGTCCC |
| CCR5_g6 | TAGGGCAACCTTATGAGGC |
| CCR5_g7 | TAATAATTAAGAGGCCTCG |
| CCR5_g8 | CAAGCCAGTAATAATTAAG |
| CCR5_g9 | GACCATGACAAGCAGCGGC |
| CCR5_g10 | ATGTGAAGGGGAGATAAAA |
| AAVS1_g1 | CTCCCTGGCCACTTTGCAC |
| AAVS1_g2 | ATGTGCGCACCCTGGAGCC |
| AAVS1_g3 | GCTGTAGTCAGGGACTGGG |
| AAVS1_g4 | ATCGTTCACAATAGCCCAG |
| AAVS1_g5 | GGCCGGAAAAGGCCATTTG |
| AAVS1_g6 | CATGATGCAGAATTCCCCG |
| AAVS1_g7 | CTGCACCCCAGCTCTAAGG |
| AAVS1_g8 | TGCGCAGGACCTGGATCAC |
| AAVS1_g9 | ACTTGCATCAGCTTCTCAG |
| AAVS1_g10 | TTTCCCTTGGACCCCAGGG |
| EGFP_g1 | GGGCGAGGAGCTGTTCACCG |
| EGFP_g2 | GAGCTGGACGGCGACGTAAA |
| EGFP_g3 | GGCCACAAGTTCAGCGTGTC |
| EGFP_g4 | GGAGCGCACCATCTTCTTCA |
| EGFP_g5 | GAAGTTCGAGGGCGACACCC |
| EGFP_g6 | GGTGAACCGCATCGAGCTGA |
| mKate2_g1 | CTACAACGTCAAGATCAGAG |
| mKate2_g2 | GTCACCACATACGAAGACGG |
| mKate2_g3 | GCACATGAAGCTGTACATGG |
| mKate2_g4 | GTGATGCAGAAGAAAACACT |
| mKate2_g5 | ACCTTCATCAACCACACCCA |
| mKate2_g6 | CACTTCAAGTGCACATCCGA |

Following normalization, it was expected that sgRNAs targeting genes that promote stem cell signaling to be enriched in the bottom 20% and GFP negative sorted populations, with GFP and SOX9 sgRNAs serving as positive controls. Indeed, only GFP and SOX9 sgRNAs were enriched in these sorted fractions (FIG. 17E), validating the stem cell reporter line in the genetic screening format. Furthermore, when the distribution of sgRNAs in the top 2.5% GFP+ compared to the bottom 2.5%, while most sgRNAs distributed evenly across the x/y=1 axis, GFP and SOX9 sgRNAs were preferentially abundant in the bottom 2.5%. Three endogenous stem cell reporter CRC cell lines with similar properties were established as described below. Together, these data support use of the endogenous stem cell reporter system for functional genetic screens that may lead to the identification novel regulators of stem cell behavior in CRC.

### Example 11: Endogenous Intestinal Differentiation Reporter System by Genome Editing KRT20 Locus

A principle reason that aberrant stem cell activity is selected as an early event in CRC pathogenesis is to block differentiation and prevent death of intestinal cells. In other words, aberrant stem cell signaling antagonizes intestinal differentiation in colorectal cancer initiation, enabling the persistence of neoplastic intestinal cells rather than their turnover in a rapidly regenerative organ. As disclosed herein, an endogenous reporter platform that captures intestinal differentiation activity was therefore developed. KRT20 is a well-recognized marker of differentiated intestinal cells that is notably absent in stem cells (FIG. 20A). To engineer an intestinal differentiation reporter, the GFP fluorescent cassette was integrated into the KRT20 genomic locus of HT-29 CRC cells (FIG. 19A). To biologically validate the system, it was determined whether disrupting stem cell signaling through SOX9 suppression induced differentiation reporter activity based on the results disclosed herein. HT-29^{KRT20-GFP} reporter line stably expressing a shRNA against SOX9 led to a marked shift in the number of GFP+ cells (FIG. 19B, 0.3% to 62.4%), validating that induction of intestinal differentiation by suppressing stem cell signaling is captured by this endogenous differentiation reporter system.

Given that HT-29^{KRT20-GFP} reporter captured both directions of the intestinal stem cell and differentiation spectrum, the endogenous reporter platform was further optimized to improve the signal to noise discrimination. As the size of a CRISPR library increases, a longer sorting time was anticipated to achieve necessary guide recovery for quantitation. different gating cut-offs were tested to optimize guide recovery from sorted fractions, searching for a wide gating strategy, and therefore greater cell/guide recovery for a given duration of flow sorting, that preserves signal to noise discrimination. Comparing the top 2.5% to the next 2.5% GFP+ fraction did not demonstrate a difference in sgRNA distribution (FIG. 20B, left panel), suggesting that 5% may be an optimal cut-off. Consistent with this conclusion, analyzing the top 2.5% GFP+ fraction compared to the bottom 5% (FIG. 20B, middle panel) demonstrated the same discriminatory power as if compared to the bottom 2.5% (FIG. 20B, right panel). Next, it was determined whether timing of sgRNA recovery following library infection impacts distribution. Compared to day 3, sgRNA recovery on day 7 following library infection generated greater discrimination of GFP, SOX9, and KRT20 guides (FIG. 19E). This experiment was extended and measured sgRNA recovery 14 days following library infection, which showed no improvement in sgRNA enrichment and depletion. Reassuringly, SOX9 sgRNA enrichment trended in opposite directions when analyzing day 3 and day 7 of CRISPR library experiments involving the endogenous stem cell (i.e., LS180^{SOX9-GFP}) and differentiation (i.e., HT-29^{KRT20-GFP}) reporter systems (FIG. 20C). The combination of flow gating and time-course optimization is readily demonstrated when sgRNA enrichment and depletion is represented by beat-map visualization (FIG. 201)). These results demonstrate successful engineering of an endogenous differentiation reporter that is optimized to detect genetic perturbations that impact intestinal differentiation in CRC.

### Example 12: Dual Stem Cell and Differentiation Reporter System

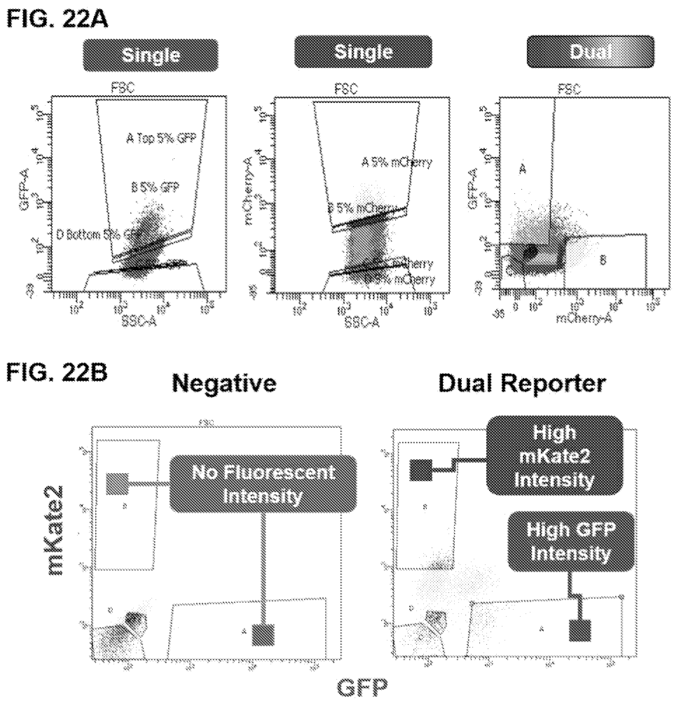

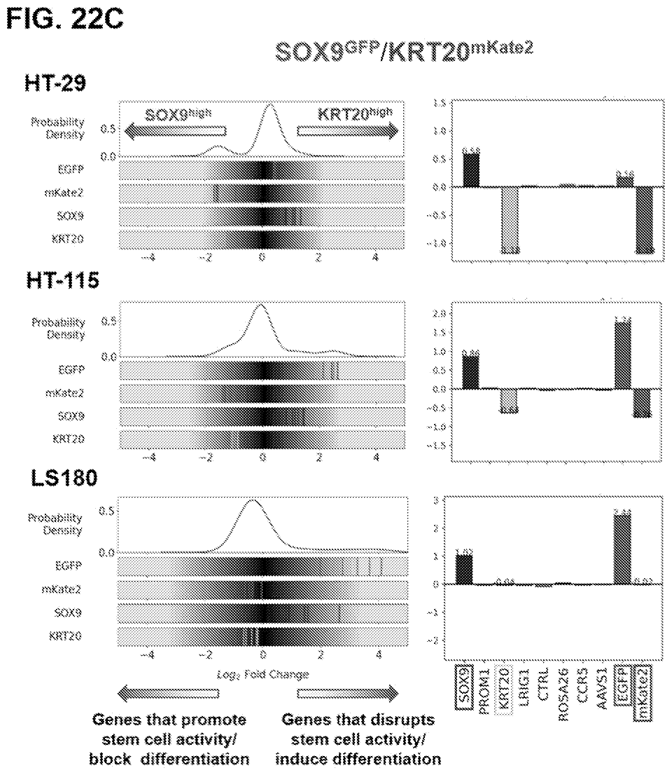

After successful functional validation of the single endogenous stem cell and differentiation reporter systems, a dual reporter system was engineered where two different fluorescent probes were introduced to simultaneously monitor stem cell and differentiation programs within the same CRC cell line (FIG. 21A). The genomes of two CRC cell lines were edited to express mKate2 (an RFP derivative) from the SOX9 locus and GFP from the KRT20 locus. After introduction of the focused CRISPR/Cas9 library, a two-color flow was performed at day 7, adopting a gating scheme that converts the single reporter activity into a dual reporter system (FIG. 22A-13). In this system, sgRNAs that inactivate genes that promote stem cell activity and block differentiation will be found in the mKate2^{low}/GFP^{high} quadrant, whereas sgRNAs that inactivate genes that disrupt stem cell activity and induce differentiation will be found in the mKate2^{high}/GFP^{low} quadrant. sgRNAs against genes that are candidate therapeutic targets would therefore be recovered from the mKate2^{low}/GFP^{high} quadrant as genetic perturbation would impair stem cell signaling and induce differentiation. Consistent with this idea, SOX9 and mKate2 sgRNAs were enriched in the mKate2^{low}/GFP^{high} fraction of HT-29 and HT-115 dual reporter CRC lines, thereby serving as positive controls for genes whose function would normally support stem cell activity and block differentiation (FIG. 21B-C). By contrast, KRT20 and mKate2 guides were enriched in the mKate2^{high}/GFP^{low} fraction, thereby serving as positive controls for genes that normally impair stem cell signaling and induce differentiation in CRC (FIG. 21B-C). Notably, the magnitude of sgRNA discrimination is greater in the endogenous dual reporter compared to single reporter system, suggesting that utilization of a dual stem cell and differentiation reporter improves the ability to identify genes that regulate this axis in CRC (FIG. 21B compared to 19E).

To control for potential technical fluorescent color bias, dual reporter lines were engineered with swapped fluorescent probes; therefore, GFP was expressed from the SOX9 locus and mKate2 from the KRT20 locus (FIG. 22C). A third CRC cell line LS180 for these dual reporters was also included. Consistent with the original dual reporter CRC cell lines, the ones with reversed fluorescent probes showed the expected distribution of SOX9, GFP, KRT20, and mKate2 sgRNAs relative to controls. While the LS180 dual reporter was able to capture the mKate2 and SOX9 sgRNAs in the mKate2^{low}/GFP^{high} fraction, it was unable to enrich for KRT20 and GFP sgRNAs, potentially due to low endogenous KRT20 expression in the cell line, which can complicate development of the endogenous differentiation reporter. These data describe and validate the successful development of an endogenous dual stem cell and differentiation reporter system in CRC.

### Example 13: Application of Endogenous Reporter Systems to Identify Therapeutic Agents in CRC

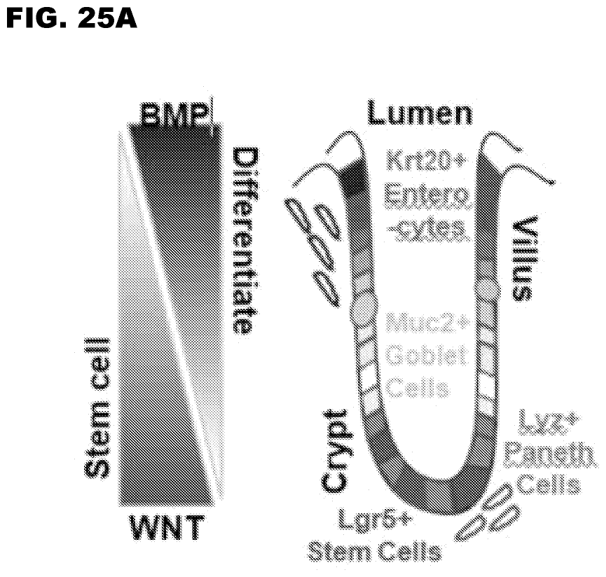

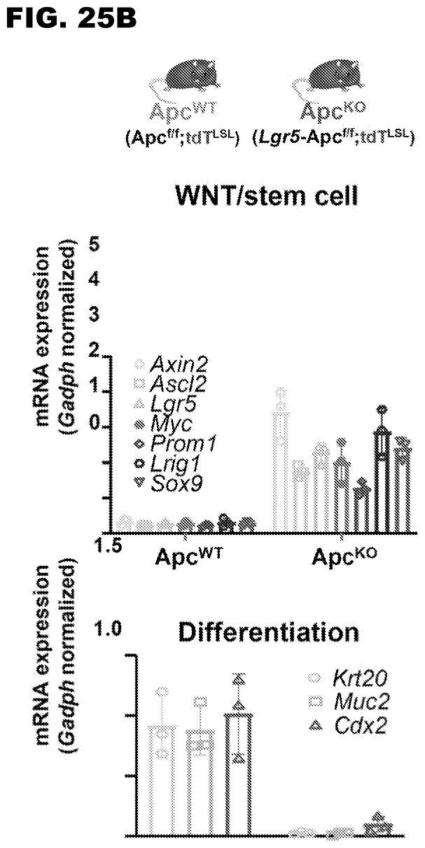

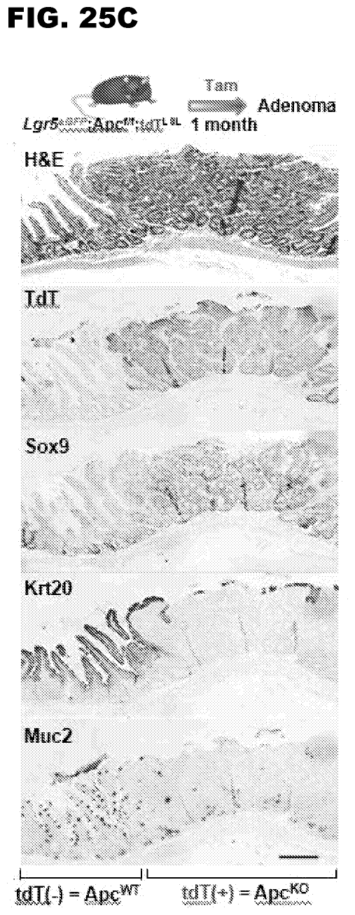

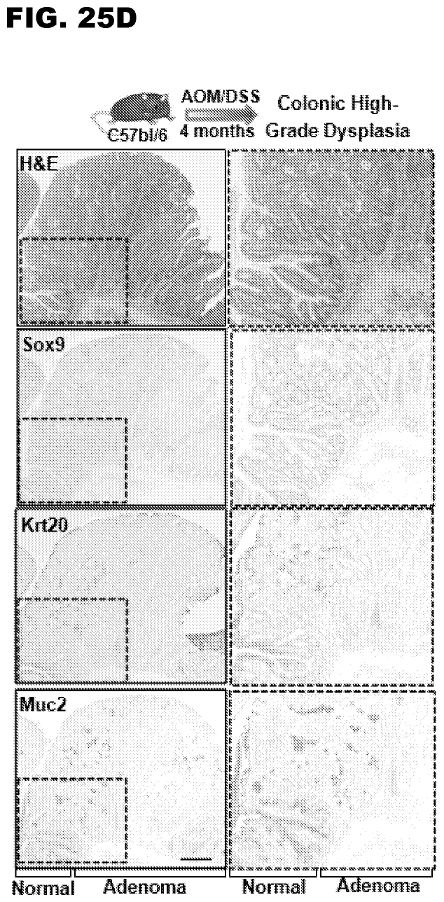

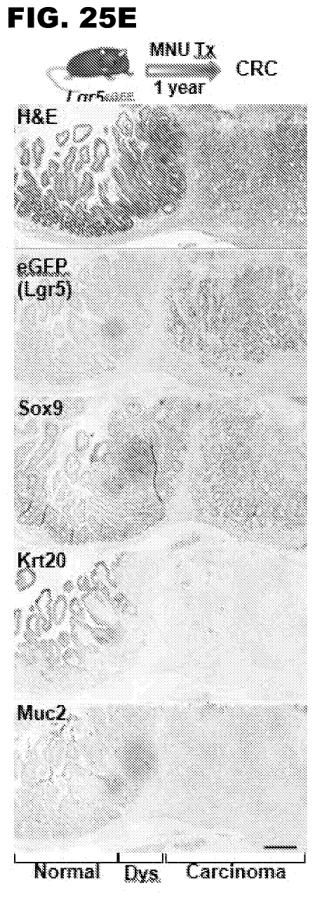

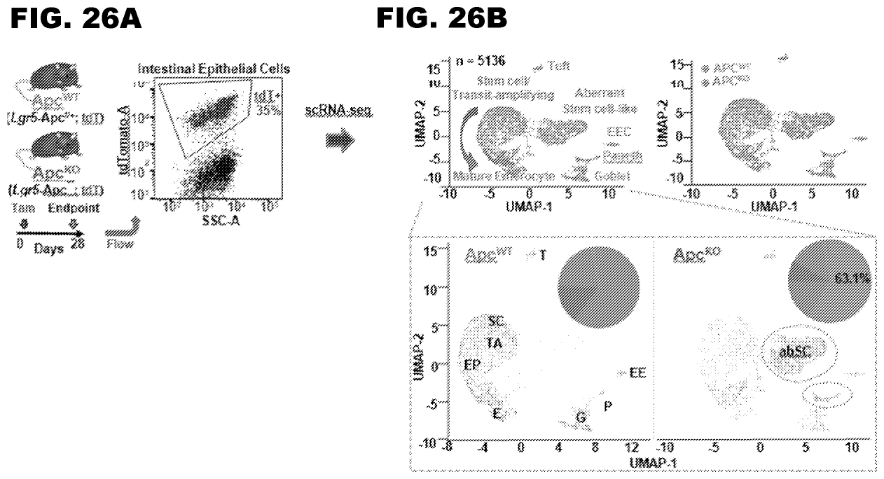

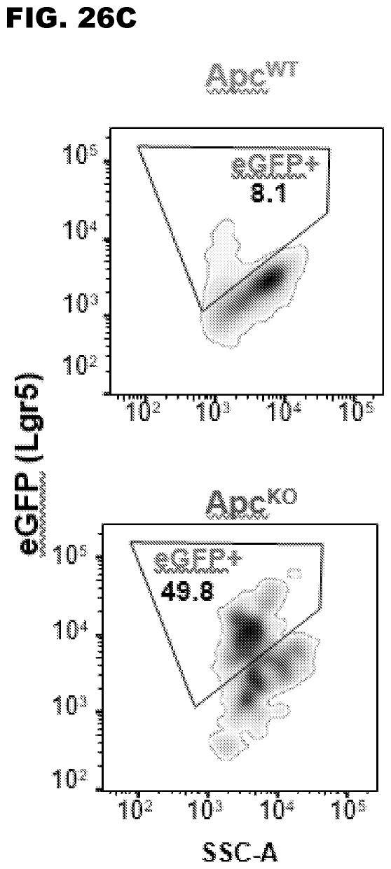

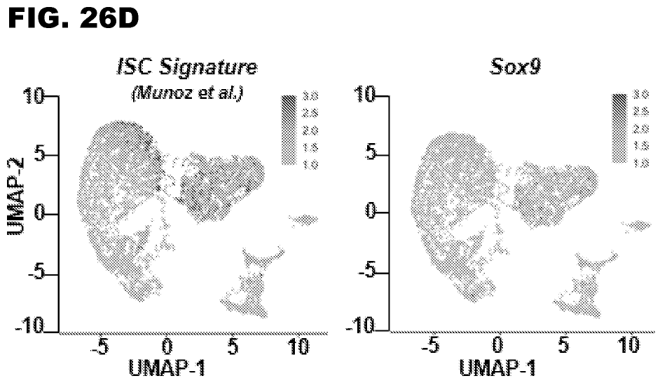

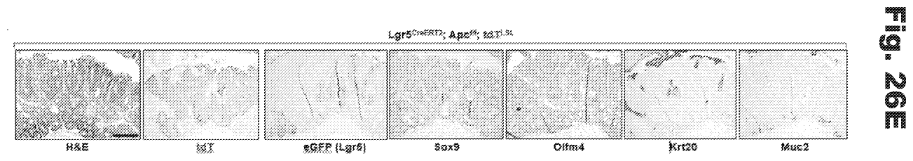

Next, the endogenous stem cell and differentiation platform was utilized to identify chemical compounds that overcome the intrinsic differentiation block in CRC, starting with a well-annotated epigenetic drug library. Epigenetic regulators are important in stem cell and differentiation programs and therefore inhibitors of these factors may be able to reprogram CRC cells. A 31-compound library of well-annotated small molecules against epigenetic regulators was obtained through a collaborator. The library consists of synthesized compounds that either degrade or disrupt specific enzyme activity of ATP-dependent (e.g. SNI/SWF family members) and covalent modifying (e.g. histone deacetylases (HDAC)) chromatin remodelers among other epigenetic regulators. An initial screen was performed at a relatively high concentration of 10 μM for each compound. After 4 days of treatment, stem cell activity was measured in LS180^{SOX9-GFP} by GFP, intestinal differentiation in HT-115^{KRT20-mKate2} by mKate2, and cell viability in all three parental CRC cell lines (i.e., HT-29, LS180, and HT-115) using automated cell counting (i.e., directly counts viable cells without requiring an end-point assay) (FIGS. 25A and 26A).

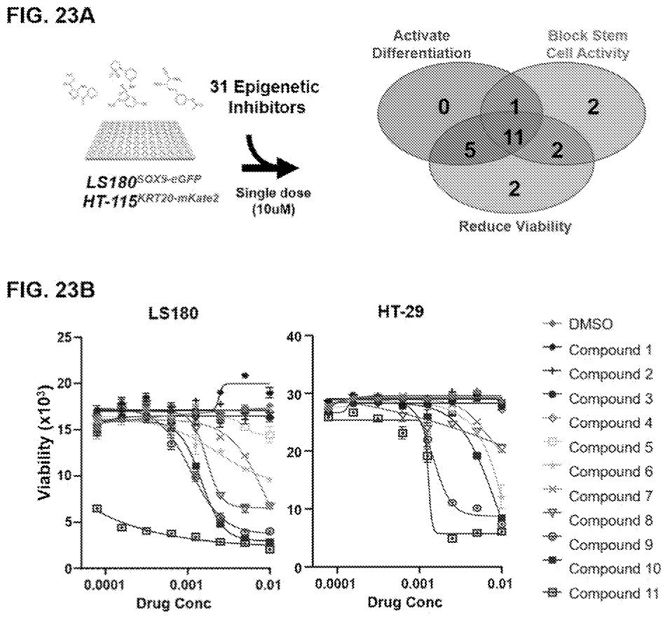

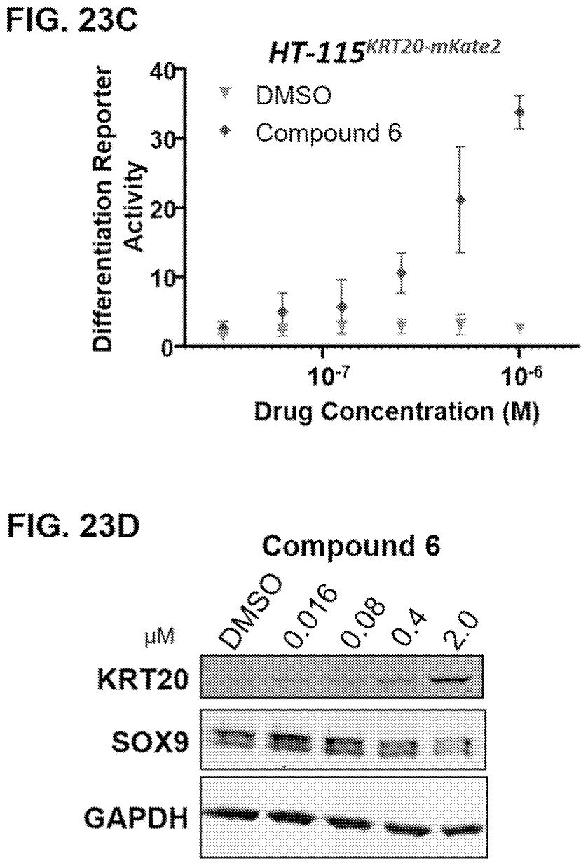

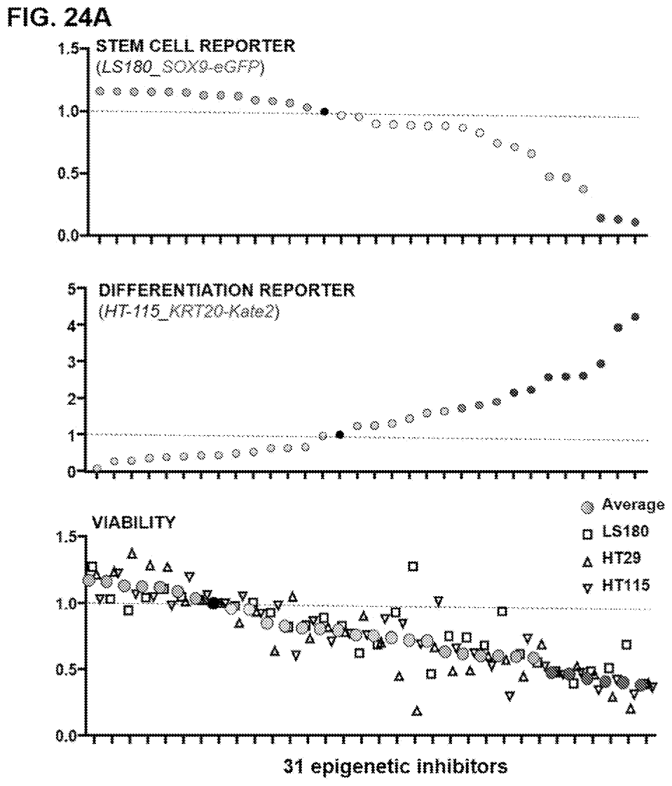

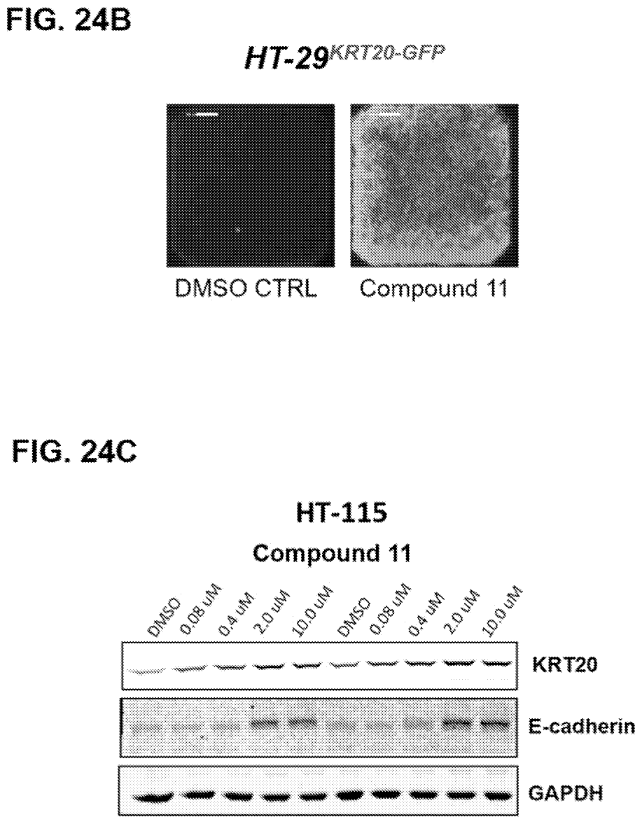

Among the 31 epigenetic inhibitors, 11 compounds decreased stem cell activity, induced intestinal differentiation, and reduced cell viability in CRC. These 11 small molecules were then used for a secondary screen (FIG. 23A). The secondary screen was performed at a range of drug concentrations for each of the 11 compounds to establish dose-response curves. Of the 11 compounds, 6 compounds consistently lead to reduced survival in two CRC cell lines in a dose-dependent fashion (FIG. 23B). Of the 6 compounds, 2 are categorized as one class of epigenetic inhibitors while 3 fall under a second class, providing some confidence that these are on-target effects. It was then determined whether these 6 compounds could block stem cell signaling (i.e., reduce SOX9 expression) and induce differentiation (i.e., activate KRT20) using gold-standard methods (e.g., Western blot and RT-PCR for measuring protein and mRNA expression). Two compounds, noted here as Compound 6 and Compound 11, were validated. In a dose-dependent fashion, Compound 6 activated the endogenous differentiation reporter as measured by a fluorescent plate reader (FIG. 23C) and induced KRT20 expression by immunoblot (FIG. 23D). By contrast, Compound 6 reduced SOX9 expression in a dose-dependent fashion. Compound 11 was able to turn on the differentiation reporter (FIG. 24C) and induce KRT20 and E-Cadherin, a more general marker of epithelial cell differentiation. These experiments indicate that small molecule inhibitors that induce intestinal differentiation and reduce viability with favorable dose-response profiles can be identified using the platform disclosed herein

Aberrant activation of stem cell-like programs and their ability to block proper differentiation are central to the pathogenesis of CRC. As disclosed herein, the development of an endogenous stem cell and differentiation reporter system in CRC is shown. This system was genetically and biologically validated and its utility was demonstrated in the genetic and chemical compound screening format. Of translational importance, this platform was and can be further utilized to discover novel therapeutic agents that reduce CRC viability by inducing intestinal differentiation

### Example 14: Materials and Methods for Examples 15-21

Animal studies. All procedures involving mice and experimental protocols were approved by Institutional Animal Care and Use Committee (IACUC) of Dana-Farber Cancer Institute (11-009). The generation of Lgr5eGFP-IRES-CreERT2 mouse was described earlier (Barker, N., van Es, J. H., Kuipers, J., Kujala, P., van den Born, M., Cozijnsen, M., Haegebarth, A., Korving, J., Begthel, H., Peters, P. J., et al. (2007). Identification of stem cells in small intestine and colon by marker gene Lgr5. Nature 449, 1003-1007). Lgr5eGFP-IRES-CreERT2 mice were backcrossed in C57BL/6J mice and subsequently SNP tested to ensure >97% pure background (Taconic). To inactivate Apc in intestinal tissue, Apc^{f/f} mice were crossed to Lgr5eGFP-IRES-CreERT2. These mice were further crossed to R26-LSL-tdTomato purchased from Jackson Laboratory (JAX stock #007905) (Madisen, L., Zwingman, T. A., Sunkin, S. M., Oh, S. W., Zariwala, H. A., Gu, H., Ng, L. L., Palmiter, R. D., Hawrylycz, M. J., Jones, A. R., et al. (2010). A robust and high-throughput Cre reporting and characterization system for the whole mouse brain. Nat Neurosci 13, 133-140) for conditional tdT labeling of Lgr5+ stem cells and their progeny. To delete Sox9, mice were crossed to Sox9flox mice (JAX stock #013106) (Akiyama, H., Chaboissier, M. C., Martin, J. F., Schedl, A., and de Crombrugghe, B. (2002). The transcription factor Sox9 has essential roles in successive steps of the chondrocyte differentiation pathway and is required for expression of Sox5 and Sox6. Genes Dev 16, 2813-2828). To activate conditional alleles, experimental mice aged 6-8 weeks were injected intraperitoneally with a single dose tamoxifen (50 ul of a sunflower oil at 10 mg/ml) unless otherwise indicated. For MNU model, Lgr5eGFP-IRES-CreERT2 mice were treated with drinking water containing 240 ppm of N-methyl-N-nitrosourea (MNU; biokemix) scheduled every other week for 10 weeks and followed for 1 year. For AOM/DSS model, AOM/DSS treatment was performed as previously described (Neufert, C., Becker, C., and Neurath, M. F. (2007). An inducible mouse model of colon carcinogenesis for the analysis of sporadic and inflammation-driven tumor progression. Nat Protoc 2, 1998-2004). Briefly, at 8-10 weeks of age mice were injected intraperitoneally with 10 mg/kg AOM (Sigma-Aldrich) and treated with 2% DSS (30-50 kDa, colitis grade, MP Biomedicals) in drinking water for 5 days. DSS treatment was repeated 3 times once per 4 weeks. Mice were sacrificed 11 weeks after AOM injection.

Mice were euthanized at the endpoint of the experiment and small intestine and colon were removed from mice, flushed with PBS, and incubated in 10% formalin for 5 min. Subsequently, intestines were opened longitudinally, ‘swiss-rolled’, incubated overnight in 10% formalin at room temperature and processed for paraffin embedding. Fresh tissue was collected for organoid generation, RNA isolation, protein collection and flash-frozen for long-term storage.

Human specimen. Adenoma and paired normal-appearing tissue from a patient with familial adenomatous polyposis were collected post colectomy under approval (protocol 13-189) by the Internal Review Board of the Dana FarberCancer Institute, Boston, Massachusetts, USA.

Histopathology. Paraffin-embedded intestines, organoids or xenograft tumors were serially sectioned and mounted on a slide. Sections were subjected to hematoxylin and eosin (H&E), Alcian blue-Periodic Acid Schiff (AB-PAS), as well as immunostaining, using standard procedures. For morphological analysis, sections were serially dehydrated in xylene and ethanol, stained with H&E for histological assessment or AB-PAS to identify goblet cells and mucus.

Immunofluorescence and immunohistochemistry. For immunostaining, antigen retrieval was performed using a sodium citrate buffer (pH6), Trilogy (Sigma Aldrich Cell Marque) or Tris-EDTA pH9. Slides were permeabilized using a 0.2% Triton X100 for 30 minutes at room temperature and blocked with donkey serum for 1 hour. The primary antibodies used for immunohistochemistry were rabbit anti-Sox9 (1:600, CST, #82630, Trilogy antigen-retrieval), rabbit anti-Mucin2 (1:200, Santa Cruz, sc-15334, Tris-EDTA), anti-Krt20 (1:500, CST, #13063, Tris-EDTA), anti-RFP (1:500, Rockland, 600-401-379, sodium Citrate pH6), anti-eGFP (1:1000, Abcam, ab290, Tris-EDTA), anti-Olfm4 (1: 1000, CST, #39141, sodium Citrate pH6), anti-Lyz, anti-Sca-1/Ly6a (1:150, Abcam, ab109211, sodium Citrate pH6), anti-Axin2 (1:800, Abcam, ab32197, sodium Citrate pH6) and rabbit anti-Ki67 (1:1,000, CST, #12202, sodium Citrate pH6). Binding of primary antibody was detected with 3,39-diamino-benzidine-tetrahydrochloride-dihydrate and counterstained with hematoxylin. IHC quantification was performed using Fiji Is Just Image J (FIJI) application on 10× images from Leica DM750 microscope. Ly6a or Sox9 positive cells were quantified and expressed as a percentage of total tdT+ cells in intestinal lesions. For organoids, the percentage of Ki67 positive cells were quantified and expressed as a percentage of total cells in an organoid.

Flow analysis, sorting, and staining. Isolated intestines from euthanized mice were washed with ice-cold PBS; villi were scraped using glass slides and dissociated in 5 mM EDTA in PBS at 4° C. for 20 minutes, shaking every 5-7 minutes for about 30 seconds. The epithelial fraction was collected by centrifugation and incubated at 37° C. for 30 minutes in pre-warmed 4× TrypLE. Single cells filtered through 70 um filter post trypsinization, washed with complete media, and collected in FACS buffer.

For antibody staining, 100 ul of resuspended cells in FACS buffer were incubated with 1 ul APC labelled Sca-1 antibody (Thermofisher scientific, Catalog #17-5981-82) for 20 mins on Ice, washed with PBS and collected in FACS buffer. Cells were resuspended in 1-2 mL FACS buffer with DAPI and passed through another 70-micron filter before transferring to 40 micron filtered FACS tubes and sorted. Sorted cells were collected in 5 mL of 50% FBS+50% DMEM complete with 10 uM Y27632 ROCK inhibitor.

Single cell RNA sequencing. Murine intestines and human adenoma samples were processed as described above and then subjected to single cell RNA sequencing (scRNAseq) with or without cell hashing. If hashed, the mouse cells were stained with TotalSeg™-30301 Hashtag 1 (BioLegend #155861) and TotalSeg™-30302 Hashtag 2 (BioLegend #155863) antibodies; no hashing was used for human samples. Viable cells were washed and resuspended in PBS with 0.04% BSA at a cell concentration of 1000 cells/μL. About 17,000 viable mouse cells were loaded onto a 10× Genomics Chromium™ instrument (10× Genomics) according to the manufacturer's recommendations. The scRNAseq libraries were processed using Chromium™ single cell 5′ library & gel bead kit (10× Genomics).

Matched cell hashing libraries were prepared using single cell 5′ feature barcode library kit. Quality controls for amplified cDNA libraries, cell hashing libraries, and final sequencing libraries were performed using Bioanalyzer High Sensitivity DNA Kit (Agilent). The sequencing libraries for scRNAseq and scTCRseq were normalized to 4 nM concentration and pooled using a volume ratio of 4:1. The pooled sequencing libraries were sequenced on Illumina NovaSeq S4 300 cycle platform.

The sequencing parameters were: Read 1 of 150 bp, Read 2 of 150 bp and Index 1 of 8 bp. The sequencing data were demultiplexed and aligned to mm10-3.0.0 using cell ranger version 3.1.0 pipeline (10× Genomics).

### Single Cell RNA Sequencing Analysis.

Pre-processing, alignment and gene counts: De-multiplexing, alignment to the transcriptome, and unique molecular identifier (UMI) collapsing were performed using the Cellranger toolkit provided by 10× Genomics.

General Clustering: Standard procedures for QC filtering, data scaling and normalization, detection of highly variable genes, and hashtag oligo (HTO) demultiplexing were followed using the Seurat v3 in RStudio. Cells with unique feature counts lower than 100 and greater than 25,000 as well as cells with greater than 25% mitochondrial DNA were excluded. Counts were log-normalized and scaled by a factor of 10,000 according to the default parameters when using the Seurat LogNormalize function. Variable features were identified, and the data were scaled using the default parameters (Ngenes=2000) of the FindVariableFeatures and ScaleData Seurat functions, respectively. HTOs were demultiplexed using the HTODemux function, and cells were identified as containing HTO-1 or HTO-2 based on their maximal HTO-ID signal. The cell population was filtered to contain only HTO-positive, singlet cells for further analysis. Principle component analysis (PCA) was completed on the remaining cells and 10 principle components were selected for clustering, tSNE, and UMAP analyses. Cells were visualized primarily using UMAP non-linear dimensional reduction (dims 1:10, resolution=0.3), from which feature plots were generated to demonstrate distribution of gene expression and APC^{WT} versus APC^{KO} cells and expression levels of various marker genes throughout the population. Marker genes for each resulting cluster were found using the FindMarkers function with the minimum prevalence set to 25%. One cluster was determined to be immune cells based off of top marker genes and excluded from further analysis. Cluster identities were defined using known marker genes for intestinal epithelial cell types.

General analysis: scRNA-seq IntegrateData function in Seuratv4 was used to counteract batch effects among human tissue (Paired Normal A, Paired Normal B, Adenoma) and mouse model samples.

Principal Component Analysis (PCA) was then completed on the integrated object and the quantity of principal components selected for clustering was determined using the integrated object's elbow plot. Cells were then visualized primarily using UMAP non-linear dimensional reduction from which feature and violin plots were generated to demonstrate distribution of gene expression and expression levels of various marker genes and gene signatures throughout the population.

scRNA-seq gene signature analysis: To analyze existing gene signatures on the scRNA-seq data, the Seurat AddModuleScore function in Seurat v4 was used to calculate the average normalized and scaled gene expression of a given gene list in each individual cell. Specific cell types were identified using established marker genes and gene signatures (Haber, A. L., Biton, M., Rogel, N., Herbst, R. H., Shekhar, K., Smillie, C., Burgin, G., Delorey, T. M., Howitt, M. R., Katz, Y., et al. (2017). A single-cell survey of the small intestinal epithelium. Nature 551, 333-339). Gene signature scoring was then visualized with feature and violin plots. To generate novel gene signatures, the Seurat FindMarkers function was used to create lists of genes differentially expressed in one specified subset in comparison to another given subset. Minimum prevalence was set to 25%.

### Assay for Transposase-Accessible Chromatin Sequencing (ATAC-Seq).

ATAC libraries were prepared as described previously (Corces, M. R., Trevino, A. E., Hamilton, E. G., Greenside, P. G., Sinnott-Armstrong, N. A., Vesuna, S., Satpathy, A. T., Rubin, A. J., Montine, K. S., Wu, B., et al. (2017). An improved ATAC-seq protocol reduces background and enables interrogation of frozen tissues. Nature methods 14, 959-962). Briefly, flow-sorted tdT+ cells (25,000 cells in duplicates) were lysed to prepare nuclear pellets which then underwent transposition with TDE1 Enzyme (Illumina, 20034197). Tagmented DNA was purified using Zymo DNA Clean and Concentrator-5 Kit (cat #D4014) and the purified DNA was PCR amplified with NEBNext 2× MasterMix and Illumina adapters (Table 9). The libraries were purified post-PCR using AMPure XP beads (Beckman Coulter). 150-bp paired-end reads were sequenced on a Nextseq instrument (Illumina).

Intestinal Organoid Culture. Colonic glands were isolated, treated with EDTA, and then resuspended in 30-50 ul of Matrigel (BD Bioscience) and plated in 24-well plates. WNT/R-spondin/Noggin (WRN) containing DMEM/F12 with HEPES (Sigma-Aldrich) containing 20% FBS, 1% penicillin/streptomycin and 50 ng/ml recombinant mouse EGF (Life Technologies) was used for culturing ApcKO colon organoids. For the first 2-3 days after seeding, the media was also supplemented with 10 mM ROCK inhibitor Y-27632 (Sigma Aldrich) and 10 mM SB431542 (Sigma Aldrich), an inhibitor for the transforming growth factor (TGF)-β type I receptor to avoid anoikis. For passage, colon organoids were dispersed by trypsin-EDTA and transferred to fresh Matrigel. Passage was performed every 3-4 days with a 1:3-1:5 split ratio. For human colon organoid culture, the previous media was supplemented with antibiotics 100 ug/ml Primocin (Invivogen), 100 ug/ml Normocin (Invivogen); serum-free supplements 1× B27 (Thermo Fisher (Gibco)), 1× N2 (Thermo Fisher (Gibco)); chemical supplements 10 mM Nicotinamide (Sigma), 500 mM N-acetylcysteine (Sigma), hormone 50 mM [Leu15]-Gastrin (Sigma), growth factor 100 ug/ml FGF10 (recombinant human) (Thermo Fisher) and 500 nM A-83-01 (Sigma), which is an inhibitor of the TGF-β Receptors ALK4, 5, and 7.

Organoid fixation for FFPE. Confluent organoids in 6-well plate were fixed in 10% formalin at 4° C. on rocker overnight. The fixed organoids were washed with PBS, collected by centrifugation at 2000 rpm, 3 mins. The supernatant was carefully aspirated; cell pellets were resuspended in 50-80 ul of 2% agar and then immediately kept on ice. The solidified agar with organoids was kept in a cassette and processed to make FFPE blocks.

Cell Proliferation Assays. Cell viability was quantified by measuring cellular ATP content using the CellTiterGlo Cell Viability assay (Promega) according to the manufacturer's instructions. All experiments were performed in triplicate in 96-well plates.

SOX9 recombination PCR. Mouse tails were incubated at 95° C. in 75 μl of the cell lysis buffer (5 mL sterile ddH2O, 7 μl 50% NaOH, 7 μl 0.5M EDTA, pH 8.0) followed by 15 minutes at 4° C. and neutralized with 75 μl of neutralization buffer (40 mM Tris-HCl, pH ˜5). 2 μl of supernatant was used for PCR amplification using primers designed for WT SOX9; Unfloxed SOX9 and Floxed SOX9 (Table 9) using Phire Tissue Direct PCR master mix (Thermo Fisher Scientific #F170S). PCR cycling conditions were initial denaturation 94° C.—120 s; 10× touchdown cycles with the following three steps: 94° C.—10 s, 65° C. (0.5° C. increment each cycle)—10 s, 72° C.—90 s; 25× cycles with the following three steps: 94° C. —10 s, 60° C.—10 s, 72° C.—90 s; the last annealing 72° C.-180 s. The PCR products were analysed on 1% agarose gel.

Organoid Adenoviral Transduction. To transduce colonic organoids, 0.5 M organoids were suspended in 500 ul of WRN media with 10 uM Y27632 ROCK inhibitor in a 24 well ultra-low attachment plate. Adenovirus carrying GFP or ^{GFP-Cre} (1 ul) was added to the organoids and the sealed plate is centrifuged 600 g×1 hr at 32 C followed by incubation at 37 C for 5-6 hrs. Post-incubation, the transduced organoids were washed with complete media and resuspended in about 200 ul Matrigel and plated in nunclon delta plates with WRN conditioned media with 10 uM ROCK inhibitor.

Generation of stable colon organoids. All genetically manipulated colon organoid lines were generated using the protocol described here (Shalem, O., Sanjana, N. E., Hartenian, E., Shi, X., Scott, D. A., Mikkelson, T., Heckl, D., Ebert, B. L., Root, D. E., Doench, J. G., et al. (2014). Genome-scale CRISPR-Cas9 knockout screening in human cells. Science 343, 84-87). shRNAs against SOX9 were cloned into PLKO.1, TET-PLKO and TET-Cellecta vectors. To generate lentiviruses, expression vectors were co-transfected into HEK293T cells with the lentiviral packaging constructs psPAX2 and pMD2.G (VSV-G) in a 1:1:1 ratio using X-tremeGENE 9 DNA Transfection Reagent (Roche) according to the manufacturer's instructions. Cell culture media was changed the following day and lentiviral supernatant was harvested 48 h and 72 h later and filtered through a 0.45 m filter (Millipore). Lentiviruses were aliquoted and stored at −80° C. until use.

To transduce colonic organoids, spheroids in one well (24-well plate) were trypsinized and a one-fourth to one-eighth volume of cell suspension was used for each infection. Cells were resuspended in 500 l lentiviral supernatant with 8 μg/mL polybrene and 10 mM Y-27632, centrifuged at 600 g 32° C. 1 hours, and incubated for 6 hours in cell culture incubator. The infected cells were suspended in 30-50 ul of Matrigel and cultured with Wnt/R-spondin-deprived medium containing 10 mM Y-27632 and 10 mM SB431542. Colon organoids were selected with 3 μg/ml puromycin at 24 hours post-infection.

RNA isolation and qPCR. Total RNA was isolated using the RNeasy Mini Kit (Qiagen, Germantown, MD, USA) and cDNA was synthesized using the iScript™ Reverse Transcription Supermix for RT-qPCR (Bio-Rad) according to the manufacturer's instructions. Gene-specific primers for SYBR Green real-time PCR were either obtained from previously published sequences or designed by PrimerBLAST (found on the World Wide Web at ncbi.nlm.nih.gov/tools/primer-blast/) and synthesized by Integrated DNA Technologies or ETON biosciences. Real-time PCR was performed and analyzed using CFX96 Real-Time PCR Detection System (Bio-Rad Laboratories, Inc., Hercules, CA) and using Power SYBR Green PCR Master Mix (Thermo Fisher Scientific) according to the manufacturer's instructions. Relative mRNA expression was determined by normalizing to GAPDH expression, which served as an internal control. See Table 9 for primers used for qPCR.

Immunoblot, antibodies and inhibitors. Immunoblot analysis was performed as previously described (Wong, G. S., Zhou, J., Liu, J. B., Wu, Z., Xu, X., Li, T., Xu, D., Schumacher, S.E., Puschhof, J., McFarland, J., et al. (2018). Targeting wild-type KRAS-amplified gastroesophageal cancer through combined MEK and SIP2 inhibition. Nature medicine 24, 968-977). Briefly, cells were lysed in RIPA buffer supplemented with a protease inhibitor cocktail (Roche). Whole cell extracts were resolved by SDS-PAGE, transferred to PVDF membranes, and probed with indicated primary antibodies. Bound antibodies were detected with horseradish peroxidase (HRP)-conjugated secondary antibodies and chemiluminescent HRP substrate. The following primary antibodies were used for western blotting (all from Cell Signaling Technologies, Beverly, MA, USA, unless otherwise indicated): anti-SOX9 (#82630, 1:1,000), anti-Vinculin (#13901, 1:1,000), anti-RFP (Rockland, 600-401-379, 1:500).

Statistical Analysis and reproducibility. Experiments were performed in triplicate. Data are represented as mean±s.d unless indicated otherwise. For each experiment, either independent biological or technical replicates are as noted in the figure legends and were repeated with similar results. Statistical analysis was performed using Microsoft Office, Prism 7.0 (GraphPad), or RStudio statistical tools. Pairwise comparisons between groups (that is, experimental versus control) were performed using an unpaired two-tailed Student's t-test or Kruskal-Wallis test as appropriate unless otherwise indicated. For all experiments, the variance between comparison groups was found to be equivalent. Animals were excluded from analysis if they were euthanized due to health reasons unrelated to tumor volume endpoint.

### Example 15: Single Cell RNA Sequencing Reveals Developmental Reprogramming as a Requirement for Colorectal Cancer Initiation

Genomic alterations enable cancer initiation by imparting clonal fitness. The ensuing nongenetic factors that confer cellular advantages are poorly defined. Using genetically engineered and carcinogen-induced murine models of intestinal neoplasia, it is demonstrated herein that inappropriate Sox9 expression and loss of post-mitotic villua differentiation are early events preceding cancer development. Single cell RNA-sequencing of adenomas from a genetic mouse model and patient with familial adenomatous polyposis revealed that Apc inactivation leads to a distinct transcriptional state characterized by indiscriminate attenuation of differentiated lineages, and aberrant activation of genes associated with fetal intestinal development, including Ly6a/Sca-1. Genetic inactivation of Sox9 prevented adenoma formation in ApcKO mice, obstructed emergence of the aberrant transcriptional state, including genes reserved for fetal intestinal development, and restored multi-lineage differentiation by singe cellRNA-sequencing. SOX9 knockdown in human adenoma organoids compromised expression of fetal genes and induced differentiation.. These studies indicate that developmental reprogramming is a requirement for colorectal cancer initiation. Furthermore, these findings carry important implications for developing CRC therapeutics directed at restoring differentiation.

Herein, it is demonstrated that reactivation of a fetal intestinal program is a critical component of an aberrant transcriptional state that restricts differentiation during colorectal cancer initiation. Genetic disruption of Sox9obstructs aberrant transcriptional activity, suppresses fetal gene reactivation, prevents adenoma formation, and promotes differentiation, inspiring therapeutic strategies directed at restoring differentiation in colorectal cancer.

The most rapidly renewing epithelium in the human body lines the intestines. This renewal process depends on crypt-restricted Lgr5+ intestinal stem cells (ISC) and their immediate progeny to replicate frequently and spawn a steady stream of postmitotic differentiated cells (Sato, T., Vries, R. G., Snippert, H. J., van de Wetering, M., Barker, N., Stange, D. E., van Es, J. H., Abo, A., Kujala, P., Peters, P. J., et al. (2009). Single Lgr5 stem cells build crypt-villus structures in vitro without a mesenchymal niche. Nature 459, 262-265). These specialized cells function in barrier protection, absorption, hormone secretion, and other critical roles that facilitate digestion and intestinal function in the villus before dying. The key pathways that regulate renewal and differentiation include wingless/integrated (WNT) and bone morphogenetic protein (BMP) signaling cascades, respectively. WNT pathway activity maintains stem cell reservoirs and crypt homeostasis whereas BMP signaling supports differentiation of progenitors into mature enterocytes, establishing a crypt-villus gradient (Chen, L., Toke, N. H., Luo, S., Vasoya, R. P., Fullem, R. L., Parthasarathy, A., Perekatt, A. O., and Verzi, M. P. (2019). A reinforcing HNF4-SMAD4 feed-forward module stabilizes enterocyte identity. Nat Genet 51, 777-785; Sancho, E., Batlle, E., and Clevers, H. (2004). Signaling pathways in intestinal development and cancer. Annu Rev Cell Dev Biol 20, 695-723) (FIG. 25A). Disrupting this balance between stem cell and differentiation programs is a defining property of colorectal cancer (CRC), which remains the third most common and second most deadly malignancy worldwide, accounting for 1.8 million new cases and greater than 860,000 deaths each year, respectively (Siegel, R. L., Miller, K. D., and Jemal, A. (2018). Cancer statistics, 2018. CA: a cancer journal for clinicians 68, 7-30).

Majority of sporadic CRCs initiate as a premalignant adenoma harboring genomic alterations that constitutively activate WNT signaling (Rustgi, A. K. (1993). Molecular genetics and colorectal cancer. Gastroenterology 104, 1223-1225), most often through loss-of-function APC mutations (Fearon, E. R., and Vogelstein, B. (1990). A genetic model for colorectal tumorigenesis. Cell 61, 759-767; Kinzler, K. W., Nilbert, M. C., Su, L. K., Vogelstein, B., Bryan, T. M., Levy, D. B., Smith, K. J., Preisinger, A. C., Hedge, P., McKechnie, D., et al. (1991). Identification of FAP locus genes from chromosome 5q21. Science 253, 661-665; Kinzler, K. W., and Vogelstein, B. (1996). Lessons from hereditary colorectal cancer. Cell 87, 159-170). A key component of a cytoplasmic destruction complex, APC restricts WNT signaling by facilitating ubiquitin-mediated degradation of 3-catenin (Dajani, R., Fraser, E., Roe, S. M., Yeo, M., Good, V. M., Thompson, V., Dale, T. C., and Pearl, L. H. (2003). Structural basis for recruitment of glycogen synthase kinase 3beta to the axin-APC scaffold complex. EMBO J 22, 494-501; Ha, N.C., Tonozuka, T., Stamos, J. L., Choi, H. J., and Weis, W. I. (2004). Mechanism of phosphorylation-dependent binding of APC to beta-catenin and its role in beta-catenin degradation. Mol Cell 15, 511-521; Korswagen, H. C., Coudreuse, D. Y., Betist, M. C., van de Water, S., Zivkovic, D., and Clevers, H. C. (2002). The Axin-like protein PRY-1 is a negative regulator of a canonical Wnt pathway in C. elegans. Genes Dev 16, 1291-1302; Munemitsu, S., Albert, I., Souza, B., Rubinfeld, B., and Polakis, P. (1995). Regulation of intracellularbeta-catenin levels by the adenomatous polyposis coli (APC) tumor-suppressor protein. Proc Natl Acad Sci USA 92, 3046-3050; Rubinfeld, B., Albert, I., Porfiri, E., Fiol, C., Munemitsu, S., and Polakis, P. (1996). Binding of GSK3beta to the APC-beta-catenin complex and regulation of complex assembly. Science 272, 1023-1026; Xing, Y., Clements, W. K., Kimelman, D., and Xu, W. (2003). Crystal structure of a beta-catenin/axin complex suggests a mechanism for the beta-catenin destruction complex. Genes Dev 17, 2753-2764). Once APC function is compromised, β-catenin accumulates in the nucleus, where it can operate as the primary effector of aberrant WNT signaling (Korswagen, H. C., Coudreuse, D. Y., Betist, M. C., van de Water, S., Zivkovic, D., and Clevers, H. C. (2002). The Axin-like protein PRY-1 is a negative regulator of a canonical Wnt pathway in C. elegans. Genes Dev 16, 1291-1302; van Noort, M., Meeldijk, J., van der Zee, R., Destree, O., and Clevers, H. (2002). Wnt signaling controls the phosphorylation status of beta-catenin. The Journal of biological chemistry 277, 17901-17905). ISC expansion and imbalanced crypt-villus homeostasis are known outcomes Apc deletion and constitutive β-catenin activity in mouse models of intestinal neoplasia (Korinek, V., Barker, N., Moerer, P., van Donselaar, E., Huls, G., Peters, P. J., and Clevers, H. (1998). Depletion of epithelial stem-cell compartments in the small intestine of mice lacking Tcf-4. Nat Genet 19, 379-383; Korinek, V., Barker, N., Morin, P. J., van Wichen, D., de Weger, R., Kinzler, K. W., Vogelstein, B., and Clevers, H. (1997). Constitutive transcriptional activation by a beta-catenin-Tcf complex in APC−/− colon carcinoma. Science 275, 1784-1787; Morin, P. J., Sparks, A. B., Korinek, V., Barker, N., Clevers, H., Vogelstein, B., and Kinzler, K. W. (1997). Activation of beta-catenin-Tcf signaling in colon cancer by mutations in beta-catenin or APC. Science 275, 1787-1790). Beyond these cellular outcomes, the nongenetic transcriptional consequences and cell state heterogeneity following deleterious APC mutations are poorly understood. This line of investigation will not only provide new insights into the development of sporadic adenomas and hereditary polyposis, but also the initiation of CRC.

Restoring expression of functional APC can reinstate WNT pathway regulation and suppress cancer initiation (Dow, L.E., O'Rourke, K. P., Simon, J., Tschaharganeh, D. F., van Es, J. H., Clevers, H., and Lowe, S. W. (2015). Apc Restoration Promotes Cellular Differentiation and Reestablishes Crypt Homeostasis in Colorectal Cancer. Cell 161, 1539-1552; Faux, M. C., Ross, J. L., Meeker, C., Johns, T., Ji, H., Simpson, R. J., Layton, M. J., and Burgess, A. W. (2004). Restoration of full-length adenomatous polyposis coli (APC) protein in a colon cancer cell line enhances cell adhesion. J Cell Sci 117, 427-439; Groden, J., Joslyn, G., Samowitz, W., Jones, D., Bhattacharyya, N., Spirio, L., Thliveris, A., Robertson, M., Egan, S., Meuth, M., et al. (1995). Response of colon cancer cell lines to the introduction of APC, a colon-specific tumor suppressor gene. Cancer Res 55, 1531-1539). Inhibiting WNT-dependent Lgr5+ stem cells can impair tumor growth and metastasis (de Sousa e Melo, F., Kurtova, A. V., Harnoss, J. M., Kljavin, N., Hoeck, J. D., Hung, J., Anderson, J. E., Storm, E. E., Modrusan, Z., Koeppen, H., et al. (2017). A distinct role for Lgr5(+) stem cells in primary and metastatic colon cancer. Nature 543, 676-680; Shimokawa, M., Ohta, Y., Nishikori, S., Matano, M., Takano, A., Fujii, M., Date, S., Sugimoto, S., Kanai, T., and Sato, T. (2017). Visualization and targeting of LGR5(+) human colon cancer stem cells. Nature 545, 187-192). Translating these concepts into effective therapy, however, has proven difficult as WNT pathway inhibitors have not advanced in preclinical and clinical testing (Barker, N., and Clevers, H. (2006). Mining the Wnt pathway for cancer therapeutics. Nat Rev Drug Discov 5, 997-1014). By defining key mediators that facilitate neoplastic initiation, new therapeutic strategies for CRC can be uncovered. Here, critical early molecular events of intestinal neoplasia are characterized by using single cell transcriptomics, histopathological analyses, and organoid experiments in mouse models and human specimens. The requirement for an aberrant transcriptional state the involves developmental reprograming in CRC initiation is also shown to be required for pathogenesis.

### Example 16: Impaired Differentiation is a Conserved Event in Multiple Mouse Models of Intestinal Neoplasia

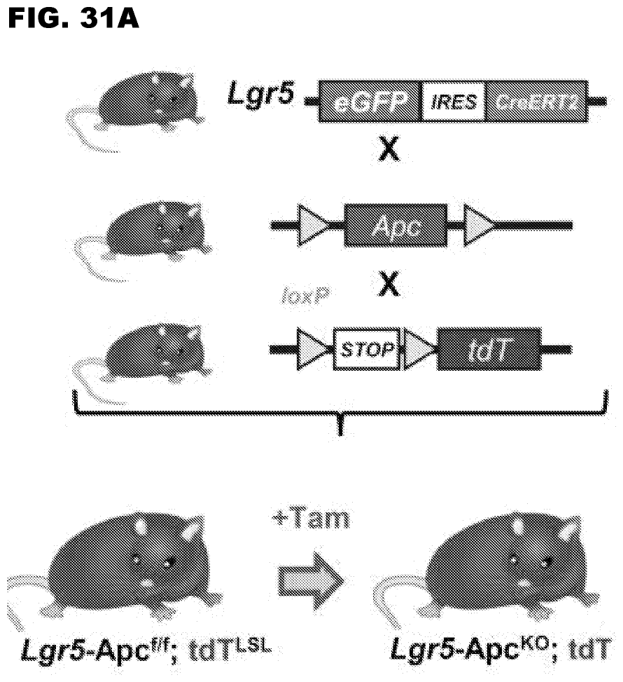

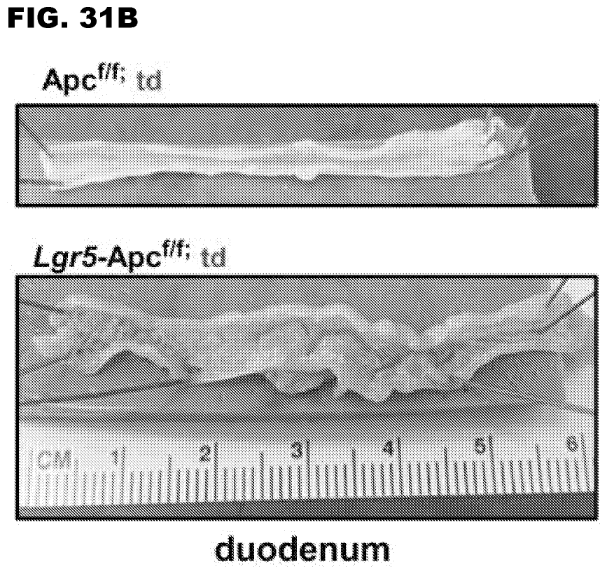

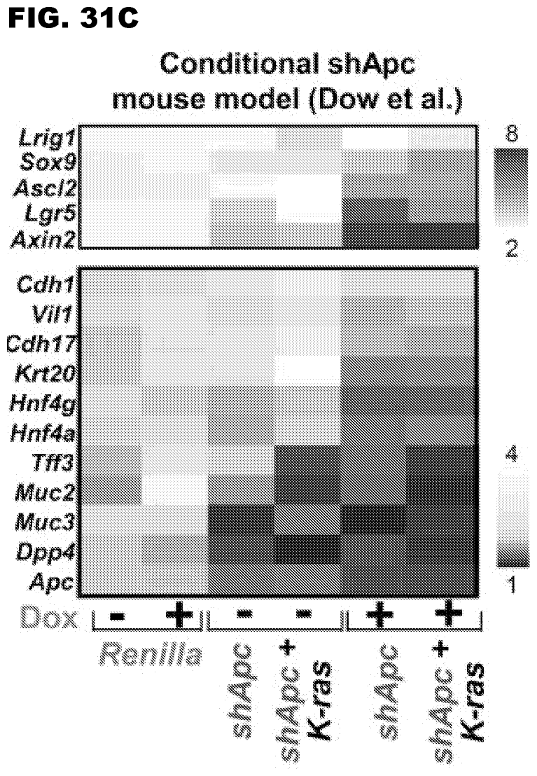

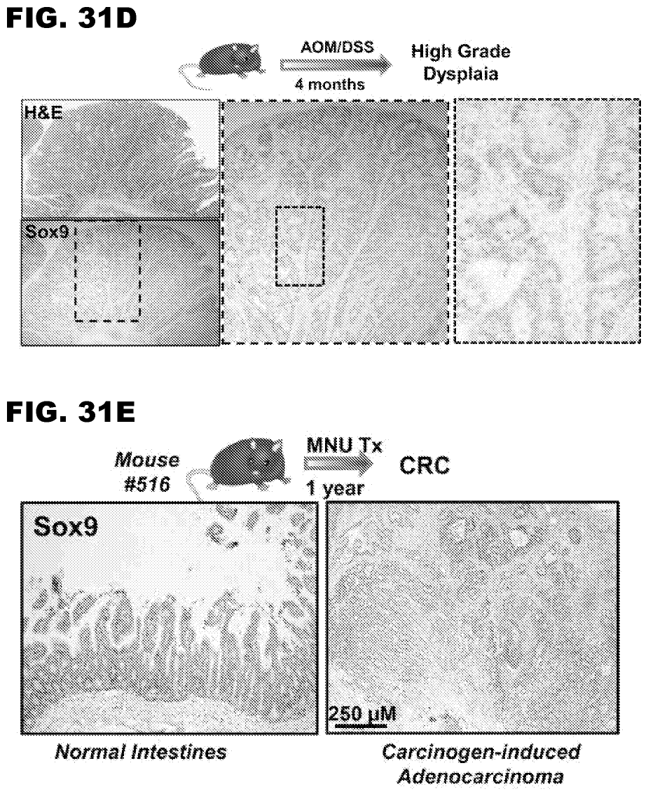

To define conserved molculat events during CRC initiation, two genetically engineered and two carcinogen-induced murine models of intestinal neoplasia were evaluated. Lgr5eGFP-IRES-CreERT2;ApcloxP-exon14-loxP(Lgr5-Apc^{f/f}) genetically engineered mice develop hundreds of adenomas in the small intestines following tamoxifen-induced conditional deletion of Apc (Colnot, S., Decaens, T., Niwa-Kawakita, M., Godard, C., Hamard, G., Kahn, A., Giovannini, M., and Perret, C. (2004a). Liver-targeted disruption of Apc in mice activates beta-catenin signaling and leads to hepatocellular carcinomas. Proc Natl Acad Sci USA 101, 17216-17221; Colnot, S., Niwa-Kawakita, M., Hamard, G., Godard, C., Le Plenier, S., Houbron, C., Romagnolo, B., Berrebi, D., Giovannini, M., and Perret, C. (2004b). Colorectal cancers in a new mouse model of familial adenomatous polyposis: influence of genetic and environmental modifiers. Lab Invest 84, 1619-1630) in Lgr5+ intestinal stem cells (ISC) (Barker, N., van Es, J. H., Jaks, V., Kasper, M., Snippert, H., Toftgard, R., and Clevers, H. (2008). Very long-term self-renewal of small intestine, colon, and hair follicles from cycling Lgr5+ve stem cells. Cold Spring Harb Symp Quant Biol 73, 351-356; Sato, T., Vries, R. G., Snippert, H. J., van de Wetering, M., Barker, N., Stange, D. E., van Es, J. H., Abo, A., Kujala, P., Peters, P. J., et al. (2009). Single Lgr5 stem cells build crypt-villus structures in vitro without a mesenchymal niche. Nature 459, 262-265; Schepers, A. G., Snippert, H. J., Stange, D. E., van den Born, M., van Es, J. H., van de Wetering, M., and Clevers, H. (2012). Lineage tracing reveals Lgr5+ stem cell activity in mouse intestinal adenomas. Science 337, 730-735) (FIG. 31A-FIG. 31B). Compared to controls, adenomas from Lgr5-Apc^{f/f} mice displayed elevated mRNA expression of canonical WNT downstream targets (Axin2, Myc) and stem cell genes (Ascl2, Lgr5, Prom1, Lrig1), including Sox9 (FIG. 25B), consistent with the known biology (Powell, A. E., Wang, Y., Li, Y., Poulin, E. J., Means, A. L., Washington, M. K., Higginbotham, J. N., Juchheim, A., Prasad, N., Levy, S. E., et al. (2012). The pan-ErbB negative regulator Lrig1 is an intestinal stem cell marker that functions as a tumor suppressor. Cell 149, 146-158; Sansom, O. J., Reed, K. R., Hayes, A. J., Ireland, H., Brinkmann, H., Newton, I. P., Batlle, E., Simon-Assmann, P., Clevers, H., Nathke, I. S., et al. (2004). Loss of Apc in vivo immediately perturbs Wnt signaling, differentiation, and migration. Genes Dev 18, 1385-1390). In contrast, markers of differentiated intestinal cells (e.g. Cdx2) of both absorptive (e.g. Krt20) and secretory lineages (e.g. Muc2) (Batlle, E., Henderson, J. T., Beghtel, H., van den Born, M. M., Sancho, E., Huls, G., Meeldijk, J., Robertson, J., van de Wetering, M., Pawson, T., et al. (2002). Beta-catenin and TCF mediate cell positioning in the intestinal epithelium by controlling the expression of EphB/ephrinB. Cell 111, 251-263; Sancho, E., Batlle, E., and Clevers, H. (2003). Live and let die in the intestinal epithelium. Curr Opin Cell Biol 15, 763-770; van Es, J. H., van Gijn, M. E., Riccio, O., van den Born, M., Vooijs, M., Begthel, H., Cozijnsen, M., Robine, S., Winton, D. J., Radtke, F., et al. (2005). Notch/gamma-secretase inhibition turns proliferative cells in intestinal crypts and adenomas into goblet cells. Nature 435, 959-963) were downregulated in Lgr5-Apc^{f/f} intestinal adenomas (FIG. 25B). These findings were validated by examining other genetically engineered mouse models in which inducible Apc knockdown (KD) with or without mutant K-ras^{G12d} activation in Lgr5+ cells displayed robust formation of colonic adenomas (Dow, L.E., O'Rourke, K. P., Simon, J., Tschaharganeh, D. F., van Es, J. H., Clevers, H., and Lowe, S. W. (2015). Apc Restoration Promotes Cellular Differentiation and Reestablishes Crypt Homeostasis in Colorectal Cancer. Cell 161, 1539-1552). Genes associated with stem cell activity were upregulated whereas intestinal differentiation genes were suppressed in Apc KD colonic adenomas by bulk RNA-seq (FIG. 31C).

Protein expression of Sox9 was examined in early intestinal adenomas from Lgr5-Apc^{f/f} mice that were crossed with R26LSL-tdTomato (tdT) mice to label ApcKO cells. Compared to adjacent normal intestinal villi in which Sox9 expression is restricted to the crypt base, tdT+ ApcKO lesions demonstrated ectopic and elevated expression of Sox9 (FIG. 25C). Increased Sox9 expression in Lgr5-Apc^{f/f} adenomas coincided with absence of differentiation markers Krt20 and Muc2 (FIG. 25C). These observations suggest that Apc inactivation is sufficient to prevent intestinal differentiation and leads to inappropriate Sox9 expression in adenomas.

Two mouse models of carcinogen-induced small intestinal and colonic neoplastic lesions were studied to ask whether Sox9 activation and impaired differentiation are universal events in cancer initiation that can be achieved by random mutagenesis in vivo. The first, well-established model consists of exposing mice to the procarcinogen azoxymethane (AOM) and irritant dextran sodium sulfate (DSS) (please see methods in Example 14), which leads to colonic lesions ranging from low-grade dysplasia to intramucosal adenocarcinoma after several months. Consistent with the observations in the Lgr5-Apc^{f/f} model, high-grade dysplastic lesions demonstrated robust Sox9 overexpression and loss of Krt20+ enterocytes and Muc2+ goblet cells (FIGS. 25D and 31D). In the second carcinogen model, mice were exposed to drinking water containing N-methyl-N-nitrosourea (MNU), a potent carcinogen with gastrointestinal tropism, and a subset developed poorly differentiated intestinal carcinomas after one year. Lgr5eGFP mice treated with MNU showed marked upregulation of Sox9 in premalignant and adenocarcinoma lesions (FIGS. 25E and 311E), consistent with an early role in cancer development. Differentiation markers Krt20 and Muc2 were absent in premalignant and adenocarcinoma lesions. Notably, robust expression of Lgr5 as indicted by eGFP immunohistochemistry (IHC) was found in intestinal adenocarcinomas but not their associated premalignant lesions, underscoring the central importance of stem cell activation in progression to malignancy.

These striking results implicate impaired differentiation and Sox9 activation as a common pathway to CRC initiation.

### Example 17: scRNA-Seq of Apc^{KO} Adenomas Reveals Upregulation of Selective ISC Activity

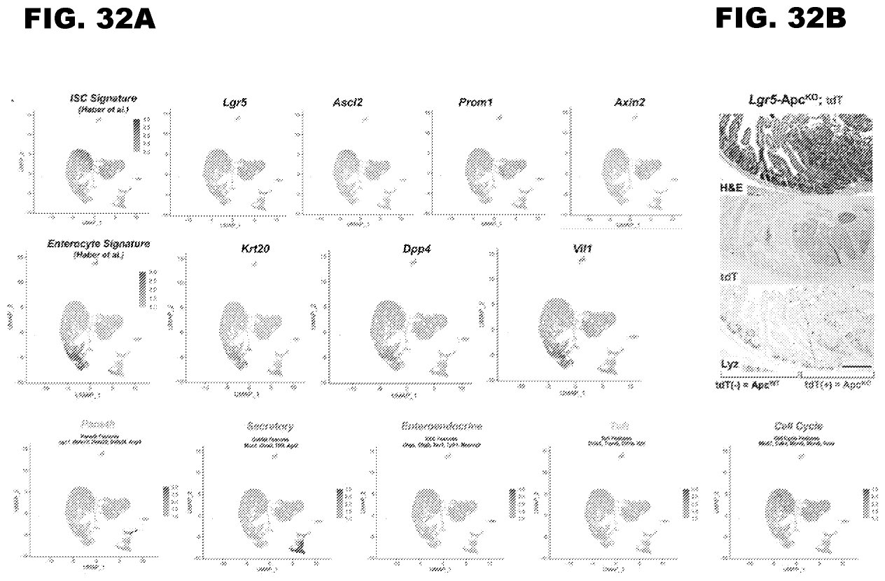

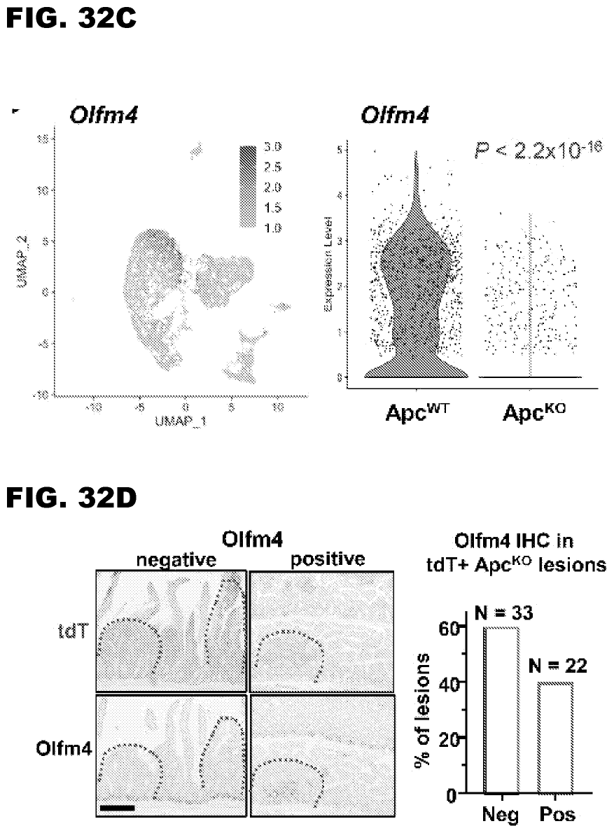

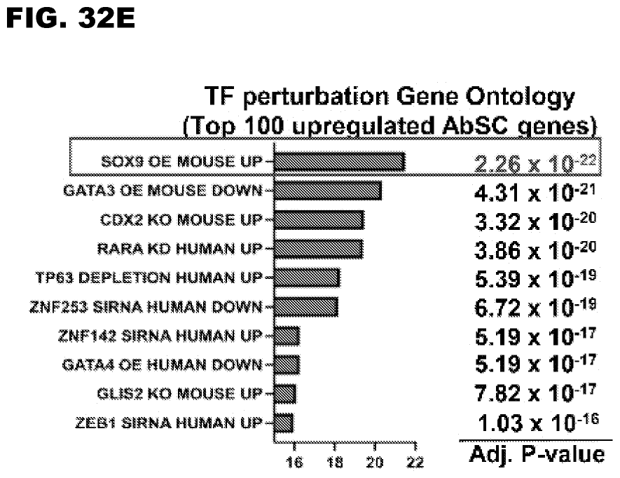

To further characterize cellular and transcriptional alterations during cancer initiation, Lgr5-Apc^{f/f}-tdT+ genetic model was evaluated by performing single cell RNA-sequencing (scRNA-seq). Lgr5-Apcf/+-tdT mice were used as controls as they were phenotypically identical to Lgr5-tdT mice, living greater than 1.5 years without evidence of intestinal neoplasia. tdT+ epithelial cells from Lgr5-Apcf/+-tdT and Lgr5-Apc^{f/f}-tdT mice were isolated by fluorescence-activated cell sorting (FACS) 28 days following tamoxifen induction (FIG. 26A). Unsupervised clustering followed by uniform manifold approximation and projection (UMAP) representation of single-cell gene expression profiles revealed that control ApcWT cells displayed the expected distribution of cell lineages based on intestinal markers (Haber, A. L., Biton, M., Rogel, N., Herbst, R. H., Shekhar, K., Smillie, C., Burgin, G., Delorey, T. M., Howitt, M. R., Katz, Y., et al. (2017). A single-cell survey of the small intestinal epithelium. Nature 551, 333-339) (FIG. 26A). In contrast, Apc inactivation led to a 4-fold enrichment in Paneth cells (5.9% vs 1.5%) and the emergence of cells that exhibited a new transcriptional state (63.1% vs 0%) at the expense of normal ISCs and differentiated cell types, including absorptive progenitors, mature enterocytes, goblet cells, and, to a lesser extent, enteroendocrine cells (FIG. 26B). These findings are consistent with impaired differentiation observed in histopathological analyses of intestinal precancerous and malignant lesions in the genetic and carcinogen mouse models (FIG. 25C-FIG. 25E). To confirm that scRNA-seq faithfully represents in vivo cellular compositions, it was asked whether a greater number of Paneth (Lyz+) cells are observed in ApcKO lesions. Indeed, all tdT+ ApcKO lesions demonstrated increased ectopic Paneth cells by Lyz IHC (FIG. 32B), which can also be found at earlier timepoints of Apc inactivation (Sansom, O. J., Reed, K. R., Hayes, A. J., Ireland, H., Brinkmann, H., Newton, I. P., Batlle, E., Simon-Assmann, P., Clevers, H., Nathke, I. S., et al. (2004). Loss of Apc in vivo immediately perturbs Wntsignaling, differentiation, and migration. Genes Dev 18, 1385-1390). These results provided confidence to pursue the evaluation of the ApcKO transcriptional cluster.

Given the importance of WNT signaling in stem cell biology (Clevers, H. (2006). Wnt/beta-catenin signaling in development and disease. Cell 127, 469-480; van Amerongen, R., and Nusse, R. (2009). Towards an integrated view of Wnt signaling in development. Development 136, 3205-3214), the impact of Apc inactivation was next examined on the ISC transcriptional program. Apc deletion led to a greater percentage of Lgr5+ cells as indicated by eGFP FACS (49.8% versus 8.1% of cells; FIG. 26C), suggesting that ISC activity is upregulated in ApcKO cells. Consistent with this, expression of two previously published ISC signatures (Haber, A. L., Biton, M., Rogel, N., Herbst, R. H., Shekhar, K., Smillie, C., Burgin, G., Delorey, T. M., Howitt, M. R., Katz, Y., et al. (2017). A single-cell survey of the small intestinal epithelium. Nature 551, 333-339; Munoz, J., Stange, D. E., Schepers, A. G., van de Wetering, M., Koo, B. K., Itzkovitz, S., Volckmann, R., Kung, K. S., Koster, J., Radulescu, S., et al. (2012). The Lgr5 intestinal stem cell signature: robust expression of proposed quiescent ‘+4’ cell markers. EMBO J 31, 3079-3091) (Table 6) was found in the ApcKO transcriptional cluster in addition to normal ISCs (FIGS. 26D and 32A). A deeper evaluation revealed that select canonical ISC markers were excluded from the ApcKO transcriptional cluster. Among these markers, Olfm4 expression was unexpectedly confined to normal ISC and not seen within the emergent ApcKO cluster (FIGS. 26E and 32C). Evaluation of tdT+ ApcKO lesions demonstrated heterogeneity with 40% of lesions expressing Olfm4 while 60% did not by IHC (FIG. 32D). By contrast, the ApcKO transcriptional cluster expressed higher levels of Sox9 compared to normal ISC (FIG. 32D), which was supported by the universal expression of Sox9 in tdT+ ApcKO lesions (FIGS. 25C and 26E). Furthermore, evaluating transcription factor perturbation gene-sets that were enriched among the top 100 upregulated genes in the ApcKO transcriptional cluster (Table 6) revealed a Sox9 overexpression transcriptional program scored the best (FIG. 32E, adjusted p-value=3.26×10-22, Table S2). These observations suggest that Apc deletion leads to the activation of a selective ISC transcriptional program enriched for functional Sox9 activity.

### Example 18: Reactivation of Genes Associated with Intestinal Development Upon Apc Loss

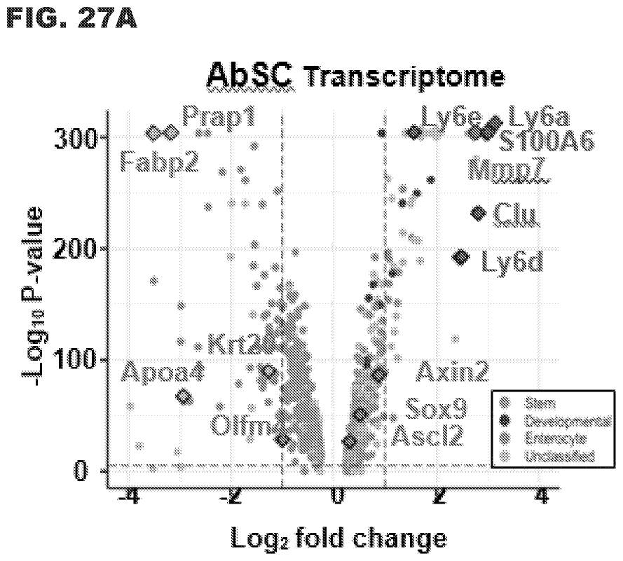

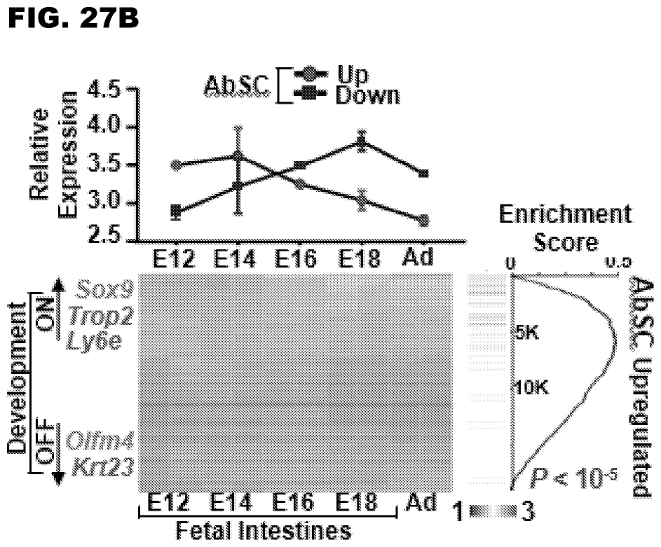

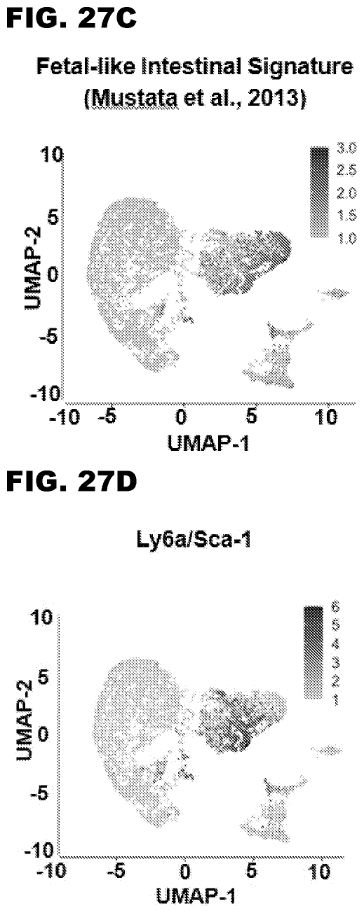

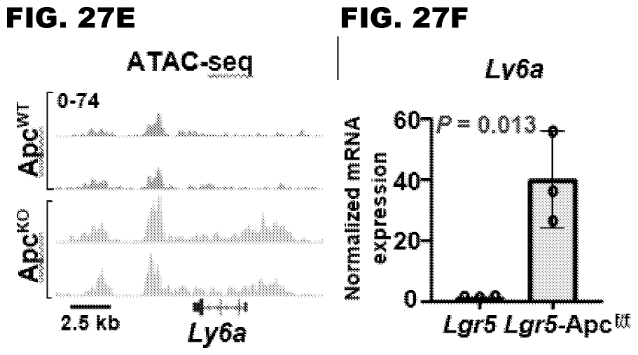

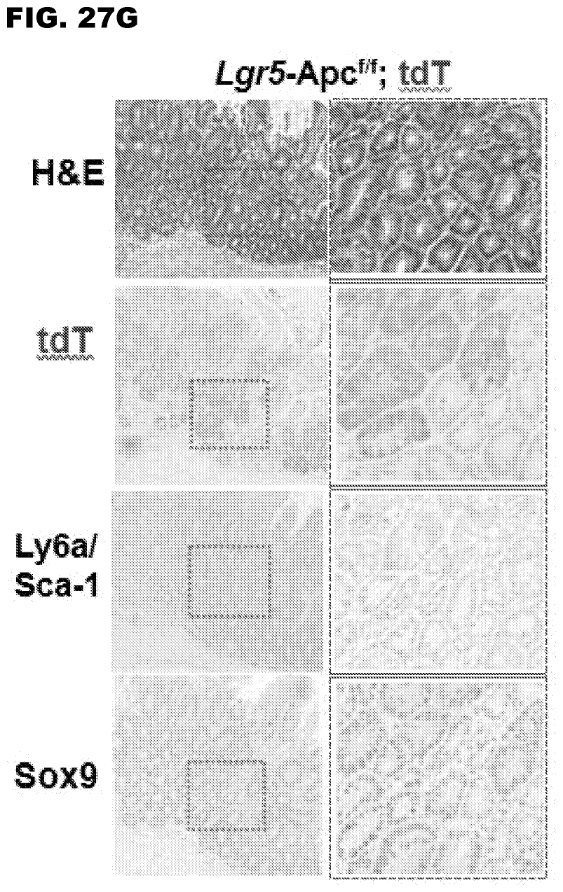

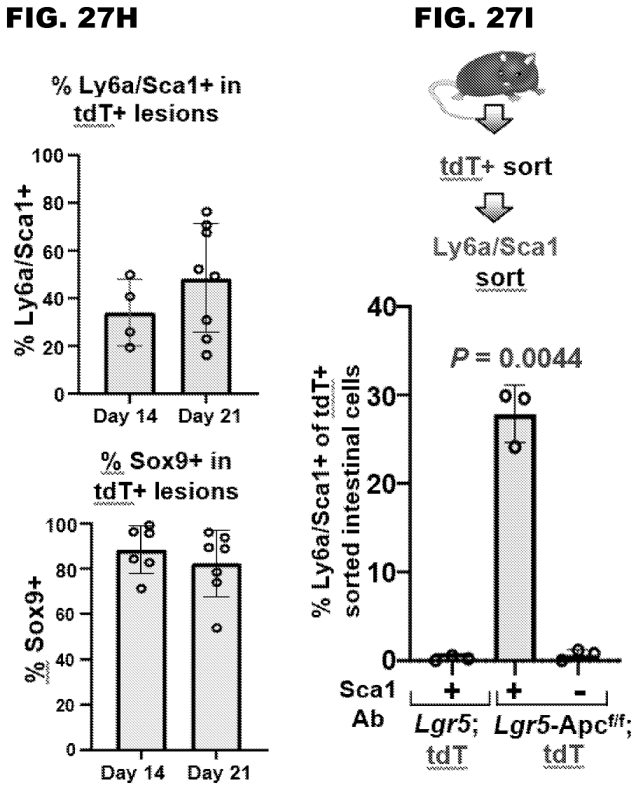

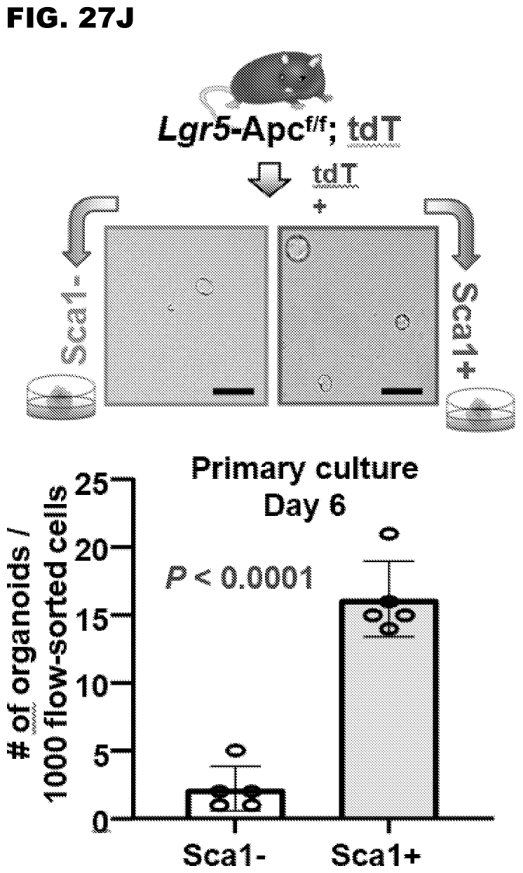

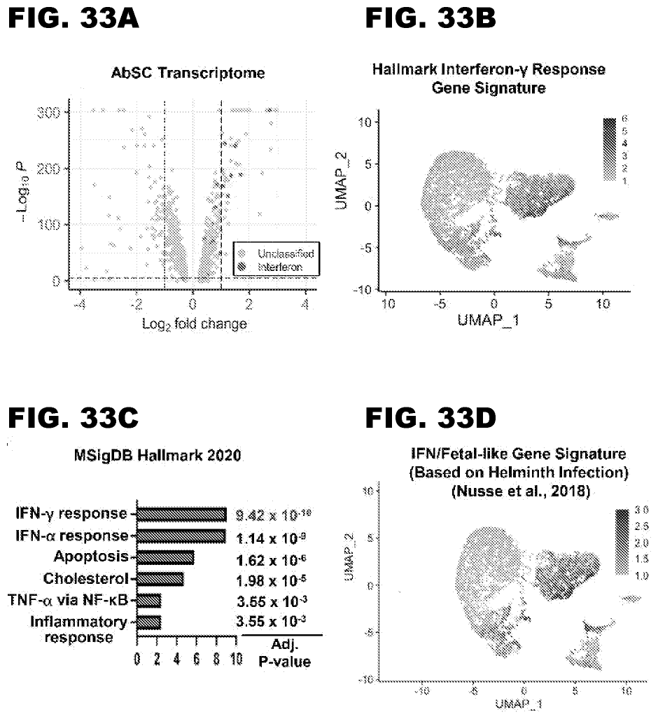

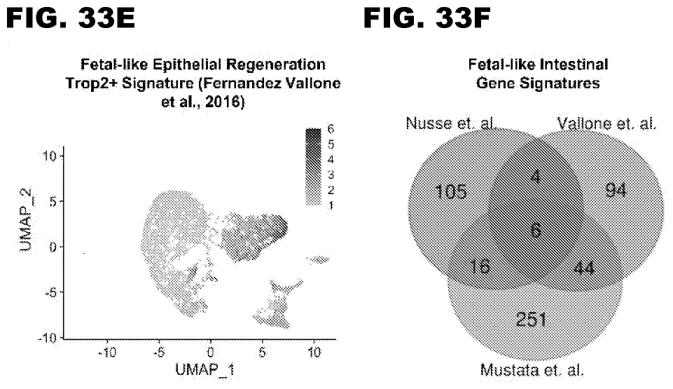

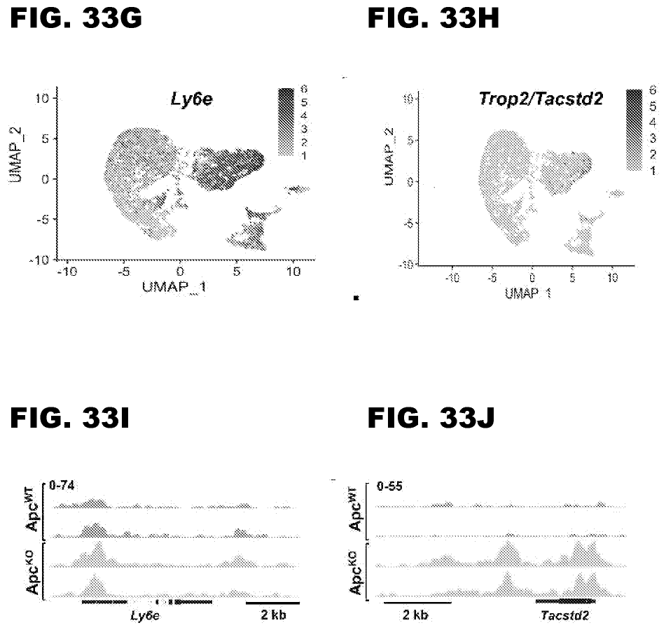

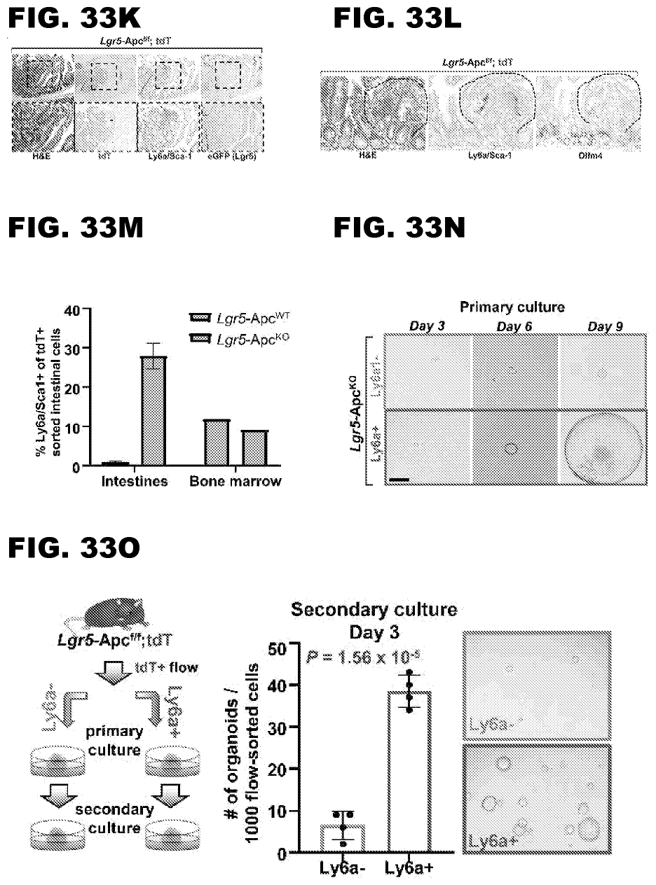

Despite showing an enrichment of most canonical ISC genes, the bulk of ApcKO cells are found in one distinct cluster (FIG. 26B), expressing genes not otherwise found in normal intestinal cells; these cells are referred to as aberrant stem cell-like (AbSC). It was hypothesized that the aberrant nature of ApcKO cells is not entirely explained by selective ISC activity. It was therefore decided to characterize the AbSC transcriptional program in greater depth, searching for features that explain its distinguished gene expression profile (Table 6). The differentially expressed genes in the AbSC cluster were examined relative to all other intestinal clusters. Genes associated with differentiated enterocytes (blue) were downregulated whereas ISC markers (excluding Olfm4) and WNT pathway targets (green) were upregulated in the AbSC cluster (FIG. 27A). Genes associated with interferon (IFN) signaling (FIG. 33A-FIG. 33B) and fetal intestinal programs (FIG. 27A, purple) were also uniquely and significantly upregulated in the AbSC cluster. Gene ontology analyses confirmed an enrichment for pathways and transcription factors (e.g. RELA, NF-κB) linked with IFN signaling (FIG. 33C, Table S2). Interestingly, enrichment of a gene expression signature derived from an experimental intestinal Helminth infection model (Table 6) demonstrated an IFN-7 transcriptional program (Nusse, Y. M., Savage, A. K., Marangoni, P., Rosendahl-Huber, A. K. M., Landman, T. A., de Sauvage, F. J., Locksley, R. M., and Klein, O.D. (2018). Parasitic helminths induce fetal-like reversion in the intestinal stem cell niche. Nature 559, 109-113) that was specifically upregulated in the AbSC cluster (FIG. 33D). Notably, this gene expression signature was also shown to be associated with fetal intestinal programs, indicating a potential link between IFN-7 signaling and reactivation of developmental intestinal genes (Nusse, Y. M., Savage, A. K., Marangoni, P., Rosendahl-Huber, A. K. M., Landman, T. A., de Sauvage, F. J., Locksley, R. M., and Klein, O.D. (2018). Parasitic helminths induce fetal-like reversion in the intestinal stem cell niche. Nature 559, 109-113).

To determine whether fetal intestinal programs are reactivated upon Apc inactivation, gene expression data was directly extracted from intestines at different stages of embryonic development (Banerjee, K. K., Saxena, M., Kumar, N., Chen, L., Cavazza, A., Toke, N. H., O'Neill, N. K., Madha, S., Jadhav, U., Verzi, M. P., et al. (2018). Enhancer, transcriptional, and cell fate plasticity precedes intestinal determination during endoderm development. Genes Dev 32, 1430-1442). AbSC downregulated genes had stronger expression in late development (E18) and adult intestines compared to earlier stages of intestinal development (FIG. 27B, E12-E16). Olfm4 is among the genes expressed at significantly higher levels in the adult relative to embryonic intestines. By contrast, AbSC upregulated genes are significantly enriched among genes preferentially expressed in embryonic relative to adult intestines (FIG. 27B), which include Sox9, Trop2 Tacstd2, and Ly6e Sca-2. Indeed, two additional, and relatively nonoverlapping, fetal-like intestinal gene signatures (Fernandez Vallone, V., Leprovots, M., Strollo, S., Vasile, G., Lefort, A., Libert, F., Vassart, G., and Garcia, M. I. (2016). Trop2 marks transient gastric fetal epithelium and adult regenerating cells after epithelial damage. Development 143, 1452-1463; Mustata, R. C., Vasile, G., Fernandez-Vallone, V., Strollo, S., Lefort, A., Libert, F., Monteyne, D., Perez-Morga, D., Vassart, G., and Garcia, M. I. (2013). Identification of Lgr5-independent spheroid-generating progenitors of the mouse fetal intestinal epithelium. Cell reports 5, 421-432) (Table S2) are selectively expressed in the AbSC cluster (FIG. 27C and FIG. 33E-FIG. 33F). These analyses suggest that IFN signaling and reactivation of fetal intestinal genes contributes to AbSC transcriptional activity.

A subset of cells within the AbSC cluster showed elevated and selective expression of genes associated with fetal intestinal programs, including Ly6a Sca-1, Ly6e Sca-2, and Trop2 Tacstd2 (FIG. 27D and FIGS. 33G-H). Furthermore, suggesting that nongenetic factors enable reactivation of developmental genes. Consistent with these implication, Apc inactivation led to increased chromatin accessibility at the genomic loci harboring these genes as measured by ATAC-sequencing (FIG. 27E and FIG. 33I-33J). Ly6a/Sca-1 was examined as a marker for validation studies due to its (1) selective high-level expression in a substantial fraction of AbSC cells (63.1%), (2) absence from normal intestinal clusters, and (3) association with stem cell properties in the hematopoietic system (Fenske, T. S., Pengue, G., Mathews, V., Hanson, P. T., Hamm, S.E., Riaz, N., and Graubert, T. A. (2004). Stem cell expression of the AML1/ETO fusion protein induces a myeloproliferative disorder in mice. Proc Natl Acad Sci USA 101, 15184-15189; Joosten, M., Valk, P. J., Jorda, M. A., Vankan-Berkhoudt, Y., Verbakel, S., van den Broek, M., Beijen, A., Lowenberg, B., and Delwel, R. (2002). Leukemic predisposition of pSca-1/Cb2 transgenic mice. Exp Hematol 30, 142-149; McKinstry, W. J., Li, C. L., Rasko, J. E., Nicola, N. A., Johnson, G. R., and Metcalf, D. (1997). Cytokine receptor expression on hematopoietic stem and progenitor cells. Blood 89, 65-71). In addition to fetal intestinal programs, transient Ly6a expression is also associated with other pathological states within the intestines including helminth infections (Nusse, Y. M., Savage, A. K., Marangoni, P., Rosendahl-Huber, A. K. M., Landman, T. A., de Sauvage, F. J., Locksley, R. M., and Klein, O.D. (2018). Parasitic helminths induce fetal-like reversion in the intestinal stem cell niche. Nature 559, 109-113), epithelial injury and inflammation (e.g. colitis) (Flanagan, K., Modrusan, Z., Cornelius, J., Chavali, A., Kasman, I., Komuves, L., Mo, L., and Diehl, L. (2008). Intestinal epithelial cell up-regulation of LY6 molecules during colitis results in enhanced chemokine secretion. J Immunol 180, 3874-3881), and epithelial regeneration following injury (Yui, S., Azzolin, L., Maimets, M., Pedersen, M. T., Fordham, R. P., Hansen, S. L., Larsen, H. L., Guiu, J., Alves, M. R. P., Rundsten, C. F., et al. (2018). YAP/TAZ-Dependent Reprogramming of Colonic Epithelium Links ECM Remodeling to Tissue Regeneration. Cell Stem Cell 22, 35-49 e37) or stem cell ablation (Murata, K., Jadhav, U., Madha, S., van Es, J., Dean, J., Cavazza, A., Wucherpfennig, K., Michor, F., Clevers, H., and Shivdasani, R. A. (2020). Ascl2-Dependent Cell Dedifferentiation Drives Regeneration of Ablated Intestinal Stem Cells. Cell Stem Cell 26, 377-390 e376). Greater chromatin accessibility at the Ly6a genomic locus (FIG. 27E) upon Apc deletion corresponded to elevated Ly6a mRNA expression by RT-PCR (FIG. 27F). Lgr5-Apc^{f/f}-tdT mice displayed selective Ly6a expression in tdT+ lesions and within these lesions Ly6a was expressed in 30-40% of tdT+ cells. Ly6a+ cells also expressed Sox9, which was found in approximately 85% of tdT+ cells (FIG. 27G), but did not consistently express Lgr5/eGFP (FIG. 33K) or Olfm4 (FIG. 33L). In the AOM/DSS mouse model, Ly6a expression was selectively found in neoplastic lesions and not neighboring epithelium involved in colitis (FIG. 27H). These data indicate that nongenetics factors enable a subset of AbSCs to undergo developmental reprogramming, and that Ly6a/Sca-1 can be a selective murine marker for these cells.

To examine phenotypic properties of Ly6a/Sca-1+ cells within the AbSC cluster, it was decided to isolate them by FACS. Consistent with the in situ findings, ˜28% of tdT+ cells from Lgr5-Apc^{f/f}-tdT mice demonstrated Ly6a protein expression by FACS, whereas tdT+ cells from Lgr5-tdT mice showed no expression (FIG. 3I). As a control, Ly6a+ cells in bone marrow preparations from these mice by FACS were quantified, which showed comparable distributions (FIG. 33M). It was then asked whether Ly6a+ cells carried a distinct ability to form organoids as a measure of self-renewal properties. Per 1000 cells plated, Ly6a+ cells showed a 7.4-fold greater organoid forming ability than Ly6a− cells isolated from Lgr5-Apc^{f/f}-tdT mice (FIGS. 27J and 33 N). Dissociated Ly6a+ organoids passaged into secondary cultures maintained a 5.9-fold greater organoid forming ability than Ly6a− cells (FIG. 33O), suggesting population stability and preserved regenerative properties. These data suggest that Ly6a+ AbSCs harbor a robust self-renewal capacity that may facilitate neoplastic progression.

### Example 19: Human FAP Adenoma and Derivative Organoids Display Impaired Differentiation and AbSC Activity

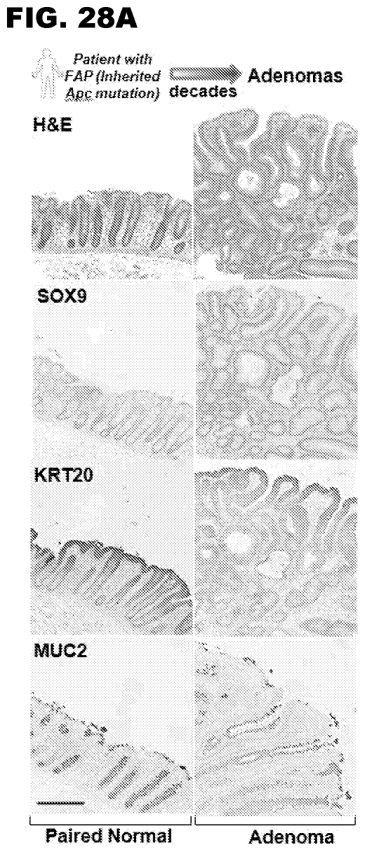

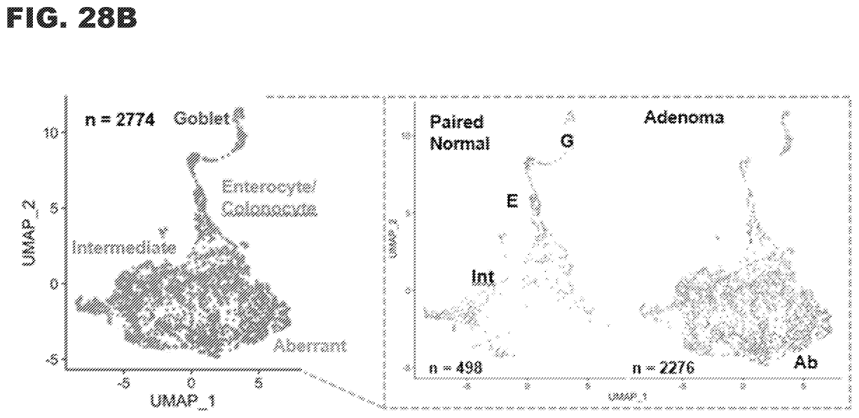

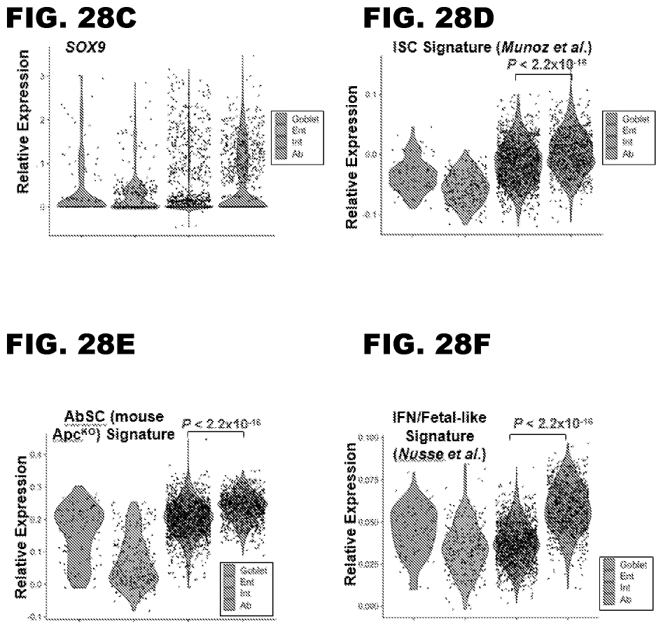

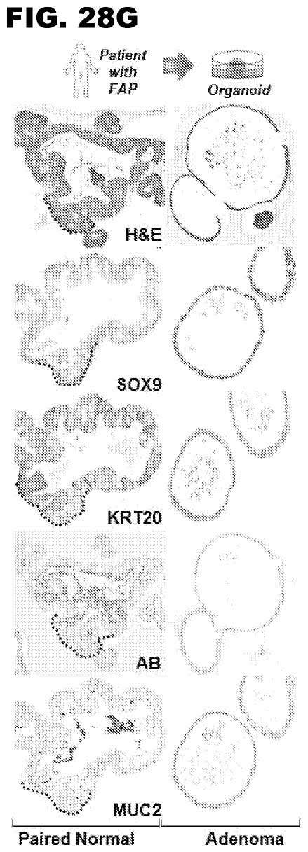

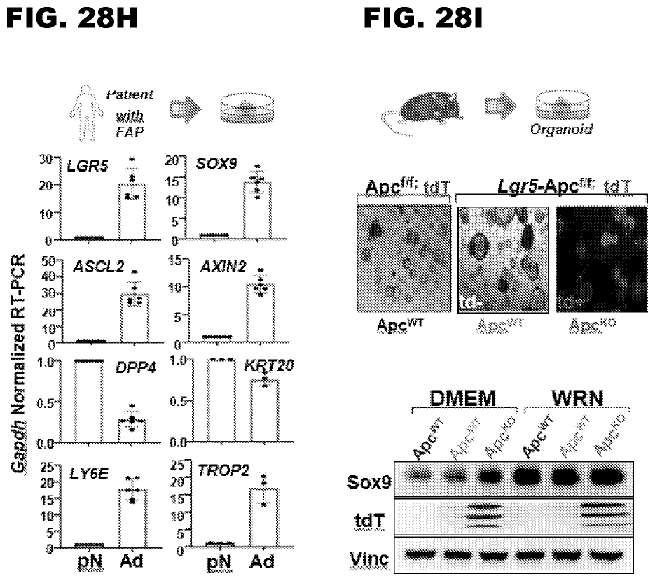

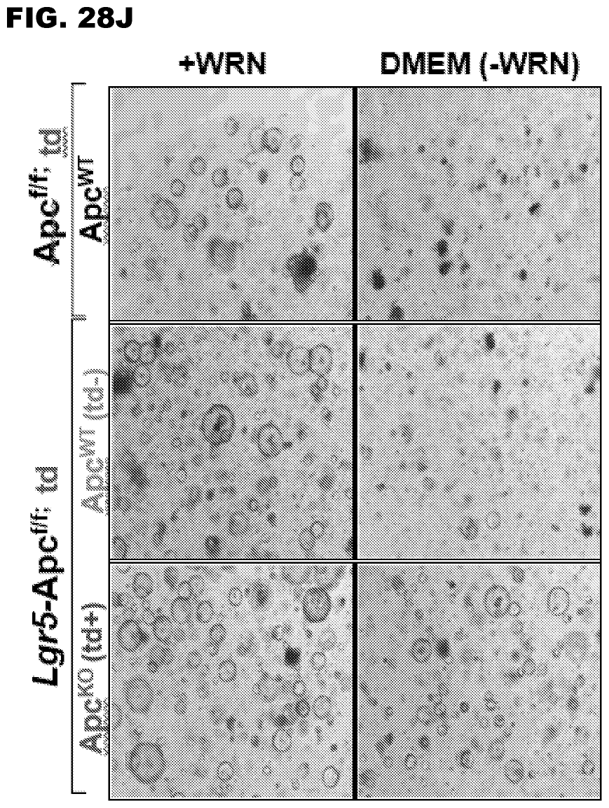

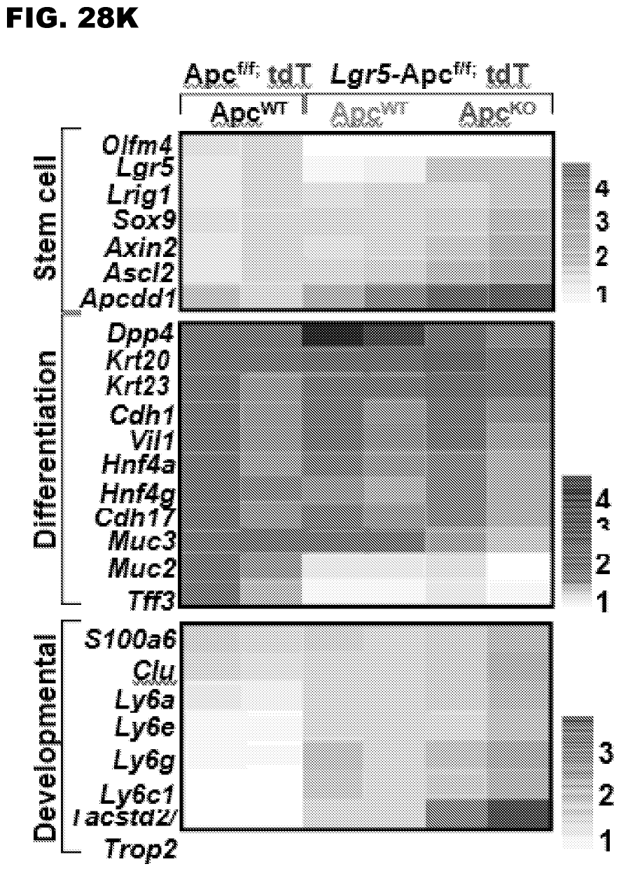

To validate these findings in humans, adenoma and paired normal-appearing tissue was evaluated (herein referred to as normal) from a 39 year-old patient with familial adenomatous polyposis (FAP), a hereditary condition in which a mutant copy of APC is inherited, hundreds to thousands of intestinal adenomas develop, and prophylactic colectomy is required to prevent progression to cancer (Vasen, H. F., Moslein, G., Alonso, A., Aretz, S., Bernstein, I., Bertario, L., Blanco, I., Bulow, S., Burn, J., Capella, G., et al. (2008). Guidelines for the clinical management of familial adenomatous polyposis (FAP). Gut 57, 704-713). In agreement with the mouse models (FIG. 25), colon adenomas displayed elevated expression of Sox9 compared to adjacent normal tissue (FIG. 28A). Markers of differentiated absorptive and secretory colonic cells were partially suppressed as shown by KRT20 and MIUC2 IHC, respectively (FIG. 28A). This observation could reflect limited WNT pathway restriction due to hypomorphic mutant APC activity (Ranes, M., Zaleska, M., Sakalas, S., Knight, R., and Guettler, S. (2021). Reconstitution of the destruction complex defines roles of AXIN polymers and APC in beta-catenin capture, phosphorylation, and ubiquitylation. Mol Cell 81, 3246-3261 e3211) or admixture of normal colonic tissue.

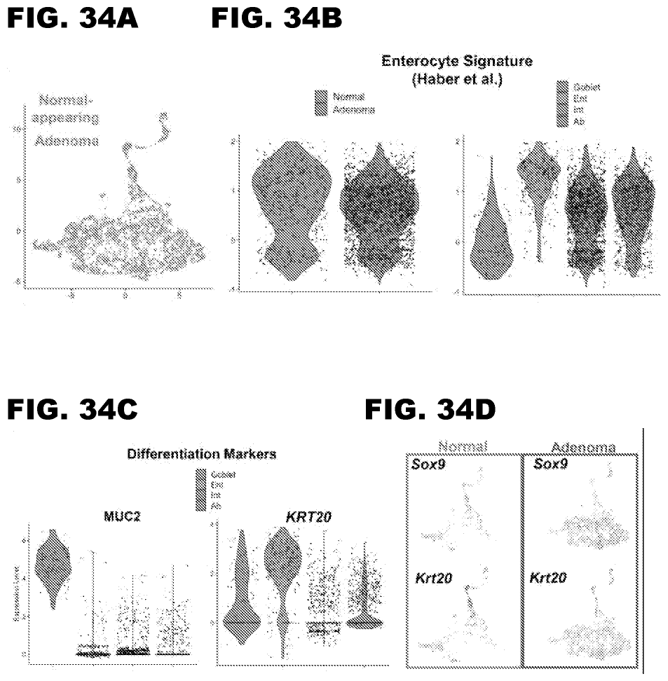

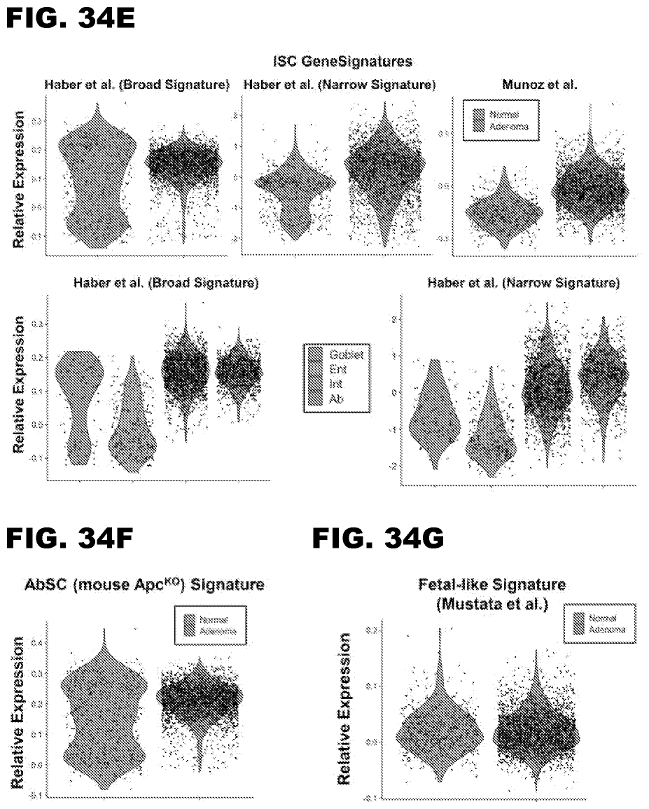

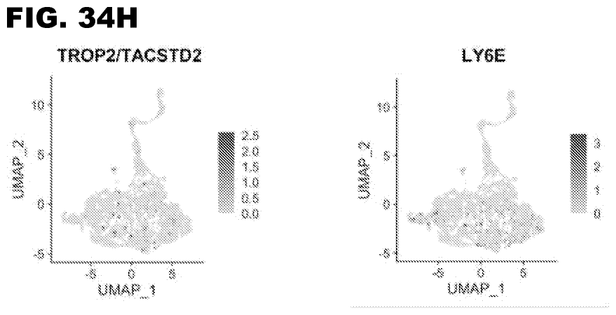

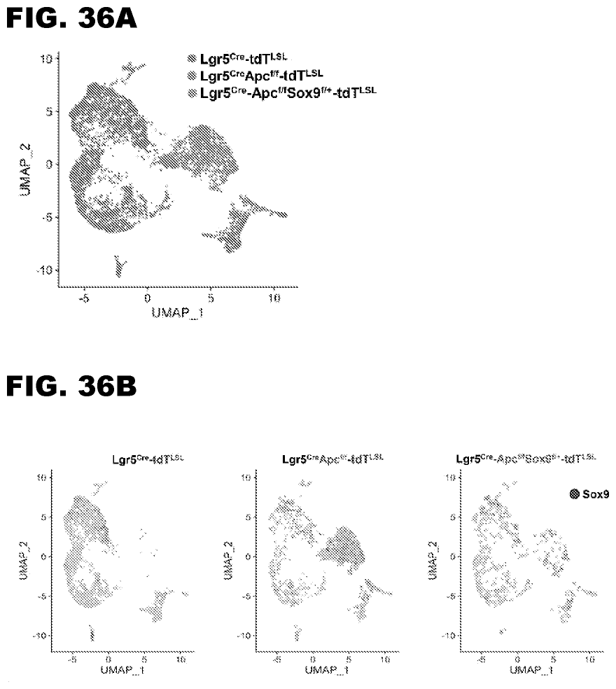

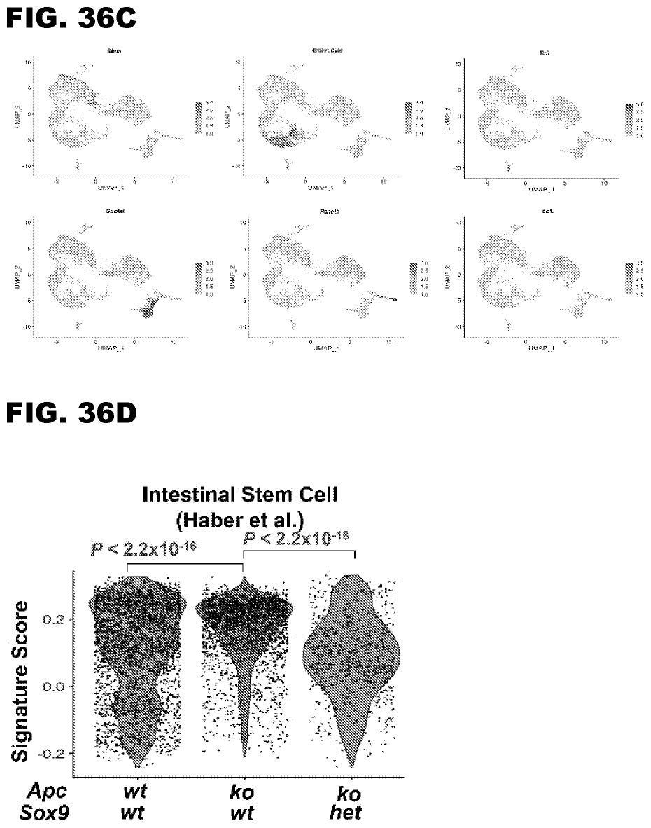

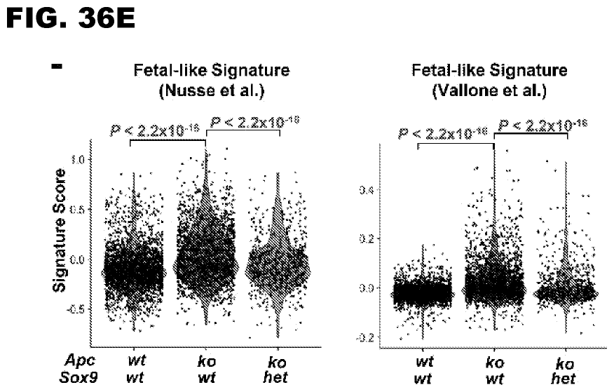

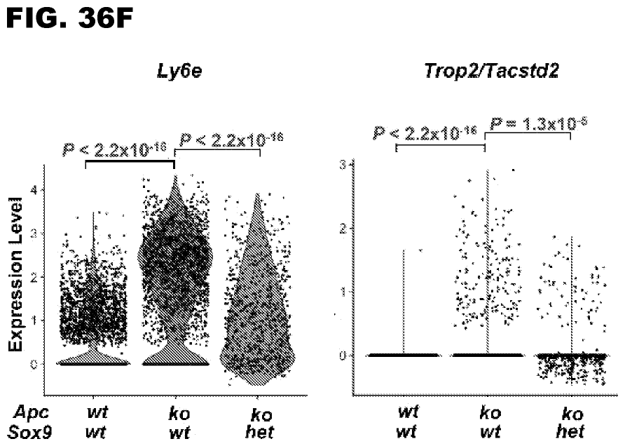

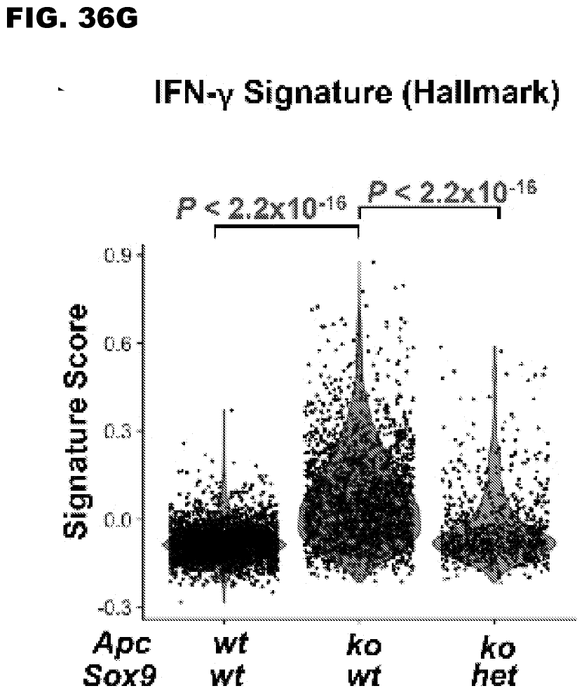

To further characterize human adenomas, scRNA-seq was performed on cryopreserved adenoma and paired normal tissue. UMAP representation of single-cell gene expression profiles revealed 4 epithelial clusters derived from adenoma and normal tissue (FIG. 28B and FIG. 36); however, unlike the mouse models and consistent with human histopathology, the separation between adenomatous and normal tissue was less distinct. To define these clusters, established gene signatures were first utilized to identify normal enterocyte and goblet cell clusters (FIG. 28B-FIG. 28C and FIG. 34B-FIG. 3D). The cluster was found only in the adenoma sample as ‘Aberrant’ (FIG. 28B). The remaining cluster was labelled ‘Intermediate’ since (1) it was found in both normal and adenoma tissue and (2) it did not engender a normal cell type gene expression profile (Haber, A. L., Biton, M., Rogel, N., Herbst, R. H., Shekhar, K., Smillie, C., Burgin, G., Delorey, T. M., Howitt, M. R., Katz, Y., et al. (2017). A single-cell survey of the small intestinal epithelium. Nature 551, 333-339) (FIG. 28B). Reassuringly, SOX9 expression was highest in the Aberrant cluster followed by the Intermediate cluster, whereas KRT20 expression was lowest in these two clusters (FIG. 28C and FIG. 34C). In general, expression of ISC signatures (Table 6) was greatest in the Aberrant cluster relative to Intermediate, enterocyte, and goblet clusters (FIG. 28D and FIG. 34E), further building confidence in the classification. Notably, the genetic mouse model derived AbSC expression signature was greatest in the Aberrant cluster and adenoma tissue (FIG. 28E and FIG. 34F). A subset of fetal-like intestinal signatures and genes were elevated in the Aberrant cluster (FIG. 28F and FIG. 34G-FIG. 34H). These data show that human adenomas demonstrate a partial block in differentiation and activation of an AbSC program involving developmental programs.

Organoids derived from adenoma and normal colonic tissue were evaluated from the same patient with FAP, asking whether features of impaired differentiation and AbSC activity are captured in this three-dimensional culture system that requires supplemented WNT3A, R-spondin, and Noggin (WRN) conditioned media rich in stem cell promoting factors (methods). Normal colonic cultures contained organoids that appeared folded by phase contrast and generated crypts with high SOX9 expression, absent KRT20 expression, and absent MUC2 expression/Alcian blue (AB) staining (FIG. 28G, dotted line). These organoids also displayed differentiated regions with low or no SOX9 expression, high KRT20 expression, intermittent MUC2 expression, and strong AB staining; MUC2 and AB also stained the lumen of these organoids as mucins are secreted (FIG. 28G). In contrast, the majority of organoids in adenomas cultures (greater than 95%) did not display differentiated regions as indicated by unfolded, spheroid morphology by phase contrast and weak KRT20 levels and absent MUC2 expression by IHC. Rather, adenoma organoids expressed uniform high SOX9 levels (FIG. 28G). These findings were confirmed in a broader survey of stem cell/WNT (LGR5, SOX9, ASCL2, AXIN2) and developmental genes (LY6E, TROP2), which demonstrated markedly higher mRNA expression in adenoma compared to normal organoid cultures (FIG. 28H) [Ly6a does not have a direct human homolog]. mRNA expression of colonic differentiation markers DPP4 and KRT20 were correspondingly reduced, albeit modestly due to enhanced stem cell cures conferred by WRN media (FIG. 28H).

To validate these observations, ApcWT and Apc^{KO} organoids were generated from the Lgr5-Apc^{f/f}-tdT mouse model (FIG. 28I). Apc inactivation in tdT+ organoids conferred niche independence: the ability to grow without WRN media (FIG. 28J). Compared to two ApcWT controls, tdT+ Apc^{KO} organoids displayed greater Sox9 expression when grown in media without WRN for 24 hours (FIG. 28I). Upon WRN stimulation, all organoids potently express Sox9 (FIG. 28H). Bulk RNA-sequencing demonstrated activation of stem cell genes and partial reduction in intestinal differentiation in Apc^{KO} organoids (FIG. 28K). Moreover, Apc^{KO} organoids demonstrated robust activation of developmental intestinal genes, especially Tacstd2/Trop2. These data suggest human adenoma and mouse Apc^{KO} organoids display impaired differentiation and strong expression of genes associated with the AbSC transcriptional program.

### Example 20: Sox9 is Essential for Apc^{KO} Adenomas

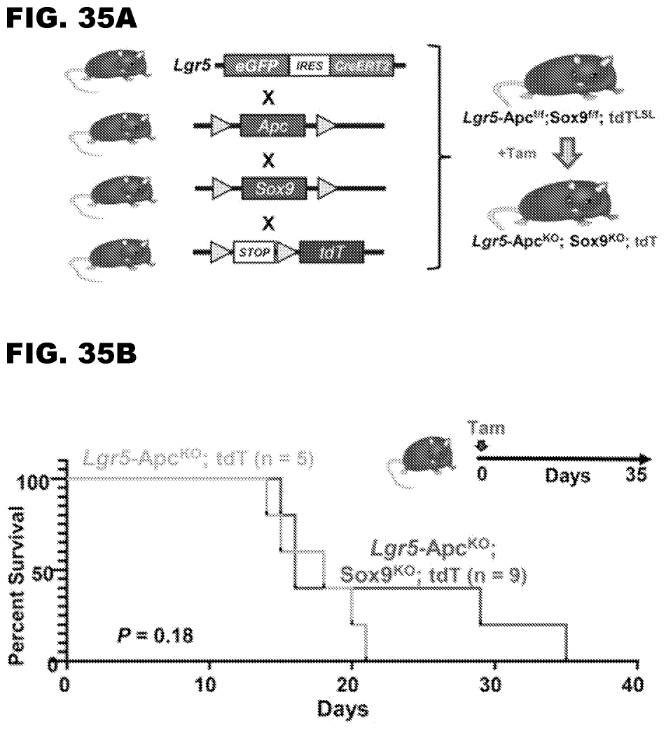

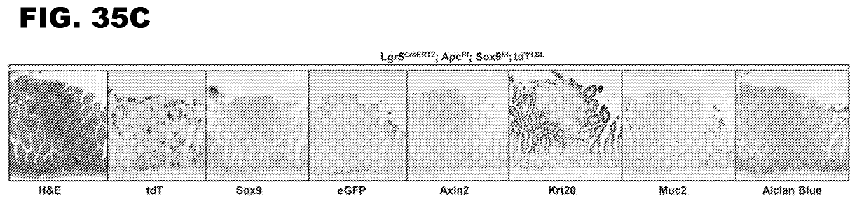

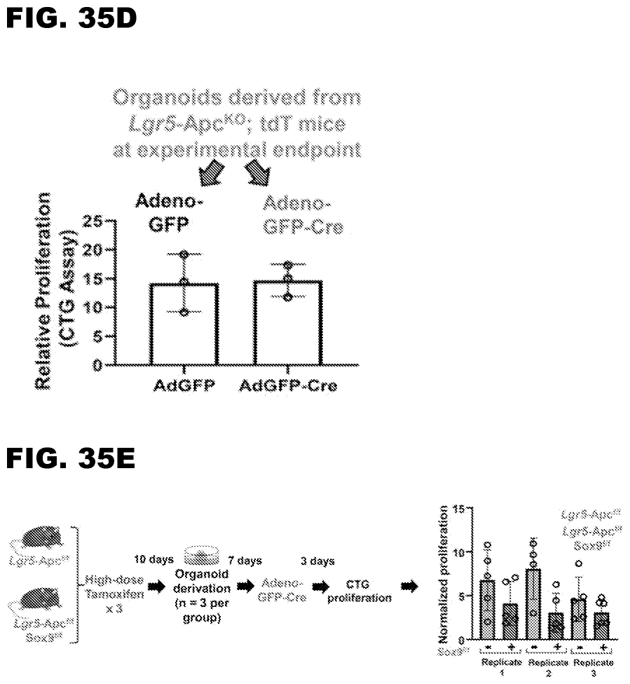

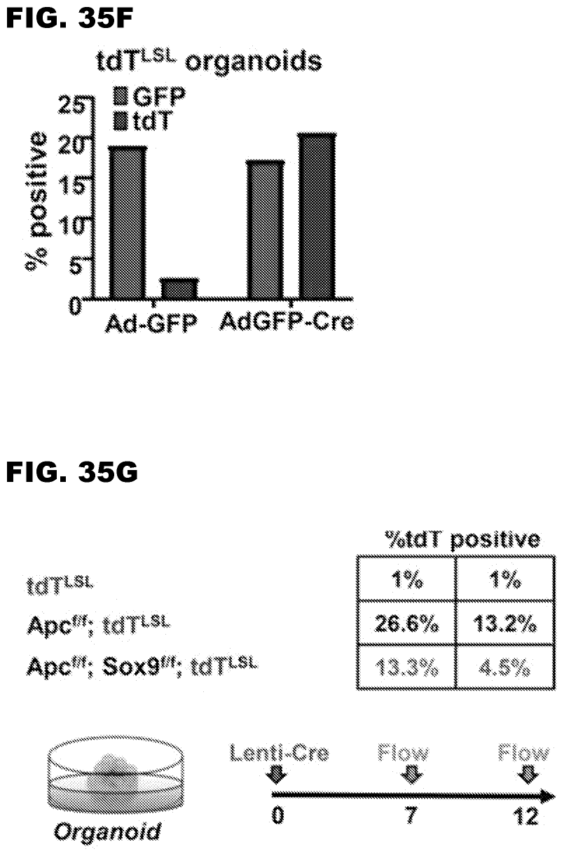

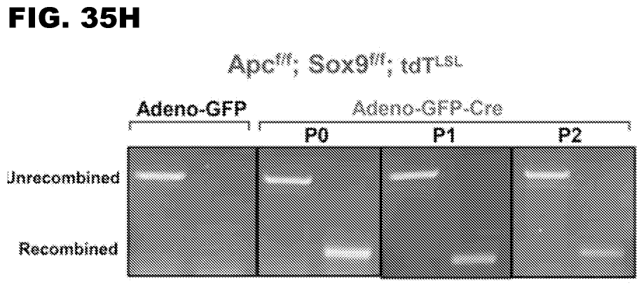

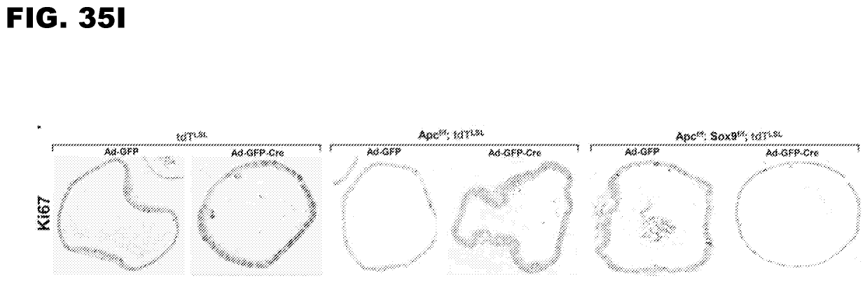

It was hypothesized that Sox9 is required for adenoma formation given its (1) enriched transcriptional activity in the AbSC cluster, (2) ability to functionally block differentiation in models of CRC, and (3) stronger expression in fetal intestines compared to adult intestines. To determine whether Sox9 is necessary for adenoma formation, Lgr5-Apc^{f/f}-tdT mice with Sox9^{f/f} mice were breed (Akiyama, H., Chaboissier, M. C., Martin, J. F., Schedl, A., and de Crombrugghe, B. (2002). The transcription factor Sox9 has essential roles in successive steps of the chondrocyte differentiation pathway and is required for expression of Sox5 and Sox6. Genes Dev 16, 2813-2828) to homozygosity, enabling conditional biallelic deletion of Sox9 in the ApcKO setting upon tamoxifen induction (FIG. 35A). Initial experiments with a single injection of standard dose (10 mg/ml) tamoxifen did not yield a survival benefit for Lgr5-Apc^{f/f}-Sox9^{f/f} mice relative to Lgr5-Apc^{f/f} controls (FIG. 35B).

Histopathological analysis demonstrated discordance of allele recombination with evidence of Apc deletion without Sox9 inactivation and/or tdT activation (FIG. 35C). To address this technical hurdle, experiments were initiated with three daily high-dose (40 mg/ml) tamoxifen injections followed by weekly single injection maintenance treatments, attempting to force robust genetic recombination.

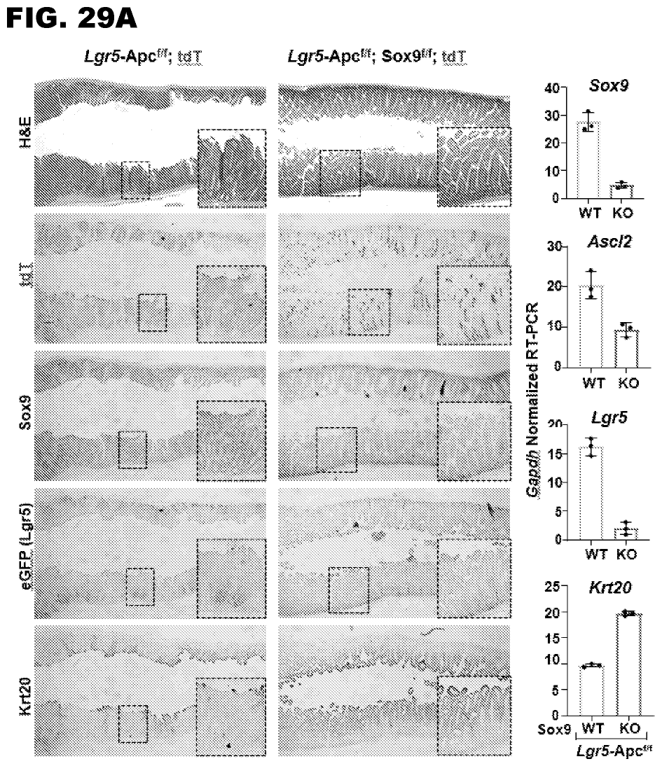

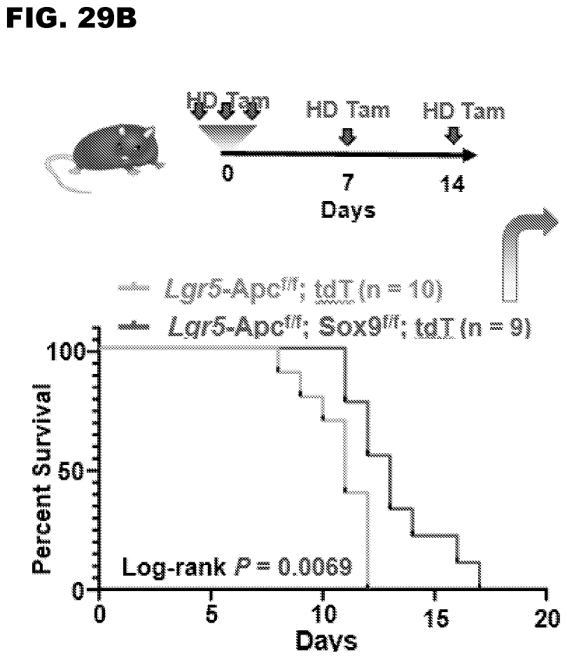

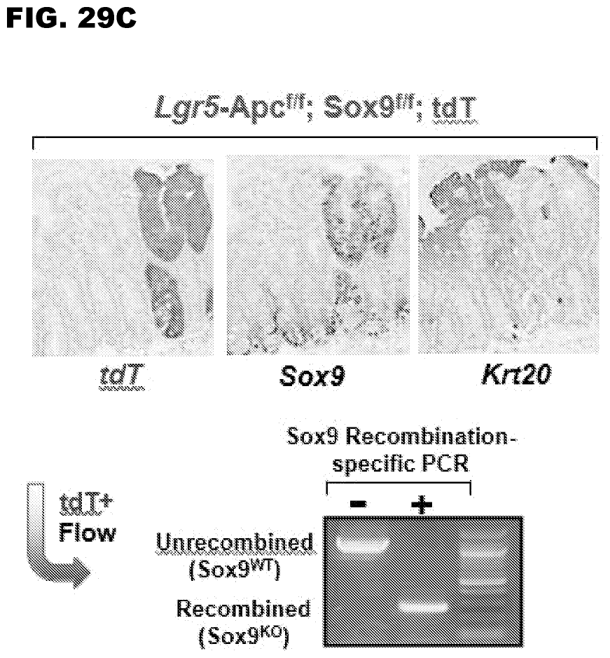

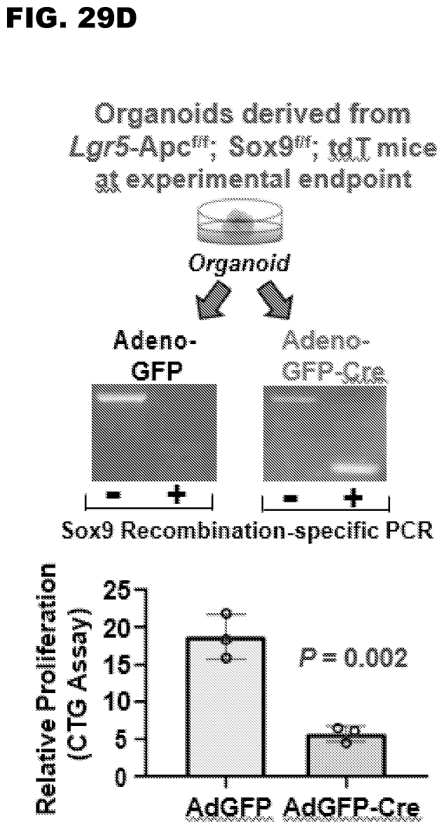

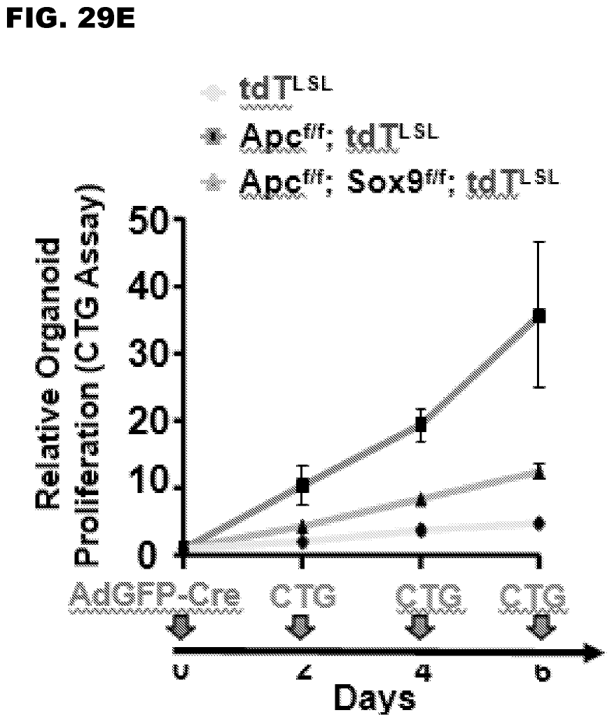

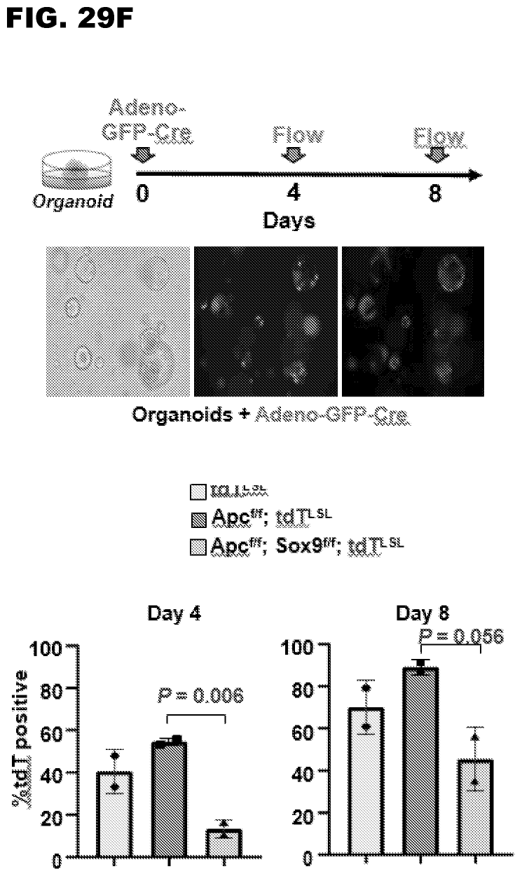

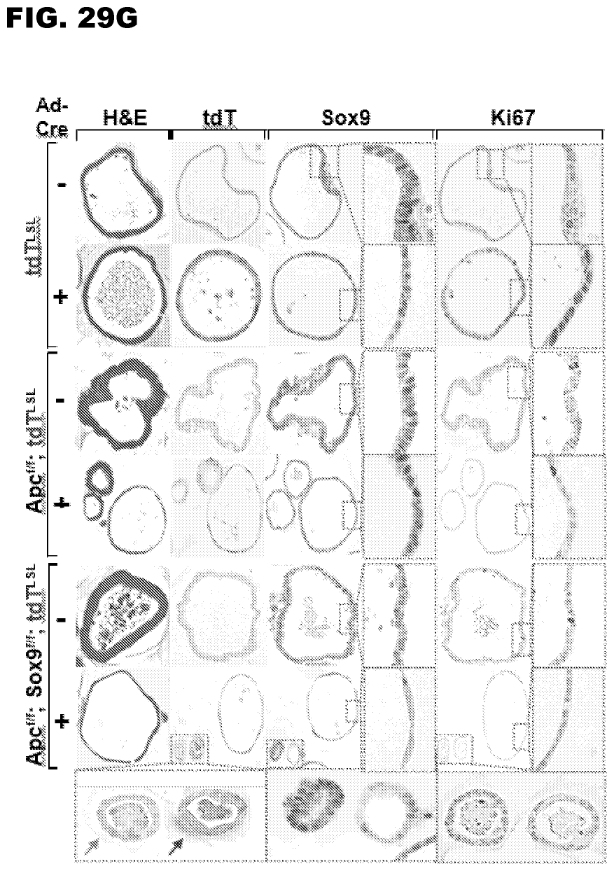

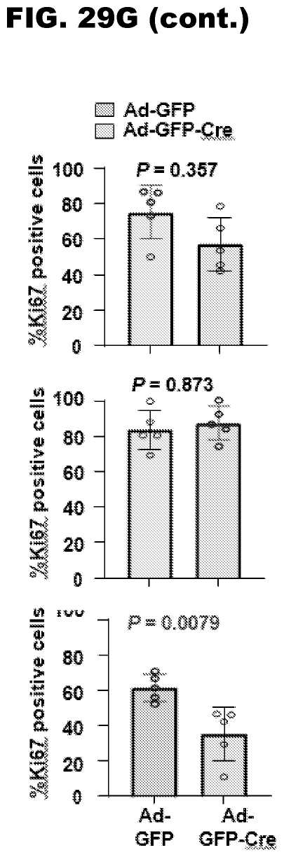

Successful Sox9 inactivation prevented tdT+ adenoma formation, reduced mRNA levels of stem cell genes (Ascl2, Lgr5) and maintained intestinal homeostasis with crypt restricted Lgr5eGFP+ stem cells and Krt20+ differentiated villi (FIG. 29A). While Lgr5-Apc^{f/f}-Sox9^{f/f} mice survived significantly longer than Lgr5-Apc^{f} f controls using the high-dose tamoxifen regimen (FIG. 29B), they eventually developed intestinal obstruction due to adenomas. These adenomas invariably expressed Sox9 despite tdT+ activation and Apc deletion (FIG. 29C) indicating escape of Sox9 deletion. Indeed, tdT+ cells isolated by FACS demonstrated a significant fraction of unrecombined Sox9 by semi-quantitative PCR (FIG. 29C). To show that tdT+ adenomas from Lgr5-Apc^{f/f}-Sox9^{f/f} mice were still dependent on Sox9, organoids were generated at the experimental endpoint and subjected them to ex vivo recombination using specific adenovirus (Ad) infections. Compared to organoids treated with mock Ad^{GFP} controls, Ad^{GFP-Cre} treated organoids displayed impaired proliferation by CellTiterGlo (CTG), which corresponded to robust Sox9 inactivation by recombination (FIG. 29D). Organoids derived from Lgr5-Apc^{f/f} control mice at experimental endpoint did not show different growth properties (FIG. 35D). These results were confirmed by performing a 10-day short-term experiment, isolating organoids from three mice from each genotype, and subjecting them to Ad^{GFP-Cre} infections followed by CTG (FIG. 35E). Collectively, these experiments indicate that successful Sox9 inactivation prevents intestinal ApcKO adenomas using genetic engineered mice and derivative organoids.

Colon organoids were next established without endogenous Cre recombinase for controlled ex vivo Ad^{GFP-Cre} experiments, seeking further validation of Sox9 dependency in colonic adenomas. Colon organoids from tdT, Apc^{f/f}-tdT, and Apc^{f/f}-Sox9^{f/f}-tdT mice were infected with Ad^{GFP-Cre} and subjected to proliferation assays by CTG. While Apc inactivation led to a 7.5-fold increase in organoid proliferation, concomitant Sox9 deletion reduced this proliferative advantage by 3-fold (FIG. 29E). To support these findings, tdT+ quantification was performed by FACS as a surrogate for cell viability following Ad^{GFP-Cre} treatment (FIG. 35F). tdT was activated in 54% of Apc^{f/f}-tdT organoids 4 days after Ad^{GFP-Cre} infection, which was reduced by 4-fold to 13% in Apc^{f/f}-Sox9^{f/f}-tdT organoids (FIG. 29F); similar results were achieved with lentiviral Cre activation (FIG. 35G. The growth advantage of Apc^{f/f}-tdT organoids was reduced at 8 days following Ad^{GFP-Cre} infection, largely attributable to an increase in percent tdT+ in Apc^{f/f}-Sox9^{f/f}-tdT organoids (13% at day 4 versus 45.3% at day 8), which may be due to the outgrowth of tdT+ organoids without Sox9 deletion. To evaluate this possibility, serial Sox9 recombination PCR was performed on Apc^{f/f}-Sox9^{f/f}-tdT organoids following Ad^{GFP-Cre} infection. Indeed, it was observed that deletion of Sox9 was selected against with each consecutive organoid passage by semi-quantitative PCR (FIG. 35H), underscoring the necessity of Sox9 for adenoma organoid growth. Finally, compared to AdGFP treated controls, AdGFP-Cre treated Apc^{f/f}-Sox9^{f/f}-tdT organoids demonstrated impaired proliferation by Ki67 IHC, which was not observed in tdT or Apc^{f/f}-tdT organoids (FIG. 29G and FIG. 35I). Apc^{f/f}-Sox9^{f/f}-tdT organoids with more efficient Ad^{GFP-Cre} infection marked by increased tdT activation demonstrated reduced Sox9 expression and lower proportion of Ki67+ cells relative to poorly infected ones (FIG. 29G, red vs. gray arrow). These experiments demonstrate a requirement for Sox9 in the formation of Apc^{KO} adenomas using a genetically engineered mouse model.

### Example 21: scRNA-Seq Reveals Sox9 is Required for AbSC

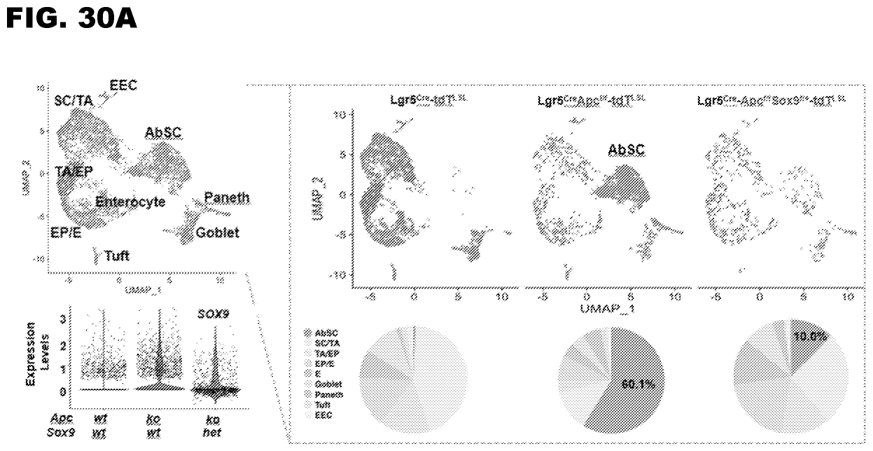

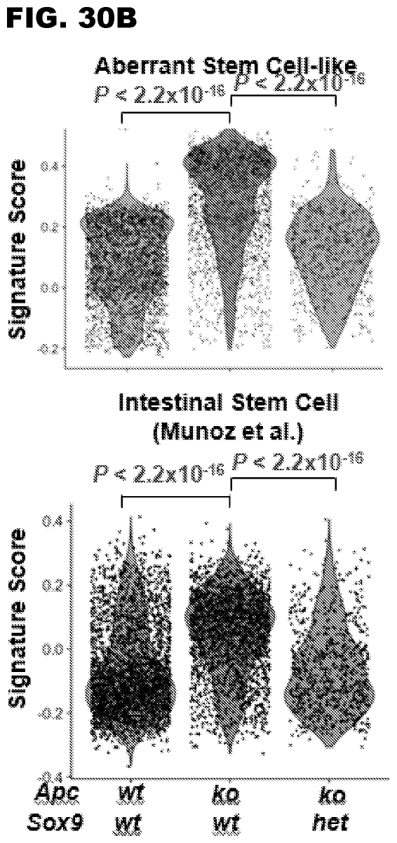

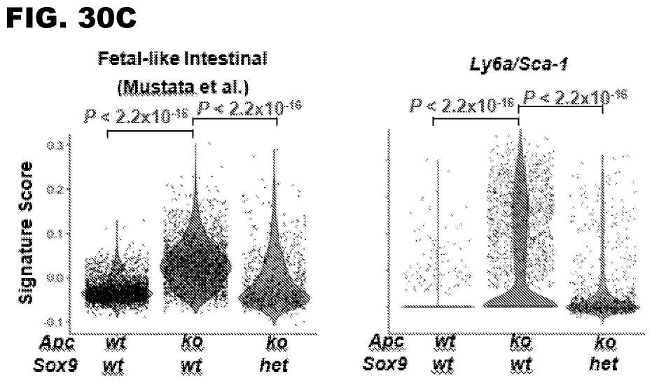

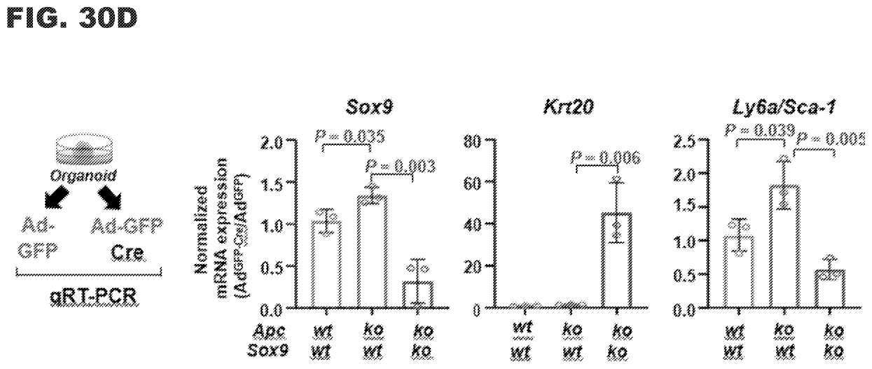

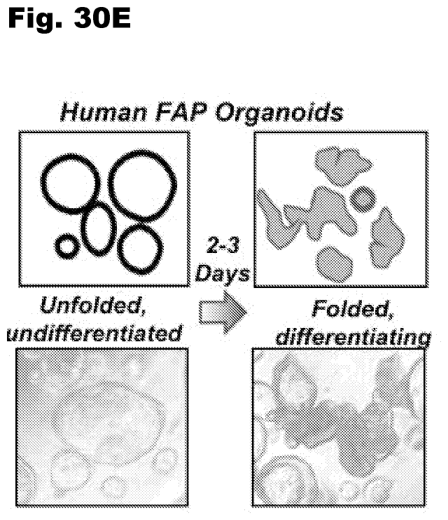

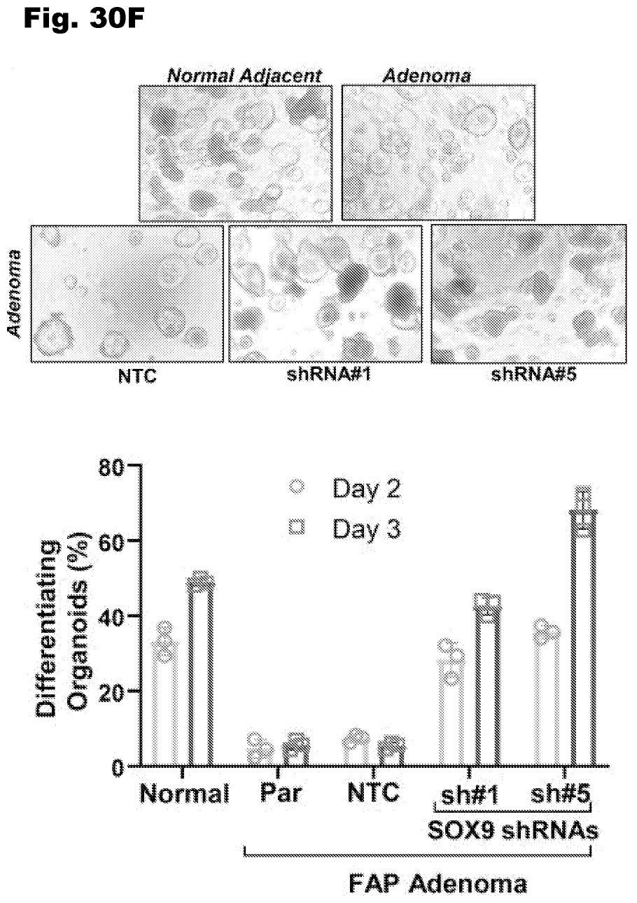

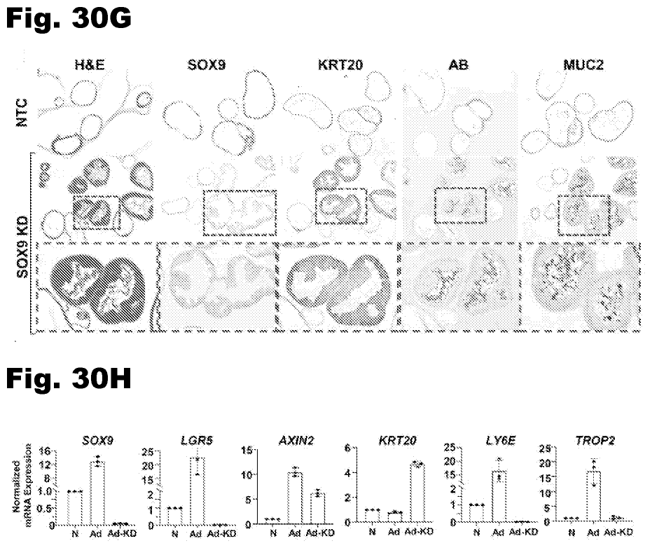

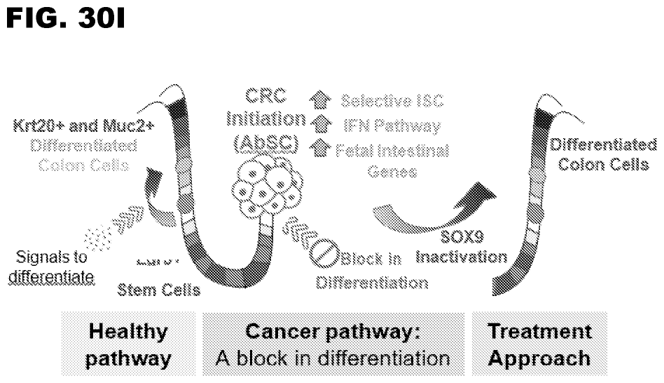

To determine whether Sox9 is required for AbSC transcriptional activity, scRNA-seq on FACS-isolated tdT+ cells from Lgr5-Apc^{f/f}-Sox9f/+-tdT mice one month after tamoxifen induction was performed. Lgr5-Apc^{f/f}-Sox9f/+-tdT mice were analyzed and showed tdT+ cells with consistent and reliable reduction in Sox9 expression given the difficulty to isolate viable tdT+ cells with efficient homozygous deletion of Sox9 (FIG. 29C). Reassuringly, viable tdT+ cells from Lgr5-Apc^{f/f}-Sox9f/+-tdT mice demonstrated a considerable reduction in Sox9 levels compared to Lgr5-Apc^{f/f}-tdT mice, which displayed higher Sox9 expression relative to Lgr5-tdT controls (FIGS. 30A and 36A-B). UMAP representation of single-cell gene expression profiles revealed clusters representing the normal epithelial cell types in addition to the ApcKO AbSC cluster (FIGS. 30A and 36C). Notably, single copy deletion of Sox9 reduced the percentage of AbSC cells from 60% to 10% in tdT+ Lgr5-ApcKO cells (FIG. 30A and Table 7) and significantly reduced AbSC gene signature activity (FIG. 30B), indicating a requirement for Sox9 in maintaining an AbSC transcriptional program. ISC gene signatures were also reduced in tdT+ cells from Lgr5-Apc^{f/f}-Sox9f/+-tdT mice (FIGS. 30B and 36D). The 6-fold reduction in AbSC cells was accompanied by restoration of a normal distribution of ISC and differentiated intestinal cell types (FIG. 30A and Table 7). Reduced expression of IFN-γ and fetal-like intestinal gene signatures was also observed as well as developmental genes, including Ly6a Sca-1, in tdT+ cells from Lgr5-Apc^{f/f}-Sox9f/+-tdT mice (FIGS. 30C and 36E-G). These results were confirmed in tdT, Apc^{f/f}-tdT, and Apc^{f/f}-Sox9^{f/f}-tdT organoids using ex vivo Ad^{GFP-Cre} infections followed by RT-PCR (FIG. 30D). These results demonstrate a requirement of Sox9 in AbSC activity including regulation of fetal intestinal gene reactivation.

### Example 22: SOX9 Knockdown in FAP Adenoma Organoid Induces Differentiation

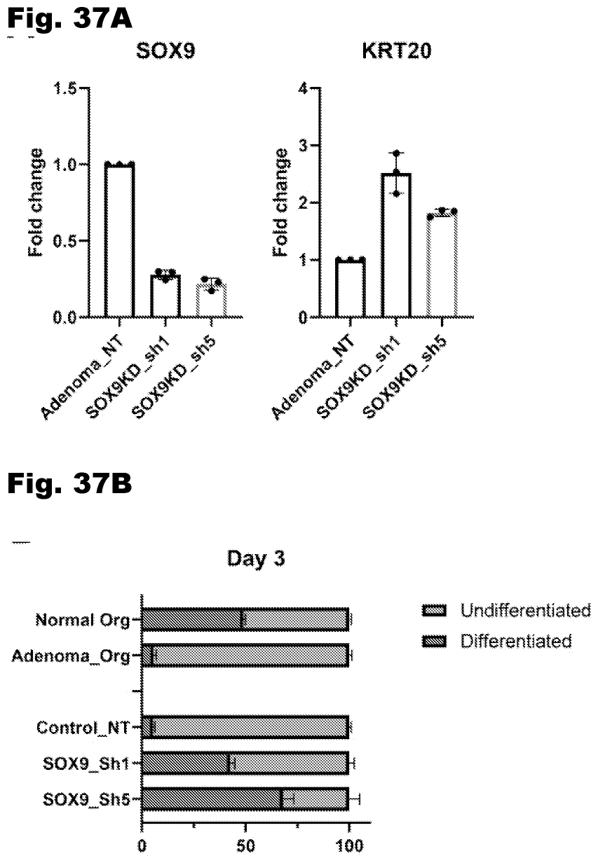

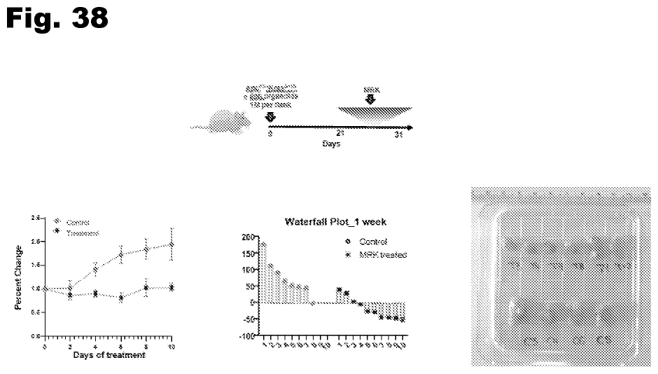

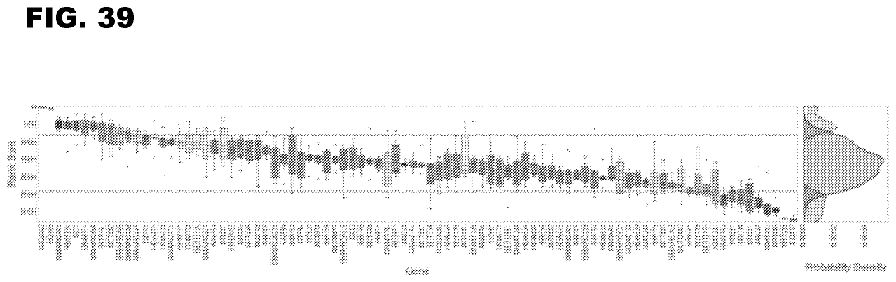

It was next asked whether SOX9 is required for AbSC activity and impaired differentiation in human FAP adenomas using the previously derived organoids (FIG. 28G). Human adenoma organoid cultures showed few differentiating organoids (˜5%), which appear as folded structures by phase contrast, whereas organoid cultures derived from normal adjacent tissue show evidence of a differentiating population that amount to about 50% by day 3 (FIGS. 30E and 30F). Using this system, we examined whether shRNA mediated SOX9 knockdown (KD) can force differentiation in human adenoma organoids. Stable SOX9 KD shifted the adenoma culture to consist of 40-70% differentiating organoids, which was up to a 14-fold increase compared to parental and non-targeting control (NTC) by day 3 (FIG. 30F; Supplementary FIGS. 37A and 37B). Histopathology of fixed organoids demonstrated that SOX9 KD induced robust KRT20 expression by IHC and mucin production by AB staining and MUC2 IHC (FIG. 30G). Consistently, SOX9 KD reduced stem cell activity (LGR5, SOX9, AXIN2) and developmental reprogramming (LY6E, TROP2) while inducing differentiation (KRT20) (FIG. 30H). These data indicate that human FAP adenomas depend on SOX9 to maintain developmental reprogramming and prevent differentiation (FIG. 30I).

TABLE 6

| AbSC Cluster Positively Expressed Genes | | | AbSC Cluster Differentially Expressed Genes | | |
|---|---|---|---|---|---|
| Bala-Rennhack-Duoronio-Morris et al. | | | Bala-Rennhack-Duoronio-Morris et al. | | |
| Mouse | | | Mouse | | |
| 715 genes | | | 1200 genes | | |
| Gene | Avg_log2FC | Adj. p-value | Gene | Avg_log2FC | Adj. p-value |
| Ly6a | 2.97354914 | 0 | Bex1 | 1.399494253 | 0 |
| S100a6 | 2.908971172 | 0 | S100a6 | 2.908971172 | 0 |
| Wfdc18 | 2.77461541 | 6.56E−281 | S100a11 | 1.978178498 | 0 |
| Clu | 2.758273456 | 9.14E−235 | Fabp2 | −3.529365491 | 0 |
| Ifitm3 | 2.729605793 | 0 | Marcksl1 | 1.711658702 | 0 |
| Ifitm1 | 2.611367209 | 0 | Tmsb10 | 1.881019365 | 0 |
| Ly6d | 2.453189269 | 1.01E−192 | Prap1 | −3.187914245 | 0 |
| Lcn2 | 2.369225919 | 1.65E−119 | Ifitm1 | 2.611367209 | 0 |
| Ly6e | 2.00137661 | 0 | Ifitm3 | 2.729605793 | 0 |
| S100a11 | 1.978178498 | 0 | Smim24 | −2.622009662 | 0 |
| Ier3 | 1.889450789 | 2.60E−262 | Nkd1 | 1.445759336 | 0 |
| Tmsb10 | 1.881019365 | 0 | Cyba | 1.736231771 | 0 |
| Cyba | 1.736231771 | 0 | Mmp7 | 1.704530612 | 0 |
| Marcksl1 | 1.711658702 | 0 | Tm4sf5 | −2.452190622 | 0 |
| Mmp7 | 1.704530612 | 0 | Igfbp4 | 1.644442651 | 0 |
| Isg15 | 1.688288523 | 1.15E−189 | Ly6e | 2.00137661 | 0 |
| Igfbp4 | 1.644442651 | 0 | Ly6a | 2.97354914 | 0 |
| Cldn4 | 1.642324308 | 5.86E−208 | Krt18 | 0.933163216 | 0 |
| Fxyd3 | 1.620594486 | 2.72E−250 | Ecscr | 1.553303418 | 0 |
| Cd9 | 1.565251669 | 2.69E−208 | H2-Q7 | 1.409946427 | 2.43E−303 |
| Ecscr | 1.553303418 | 0 | Phgr1 | −1.545382822 | 1.03E−292 |
| Aspscr1 | 1.532821871 | 3.59E−245 | Wfdc18 | 2.77461541 | 6.56E−281 |
| Phlda1 | 1.521793184 | 1.58E−210 | Ccl25 | −1.807425975 | 1.58E−271 |
| Bst2 | 1.500122558 | 7.43E−241 | Aldob | −2.164881924 | 2.16E−269 |
| Nkd1 | 1.445759336 | 0 | Msx1 | 1.051735812 | 2.44E−263 |
| H2-Q7 | 1.409946427 | 2.43E−303 | Ier3 | 1.889450789 | 2.60E−262 |
| Bex1 | 1.399494253 | 0 | Mgst3 | −1.716283579 | 6.47E−262 |
| Stat1 | 1.34544148 | 3.11E−188 | Mif | 1.344439076 | 4.04E−254 |
| Mif | 1.344439076 | 4.04E−254 | Lgals4 | −1.098498752 | 3.03E−252 |
| Tubb5 | 1.343454944 | 6.74E−215 | Fxyd3 | 1.620594486 | 2.72E−250 |
| Krt7 | 1.335023081 | 3.27E−241 | Cd74 | 1.129794114 | 1.66E−245 |
| Pglyrp1 | 1.260829148 | 1.34E−179 | Aspscr1 | 1.532821871 | 3.59E−245 |
| Cebpd | 1.241350566 | 1.72E−102 | Krt7 | 1.335023081 | 3.27E−241 |
| Ifitm2 | 1.235829929 | 6.46E−152 | Adh1 | −1.984549293 | 3.64E−241 |
| H2-Eb1 | 1.220181633 | 6.12E−140 | Lgals2 | −1.718809998 | 5.48E−241 |
| Sox4 | 1.183424744 | 5.21E−126 | Bst2 | 1.500122558 | 7.43E−241 |
| Rbm3 | 1.159916132 | 4.79E−185 | Pigr | −1.389600518 | 2.18E−240 |
| 2210407C18Rik | 1.158055789 | 9.41E−49 | Crip1 | −2.441495387 | 4.31E−238 |
| S100a14 | 1.144908856 | 2.35E−178 | Clu | 2.758273456 | 9.14E−235 |
| Cd74 | 1.129794114 | 1.66E−245 | Tubb5 | 1.343454944 | 6.74E−215 |
| Ctsl | 1.121268837 | 1.01E−112 | Phlda1 | 1.521793184 | 1.58E−210 |
| Scd2 | 1.075968888 | 2.13E−191 | Cd9 | 1.565251669 | 2.69E−208 |
| Psmb8 | 1.075786457 | 3.45E−169 | Cldn4 | 1.642324308 | 5.86E−208 |
| Hmgn1 | 1.064895741 | 1.33E−154 | Ckmt1 | −1.536264935 | 3.35E−204 |
| H2-D1 | 1.059015781 | 1.98E−197 | Cox8a | −0.743969225 | 1.90E−197 |
| Msx1 | 1.051735812 | 2.44E−263 | H2-D1 | 1.059015781 | 1.98E−197 |
| Blvrb | 0.998577642 | 1.30E−89 | Dmbt1 | −2.006964598 | 2.95E−193 |
| Nfkbia | 0.967596339 | 4.86E−50 | Hsp90ab1 | 0.801374259 | 5.83E−193 |
| Myl12a | 0.962896727 | 5.03E−91 | Ly6d | 2.453189269 | 1.01E−192 |
| Tceal9 | 0.944060427 | 2.65E−119 | Scd2 | 1.075968888 | 2.13E−191 |
| Pla2g12a | 0.943091065 | 3.94E−136 | Isg15 | 1.688288523 | 1.15E−189 |
| Tnfrsf12a | 0.940408607 | 2.06E−168 | Stat1 | 1.34544148 | 3.11E−188 |
| Nap1l1 | 0.93731867 | 1.66E−109 | Arg2 | −1.560945208 | 2.72E−185 |
| Krt18 | 0.933163216 | 0 | Rbm3 | 1.159916132 | 4.79E−185 |
| Prmt1 | 0.922713495 | 7.17E−114 | Lypd8 | −1.133480633 | 6.92E−184 |
| Chmp4b | 0.922684561 | 5.03E−126 | Pglyrp1 | 1.260829148 | 1.34E−179 |
| Tmsb4x | 0.91197527 | 1.64E−106 | S100a14 | 1.144908856 | 2.35E−178 |
| Ets2 | 0.910760796 | 3.48E−94 | Actg1 | 0.778876479 | 1.12E−177 |
| Krt23 | 0.900295388 | 4.43E−150 | Khk | −1.346283925 | 7.89E−177 |
| Ncl | 0.872926361 | 7.32E−86 | Rtp4 | 0.857096678 | 5.68E−172 |
| Sprr2a3 | 0.864882606 | 1.38E−41 | Fabp1 | −3.494805126 | 1.20E−171 |
| H2-Ab1 | 0.859672866 | 2.23E−115 | Psmb8 | 1.075786457 | 3.45E−169 |
| Ccnd1 | 0.859659496 | 1.51E−79 | Tnfrsf12a | 0.940408607 | 2.06E−168 |
| Prox1 | 0.857511949 | 1.21E−154 | Emp2 | 0.768138841 | 2.22E−168 |
| Rtp4 | 0.857096678 | 5.68E−172 | Cox7b | −0.864438432 | 6.03E−168 |
| Iigp1 | 0.853633401 | 3.00E−152 | Pycard | −1.49638953 | 3.22E−167 |
| H2-Q4 | 0.850264033 | 2.78E−94 | Gsto1 | −1.04441494 | 4.26E−165 |
| Gstm5 | 0.839519107 | 3.54E−46 | mt-Nd4 | −0.870722332 | 1.01E−164 |
| Ceacam10 | 0.839442139 | 4.86E−25 | Cox6a1 | −0.747516939 | 2.93E−159 |
| Bok | 0.838296362 | 2.04E−149 | Atp5g3 | −0.828133409 | 4.28E−158 |
| Dynll1 | 0.835455662 | 2.93E−102 | Otc | −0.848253681 | 4.48E−158 |
| S100a16 | 0.817206778 | 3.45E−68 | Pdlim7 | 0.682453518 | 4.42E−156 |
| Zbp1 | 0.811371912 | 8.86E−132 | Cox7a2 | −0.807984447 | 6.59E−155 |
| Lgals3 | 0.810744531 | 3.65E−58 | Prox1 | 0.857511949 | 1.21E−154 |
| H2-K1 | 0.807343205 | 4.06E−148 | Hmgn1 | 1.064895741 | 1.33E−154 |
| Hsp90ab1 | 0.801374259 | 5.83E−193 | Iigp1 | 0.853633401 | 3.00E−152 |
| Axin2 | 0.795427844 | 4.83E−80 | Ifitm2 | 1.235829929 | 6.46E−152 |
| Igtp | 0.794385403 | 7.93E−119 | Oat | −1.03060655 | 8.42E−152 |
| Tspo | 0.785745889 | 8.12E−89 | Glod5 | −1.200867771 | 1.94E−151 |
| Skp1a | 0.780805043 | 1.98E−63 | Hadh | −1.03669317 | 2.01E−150 |
| Actg1 | 0.778876479 | 1.12E−177 | Krt23 | 0.900295388 | 4.43E−150 |
| Eif3c | 0.776495464 | 6.77E−67 | Calml4 | −0.983438204 | 6.41E−150 |
| Hes6 | 0.776177338 | 2.18E−62 | Bok | 0.838296362 | 2.04E−149 |
| Emp2 | 0.768138841 | 2.22E−168 | Rbp2 | −2.969444327 | 2.44E−149 |
| Anxa2 | 0.76566358 | 4.80E−45 | Cox4i1 | −0.559790505 | 2.52E−149 |
| Selenoh | 0.762564785 | 3.44E−64 | H2-K1 | 0.807343205 | 4.06E−148 |
| Nhp2 | 0.757245126 | 3.27E−77 | Cps1 | −0.998772516 | 2.48E−147 |
| Xaf1 | 0.751316726 | 1.69E−129 | Rpl41 | 0.533719492 | 3.50E−146 |
| App | 0.74720175 | 9.56E−58 | Cox6c | −0.680651535 | 4.03E−145 |
| Hnrnpa1 | 0.73982372 | 2.14E−77 | Atp5j2 | −0.702755156 | 1.13E−144 |
| Eif2s2 | 0.737585556 | 1.22E−70 | Ctxn1 | 0.665198696 | 1.15E−142 |
| Cxadr | 0.736630652 | 1.60E−15 | Hebp2 | 0.619175301 | 3.02E−142 |
| Duoxa2 | 0.73409713 | 1.09E−31 | Cda | −1.290987447 | 5.14E−142 |
| Metap2 | 0.734004144 | 7.89E−66 | Klk1 | −1.567734048 | 3.37E−141 |
| Slc25a4 | 0.727730785 | 3.20E−127 | Cfl1 | 0.702492291 | 1.01E−140 |
| Stmn1 | 0.726908242 | 3.99E−74 | Serf2 | −0.693470856 | 5.41E−140 |
| Aldoa | 0.725675547 | 4.44E−63 | Dgat1 | −1.350448022 | 5.58E−140 |
| Pebp1 | 0.723078903 | 2.37E−90 | H2-Eb1 | 1.220181633 | 6.12E−140 |
| Irf7 | 0.721373965 | 1.63E−70 | Aadac | −0.843271315 | 8.82E−140 |
| Sdc1 | 0.708016221 | 7.44E−74 | Plac8 | −1.093636031 | 6.54E−137 |
| Prdx2 | 0.703059744 | 3.16E−103 | Pla2g12a | 0.943091065 | 3.94E−136 |
| Cfl1 | 0.702492291 | 1.01E−140 | Serpinb1a | −0.812275137 | 1.41E−135 |
| Mt2 | 0.697900388 | 2.14E−55 | Fth1 | −0.993212903 | 7.40E−133 |
| Rnf213 | 0.695584225 | 7.00E−69 | Sdhb | −0.872912957 | 2.56E−132 |
| Psme1 | 0.692640201 | 1.35E−101 | Zbp1 | 0.811371912 | 8.86E−132 |
| Pdlim7 | 0.682453518 | 4.42E−156 | Cldn15 | −0.815387332 | 4.54E−131 |
| Clic1 | 0.680763673 | 3.30E−65 | Gstm3 | −1.251069384 | 2.33E−130 |
| Cela1 | 0.673141276 | 9.34E−86 | Xaf1 | 0.751316726 | 1.69E−129 |
| Ctxn1 | 0.665198696 | 1.15E−142 | Fuom | −0.796262552 | 2.33E−129 |
| Npm1 | 0.664351652 | 3.62E−74 | Tm6sf2 | −1.046707816 | 3.35E−128 |
| Hnrnpab | 0.656348436 | 8.68E−79 | Fam213b | −0.942318341 | 2.05E−127 |
| Id3 | 0.655528295 | 1.46E−13 | Cox6b1 | −0.593102783 | 2.06E−127 |
| Ifi47 | 0.653662518 | 8.16E−89 | Slc25a4 | 0.727730785 | 3.20E−127 |
| Sypl | 0.652878427 | 4.03E−53 | Cgref1 | −1.247747387 | 1.09E−126 |
| Atp6v1g1 | 0.647839 | 3.74E−60 | | | |
| Pmepa1 | 0.64742069 | 3.19E−95 | Chchd10 | −0.842915719 | 1.96E−126 |
| Vars | 0.643884682 | 2.61E−14 | Tspan8 | −0.946988018 | 2.35E−126 |
| B2m | 0.635060879 | 9.75E−85 | Chmp4b | 0.922684561 | 5.03E−126 |
| Eif4a1 | 0.629422681 | 2.73E−80 | Sox4 | 1.183424744 | 5.21E−126 |
| Cd44 | 0.629383712 | 2.73E−100 | Cideb | −1.107972336 | 3.10E−124 |
| Nop56 | 0.628904677 | 2.38E−37 | Cryl1 | −0.83177431 | 3.21E−123 |
| Lamp2 | 0.626643252 | 7.69E−49 | Atp5o.1 | −0.758777517 | 1.26E−122 |
| Smarca4 | 0.625119245 | 6.51E−27 | Sult1d1 | −0.870840132 | 9.24E−122 |
| Oas1a | 0.620836388 | 1.53E−87 | Dhrs11 | −0.850380686 | 1.61E−121 |
| Hebp2 | 0.619175301 | 3.02E−142 | Aamdc | −0.863368162 | 1.91E−120 |
| Anp32a | 0.618102881 | 3.22E−49 | Lcn2 | 2.369225919 | 1.65E−119 |
| Taf1d | 0.61372046 | 1.29E−25 | Tceal9 | 0.944060427 | 2.65E−119 |
| Hmgn2 | 0.611670656 | 5.14E−35 | Igtp | 0.794385403 | 7.93E−119 |
| H2-DMb1 | 0.610502368 | 1.15E−54 | Ces2e | −1.291245601 | 1.91E−118 |
| Psma3 | 0.607520278 | 1.33E−65 | B4galnt1 | −0.714127991 | 1.30E−117 |
| Cstb | 0.599545749 | 2.82E−32 | Reg1 | −2.975408487 | 3.56E−117 |
| Fus | 0.597738937 | 6.21E−38 | H2-Ab1 | 0.859672866 | 2.23E−115 |
| Eif5b | 0.597518054 | 1.34E−29 | Gm1123 | −0.935202738 | 5.23E−115 |
| Ube2i | 0.594811198 | 3.18E−48 | Atpif1 | −0.598817795 | 7.02E−114 |
| Xrn2 | 0.591795757 | 3.07E−47 | Prmt1 | 0.922713495 | 7.17E−114 |
| Rpsa | 0.590598221 | 1.96E−109 | Maoa | −0.97459659 | 9.77E−114 |
| Cirbp | 0.589885211 | 2.77E−57 | Cd24a | 0.495506629 | 5.24E−113 |
| Snhg12 | 0.589250403 | 7.89E−39 | Atp5g1 | −0.717888003 | 6.37E−113 |
| Ktn1 | 0.588489789 | 4.79E−46 | Ctsl | 1.121268837 | 1.01E−112 |
| Ccdc34 | 0.587930115 | 2.10E−36 | St3gal4 | −1.179747332 | 1.04E−112 |
| Rtn4 | 0.587091679 | 2.40E−83 | Ms4a10 | −1.206241651 | 2.17E−112 |
| Cd47 | 0.58233215 | 2.44E−31 | Spink1 | −2.639145334 | 3.00E−112 |
| Fermt1 | 0.58152364 | 2.99E−25 | Sult1b1 | −1.060229635 | 2.05E−111 |
| Snhg6 | 0.578527996 | 2.52E−71 | Adh6a | −1.444000176 | 4.94E−111 |
| Trmt112 | 0.576512291 | 4.44E−48 | Fbp2 | −0.80694923 | 5.69E−110 |
| Gapdh | 0.573839966 | 9.49E−89 | Ndufb9 | −0.665224461 | 1.06E−109 |
| Avpi1 | 0.573745903 | 1.70E−53 | Nap1l1 | 0.93731867 | 1.66E−109 |
| Dsg2 | 0.573498239 | 6.71E−26 | Rpsa | 0.590598221 | 1.96E−109 |
| Tgif1 | 0.570306264 | 8.51E−39 | Mttp | −1.287631908 | 4.35E−109 |
| Rps15 | 0.569011493 | 1.79E−82 | Atp5l | −0.580107818 | 1.33E−107 |
| Shisa5 | 0.566703921 | 2.50E−58 | Cyb5a | −0.901400038 | 1.86E−107 |
| Glul | 0.564517314 | 1.46E−19 | Tmsb4x | 0.91197527 | 1.64E−106 |
| Tes | 0.562130172 | 7.20E−41 | Cox5b | −0.551636907 | 2.87E−105 |
| Bex3 | 0.559969091 | 3.67E−71 | Calm1 | −0.513418396 | 4.40E−104 |
| Rpl36 | 0.55947541 | 1.27E−62 | Rnf186 | −0.715171633 | 1.15E−103 |
| Psmd7 | 0.559370416 | 9.59E−43 | Muc13 | −0.84464839 | 2.21E−103 |
| Set | 0.559146201 | 7.08E−41 | Rpl19 | 0.460947369 | 2.22E−103 |
| Llph | 0.552175406 | 5.75E−39 | Ppp1r14d | −0.749980952 | 2.40E−103 |
| Atp6v1f | 0.55043876 | 2.10E−49 | Prdx2 | 0.703059744 | 3.16E−103 |
| Ddit4 | 0.549892093 | 6.86E−52 | Cebpd | 1.241350566 | 1.72E−102 |
| Arpc1b | 0.546994413 | 1.44E−63 | Dynll1 | 0.835455662 | 2.93E−102 |
| Ddx24 | 0.546297473 | 6.60E−32 | Rpl11 | 0.504969775 | 6.98E−102 |
| Gpx2 | 0.545160612 | 9.65E−15 | Psme1 | 0.692640201 | 1.35E−101 |
| Actn1 | 0.543949709 | 3.55E−21 | Ndufb10 | −0.742766684 | 3.33E−101 |
| Hnrnpa3 | 0.543402646 | 3.49E−55 | Txn1 | −0.563999006 | 1.36E−100 |
| Urah | 0.543121281 | 6.50E−26 | Cd44 | 0.629383712 | 2.73E−100 |
| Ntmt1 | 0.542391379 | 2.89E−50 | Rpl6 | 0.525228416 | 1.43E−99 |
| Ppp1ca | 0.540581317 | 1.35E−50 | Acaa2 | −0.82798311 | 2.18E−98 |
| Ptms | 0.537764589 | 5.26E−35 | Vdr | −0.710261291 | 1.34E−97 |
| Psma7 | 0.536626257 | 2.12E−66 | Krtcap2 | −0.748700824 | 6.52E−97 |
| Rpl41 | 0.533719492 | 3.50E−146 | Chp2 | −0.814633597 | 1.19E−96 |
| Dnajc2 | 0.531896529 | 1.99E−35 | Tspan3 | −0.693179278 | 2.78E−96 |
| Cdk4 | 0.531596598 | 4.83E−39 | Pmepa1 | 0.64742069 | 3.19E−95 |
| Snrpe | 0.531094585 | 1.94E−51 | Cmbl | −0.674697646 | 9.02E−95 |
| H2afy | 0.530401365 | 3.13E−28 | Minos1 | −0.699525334 | 9.40E−95 |
| Smarcc1 | 0.526590873 | 7.76E−20 | Sis | −1.424142334 | 1.27E−94 |
| 1810022K09Rik | 0.525873105 | 1.24E−40 | Clca4b | −0.695579413 | 2.60E−94 |
| Sox9 | 0.525555927 | 1.07E−33 | H2-Q4 | 0.850264033 | 2.78E−94 |
| Rpl6 | 0.525228416 | 1.43E−99 | Slc25a5 | −0.606556846 | 3.06E−94 |
| Rpl31 | 0.524322462 | 7.17E−68 | Ets2 | 0.910760796 | 3.48E−94 |
| Psmd4 | 0.523779324 | 4.49E−34 | Cdhr5 | −1.625395509 | 1.11E−93 |
| Rnh1 | 0.52364773 | 7.37E−33 | Apoc3 | −2.585477655 | 2.78E−93 |
| Mafg | 0.52307048 | 2.22E−35 | mt-Nd2 | −0.486291989 | 1.32E−92 |
| Csrp1 | 0.522281379 | 3.98E−56 | Uqcr11 | −0.526119999 | 1.49E−91 |
| Knop1 | 0.52211339 | 2.08E−25 | Myl12a | 0.962896727 | 5.03E−91 |
| Gnl3 | 0.52119811 | 2.01E−22 | Mdh2 | −0.836682562 | 1.87E−90 |
| Chd4 | 0.520991265 | 1.20E−11 | Pebp1 | 0.723078903 | 2.37E−90 |
| Ehf | 0.519482609 | 5.42E−39 | Acp5 | −0.654439532 | 6.12E−90 |
| H2-T22 | 0.519290747 | 3.23E−50 | Blvrb | 0.998577642 | 1.30E−89 |
| Nedd4 | 0.519033243 | 1.09E−22 | Krt20 | −1.317636353 | 1.60E−89 |
| 1110004F10Rik | 0.518892113 | 7.75E−39 | Mpc2 | −0.69530557 | 2.16E−89 |
| Bcas2 | 0.517987714 | 4.14E−33 | Rps14 | 0.456190071 | 2.73E−89 |
| Mbd3 | 0.516456778 | 5.98E−43 | Gstp1 | −0.67085851 | 3.61E−89 |
| Cct5 | 0.516284245 | 4.73E−24 | Uqcrfs1 | −0.658937734 | 5.73E−89 |
| Psmb5 | 0.516137256 | 1.84E−49 | Tspo | 0.785745889 | 8.12E−89 |
| 1500011B03Rik | 0.515720521 | 1.00E−66 | Ifi47 | 0.653662518 | 8.16E−89 |
| H3f3b | 0.514920858 | 3.85E−81 | Gapdh | 0.573839966 | 9.49E−89 |
| Parp14 | 0.514881917 | 6.52E−31 | Scp2 | −1.124898714 | 2.67E−88 |
| Ptma | 0.514875206 | 1.01E−58 | Oas1a | 0.620836388 | 1.53E−87 |
| Irf1 | 0.514594688 | 9.04E−11 | Cd302 | −0.551884836 | 4.91E−87 |
| Epb41l4aos | 0.511645703 | 1.41E−23 | Fahd1 | −0.717791914 | 1.36E−86 |
| 2010111I01Rik | 0.510842179 | 2.32E−26 | Bpnt1 | −0.785622806 | 1.70E−86 |
| Tubb2b | 0.510496195 | 9.74E−49 | Aldh1b1 | −0.658372053 | 3.57E−86 |
| Cenpm | 0.505571237 | 4.18E−40 | Akr1c19 | −0.683857405 | 5.70E−86 |
| Polr2f | 0.505391369 | 5.52E−29 | Mapk13 | −0.596013843 | 6.20E−86 |
| Rpl11 | 0.504969775 | 6.98E−102 | Rps23 | 0.489069009 | 6.59E−86 |
| Tap1 | 0.504238233 | 1.77E−28 | Ncl | 0.872926361 | 7.32E−86 |
| Hnrnpdl | 0.503244887 | 1.31E−30 | BC030870 | −0.559660158 | 8.39E−86 |
| Ube2s | 0.502935149 | 1.51E−19 | Cela1 | 0.673141276 | 9.34E−86 |
| Nenf | 0.500376562 | 3.36E−37 | B2m | 0.635060879 | 9.75E−85 |
| Srrm2 | 0.49979301 | 1.71E−31 | Chchd7 | −0.674092439 | 2.80E−84 |
| Mt1 | 0.499150571 | 3.24E−05 | Sri | −0.625687589 | 9.19E−84 |
| Rpl38 | 0.499046409 | 3.27E−71 | Rtn4 | 0.587091679 | 2.40E−83 |
| Eif5a | 0.4983944 | 4.43E−50 | Cdh17 | −0.772760474 | 2.61E−83 |
| Cd24a | 0.495506629 | 5.24E−113 | Prlr | −0.584961136 | 2.84E−83 |
| Snrpb | 0.494797051 | 6.45E−39 | Clec2h | −1.191054818 | 6.01E−83 |
| Lsm7 | 0.493557328 | 7.44E−24 | Ndufa5 | −0.645755263 | 7.52E−83 |
| Gipc1 | 0.493097036 | 8.20E−36 | Rps15 | 0.569011493 | 1.79E−82 |
| Kcne3 | 0.491572881 | 2.35E−19 | Alpi | −1.430760402 | 2.24E−82 |
| Arid5b | 0.491114705 | 1.64E−25 | Chpt1 | −0.540585934 | 2.65E−82 |
| Tuba1b | 0.491039759 | 7.17E−20 | Aoc1 | −0.673196115 | 3.13E−82 |
| Sssca1 | 0.490692739 | 8.74E−30 | Cst6 | −1.150585353 | 5.31E−82 |
| Rps23 | 0.489069009 | 6.59E−86 | Ndufc1 | −0.643726943 | 8.23E−82 |
| Tra2a | 0.4882779 | 5.61E−08 | H3f3b | 0.514920858 | 3.85E−81 |
| Fdft1 | 0.488041241 | 1.26E−43 | Cyc1 | −0.65213637 | 4.04E−81 |
| Id1 | 0.482451874 | 7.49E−11 | Mdh1 | −0.784110972 | 1.09E−80 |
| H2-Aa | 0.482291221 | 9.77E−33 | Gstt1 | −0.711958983 | 2.57E−80 |
| Rpl27 | 0.481831155 | 1.38E−77 | Eif4a1 | 0.629422681 | 2.73E−80 |
| Ptov1 | 0.481659068 | 3.95E−21 | Axin2 | 0.795427844 | 4.83E−80 |
| Grn | 0.478121965 | 9.92E−19 | Cdhr2 | −1.364265037 | 5.70E−80 |
| Fbl | 0.477623403 | 2.52E−29 | Tcea3 | −0.528817431 | 1.07E−79 |
| Ube216 | 0.474559209 | 2.89E−54 | Ccnd1 | 0.859659496 | 1.51E−79 |
| Myl12b | 0.473766996 | 7.53E−20 | Anpep | −1.847346389 | 2.43E−79 |
| Serbp1 | 0.473698884 | 3.00E−45 | Uqcrq | −0.46991554 | 4.80E−79 |
| Aplp2 | 0.473379922 | 0.000353321 | Hnrnpab | 0.656348436 | 8.68E−79 |
| Mybbp1a | 0.473164565 | 1.05E−15 | Rpl26 | 0.462741702 | 1.68E−78 |
| Psmc4 | 0.471117632 | 7.52E−23 | Gna11 | −0.869477083 | 2.12E−78 |
| Qtrt1 | 0.46850658 | 4.44E−31 | Epcam | 0.422886256 | 2.50E−78 |
| Med21 | 0.468205761 | 4.62E−30 | Rpl27 | 0.481831155 | 1.38E−77 |
| Map1lc3b | 0.464727736 | 1.90E−20 | Cox5a | −0.559492834 | 1.54E−77 |
| Prom1 | 0.464681053 | 6.93E−07 | Hnrnpa1 | 0.73982372 | 2.14E−77 |
| Lmo7 | 0.464053931 | 9.99E−25 | Nhp2 | 0.757245126 | 3.27E−77 |
| Tpi1 | 0.463137237 | 1.75E−55 | Atp5c1 | −0.55824743 | 8.06E−77 |
| Rpl26 | 0.462741702 | 1.68E−78 | Atp5d | −0.42569121 | 9.47E−77 |
| Srsf11 | 0.461610566 | 4.14E−21 | Atp5h | −0.483478535 | 1.13E−76 |
| Rpl19 | 0.460947369 | 2.22E−103 | Aqp11 | −0.593831276 | 1.80E−76 |
| Nme4 | 0.459682448 | 6.13E−51 | Uqcrh | −0.468061743 | 2.28E−76 |
| Erh | 0.459585899 | 8.48E−30 | Apob | −1.384068465 | 6.38E−76 |
| Rbm25 | 0.458969126 | 3.35E−18 | Cox7c | −0.438355337 | 1.42E−75 |
| Npc2 | 0.458566649 | 1.58E−22 | Gstm2 | −0.433949694 | 1.68E−75 |
| Chic2 | 0.458178143 | 3.10E−30 | Mtch2 | −0.669464707 | 1.89E−75 |
| Itga6 | 0.457592241 | 5.73E−14 | Ass1 | 0.434745537 | 2.71E−75 |
| Rps14 | 0.456190071 | 2.73E−89 | P4hb | −0.970424433 | 3.84E−75 |
| Rps19 | 0.452209415 | 2.17E−60 | Lysmd2 | 0.402822246 | 2.09E−74 |
| Lgals9 | 0.452114357 | 1.34E−23 | Cyb5r3 | −0.864174138 | 2.54E−74 |
| Tagln2 | 0.451557877 | 7.28E−22 | Tnfsf13os | −0.549591661 | 2.59E−74 |
| Cct7 | 0.450995737 | 5.24E−14 | Npm1 | 0.664351652 | 3.62E−74 |
| Ppp1r2 | 0.450885569 | 6.73E−26 | Stmn1 | 0.726908242 | 3.99E−74 |
| Rpl14 | 0.450173409 | 7.06E−65 | Sdc1 | 0.708016221 | 7.44E−74 |
| Raly | 0.450020983 | 1.10E−23 | Ephx2 | −0.815220779 | 8.30E−74 |
| Tpm3 | 0.449198494 | 4.67E−19 | Car4 | −0.700743947 | 1.03E−73 |
| Ctsd | 0.44749562 | 3.67E−10 | Sdhd | −0.614002428 | 7.21E−73 |
| Btf3 | 0.447385325 | 3.36E−59 | Ces1f | −0.628961279 | 7.32E−73 |
| Arpp19 | 0.447186675 | 1.07E−25 | Hdhd3 | −0.53610336 | 5.64E−72 |
| Cdca7 | 0.446686738 | 6.87E−12 | Ndufb11 | −0.525959847 | 1.61E−71 |
| Bola2 | 0.446510589 | 1.02E−16 | Ahcyl2 | −0.660728026 | 1.62E−71 |
| Nolc1 | 0.446349718 | 1.84E−11 | Snhg6 | 0.578527996 | 2.52E−71 |
| Psma2 | 0.44590197 | 5.28E−32 | Ces2c | −0.857784294 | 3.25E−71 |
| Acer2 | 0.445464975 | 1.62E−51 | Rpl38 | 0.499046409 | 3.27E−71 |
| Srsf7 | 0.444749026 | 2.12E−22 | Msra | −0.534871688 | 3.28E−71 |
| Bnip3l | 0.443980481 | 1.66E−17 | Bex3 | 0.559969091 | 3.67E−71 |
| Tcof1 | 0.442849646 | 9.94E−16 | Eif2s2 | 0.737585556 | 1.22E−70 |
| Sf3b3 | 0.442486374 | 5.58E−12 | Cyp4f14 | −0.885836581 | 1.39E−70 |
| Eif3d | 0.441386655 | 6.75E−14 | Ndufa4 | −0.508070288 | 1.49E−70 |
| Ilf2 | 0.441261082 | 9.32E−17 | Aldh9a1 | −0.618483841 | 1.53E−70 |
| Bclaf1 | 0.440400513 | 7.30E−11 | Irf7 | 0.721373965 | 1.63E−70 |
| Snu13 | 0.439963161 | 8.62E−27 | Ddc | −0.663928404 | 4.83E−70 |
| Ebna1bp2 | 0.438476224 | 6.05E−23 | Rpl18a | 0.413923557 | 7.66E−70 |
| Snrpd1 | 0.437807356 | 1.18E−24 | Fmo5 | −0.753746325 | 2.65E−69 |
| Snrnp70 | 0.437492847 | 3.40E−12 | Myo1a | −0.889481642 | 5.06E−69 |
| Ranbp1 | 0.436052632 | 2.27E−24 | Abcg2 | −0.579609045 | 5.63E−69 |
| Ift43 | 0.435654376 | 7.55E−30 | Rpl34 | 0.417329257 | 6.44E−69 |
| Rps18 | 0.434793277 | 3.90E−56 | Rnf213 | 0.695584225 | 7.00E−69 |
| Ass1 | 0.434745537 | 2.71E−75 | Ndufb5 | −0.549036503 | 9.15E−69 |
| Siva1 | 0.434539718 | 1.49E−24 | Erich4 | −0.713966366 | 1.48E−68 |
| Rplp0 | 0.434293144 | 3.84E−68 | S100a16 | 0.817206778 | 3.45E−68 |
| Lsr | 0.432650101 | 4.36E−22 | Rplp0 | 0.434293144 | 3.84E−68 |
| Rpl13a | 0.432398598 | 2.58E−29 | Rpl31 | 0.524322462 | 7.17E−68 |
| Rpl12 | 0.432177175 | 1.36E−53 | Cox7a1 | −0.785911502 | 1.68E−67 |
| Tpm1 | 0.431354322 | 5.60E−15 | Tmem37 | −0.660357437 | 1.83E−67 |
| Vcp | 0.429896338 | 0.033914108 | Fos | −0.761070936 | 3.07E−67 |
| Pabpc1 | 0.429235751 | 2.41E−16 | Uqcrc1 | −0.574145229 | 4.17E−67 |
| Cnbp | 0.429189553 | 7.63E−29 | Usmg5 | −0.517971083 | 6.70E−67 |
| Mak16 | 0.427801643 | 1.10E−20 | Eif3c | 0.776495464 | 6.77E−67 |
| Psmb4 | 0.427589034 | 8.97E−31 | 1500011B03Rik | 0.515720521 | 1.00E−66 |
| Srsf10 | 0.425738777 | 1.66E−06 | Apoa4 | −2.914131298 | 1.98E−66 |
| Sumo2 | 0.425657908 | 3.82E−25 | Psma7 | 0.536626257 | 2.12E−66 |
| Ezh2 | 0.423108166 | 6.45E−16 | Slc39a5 | −0.500812412 | 2.29E−66 |
| Epcam | 0.422886256 | 2.50E−78 | Ogdh | −0.75876195 | 3.34E−66 |
| Ppp1r14b | 0.422633575 | 1.29E−26 | Dhrs1 | −0.753821491 | 3.67E−66 |
| Gar1 | 0.422352807 | 3.39E−25 | Myo15b | −0.983398753 | 4.29E−66 |
| G3bp2 | 0.421558109 | 2.29E−23 | Metap2 | 0.734004144 | 7.89E−66 |
| Fasn | 0.421266466 | 8.67E−26 | Psma3 | 0.607520278 | 1.33E−65 |
| Psip1 | 0.421154817 | 2.50E−30 | Reep6 | −0.61315619 | 1.65E−65 |
| Pdap1 | 0.420709617 | 3.95E−17 | Atp5f1 | −0.517268776 | 1.83E−65 |
| Rpl4 | 0.418367838 | 2.04E−47 | Clic1 | 0.680763673 | 3.30E−65 |
| Rps20 | 0.417503353 | 5.55E−64 | Ech1 | −0.529712605 | 4.21E−65 |
| Rpl34 | 0.417329257 | 6.44E−69 | Acat1 | −0.484071573 | 6.06E−65 |
| Clic4 | 0.416788418 | 2.48E−23 | Rpl14 | 0.450173409 | 7.06E−65 |
| Laptm4a | 0.416484811 | 1.02E−14 | Ndufs7 | −0.530662085 | 8.79E−65 |
| Rpl10a | 0.416198609 | 1.58E−59 | Selenoh | 0.762564785 | 3.44E−64 |
| Ddx21 | 0.415499844 | 2.96E−08 | Rps20 | 0.417503353 | 5.55E−64 |
| Exosc4 | 0.414678434 | 2.06E−14 | Creb3l3 | −0.90123993 | 1.00E−63 |
| Rpl18a | 0.413923557 | 7.66E−70 | Apoa1 | −2.800411367 | 1.41E−63 |
| Rpl7 | 0.412394499 | 6.60E−54 | Arpc1b | 0.546994413 | 1.44E−63 |
| Rps5 | 0.411324908 | 2.77E−60 | Skp1a | 0.780805043 | 1.98E−63 |
| Ssrp1 | 0.410703481 | 3.34E−08 | Cenpx | −0.564008768 | 2.98E−63 |
| Tst | 0.410595687 | 1.58E−35 | Aldoa | 0.725675547 | 4.44E−63 |
| Zfp36l1 | 0.410096152 | 1.37E−07 | Ndufa3 | −0.54244358 | 5.52E−63 |
| Mbnl1 | 0.409785421 | 9.19E−07 | Rpl36 | 0.55947541 | 1.27E−62 |
| Map1lc3a | 0.409624614 | 1.86E−19 | Ifi27l2b | −1.269870279 | 1.38E−62 |
| Cct2 | 0.407470261 | 1.09E−17 | Hes6 | 0.776177338 | 2.18E−62 |
| Nmi | 0.406945591 | 3.90E−19 | Hadha | −0.574060046 | 2.40E−62 |
| Rpl27a | 0.405309185 | 2.80E−58 | Mall | −0.588253629 | 2.46E−62 |
| Mrto4 | 0.404720969 | 1.32E−21 | mt-Nd4l | −0.374895231 | 4.23E−62 |
| Pfdn2 | 0.404554694 | 1.10E−17 | H2afv | −0.588424441 | 4.25E−62 |
| Eif2ak2 | 0.404404965 | 4.90E−25 | Rps19 | 0.452209415 | 2.17E−60 |
| Cuedc2 | 0.404126479 | 1.80E−22 | Atp5k | −0.51356276 | 2.64E−60 |
| Psmb9 | 0.403486064 | 8.26E−16 | Rps5 | 0.411324908 | 2.77E−60 |
| Mtdh | 0.403293096 | 7.84E−15 | Atp6v1g1 | 0.647839 | 3.74E−60 |
| Nop58 | 0.403248257 | 6.22E−11 | Cdk2ap2 | −0.629987601 | 4.54E−60 |
| Lysmd2 | 0.402822246 | 2.09E−74 | Rpl10a | 0.416198609 | 1.58E−59 |
| Rps17 | 0.402729697 | 3.23E−44 | Rpl24 | 0.38871485 | 2.09E−59 |
| Tnfaip8l1 | 0.401411337 | 1.93E−49 | Btf3 | 0.447385325 | 3.36E−59 |
| Tuba1c | 0.400510568 | 3.41E−10 | Spink4 | −3.958414841 | 3.87E−59 |
| Smarce1 | 0.400204968 | 9.32E−18 | Trim31 | −0.595135282 | 8.15E−59 |
| Slc38a2 | 0.399854091 | 0.008030202 | Ptma | 0.514875206 | 1.01E−58 |
| Eif4ebp1 | 0.399544867 | 2.51E−10 | Guca2b | −2.215585598 | 1.33E−58 |
| Vgll4 | 0.399229365 | 4.23E−11 | Shisa5 | 0.566703921 | 2.50E−58 |
| Eef2 | 0.398465 | 2.92E−14 | Rpl27a | 0.405309185 | 2.80E−58 |
| Rps9 | 0.39842011 | 1.54E−55 | Adtrp | −0.497882366 | 3.00E−58 |
| H2afj | 0.398143977 | 7.55E−23 | Sfxn1 | −0.556771958 | 3.47E−58 |
| Zfp593 | 0.397842204 | 3.70E−23 | Lgals3 | 0.810744531 | 3.65E−58 |
| Arl6ip4 | 0.397305537 | 4.74E−12 | Coa3 | −0.502767226 | 4.37E−58 |
| Eef1e1 | 0.396901383 | 4.70E−19 | Enpep | −0.665693409 | 5.02E−58 |
| Hnrnpu | 0.396536774 | 6.17E−10 | App | 0.74720175 | 9.56E−58 |
| Psmc3 | 0.396334867 | 4.75E−15 | Selenop | −1.595398614 | 1.90E−57 |
| Atrx | 0.395665438 | 0.00126614 | Cirbp | 0.589885211 | 2.77E−57 |
| Srrm1 | 0.395328441 | 6.35E−18 | Aco2 | −0.526830131 | 1.83E−56 |
| Rps25 | 0.39518314 | 1.41E−44 | Cycs | −0.547778666 | 2.33E−56 |
| Med10 | 0.394987249 | 1.53E−27 | Rps18 | 0.434793277 | 3.90E−56 |
| Tomm20 | 0.393317534 | 2.57E−20 | Csrp1 | 0.522281379 | 3.98E−56 |
| Rplp2 | 0.393288807 | 1.82E−52 | Smpdl3a | −0.452301448 | 4.19E−56 |
| Grcc10 | 0.393096389 | 1.34E−12 | Lpgat1 | −0.720246889 | 4.20E−56 |
| Znhit1 | 0.392417982 | 3.48E−12 | mt-Nd3 | −0.480581229 | 4.75E−56 |
| Akr1b3 | 0.392411619 | 4.71E−24 | Suclg1 | −0.531132298 | 5.06E−56 |
| Rpl3 | 0.392053515 | 5.05E−44 | Atp5e | −0.343102052 | 1.53E−55 |
| F11r | 0.391538559 | 5.03E−11 | Rps9 | 0.39842011 | 1.54E−55 |
| Rps12 | 0.391394992 | 3.41E−39 | Tpi1 | 0.463137237 | 1.75E−55 |
| Mcrip1 | 0.39110356 | 1.96E−19 | Mt2 | 0.697900388 | 2.14E−55 |
| Uba52 | 0.39061907 | 1.16E−38 | Cisd3 | −0.506965996 | 2.33E−55 |
| Paics | 0.389643997 | 9.49E−30 | Hnrnpa3 | 0.543402646 | 3.49E−55 |
| Rpl24 | 0.38871485 | 2.09E−59 | Slc51a | −0.493831432 | 7.29E−55 |
| Snrpf | 0.38851751 | 6.09E−23 | H2-DMb1 | 0.610502368 | 1.15E−54 |
| Mat2a | 0.388514634 | 4.41E−10 | Dpp4 | −0.67912625 | 1.19E−54 |
| Smarca5 | 0.388443156 | 7.32E−11 | Gm45716 | −0.362339123 | 2.34E−54 |
| Fcf1 | 0.388376244 | 4.73E−16 | Ube2l6 | 0.474559209 | 2.89E−54 |
| Echdc2 | 0.388234012 | 4.23E−36 | Cdkn2b | −0.529080668 | 3.61E−54 |
| Akap13 | 0.387426393 | 3.17E−11 | Atp5j | −0.379660663 | 5.67E−54 |
| Gch1 | 0.386576799 | 2.49E−33 | Rpl7 | 0.412394499 | 6.60E−54 |
| Hif1a | 0.386338308 | 3.26E−23 | Tcn2 | −0.648528704 | 6.97E−54 |
| Lars | 0.385517463 | 2.75E−18 | Slc27a4 | −1.015740164 | 1.06E−53 |
| Stard10 | 0.385465347 | 0.000278907 | Rpl12 | 0.432177175 | 1.36E−53 |
| Anp32b | 0.385439645 | 9.53E−24 | Avpi1 | 0.573745903 | 1.70E−53 |
| Ssb | 0.385381868 | 6.00E−16 | Mgst1 | −0.67295255 | 2.71E−53 |
| Hddc2 | 0.385200381 | 5.51E−31 | Sypl | 0.652878427 | 4.03E−53 |
| Ddx39 | 0.384385367 | 4.68E−13 | Tkfc | −0.603243652 | 4.09E−53 |
| Plscr1 | 0.383523491 | 5.86E−22 | Uqcrb | −0.464589764 | 5.81E−53 |
| Adrm1 | 0.383437568 | 5.89E−17 | Mogat2 | −0.721928497 | 8.80E−53 |
| Sap30 | 0.383350537 | 1.16E−30 | 2200002D01Rik | −1.02635051 | 9.52E−53 |
| Rabac1 | 0.382827516 | 2.17E−13 | Sfn | −0.616440932 | 1.02E−52 |
| Tpd52l2 | 0.381959962 | 1.79E−09 | Akr7a5 | −0.451832066 | 1.19E−52 |
| Tpm4 | 0.381565619 | 8.15E−27 | Epb41l3 | −0.506683303 | 1.75E−52 |
| Hprt | 0.381457941 | 2.27E−14 | Rplp2 | 0.393288807 | 1.82E−52 |
| Gadd45g | 0.381127599 | 5.66E−52 | Mep1b | −0.963226551 | 2.87E−52 |
| Fdps | 0.380838196 | 7.82E−16 | Gadd45g | 0.381127599 | 5.66E−52 |
| Nemf | 0.380395404 | 3.48E−10 | Ddit4 | 0.549892093 | 6.86E−52 |
| Tcp1 | 0.379372323 | 2.94E−14 | Me2 | −0.461619285 | 1.24E−51 |
| Rplp1 | 0.379346888 | 2.18E−48 | Cat | −0.566285055 | 1.51E−51 |
| Apex1 | 0.378907101 | 9.50E−21 | Acer2 | 0.445464975 | 1.62E−51 |
| Rps13 | 0.378652059 | 4.60E−51 | Snrpe | 0.531094585 | 1.94E−51 |
| Bax | 0.378400471 | 1.69E−17 | S100a1 | −0.726976137 | 2.64E−51 |
| Chd9 | 0.377817235 | 2.28E−06 | Pla2g12b | −0.493805109 | 2.95E−51 |
| Rps28 | 0.377665748 | 1.21E−43 | Rps13 | 0.378652059 | 4.60E−51 |
| Usp22 | 0.376977518 | 8.79E−20 | Nme4 | 0.459682448 | 6.13E−51 |
| Trib1 | 0.376748988 | 0.010351265 | Cbr1 | −0.720782558 | 7.92E−51 |
| Tpr | 0.376540898 | 6.40E−09 | Ak2 | −0.495496909 | 8.54E−51 |
| Cnn3 | 0.374980903 | 1.26E−16 | Isx | −0.339500629 | 9.62E−51 |
| Sars | 0.373984462 | 0.000388026 | Sec13 | −0.499336127 | 9.92E−51 |
| Rps6 | 0.373917175 | 3.72E−42 | Sdhc | −0.482388699 | 1.09E−50 |
| Rabggtb | 0.373114263 | 4.27E−09 | Ace | −1.069187502 | 1.34E−50 |
| Gtpbp4 | 0.373039031 | 1.19E−13 | Ppp1ca | 0.540581317 | 1.35E−50 |
| Fkbp3 | 0.372829105 | 3.00E−16 | Pycrl | −0.468483876 | 1.49E−50 |
| Lgals3bp | 0.372622015 | 1.03E−12 | Iqgap2 | −0.587042658 | 2.15E−50 |
| Snrpd2 | 0.371064889 | 1.42E−18 | Ntmt1 | 0.542391379 | 2.89E−50 |
| Scaf11 | 0.370298846 | 3.13E−06 | Ndufv3 | −0.447265004 | 3.01E−50 |
| Sertad1 | 0.368733524 | 6.88E−19 | mt-Cytb | −0.305171645 | 3.18E−50 |
| Psmc6 | 0.367955511 | 4.45E−12 | H2-T22 | 0.519290747 | 3.23E−50 |
| Park7 | 0.36675562 | 9.10E−24 | Eif5a | 0.4983944 | 4.43E−50 |
| Dctn3 | 0.3664681 | 5.70E−16 | Nfkbia | 0.967596339 | 4.86E−50 |
| Pgls | 0.366335327 | 7.61E−16 | Acsl5 | −0.996230684 | 5.48E−50 |
| Atox1 | 0.366187022 | 5.18E−12 | Acox1 | −0.741344637 | 5.68E−50 |
| Rbx1 | 0.365602948 | 1.90E−22 | Rab4a | −0.40446569 | 7.55E−50 |
| Sf3b2 | 0.363597224 | 0.055810021 | Atp1a1 | −0.901771938 | 8.92E−50 |
| Pomp | 0.362541456 | 2.85E−25 | Bdh1 | −0.437739425 | 1.34E−49 |
| Capns1 | 0.362005167 | 3.81E−08 | Psmb5 | 0.516137256 | 1.84E−49 |
| Nsmce1 | 0.361700132 | 3.90E−25 | Tnfaip8l1 | 0.401411337 | 1.93E−49 |
| Ran | 0.361584771 | 1.87E−18 | Slc9a3r1 | −0.91064772 | 1.97E−49 |
| Dcun1d5 | 0.361558848 | 1.97E−14 | Atp6v1f | 0.55043876 | 2.10E−49 |
| Snrpg | 0.361450593 | 4.28E−22 | Hnf4g | −0.342278658 | 2.64E−49 |
| Lyar | 0.361357624 | 1.75E−09 | Galnt6 | −0.531337117 | 2.84E−49 |
| Nubp1 | 0.361259922 | 1.51E−16 | Anp32a | 0.618102881 | 3.22E−49 |
| Bola1 | 0.361063463 | 1.31E−15 | Txndc17 | −0.54290259 | 3.29E−49 |
| Otub1 | 0.360594404 | 5.85E−12 | Tmbim6 | −0.606078659 | 3.55E−49 |
| 1810058I24Rik | 0.36052704 | 1.97E−11 | Ggt1 | −1.031979306 | 5.88E−49 |
| Prr15 | 0.35970891 | 3.74E−22 | Lamp2 | 0.626643252 | 7.69E−49 |
| Fam60a | 0.359356795 | 4.29E−17 | H2-Q2 | −0.880587237 | 8.30E−49 |
| Trp53 | 0.35926038 | 5.66E−10 | 2210407C18Rik | 1.158055789 | 9.41E−49 |
| Psmb6 | 0.358929308 | 4.11E−22 | Tubb2b | 0.510496195 | 9.74E−49 |
| Ralbp1 | 0.358824523 | 6.10E−05 | Cyp3a13 | −0.837447475 | 1.55E−48 |
| Csnk1a1 | 0.357545271 | 9.95E−07 | Rplp1 | 0.379346888 | 2.18E−48 |
| Rpl37a | 0.357064244 | 6.69E−33 | Idh3b | −0.530163781 | 2.97E−48 |
| Rpl17 | 0.356823603 | 9.23E−39 | Ube2i | 0.594811198 | 3.18E−48 |
| Sdhaf1 | 0.356584769 | 1.10E−07 | Trmt112 | 0.576512291 | 4.44E−48 |
| Crb3 | 0.356369106 | 9.23E−11 | Rpl4 | 0.418367838 | 2.04E−47 |
| Ewsr1 | 0.355998881 | 0.004961259 | Xrn2 | 0.591795757 | 3.07E−47 |
| Nono | 0.355744574 | 1.58E−09 | Acbd4 | −0.480989148 | 4.61E−47 |
| Psmc2 | 0.355407852 | 2.62E−12 | Tmem120a | −0.687036708 | 7.17E−47 |
| Pkm | 0.3553343 | 1.12E−17 | Gstm5 | 0.839519107 | 3.54E−46 |
| Rpl5 | 0.355322036 | 1.44E−34 | mt-Co3 | −0.302846031 | 3.68E−46 |
| Psmg4 | 0.354825035 | 5.18E−14 | Ktn1 | 0.588489789 | 4.79E−46 |
| Polr2g | 0.354755071 | 1.14E−20 | Ndufab1 | −0.452888234 | 5.91E−46 |
| Rtf1 | 0.354748237 | 1.08E−05 | Onecut2 | −0.279503127 | 8.42E−46 |
| Eif1ax | 0.35469496 | 5.56E−18 | Prkca | −0.371188644 | 9.29E−46 |
| Utp3 | 0.354293612 | 2.18E−06 | Ndufb6 | −0.489655839 | 9.31E−46 |
| Rbm39 | 0.353640901 | 3.98E−09 | Vkorc1 | −0.364575509 | 1.02E−45 |
| Sltm | 0.353137588 | 2.68E−08 | Etfa | −0.477027892 | 1.66E−45 |
| Eif4g2 | 0.352891091 | 2.63E−09 | Prdx5 | −0.755327504 | 1.76E−45 |
| Rpl32 | 0.352612181 | 2.69E−38 | Atp5a1 | −0.426579485 | 1.78E−45 |
| Srm | 0.352548488 | 8.94E−13 | Aldh2 | −0.783556959 | 2.01E−45 |
| Nras | 0.351909624 | 1.22E−12 | Serbp1 | 0.473698884 | 3.00E−45 |
| Esf1 | 0.350355004 | 1.02E−07 | Anxa2 | 0.76566358 | 4.80E−45 |
| Cdc37 | 0.350025139 | 1.62E−08 | Ndufb2 | −0.462572134 | 1.00E−44 |
| Psmb3 | 0.34902439 | 2.03E−22 | Hagh | −0.468658265 | 1.36E−44 |
| Puf60 | 0.348475872 | 1.71E−05 | Nlrp6 | −0.357827714 | 1.40E−44 |
| Ppan | 0.348148313 | 1.38E−06 | Rps25 | 0.39518314 | 1.41E−44 |
| Zfand5 | 0.348143396 | 5.55E−05 | Cox20 | −0.468437518 | 1.92E−44 |
| Mrpl17 | 0.348114134 | 1.21E−11 | Rps17 | 0.402729697 | 3.23E−44 |
| Fam46a | 0.347416284 | 4.53E−10 | Slc25a3 | −0.37946369 | 4.26E−44 |
| Pak2 | 0.347286126 | 9.14E−11 | Rpl3 | 0.392053515 | 5.05E−44 |
| Eif3a | 0.346966574 | 0.023778101 | Rps28 | 0.377665748 | 1.21E−43 |
| Gnai2 | 0.346645896 | 1.13E−19 | Fdft1 | 0.488041241 | 1.26E−43 |
| Tia1 | 0.346513626 | 6.81E−13 | Sdha | −0.414222381 | 2.20E−43 |
| Fkbp1a | 0.345776919 | 4.10E−14 | Ugt2b34 | −0.536850177 | 3.16E−43 |
| Ywhae | 0.345750121 | 1.60E−09 | Nfe2l2 | −0.406852638 | 4.01E−43 |
| Rpl37 | 0.344888551 | 1.71E−40 | Ndufb3 | −0.447919325 | 4.68E−43 |
| Txndc9 | 0.344552491 | 1.79E−09 | Acy1 | −0.459553283 | 5.69E−43 |
| Ufc1 | 0.344449905 | 5.43E−13 | Mbd3 | 0.516456778 | 5.98E−43 |
| Mphosph10 | 0.344417443 | 1.36E−11 | Psmd7 | 0.559370416 | 9.59E−43 |
| Rpl35 | 0.343201254 | 2.98E−35 | Surf4 | −0.468141212 | 1.57E−42 |
| Pafah1b3 | 0.34298871 | 1.22E−09 | Calm3 | −0.410152098 | 2.50E−42 |
| Hmga1 | 0.342416704 | 2.23E−13 | Ndufa11 | −0.419661802 | 3.20E−42 |
| Lmna | 0.342393928 | 2.51E−06 | Rps6 | 0.373917175 | 3.72E−42 |
| Pak1 | 0.341660735 | 6.41E−15 | Uqcrc2 | −0.471271349 | 5.78E−42 |
| Pa2g4 | 0.341365009 | 1.98E−16 | Sprr2a3 | 0.864882606 | 1.38E−41 |
| Trappc6a | 0.341354279 | 6.80E−10 | Rflnb | −0.394287376 | 1.42E−41 |
| H3f3a | 0.340501928 | 3.39E−26 | Mcrip2 | −0.379010106 | 1.58E−41 |
| Kif5b | 0.340395188 | 4.90E−06 | Gng12 | −0.558397306 | 2.86E−41 |
| Rpl13 | 0.340368867 | 7.50E−29 | Muc3 | −0.532753563 | 5.35E−41 |
| Hmgb1 | 0.339985623 | 2.77E−23 | C1qtnf12 | −0.402936511 | 5.43E−41 |
| Cct3 | 0.33974176 | 0.042095685 | Set | 0.559146201 | 7.08E−41 |
| Nol12 | 0.339482581 | 3.03E−12 | Tes | 0.562130172 | 7.20E−41 |
| Ifi35 | 0.33881065 | 1.98E−13 | 1810022K09Rik | 0.525873105 | 1.24E−40 |
| Eif3g | 0.33852835 | 4.24E−09 | Mgam | −0.735704656 | 1.26E−40 |
| Ruvbl1 | 0.338462287 | 7.36E−16 | Csrp2 | −0.560375337 | 1.33E−40 |
| U2af1 | 0.338200664 | 4.78E−18 | Abhd14b | −0.362968188 | 1.33E−40 |
| Slc25a17 | 0.338084362 | 5.79E−36 | Rpl37 | 0.344888551 | 1.71E−40 |
| Sqstm1 | 0.337178496 | 0.015003342 | Svip | −0.310834506 | 1.85E−40 |
| Rbbp4 | 0.336939609 | 6.33E−07 | Cenpm | 0.505571237 | 4.18E−40 |
| Mvb12a | 0.336588735 | 8.94E−10 | Actb | 0.316634018 | 5.01E−40 |
| Eif2s1 | 0.335954923 | 9.47E−13 | Pnkd | −0.372179138 | 5.86E−40 |
| Ube2e1 | 0.335594366 | 6.99E−18 | Dhrs4 | −0.439820418 | 7.12E−40 |
| Psma1 | 0.335396809 | 1.11E−16 | Slc6a8 | −0.745901216 | 7.88E−40 |
| Sigmar1 | 0.335361938 | 1.73E−11 | 2010107E04Rik | −0.347156133 | 2.51E−39 |
| Prrc2c | 0.334867399 | 0.083138552 | Ndufa6 | −0.383655196 | 3.01E−39 |
| Ifrd1 | 0.334734443 | 1.13E−07 | Rps12 | 0.391394992 | 3.41E−39 |
| Plp2 | 0.333759834 | 6.32E−12 | Cdk4 | 0.531596598 | 4.83E−39 |
| Eif3k | 0.333534521 | 2.46E−27 | Slc35b1 | −0.447145509 | 5.33E−39 |
| Ptrh2 | 0.332713796 | 1.70E−09 | Ehf | 0.519482609 | 5.42E−39 |
| Tmed5 | 0.332471467 | 7.18E−16 | Llph | 0.552175406 | 5.75E−39 |
| Pfdn4 | 0.332124237 | 2.25E−18 | Snrpb | 0.494797051 | 6.45E−39 |
| Exosc5 | 0.332023126 | 1.15E−06 | 1110004F10Rik | 0.518892113 | 7.75E−39 |
| Ppib | 0.331347699 | 6.95E−26 | Snhg12 | 0.589250403 | 7.89E−39 |
| Cbx3 | 0.331264531 | 0.001968256 | Tgif1 | 0.570306264 | 8.51E−39 |
| Cldn2 | 0.331186194 | 1.58E−06 | Rpl17 | 0.356823603 | 9.23E−39 |
| Ttc3 | 0.330957817 | 0.010882127 | Uba52 | 0.39061907 | 1.16E−38 |
| Ubxn1 | 0.330461333 | 7.40E−13 | Edf1 | −0.489683239 | 1.29E−38 |
| Spag7 | 0.330427988 | 1.93E−11 | Gipc2 | −0.414777529 | 1.41E−38 |
| Wars | 0.329944312 | 7.61E−11 | Mocs2 | −0.456126674 | 1.77E−38 |
| Rpl23a | 0.329650738 | 1.70E−25 | Eef1a1 | 0.3063645 | 2.26E−38 |
| Dnajc8 | 0.328305386 | 2.24E−11 | Naprt | −0.558072665 | 2.31E−38 |
| Brix1 | 0.327623057 | 1.92E−14 | Tob1 | −0.427049731 | 2.49E−38 |
| Strap | 0.326573324 | 4.49E−12 | Rpl32 | 0.352612181 | 2.69E−38 |
| Ddx18 | 0.325624426 | 1.84E−09 | Dbi | −0.499501979 | 3.05E−38 |
| Trim28 | 0.325474158 | 0.029250518 | Bola3 | −0.438659496 | 3.05E−38 |
| Acin1 | 0.325356794 | 0.001511853 | Fcgrt | −0.365553237 | 4.58E−38 |
| Dnajb6 | 0.325127127 | 3.17E−10 | Fus | 0.597738937 | 6.21E−38 |
| Bcl3 | 0.325078262 | 1.71E−12 | Hnf4a | −0.359250216 | 8.99E−38 |
| Psmb7 | 0.324796869 | 9.83E−09 | Cyb5b | −0.530452301 | 9.35E−38 |
| Junb | 0.324715832 | 0.010761727 | Papss2 | −1.144472832 | 1.16E−37 |
| Laptm4b | 0.324469966 | 3.13E−16 | Mrpl34 | −0.446366075 | 1.57E−37 |
| Psmc5 | 0.324367285 | 5.58E−07 | Vil1 | −0.849307777 | 1.86E−37 |
| Cct8 | 0.323129784 | 7.54E−05 | Nop56 | 0.628904677 | 2.38E−37 |
| Picalm | 0.322684422 | 1.62E−09 | Nenf | 0.500376562 | 3.36E−37 |
| Cd151 | 0.321897781 | 2.50E−10 | Ugdh | −0.536463292 | 3.55E−37 |
| Ddx39b | 0.321510325 | 7.45E−11 | Ociad2 | −0.452628993 | 4.76E−37 |
| Nip7 | 0.321158051 | 1.29E−17 | Coro2a | −0.420603316 | 6.31E−37 |
| Pttg1 | 0.320356298 | 2.17E−17 | Slc5a1 | −0.998301422 | 8.51E−37 |
| Rsrc2 | 0.319876808 | 0.027562641 | Sec31a | −0.425210347 | 9.54E−37 |
| Hmgb3 | 0.319371263 | 3.23E−10 | Tspan13 | −0.57994343 | 9.67E−37 |
| Comt | 0.319113036 | 5.24E−05 | Tmem253 | −0.461238263 | 1.69E−36 |
| Aimp1 | 0.319003449 | 1.76E−07 | Casp7 | −0.422348179 | 1.96E−36 |
| Nasp | 0.318696772 | 2.88E−08 | Ccdc34 | 0.587930115 | 2.10E−36 |
| Dhrs7 | 0.317475279 | 2.79E−08 | Rfk | −0.684214441 | 2.15E−36 |
| Med28 | 0.317395122 | 6.04E−14 | Echdc2 | 0.388234012 | 4.23E−36 |
| Snrpd3 | 0.317137536 | 6.06E−14 | Slc25a17 | 0.338084362 | 5.79E−36 |
| Actb | 0.316634018 | 5.01E−40 | Plcb3 | −0.319450575 | 6.84E−36 |
| Cops5 | 0.316571028 | 4.03E−10 | Gipc1 | 0.493097036 | 8.20E−36 |
| Ascc3 | 0.31633194 | 8.17E−05 | Zzef1 | −0.421625191 | 8.85E−36 |
| Leprotl1 | 0.316328201 | 1.20E−13 | Fam234a | −0.452589166 | 1.40E−35 |
| Tuba4a | 0.315844438 | 1.94E−09 | Tst | 0.410595687 | 1.58E−35 |
| Psmb1 | 0.31543236 | 3.90E−22 | mt-Nd5 | −0.344564337 | 1.71E−35 |
| Smarcb1 | 0.315345113 | 1.23E−08 | Mgat4a | −0.29749178 | 1.78E−35 |
| H2-DMa | 0.315340243 | 1.98E−05 | Dnajc2 | 0.531896529 | 1.99E−35 |
| Ascl2 | 0.31428101 | 7.58E−28 | Mafg | 0.52307048 | 2.22E−35 |
| Sptssa | 0.3140622 | 0.000449881 | Rpl35 | 0.343201254 | 2.98E−35 |
| Rnps1 | 0.313845402 | 0.00033971 | Hmgn2 | 0.611670656 | 5.14E−35 |
| Cebpz | 0.313513409 | 0.003039764 | Ptms | 0.537764589 | 5.26E−35 |
| Prpf40a | 0.313435585 | 3.20E−09 | Oaz1 | −0.327466761 | 7.78E−35 |
| Dhx9 | 0.312983121 | 0.013052942 | Cisd1 | −0.416794492 | 1.35E−34 |
| Anxa7 | 0.312845883 | 1.03E−08 | Rpl5 | 0.355322036 | 1.44E−34 |
| Snrpa | 0.312374761 | 2.10E−06 | Acadm | −0.343554677 | 1.78E−34 |
| Eif3m | 0.312002004 | 2.96E−10 | Acadvl | −0.318617788 | 3.23E−34 |
| Ubald2 | 0.311870228 | 0.000659028 | Psmd4 | 0.523779324 | 4.49E−34 |
| Nr2c2ap | 0.310686966 | 2.10E−07 | Smdt1 | −0.392155264 | 5.81E−34 |
| Rps8 | 0.310342301 | 5.83E−20 | Gjb1 | −0.307098693 | 9.90E−34 |
| Ccdc12 | 0.310323679 | 6.76E−08 | Sox9 | 0.525555927 | 1.07E−33 |
| Psmc1 | 0.30929546 | 2.40E−05 | Chchd2 | −0.309594003 | 1.35E−33 |
| Srsf5 | 0.309018357 | 7.47E−05 | Atp5b | −0.301344047 | 2.02E−33 |
| Rack1 | 0.308287523 | 3.16E−26 | Gch1 | 0.386576799 | 2.49E−33 |
| Swi5 | 0.308258264 | 2.42E−15 | Hcfc1r1 | −0.328833933 | 2.58E−33 |
| Eif5 | 0.307229994 | 7.01E−13 | Bcas2 | 0.517987714 | 4.14E−33 |
| Tsg101 | 0.306886147 | 4.38E−08 | Mrps36 | −0.422514018 | 4.34E−33 |
| Stub1 | 0.306829499 | 1.35E−10 | Tstd1 | −0.397885678 | 4.38E−33 |
| Rpl22 | 0.306672667 | 2.18E−23 | Pgrmc1 | −0.437684794 | 4.84E−33 |
| Sin3b | 0.306395208 | 1.08E−09 | Hsph1 | −0.333593162 | 6.59E−33 |
| Eef1a1 | 0.3063645 | 2.26E−38 | Rpl37a | 0.357064244 | 6.69E−33 |
| Phax | 0.305880967 | 7.30E−14 | Rnh1 | 0.52364773 | 7.37E−33 |
| Serinc2 | 0.305818819 | 3.73E−11 | Acot8 | −0.362932186 | 7.77E−33 |
| Ubap2l | 0.305693451 | 0.072408977 | Qdpr | −0.466058758 | 9.43E−33 |
| Sarnp | 0.305081441 | 2.02E−09 | Oit1 | −0.479949645 | 9.53E−33 |
| Vps72 | 0.304871638 | 1.24E−09 | H2-Aa | 0.482291221 | 9.77E−33 |
| Nsa2 | 0.303758726 | 3.46E−15 | Gpd1 | −0.533665315 | 1.06E−32 |
| Mrpl33 | 0.303389307 | 1.29E−09 | Ift20 | −0.431239634 | 1.22E−32 |
| Psmd12 | 0.303328982 | 2.04E−08 | Acot13 | −0.34675137 | 1.26E−32 |
| Tbcb | 0.303320735 | 1.41E−07 | Mlec | −0.296876184 | 1.45E−32 |
| Rps11 | 0.302983136 | 7.31E−29 | Kdelr2 | −0.407409764 | 2.05E−32 |
| Btf3l4 | 0.302650746 | 4.43E−12 | Cstb | 0.599545749 | 2.82E−32 |
| Eef1g | 0.302583649 | 7.89E−19 | Akr1c13 | −0.414596918 | 4.72E−32 |
| Mob4 | 0.302541447 | 0.002375664 | Psma2 | 0.44590197 | 5.28E−32 |
| Rab11a | 0.301692059 | 6.41E−06 | Ddx24 | 0.546297473 | 6.60E−32 |
| Smn1 | 0.301548688 | 9.06E−14 | Decr1 | −0.31800669 | 1.03E−31 |
| Hnrnpk | 0.301362792 | 0.000310239 | Ush1c | −0.370477883 | 1.08E−31 |
| Pbdc1 | 0.301009043 | 1.69E−11 | Duoxa2 | 0.73409713 | 1.09E−31 |
| Eif4a3 | 0.30087741 | 9.98E−10 | Srrm2 | 0.49979301 | 1.71E−31 |
| Eif3i | 0.300867215 | 2.70E−16 | Cd47 | 0.58233215 | 2.44E−31 |
| Utp14a | 0.300734646 | 4.16E−05 | Nr3c1 | −0.284039067 | 2.81E−31 |
| Litaf | 0.300385784 | 1.38E−14 | Qtrt1 | 0.46850658 | 4.44E−31 |
| Ube2n | 0.300170596 | 2.32E−10 | Hddc2 | 0.385200381 | 5.51E−31 |
| Psmd14 | 0.299487142 | 2.71E−08 | Parp14 | 0.514881917 | 6.52E−31 |
| Ctnnb1 | 0.299117018 | 0.00012311 | H1f0 | −0.351806643 | 6.86E−31 |
| Anp32e | 0.29883508 | 1.65E−10 | Mlx | −0.358688445 | 8.27E−31 |
| Actr10 | 0.298789009 | 2.44E−13 | Psmb4 | 0.427589034 | 8.97E−31 |
| Srsf3 | 0.29802184 | 3.70E−11 | Sap30 | 0.383350537 | 1.16E−30 |
| Ube2b | 0.297366839 | 7.47E−07 | Hnrnpdl | 0.503244887 | 1.31E−30 |
| Hmgcr | 0.29727832 | 4.84E−09 | Ckb | −0.614042802 | 2.40E−30 |
| Sdf4 | 0.296546743 | 5.27E−05 | Psip1 | 0.421154817 | 2.50E−30 |
| Ccdc59 | 0.296543738 | 5.87E−06 | Chic2 | 0.458178143 | 3.10E−30 |
| Mri1 | 0.296272237 | 2.35E−14 | Prdx6 | −0.384742391 | 4.16E−30 |
| Pdcd5 | 0.294336079 | 2.45E−10 | Acaa1a | −0.462544888 | 4.16E−30 |
| Wbp4 | 0.293739505 | 9.32E−05 | Med21 | 0.468205761 | 4.62E−30 |
| Npdc1 | 0.293703325 | 3.09E−14 | Zfyve21 | −0.290927324 | 4.99E−30 |
| Rrp1 | 0.293295131 | 0.016579435 | Ift43 | 0.435654376 | 7.55E−30 |
| Mapre1 | 0.292476151 | 0.000906864 | Clca3b | −0.623219586 | 8.22E−30 |
| Dhps | 0.291869663 | 2.47E−10 | Erh | 0.459585899 | 8.48E−30 |
| Eif6 | 0.291783132 | 3.19E−12 | Akr1c12 | −0.313659298 | 8.65E−30 |
| Krtcap3 | 0.291626008 | 3.29E−06 | Sssca1 | 0.490692739 | 8.74E−30 |
| Tmed9 | 0.29108885 | 1.05E−12 | Paics | 0.389643997 | 9.49E−30 |
| Psmd13 | 0.290946279 | 2.72E−07 | Prkar2a | −0.332855181 | 1.04E−29 |
| Pcbp2 | 0.290944421 | 2.49E−05 | Eif5b | 0.597518054 | 1.34E−29 |
| Eno1 | 0.290876393 | 4.24E−14 | Fmc1 | −0.340644337 | 1.53E−29 |
| Dtymk | 0.290677513 | 1.68E−07 | Olfm4 | −1.031013765 | 1.78E−29 |
| Rps21 | 0.290584821 | 5.23E−20 | Selenom | −1.629387915 | 1.85E−29 |
| Rnaseh2a | 0.29017094 | 5.51E−06 | Fbl | 0.477623403 | 2.52E−29 |
| Tcea1 | 0.290089616 | 0.077545527 | Rpl13a | 0.432398598 | 2.58E−29 |
| Baz1a | 0.289859889 | 2.57E−11 | Ncor1 | −0.316363293 | 3.54E−29 |
| Luc7l3 | 0.289550996 | 0.052198809 | Ndufa1 | −0.403279535 | 3.94E−29 |
| Imp3 | 0.289470656 | 8.24E−09 | Polr2f | 0.505391369 | 5.52E−29 |
| Rps27a | 0.288546449 | 2.45E−26 | Rps11 | 0.302983136 | 7.31E−29 |
| Snrpc | 0.288332568 | 7.76E−09 | Rpl13 | 0.340368867 | 7.50E−29 |
| Naxd | 0.28779077 | 0.001010318 | Cnbp | 0.429189553 | 7.63E−29 |
| Syncrip | 0.287677571 | 3.96E−06 | Ss18l2 | −0.309252124 | 8.66E−29 |
| Tsn | 0.287636289 | 3.00E−07 | Hadhb | −0.31095387 | 1.11E−28 |
| Ube2d2a | 0.287436326 | 5.47E−07 | Lasp1 | −0.377565006 | 1.23E−28 |
| Manbal | 0.287304049 | 1.06E−08 | Tap1 | 0.504238233 | 1.77E−28 |
| Psma5 | 0.286901769 | 2.62E−08 | Gda | −1.049045194 | 2.32E−28 |
| Psmb10 | 0.28689618 | 1.26E−08 | H2afy | 0.530401365 | 3.13E−28 |
| H2afz | 0.286608739 | 1.11E−08 | Pgp | −0.326276001 | 4.32E−28 |
| Tmem128 | 0.28613199 | 1.11E−09 | Magt1 | −0.264302208 | 4.44E−28 |
| Khdrbs1 | 0.284929706 | 1.30E−05 | Ndufv2 | −0.370737877 | 6.35E−28 |
| Rala | 0.284712627 | 1.81E−05 | 2610528J11Rik | −0.35398218 | 6.68E−28 |
| Id2 | 0.284398925 | 9.47E−11 | Tm4sf20 | −0.410081011 | 7.54E−28 |
| Arhgdia | 0.284340564 | 7.36E−06 | Ascl2 | 0.31428101 | 7.58E−28 |
| Rps29 | 0.283971031 | 2.16E−16 | Gmds | −0.30911867 | 1.42E−27 |
| Gnas | 0.283827428 | 2.86E−05 | Tspan15 | −0.349993937 | 1.51E−27 |
| Rere | 0.283674491 | 0.00017017 | Med10 | 0.394987249 | 1.53E−27 |
| Rpl15 | 0.283491416 | 1.73E−25 | Eif3k | 0.333534521 | 2.46E−27 |
| Rpl29 | 0.282691879 | 4.77E−27 | Rpl29 | 0.282691879 | 4.77E−27 |
| Top1 | 0.28251521 | 4.57E−07 | Smarca4 | 0.625119245 | 6.51E−27 |
| Naa20 | 0.281821784 | 2.57E−06 | Atp1b1 | −0.441092932 | 6.71E−27 |
| Tapbp | 0.28098354 | 0.000552101 | Tpm4 | 0.381565619 | 8.15E−27 |
| Psmd1 | 0.280850161 | 1.47E−06 | Snu13 | 0.439963161 | 8.62E−27 |
| Eif2b2 | 0.279949564 | 2.03E−07 | Ppp1r14b | 0.422633575 | 1.29E−26 |
| Ddx5 | 0.2799099 | 0.009913465 | 2010111I01Rik | 0.510842179 | 2.32E−26 |
| Trmt6 | 0.279761533 | 1.29E−12 | Rps27a | 0.288546449 | 2.45E−26 |
| Cks1b | 0.279744218 | 9.09E−06 | Cmpk1 | −0.381971422 | 2.50E−26 |
| Trappc4 | 0.277590408 | 8.93E−05 | Adipor2 | −0.301210162 | 3.00E−26 |
| Ube2l3 | 0.277508888 | 9.54E−09 | Grtp1 | −0.294449496 | 3.13E−26 |
| Arid4b | 0.277240367 | 0.000225064 | Rack1 | 0.308287523 | 3.16E−26 |
| Glmp | 0.2770004 | 0.007634214 | H3f3a | 0.340501928 | 3.39E−26 |
| Mrpl52 | 0.276849485 | 1.63E−13 | Hsd17b11 | −0.384074611 | 4.45E−26 |
| Pin1 | 0.276588109 | 8.54E−08 | Ndufa8 | −0.36666646 | 5.66E−26 |
| Hnrnpd | 0.276507479 | 0.000316215 | Glrx | −0.529827383 | 6.24E−26 |
| Rpl8 | 0.27608352 | 5.43E−17 | Urah | 0.543121281 | 6.50E−26 |
| Lsm4 | 0.275829238 | 1.10E−08 | Dsg2 | 0.573498239 | 6.71E−26 |
| Eif3e | 0.275156994 | 3.42E−07 | Ppp1r2 | 0.450885569 | 6.73E−26 |
| Rbbp7 | 0.274584031 | 0.022237948 | Ppib | 0.331347699 | 6.95E−26 |
| Cyth2 | 0.274551725 | 4.48E−12 | Cndp2 | −0.618329643 | 7.31E−26 |
| Lamtor1 | 0.274417718 | 1.70E−07 | Rrbp1 | −0.262220844 | 7.84E−26 |
| Eloc | 0.27412039 | 8.14E−08 | Mcfd2 | −0.299934223 | 8.19E−26 |
| Pole4 | 0.273858132 | 7.06E−07 | Fasn | 0.421266466 | 8.67E−26 |
| Eif3f | 0.273583491 | 1.01E−19 | Arpp19 | 0.447186675 | 1.07E−25 |
| Ppia | 0.272952623 | 1.11E−17 | Taf1d | 0.61372046 | 1.29E−25 |
| Uchl3 | 0.272771463 | 6.00E−07 | Arid5b | 0.491114705 | 1.64E−25 |
| Actl6a | 0.272352422 | 0.005073087 | Rpl23a | 0.329650738 | 1.70E−25 |
| Hspa8 | 0.272321923 | 0.066194338 | Rpl15 | 0.283491416 | 1.73E−25 |
| Mrpl54 | 0.271934125 | 1.50E−06 | Knop1 | 0.52211339 | 2.08E−25 |
| Hells | 0.271894126 | 6.36E−06 | Pomp | 0.362541456 | 2.85E−25 |
| Tmem234 | 0.271394284 | 1.04E−06 | Fermt1 | 0.58152364 | 2.99E−25 |
| Morf4l1 | 0.271267819 | 1.13E−06 | Gar1 | 0.422352807 | 3.39E−25 |
| Sh3bgrl2 | 0.270299652 | 0.008167256 | Sumo2 | 0.425657908 | 3.82E−25 |
| Cd320 | 0.270145227 | 1.11E−10 | Nsmce1 | 0.361700132 | 3.90E−25 |
| Irf8 | 0.269532227 | 0.009702893 | Lamtor2 | −0.335687305 | 4.66E−25 |
| Naca | 0.269191421 | 1.32E−21 | Ceacam10 | 0.839442139 | 4.86E−25 |
| Denr | 0.269003007 | 0.001276542 | Eif2ak2 | 0.404404965 | 4.90E−25 |
| Snrpb2 | 0.268425224 | 1.59E−06 | Myh14 | −0.417763713 | 8.14E−25 |
| Rps16 | 0.268162039 | 3.57E−21 | Lmo7 | 0.464053931 | 9.99E−25 |
| Nop16 | 0.268118652 | 4.43E−07 | Snrpd1 | 0.437807356 | 1.18E−24 |
| Glrx3 | 0.267211896 | 5.80E−05 | Pls1 | −0.784893116 | 1.22E−24 |
| Alyref | 0.266312927 | 0.000493358 | Siva1 | 0.434539718 | 1.49E−24 |
| Anapc16 | 0.266308747 | 0.085578683 | Smim22 | −0.505278847 | 1.56E−24 |
| Bloc1s2 | 0.266146194 | 4.90E−08 | Yif1a | −0.35354628 | 1.84E−24 |
| Lypla2 | 0.265273215 | 1.15E−07 | Ranbp1 | 0.436052632 | 2.27E−24 |
| Gm10076 | 0.264898455 | 0.082018344 | Gpx1 | −0.616669736 | 2.68E−24 |
| Psmd8 | 0.264445517 | 0.001143737 | Tmem9b | −0.31671224 | 2.78E−24 |
| H2-T23 | 0.264432203 | 2.80E−08 | Akr1b3 | 0.392411619 | 4.71E−24 |
| Rps26 | 0.263192495 | 8.33E−18 | Cct5 | 0.516284245 | 4.73E−24 |
| Rpl36a | 0.263120307 | 2.59E−18 | Lsm7 | 0.493557328 | 7.44E−24 |
| Lipt2 | 0.262600585 | 3.85E−10 | 0610012G03Rik | −0.287605778 | 7.71E−24 |
| Ciapin1 | 0.26202575 | 1.18E−09 | Park7 | 0.36675562 | 9.10E−24 |
| Sf3b4 | 0.261338475 | 6.86E−07 | Dynll2 | −0.322211671 | 9.27E−24 |
| Cdc42se1 | 0.261278433 | 1.29E−08 | Anp32b | 0.385439645 | 9.53E−24 |
| Kras | 0.260652399 | 0.026822653 | Raly | 0.450020983 | 1.10E−23 |
| Rac1 | 0.259728567 | 0.000100973 | Lgals9 | 0.452114357 | 1.34E−23 |
| Taf10 | 0.259344112 | 0.018372312 | Tcim | −0.374205739 | 1.39E−23 |
| Cetn3 | 0.259052088 | 2.67E−06 | Fcgbp | −3.780995317 | 1.39E−23 |
| Itgb1 | 0.258943099 | 0.004631202 | Epb41l4aos | 0.511645703 | 1.41E−23 |
| Akr1a1 | 0.258719174 | 2.94E−13 | Tmem41a | −0.281775011 | 1.42E−23 |
| Eif1a | 0.25782437 | 1.05E−05 | Rpl22 | 0.306672667 | 2.18E−23 |
| Fam103a1 | 0.257713322 | 7.30E−06 | G3bp2 | 0.421558109 | 2.29E−23 |
| Rps27 | 0.257664847 | 1.21E−18 | Cdc42ep5 | −0.3093568 | 2.42E−23 |
| Nop14 | 0.257540458 | 0.010133726 | Clic4 | 0.416788418 | 2.48E−23 |
| Emg1 | 0.257147521 | 9.02E−05 | Hmgb1 | 0.339985623 | 2.77E−23 |
| Srp14 | 0.257020902 | 0.010371554 | Tff3 | −4.112357926 | 3.06E−23 |
| Nfkbib | 0.256931162 | 1.18E−07 | Hif1a | 0.386338308 | 3.26E−23 |
| Dcakd | 0.25515788 | 1.82E−09 | Zfp593 | 0.397842204 | 3.70E−23 |
| Paip2 | 0.253830197 | 0.026595456 | Ost4 | −0.320453053 | 3.92E−23 |
| Psma6 | 0.253788107 | 5.42E−07 | AA467197 | −0.259595104 | 5.33E−23 |
| Tmod3 | 0.252577012 | 9.53E−05 | Ebna1bp2 | 0.438476224 | 6.05E−23 |
| Gtf3c6 | 0.252098038 | 0.00037306 | Snrpf | 0.38851751 | 6.09E−23 |
| Rfc3 | 0.252066046 | 1.71E−10 | Dpm3 | −0.313217799 | 6.47E−23 |
| Gmps | 0.251931124 | 0.000208401 | Psmc4 | 0.471117632 | 7.52E−23 |
| Sem1 | 0.251576492 | 2.81E−14 | H2afj | 0.398143977 | 7.55E−23 |
| Eef1d | 0.251256138 | 1.04E−10 | Tfg | −0.401536537 | 7.69E−23 |
| Rsl24d1 | 0.250282826 | 2.32E−07 | Chchd3 | −0.289958929 | 1.05E−22 |
| | | | Nedd4 | 0.519033243 | 1.09E−22 |
| | | | Npc2 | 0.458566649 | 1.58E−22 |
| | | | Cuedc2 | 0.404126479 | 1.80E−22 |
| | | | Rbx1 | 0.365602948 | 1.90E−22 |
| | | | Mpc1 | −0.323878516 | 1.93E−22 |
| | | | Gnl3 | 0.52119811 | 2.01E−22 |
| | | | Psmb3 | 0.34902439 | 2.03E−22 |
| | | | Srsf7 | 0.444749026 | 2.12E−22 |
| | | | Arl6ip1 | −0.390632776 | 2.30E−22 |
| | | | Hspe1 | −0.45201747 | 2.61E−22 |
| | | | Prr15 | 0.35970891 | 3.74E−22 |
| | | | Psmb1 | 0.31543236 | 3.90E−22 |
| | | | Psmb6 | 0.358929308 | 4.11E−22 |
| | | | Tmem45b | −0.304813315 | 4.13E−22 |
| | | | Snrpg | 0.361450593 | 4.28E−22 |
| | | | Lsr | 0.432650101 | 4.36E−22 |
| | | | Plscr1 | 0.383523491 | 5.86E−22 |
| | | | Fkbp2 | −0.292945794 | 6.51E−22 |
| | | | Tagln2 | 0.451557877 | 7.28E−22 |
| | | | Sec22b | −0.265747292 | 1.06E−21 |
| | | | Naca | 0.269191421 | 1.32E−21 |
| | | | Mrto4 | 0.404720969 | 1.32E−21 |
| | | | Pepd | −0.615934877 | 1.58E−21 |
| | | | Ndufb7 | −0.295078975 | 1.60E−21 |
| | | | Cd82 | −0.32308332 | 1.64E−21 |
| | | | Ppp1r1b | −0.266422205 | 2.11E−21 |
| | | | Chka | −0.419462144 | 3.09E−21 |
| | | | Gstp2 | −0.399306435 | 3.19E−21 |
| | | | Actn1 | 0.543949709 | 3.55E−21 |
| | | | Rps16 | 0.268162039 | 3.57E−21 |
| | | | 4931406C07Rik | −0.349513533 | 3.61E−21 |
| | | | Mrpl42 | −0.350908316 | 3.86E−21 |
| | | | Ptov1 | 0.481659068 | 3.95E−21 |
| | | | Srsf11 | 0.461610566 | 4.14E−21 |
| | | | Ndufa9 | −0.309859558 | 4.89E−21 |
| | | | Xdh | −0.821980512 | 7.44E−21 |
| | | | Apex1 | 0.378907101 | 9.50E−21 |
| | | | Mak16 | 0.427801643 | 1.10E−20 |
| | | | Polr2g | 0.354755071 | 1.14E−20 |
| | | | Gstm1 | −0.288261538 | 1.44E−20 |
| | | | Pitpna | −0.350127171 | 1.45E−20 |
| | | | Ipmk | −0.332761845 | 1.76E−20 |
| | | | Map1lc3b | 0.464727736 | 1.90E−20 |
| | | | Tomm20 | 0.393317534 | 2.57E−20 |
| | | | Nudt19 | −0.292246985 | 2.84E−20 |
| | | | Slc31a1 | −0.31508973 | 3.43E−20 |
| | | | Plin3 | −0.451871475 | 4.97E−20 |
| | | | Rps21 | 0.290584821 | 5.23E−20 |
| | | | Rps8 | 0.310342301 | 5.83E−20 |
| | | | Tm9sf3 | −0.282872141 | 6.00E−20 |
| | | | Slc3a2 | −0.256973252 | 6.85E−20 |
| | | | Tuba1b | 0.491039759 | 7.17E−20 |
| | | | Myl12b | 0.473766996 | 7.53E−20 |
| | | | Gpx4 | −0.605930619 | 7.59E−20 |
| | | | Smarcc1 | 0.526590873 | 7.76E−20 |
| | | | Ndufs5 | −0.336580838 | 8.77E−20 |
| | | | Usp22 | 0.376977518 | 8.79E−20 |
| | | | Ndufv1 | −0.286789801 | 9.58E−20 |
| | | | Eif3f | 0.273583491 | 1.01E−19 |
| | | | Gnai2 | 0.346645896 | 1.13E−19 |
| | | | Glul | 0.564517314 | 1.46E−19 |
| | | | Tmed4 | −0.314823509 | 1.49E−19 |
| | | | Ube2s | 0.502935149 | 1.51E−19 |
| | | | Map1lc3a | 0.409624614 | 1.86E−19 |
| | | | Mcrip1 | 0.39110356 | 1.96E−19 |
| | | | Kcne3 | 0.491572881 | 2.35E−19 |
| | | | Nmi | 0.406945591 | 3.90E−19 |
| | | | Sar1b | −0.359426333 | 4.58E−19 |
| | | | Tpm3 | 0.449198494 | 4.67E−19 |
| | | | Eef1e1 | 0.396901383 | 4.70E−19 |
| | | | Azin1 | −0.337939999 | 6.13E−19 |
| | | | Sertad1 | 0.368733524 | 6.88E−19 |
| | | | Eef1g | 0.302583649 | 7.89E−19 |
| | | | Grn | 0.478121965 | 9.92E−19 |
| | | | Plekhb2 | −0.342782801 | 1.03E−18 |
| | | | Rps27 | 0.257664847 | 1.21E−18 |
| | | | Gabarap | −0.26307422 | 1.32E−18 |
| | | | Snrpd2 | 0.371064889 | 1.42E−18 |
| | | | Mgst2 | −0.359923689 | 1.54E−18 |
| | | | Ran | 0.361584771 | 1.87E−18 |
| | | | Spcs2 | −0.281998442 | 1.91E−18 |
| | | | Pfdn4 | 0.332124237 | 2.25E−18 |
| | | | Rpl36a | 0.263120307 | 2.59E−18 |
| | | | Lars | 0.385517463 | 2.75E−18 |
| | | | Rbm25 | 0.458969126 | 3.35E−18 |
| | | | Selenos | −0.363105116 | 4.00E−18 |
| | | | U2af1 | 0.338200664 | 4.78E−18 |
| | | | Prpsap1 | −0.286637172 | 5.12E−18 |
| | | | Eif1ax | 0.35469496 | 5.56E−18 |
| | | | Cmc2 | −0.25970224 | 5.94E−18 |
| | | | Srrm1 | 0.395328441 | 6.35E−18 |
| | | | Sult2b1 | −0.458179269 | 6.98E−18 |
| | | | Ube2e1 | 0.335594366 | 6.99E−18 |
| | | | Agr2 | −3.046098227 | 7.85E−18 |
| | | | Rps26 | 0.263192495 | 8.33E−18 |
| | | | Smarce1 | 0.400204968 | 9.32E−18 |
| | | | Cct2 | 0.407470261 | 1.09E−17 |
| | | | Pfdn2 | 0.404554694 | 1.10E−17 |
| | | | Ppia | 0.272952623 | 1.11E−17 |
| | | | Pkm | 0.3553343 | 1.12E−17 |
| | | | Nip7 | 0.321158051 | 1.29E−17 |
| | | | Pnp2 | −0.314773354 | 1.35E−17 |
| | | | Bnip3l | 0.443980481 | 1.66E−17 |
| | | | Bax | 0.378400471 | 1.69E−17 |
| | | | Pttg1 | 0.320356298 | 2.17E−17 |
| | | | Cox7a2l | −0.267197543 | 3.88E−17 |
| | | | Pdap1 | 0.420709617 | 3.95E−17 |
| | | | Fam60a | 0.359356795 | 4.29E−17 |
| | | | Vapa | −0.255877115 | 5.16E−17 |
| | | | Rpl8 | 0.27608352 | 5.43E−17 |
| | | | Adrm1 | 0.383437568 | 5.89E−17 |
| | | | Dnajc3 | −0.275087269 | 8.12E−17 |
| | | | Bri3bp | −0.254369006 | 8.99E−17 |
| | | | Ilf2 | 0.441261082 | 9.32E−17 |
| | | | Bola2 | 0.446510589 | 1.02E−16 |
| | | | Selenoi | −0.280135474 | 1.03E−16 |
| | | | Psma1 | 0.335396809 | 1.11E−16 |
| | | | Cnn3 | 0.374980903 | 1.26E−16 |
| | | | Klf4 | −0.429272963 | 1.48E−16 |
| | | | Nubp1 | 0.361259922 | 1.51E−16 |
| | | | Pa2g4 | 0.341365009 | 1.98E−16 |
| | | | Rps29 | 0.283971031 | 2.16E−16 |
| | | | Pabpc1 | 0.429235751 | 2.41E−16 |
| | | | Eif3i | 0.300867215 | 2.70E−16 |
| | | | Fkbp3 | 0.372829105 | 3.00E−16 |
| | | | Laptm4b | 0.324469966 | 3.13E−16 |
| | | | Ethe1 | −0.375918347 | 3.56E−16 |
| | | | Fcf1 | 0.388376244 | 4.73E−16 |
| | | | Pnp | −0.271530119 | 5.05E−16 |
| | | | Dctn3 | 0.3664681 | 5.70E−16 |
| | | | 2210016F16Rik | −0.25002822 | 5.92E−16 |
| | | | Ssb | 0.385381868 | 6.00E−16 |
| | | | Vamp8 | −0.288648727 | 6.24E−16 |
| | | | Ezh2 | 0.423108166 | 6.45E−16 |
| | | | Tmed5 | 0.332471467 | 7.18E−16 |
| | | | Ruvbl1 | 0.338462287 | 7.36E−16 |
| | | | Pgls | 0.366335327 | 7.61E−16 |
| | | | Fdps | 0.380838196 | 7.82E−16 |
| | | | Lypla1 | −0.286801911 | 8.22E−16 |
| | | | Psmb9 | 0.403486064 | 8.26E−16 |
| | | | Hspd1 | −0.422386534 | 9.78E−16 |
| | | | Tcof1 | 0.442849646 | 9.94E−16 |
| | | | Mybbp1a | 0.473164565 | 1.05E−15 |
| | | | Mkrn1 | −0.261924976 | 1.10E−15 |
| | | | Bola1 | 0.361063463 | 1.31E−15 |
| | | | Eps8l3 | −0.276806185 | 1.34E−15 |
| | | | Serp1 | −0.263678596 | 1.40E−15 |
| | | | Reg3b | −0.321548346 | 1.52E−15 |
| | | | Cxadr | 0.736630652 | 1.60E−15 |
| | | | Ndfip2 | −0.288312546 | 2.06E−15 |
| | | | Swi5 | 0.308258264 | 2.42E−15 |
| | | | Mcu | −0.283040643 | 2.50E−15 |
| | | | Dnajc19 | −0.280723772 | 2.81E−15 |
| | | | Itpk1 | −0.299367136 | 3.30E−15 |
| | | | Nsa2 | 0.303758726 | 3.46E−15 |
| | | | Mien1 | −0.298833217 | 4.14E−15 |
| | | | Psmc3 | 0.396334867 | 4.75E−15 |
| | | | Cs | −0.297181959 | 5.40E−15 |
| | | | Tpm1 | 0.431354322 | 5.60E−15 |
| | | | Pak1 | 0.341660735 | 6.41E−15 |
| | | | Zfp36 | −0.264543061 | 7.31E−15 |
| | | | Mtdh | 0.403293096 | 7.84E−15 |
| | | | Spint1 | −0.272008674 | 9.12E−15 |
| | | | Gpx2 | 0.545160612 | 9.65E−15 |
| | | | Laptm4a | 0.416484811 | 1.02E−14 |
| | | | Tm9sf2 | −0.250077697 | 1.10E−14 |
| | | | Litaf | 0.300385784 | 1.38E−14 |
| | | | Brix1 | 0.327623057 | 1.92E−14 |
| | | | Dcun1d5 | 0.361558848 | 1.97E−14 |
| | | | Exosc4 | 0.414678434 | 2.06E−14 |
| | | | Hprt | 0.381457941 | 2.27E−14 |
| | | | Mri1 | 0.296272237 | 2.35E−14 |
| | | | Vars | 0.643884682 | 2.61E−14 |
| | | | Sem1 | 0.251576492 | 2.81E−14 |
| | | | Eef2 | 0.398465 | 2.92E−14 |
| | | | Tcp1 | 0.379372323 | 2.94E−14 |
| | | | Npdc1 | 0.293703325 | 3.09E−14 |
| | | | Ppa1 | −0.302351504 | 3.48E−14 |
| | | | Fkbp1a | 0.345776919 | 4.10E−14 |
| | | | Eno1 | 0.290876393 | 4.24E−14 |
| | | | Mrps14 | −0.277423152 | 4.68E−14 |
| | | | Psmg4 | 0.354825035 | 5.18E−14 |
| | | | Cct7 | 0.450995737 | 5.24E−14 |
| | | | Itga6 | 0.457592241 | 5.73E−14 |
| | | | Med28 | 0.317395122 | 6.04E−14 |
| | | | Snrpd3 | 0.317137536 | 6.06E−14 |
| | | | Hbegf | −0.392040025 | 6.51E−14 |
| | | | Eif3d | 0.441386655 | 6.75E−14 |
| | | | Abcd3 | −0.257665617 | 7.17E−14 |
| | | | Phax | 0.305880967 | 7.30E−14 |
| | | | Smn1 | 0.301548688 | 9.06E−14 |
| | | | Copb2 | −0.25026146 | 1.10E−13 |
| | | | Gtpbp4 | 0.373039031 | 1.19E−13 |
| | | | Leprotl1 | 0.316328201 | 1.20E−13 |
| | | | Id3 | 0.655528295 | 1.46E−13 |
| | | | Mrpl52 | 0.276849485 | 1.63E−13 |
| | | | Ifi35 | 0.33881065 | 1.98E−13 |
| | | | Exoc3l4 | −0.39623885 | 2.03E−13 |
| | | | Rabac1 | 0.382827516 | 2.17E−13 |
| | | | Hmga1 | 0.342416704 | 2.23E−13 |
| | | | Txn2 | −0.271661134 | 2.43E−13 |
| | | | Actr10 | 0.298789009 | 2.44E−13 |
| | | | Akr1a1 | 0.258719174 | 2.94E−13 |
| | | | Il18 | −0.436958556 | 3.76E−13 |
| | | | Ddx39 | 0.384385367 | 4.68E−13 |
| | | | Ufc1 | 0.344449905 | 5.43E−13 |
| | | | Tia1 | 0.346513626 | 6.81E−13 |
| | | | Eif5 | 0.307229994 | 7.01E−13 |
| | | | Ubxn1 | 0.330461333 | 7.40E−13 |
| | | | Srm | 0.352548488 | 8.94E−13 |
| | | | Eif2s1 | 0.335954923 | 9.47E−13 |
| | | | Lgals3bp | 0.372622015 | 1.03E−12 |
| | | | Tmed9 | 0.29108885 | 1.05E−12 |
| | | | Gm42031 | −0.358997169 | 1.13E−12 |
| | | | Nras | 0.351909624 | 1.22E−12 |
| | | | Trmt6 | 0.279761533 | 1.29E−12 |
| | | | Grcc10 | 0.393096389 | 1.34E−12 |
| | | | Bcl3 | 0.325078262 | 1.71E−12 |
| | | | Psmc2 | 0.355407852 | 2.62E−12 |
| | | | Nol12 | 0.339482581 | 3.03E−12 |
| | | | Eif6 | 0.291783132 | 3.19E−12 |
| | | | Snrnp70 | 0.437492847 | 3.40E−12 |
| | | | Znhit1 | 0.392417982 | 3.48E−12 |
| | | | Tmem258 | −0.252714111 | 4.07E−12 |
| | | | Btf3l4 | 0.302650746 | 4.43E−12 |
| | | | Psmc6 | 0.367955511 | 4.45E−12 |
| | | | Cyth2 | 0.274551725 | 4.48E−12 |
| | | | Strap | 0.326573324 | 4.49E−12 |
| | | | Arl6ip4 | 0.397305537 | 4.74E−12 |
| | | | Atox1 | 0.366187022 | 5.18E−12 |
| | | | Sf3b3 | 0.442486374 | 5.58E−12 |
| | | | Otub1 | 0.360594404 | 5.85E−12 |
| | | | Plp2 | 0.333759834 | 6.32E−12 |
| | | | Cdca7 | 0.446686738 | 6.87E−12 |
| | | | Chd4 | 0.520991265 | 1.20E−11 |
| | | | Mrpl17 | 0.348114134 | 1.21E−11 |
| | | | Mphosph10 | 0.344417443 | 1.36E−11 |
| | | | Pbdc1 | 0.301009043 | 1.69E−11 |
| | | | Sigmar1 | 0.335361938 | 1.73E−11 |
| | | | Nolc1 | 0.446349718 | 1.84E−11 |
| | | | Spag7 | 0.330427988 | 1.93E−11 |
| | | | 1810058I24Rik | 0.36052704 | 1.97E−11 |
| | | | Pcyt2 | −0.373104343 | 2.00E−11 |
| | | | Dnajc8 | 0.328305386 | 2.24E−11 |
| | | | Baz1a | 0.289859889 | 2.57E−11 |
| | | | Akap13 | 0.387426393 | 3.17E−11 |
| | | | Srsf3 | 0.29802184 | 3.70E−11 |
| | | | Serinc2 | 0.305818819 | 3.73E−11 |
| | | | Vgll4 | 0.399229365 | 4.23E−11 |
| | | | F11r | 0.391538559 | 5.03E−11 |
| | | | Nop58 | 0.403248257 | 6.22E−11 |
| | | | Bclaf1 | 0.440400513 | 7.30E−11 |
| | | | Smarca5 | 0.388443156 | 7.32E−11 |
| | | | Ddx39b | 0.321510325 | 7.45E−11 |
| | | | Id1 | 0.482451874 | 7.49E−11 |
| | | | Wars | 0.329944312 | 7.61E−11 |
| | | | Irf1 | 0.514594688 | 9.04E−11 |
| | | | Pak2 | 0.347286126 | 9.14E−11 |
| | | | Crb3 | 0.356369106 | 9.23E−11 |
| | | | Id2 | 0.284398925 | 9.47E−11 |
| | | | Eef1d | 0.251256138 | 1.04E−10 |
| | | | Cd320 | 0.270145227 | 1.11E−10 |
| | | | Stub1 | 0.306829499 | 1.35E−10 |
| | | | Anp32e | 0.29883508 | 1.65E−10 |
| | | | Rfc3 | 0.252066046 | 1.71E−10 |
| | | | Agpat2 | −0.344641768 | 2.11E−10 |
| | | | Ube2n | 0.300170596 | 2.32E−10 |
| | | | Pdcd5 | 0.294336079 | 2.45E−10 |
| | | | Dhps | 0.291869663 | 2.47E−10 |
| | | | Cd151 | 0.321897781 | 2.50E−10 |
| | | | Eif4ebp1 | 0.399544867 | 2.51E−10 |
| | | | Eif3m | 0.312002004 | 2.96E−10 |
| | | | Dnajb6 | 0.325127127 | 3.17E−10 |
| | | | Hmgb3 | 0.319371263 | 3.23E−10 |
| | | | Tuba1c | 0.400510568 | 3.41E−10 |
| | | | Nemf | 0.380395404 | 3.48E−10 |
| | | | Ctsd | 0.44749562 | 3.67E−10 |
| | | | Lipt2 | 0.262600585 | 3.85E−10 |
| | | | Cops5 | 0.316571028 | 4.03E−10 |
| | | | Mat2a | 0.388514634 | 4.41E−10 |
| | | | Fam46a | 0.347416284 | 4.53E−10 |
| | | | Trp53 | 0.35926038 | 5.66E−10 |
| | | | Sdf2l1 | −0.287051238 | 5.92E−10 |
| | | | Hnrnpu | 0.396536774 | 6.17E−10 |
| | | | Trappc6a | 0.341354279 | 6.80E−10 |
| | | | Mvb12a | 0.336588735 | 8.94E−10 |
| | | | Eif4a3 | 0.30087741 | 9.98E−10 |
| | | | Sin3b | 0.306395208 | 1.08E−09 |
| | | | Tmem128 | 0.28613199 | 1.11E−09 |
| | | | Ciapin1 | 0.26202575 | 1.18E−09 |
| | | | Pafah1b3 | 0.34298871 | 1.22E−09 |
| | | | Vps72 | 0.304871638 | 1.24E−09 |
| | | | Mrpl33 | 0.303389307 | 1.29E−09 |
| | | | Nono | 0.355744574 | 1.58E−09 |
| | | | Ywhae | 0.345750121 | 1.60E−09 |
| | | | Picalm | 0.322684422 | 1.62E−09 |
| | | | Ptrh2 | 0.332713796 | 1.70E−09 |
| | | | Lyar | 0.361357624 | 1.75E−09 |
| | | | Txndc9 | 0.344552491 | 1.79E−09 |
| | | | Tpd52l2 | 0.381959962 | 1.79E−09 |
| | | | Dcakd | 0.25515788 | 1.82E−09 |
| | | | Ddx18 | 0.325624426 | 1.84E−09 |
| | | | Tuba4a | 0.315844438 | 1.94E−09 |
| | | | Sarnp | 0.305081441 | 2.02E−09 |
| | | | Eif4g2 | 0.352891091 | 2.63E−09 |
| | | | Prpf40a | 0.313435585 | 3.20E−09 |
| | | | Rbm39 | 0.353640901 | 3.98E−09 |
| | | | Eif3g | 0.33852835 | 4.24E−09 |
| | | | Rabggtb | 0.373114263 | 4.27E−09 |
| | | | Hmgcr | 0.29727832 | 4.84E−09 |
| | | | Espn | −0.747697761 | 4.85E−09 |
| | | | Hsp90b1 | −0.28396515 | 5.91E−09 |
| | | | Tpr | 0.376540898 | 6.40E−09 |
| | | | Snrpc | 0.288332568 | 7.76E−09 |
| | | | Imp3 | 0.289470656 | 8.24E−09 |
| | | | Ube2l3 | 0.277508888 | 9.54E−09 |
| | | | Psmb7 | 0.324796869 | 9.83E−09 |
| | | | Anxa7 | 0.312845883 | 1.03E−08 |
| | | | Manbal | 0.287304049 | 1.06E−08 |
| | | | Lsm4 | 0.275829238 | 1.10E−08 |
| | | | H2afz | 0.286608739 | 1.11E−08 |
| | | | Smarcb1 | 0.315345113 | 1.23E−08 |
| | | | Psmb10 | 0.28689618 | 1.26E−08 |
| | | | Cdc42se1 | 0.261278433 | 1.29E−08 |
| | | | Cdc37 | 0.350025139 | 1.62E−08 |
| | | | Psmd12 | 0.303328982 | 2.04E−08 |
| | | | Psma5 | 0.286901769 | 2.62E−08 |
| | | | Sltm | 0.353137588 | 2.68E−08 |
| | | | Psmd14 | 0.299487142 | 2.71E−08 |
| | | | Dhrs7 | 0.317475279 | 2.79E−08 |
| | | | H2-T23 | 0.264432203 | 2.80E−08 |
| | | | Nasp | 0.318696772 | 2.88E−08 |
| | | | Ddx21 | 0.415499844 | 2.96E−08 |
| | | | Ssrp1 | 0.410703481 | 3.34E−08 |
| | | | Capns1 | 0.362005167 | 3.81E−08 |
| | | | Lamp1 | −0.27938939 | 3.92E−08 |
| | | | Tsg101 | 0.306886147 | 4.38E−08 |
| | | | Bloc1s2 | 0.266146194 | 4.90E−08 |
| | | | Tra2a | 0.4882779 | 5.61E−08 |
| | | | Ccdc12 | 0.310323679 | 6.76E−08 |
| | | | Lpcat3 | −0.26018088 | 7.29E−08 |
| | | | Eloc | 0.27412039 | 8.14E−08 |
| | | | Pin1 | 0.276588109 | 8.54E−08 |
| | | | Esf1 | 0.350355004 | 1.02E−07 |
| | | | Sdhaf1 | 0.356584769 | 1.10E−07 |
| | | | Ifrd1 | 0.334734443 | 1.13E−07 |
| | | | Lypla2 | 0.265273215 | 1.15E−07 |
| | | | Nfkbib | 0.256931162 | 1.18E−07 |
| | | | Zfp36l1 | 0.410096152 | 1.37E−07 |
| | | | Tbcb | 0.303320735 | 1.41E−07 |
| | | | Dtymk | 0.290677513 | 1.68E−07 |
| | | | Lamtor1 | 0.274417718 | 1.70E−07 |
| | | | Aimp1 | 0.319003449 | 1.76E−07 |
| | | | Eif2b2 | 0.279949564 | 2.03E−07 |
| | | | Nr2c2ap | 0.310686966 | 2.10E−07 |
| | | | Rsl24d1 | 0.250282826 | 2.32E−07 |
| | | | Psmd13 | 0.290946279 | 2.72E−07 |
| | | | Tsn | 0.287636289 | 3.00E−07 |
| | | | Eif3e | 0.275156994 | 3.42E−07 |
| | | | Nop16 | 0.268118652 | 4.43E−07 |
| | | | Top1 | 0.28251521 | 4.57E−07 |
| | | | Psma6 | 0.253788107 | 5.42E−07 |
| | | | Ube2d2a | 0.287436326 | 5.47E−07 |
| | | | Psmc5 | 0.324367285 | 5.58E−07 |
| | | | Uchl3 | 0.272771463 | 6.00E−07 |
| | | | Rbbp4 | 0.336939609 | 6.33E−07 |
| | | | Sf3b4 | 0.261338475 | 6.86E−07 |
| | | | Prom1 | 0.464681053 | 6.93E−07 |
| | | | Pole4 | 0.273858132 | 7.06E−07 |
| | | | Ube2b | 0.297366839 | 7.47E−07 |
| | | | Mbnl1 | 0.409785421 | 9.19E−07 |
| | | | Csnk1a1 | 0.357545271 | 9.95E−07 |
| | | | Tmem234 | 0.271394284 | 1.04E−06 |
| | | | Morf4l1 | 0.271267819 | 1.13E−06 |
| | | | Exosc5 | 0.332023126 | 1.15E−06 |
| | | | Smim6 | −0.359552538 | 1.17E−06 |
| | | | Ppan | 0.348148313 | 1.38E−06 |
| | | | Psmd1 | 0.280850161 | 1.47E−06 |
| | | | Mrpl54 | 0.271934125 | 1.50E−06 |
| | | | Cldn2 | 0.331186194 | 1.58E−06 |
| | | | Snrpb2 | 0.268425224 | 1.59E−06 |
| | | | Srsf10 | 0.425738777 | 1.66E−06 |
| | | | Snrpa | 0.312374761 | 2.10E−06 |
| | | | Utp3 | 0.354293612 | 2.18E−06 |
| | | | Chd9 | 0.377817235 | 2.28E−06 |
| | | | Lmna | 0.342393928 | 2.51E−06 |
| | | | Naa20 | 0.281821784 | 2.57E−06 |
| | | | Cetn3 | 0.259052088 | 2.67E−06 |
| | | | Scaf11 | 0.370298846 | 3.13E−06 |
| | | | Krtcap3 | 0.291626008 | 3.29E−06 |
| | | | Syncrip | 0.287677571 | 3.96E−06 |
| | | | Kif5b | 0.340395188 | 4.90E−06 |
| | | | Rnaseh2a | 0.29017094 | 5.51E−06 |
| | | | Ccdc59 | 0.296543738 | 5.87E−06 |
| | | | Hells | 0.271894126 | 6.36E−06 |
| | | | Rab11a | 0.301692059 | 6.41E−06 |
| | | | Fam103a1 | 0.257713322 | 7.30E−06 |
| | | | Arhgdia | 0.284340564 | 7.36E−06 |
| | | | Cks1b | 0.279744218 | 9.09E−06 |
| | | | Eif1a | 0.25782437 | 1.05E−05 |
| | | | Rtf1 | 0.354748237 | 1.08E−05 |
| | | | Khdrbs1 | 0.284929706 | 1.30E−05 |
| | | | Puf60 | 0.348475872 | 1.71E−05 |
| | | | Rala | 0.284712627 | 1.81E−05 |
| | | | H2-DMa | 0.315340243 | 1.98E−05 |
| | | | Psmc1 | 0.30929546 | 2.40E−05 |
| | | | Pcbp2 | 0.290944421 | 2.49E−05 |
| | | | Gnas | 0.283827428 | 2.86E−05 |
| | | | Mt1 | 0.499150571 | 3.24E−05 |
| | | | Utp14a | 0.300734646 | 4.16E−05 |
| | | | Comt | 0.319113036 | 5.24E−05 |
| | | | Sdf4 | 0.296546743 | 5.27E−05 |
| | | | Zfand5 | 0.348143396 | 5.55E−05 |
| | | | Glrx3 | 0.267211896 | 5.80E−05 |
| | | | Ralbp1 | 0.358824523 | 6.10E−05 |
| | | | Srsf5 | 0.309018357 | 7.47E−05 |
| | | | Cct8 | 0.323129784 | 7.54E−05 |
| | | | Ascc3 | 0.31633194 | 8.17E−05 |
| | | | Trappc4 | 0.277590408 | 8.93E−05 |
| | | | Emg1 | 0.257147521 | 9.02E−05 |
| | | | Wbp4 | 0.293739505 | 9.32E−05 |
| | | | Tmod3 | 0.252577012 | 9.53E−05 |
| | | | Rac1 | 0.259728567 | 0.000100973 |
| | | | Ctnnb1 | 0.299117018 | 0.00012311 |
| | | | Rere | 0.283674491 | 0.00017017 |
| | | | Lyz1 | −2.971724924 | 0.000174874 |
| | | | Gmps | 0.251931124 | 0.000208401 |
| | | | Arid4b | 0.277240367 | 0.000225064 |
| | | | Stard10 | 0.385465347 | 0.000278907 |
| | | | Hnrnpk | 0.301362792 | 0.000310239 |
| | | | Hnrnpd | 0.276507479 | 0.000316215 |
| | | | Rnps1 | 0.313845402 | 0.00033971 |
| | | | Aplp2 | 0.473379922 | 0.000353321 |
| | | | Gtf3c6 | 0.252098038 | 0.00037306 |
| | | | Sars | 0.373984462 | 0.000388026 |
| | | | Sptssa | 0.3140622 | 0.000449881 |
| | | | Alyref | 0.266312927 | 0.000493358 |
| | | | Tapbp | 0.28098354 | 0.000552101 |
| | | | Ubald2 | 0.311870228 | 0.000659028 |
| | | | Mapre1 | 0.292476151 | 0.000906864 |
| | | | Naxd | 0.28779077 | 0.001010318 |
| | | | Psmd8 | 0.264445517 | 0.001143737 |
| | | | Atrx | 0.395665438 | 0.00126614 |
| | | | Denr | 0.269003007 | 0.001276542 |
| | | | Acin1 | 0.325356794 | 0.001511853 |
| | | | Cbx3 | 0.331264531 | 0.001968256 |
| | | | Mob4 | 0.302541447 | 0.002375664 |
| | | | Lap3 | −0.304378075 | 0.002963589 |
| | | | Cebpz | 0.313513409 | 0.003039764 |
| | | | Itgb1 | 0.258943099 | 0.004631202 |
| | | | Ewsr1 | 0.355998881 | 0.004961259 |
| | | | Actl6a | 0.272352422 | 0.005073087 |
| | | | Defa30 | −3.509686375 | 0.007269587 |
| | | | Glmp | 0.2770004 | 0.007634214 |
| | | | Defa24 | −4.227250321 | 0.007857069 |
| | | | Slc38a2 | 0.399854091 | 0.008030202 |
| | | | Sh3bgrl2 | 0.270299652 | 0.008167256 |
| | | | Irf8 | 0.269532227 | 0.009702893 |
| | | | Ddx5 | 0.2799099 | 0.009913465 |
| | | | Nop14 | 0.257540458 | 0.010133726 |
| | | | Trib1 | 0.376748988 | 0.010351265 |
| | | | Srp14 | 0.257020902 | 0.010371554 |
| | | | Junb | 0.324715832 | 0.010761727 |
| | | | Ttc3 | 0.330957817 | 0.010882127 |
| | | | Dhx9 | 0.312983121 | 0.013052942 |
| | | | Sqstm1 | 0.337178496 | 0.015003342 |
| | | | Rrp1 | 0.293295131 | 0.016579435 |
| | | | Taf10 | 0.259344112 | 0.018372312 |
| | | | Rbbp7 | 0.274584031 | 0.022237948 |
| | | | Eif3a | 0.346966574 | 0.023778101 |
| | | | Paip2 | 0.253830197 | 0.026595456 |
| | | | Zg16 | −4.955906065 | 0.026742306 |
| | | | Kras | 0.260652399 | 0.026822653 |
| | | | Rsrc2 | 0.319876808 | 0.027562641 |
| | | | Trim28 | 0.325474158 | 0.029250518 |
| | | | Vcp | 0.429896338 | 0.033914108 |
| | | | Cct3 | 0.33974176 | 0.042095685 |
| | | | Luc7l3 | 0.289550996 | 0.052198809 |
| | | | Sf3b2 | 0.363597224 | 0.055810021 |
| | | | Hspa8 | 0.272321923 | 0.066194338 |
| | | | Ubap2l | 0.305693451 | 0.072408977 |
| | | | Tcea1 | 0.290089616 | 0.077545527 |
| | | | Gm10076 | 0.264898455 | 0.082018344 |
| | | | Prrc2c | 0.334867399 | 0.083138552 |
| | | | Anapc16 | 0.266308747 | 0.085578683 |

TABLE 7
scRNA-seq Cell Type Proportions
Genetically engineered mouse model

| | Lgr5- | Lgr5- | Lgr5- | |
|---|---|---|---|---|
| | Apc^{+/+} | Apc^{f/f} | Apc^{f/f} | |
| Cell Type | Sox9^{+/+} | Sox9^{+/+} | Sox9^{f/+} | Combined |
| AbSC (Aberrant Stem Cell-like) | 0.007 | 0.588 | 0.123 | 0.244 |
| ISC/TA (Intestinal Stem Cell/ | 0.441 | 0.122 | 0.265 | 0.296 |
| Transit Amplifying) | | | | |
| TA/EP (Transit Amplifying/ | 0.154 | 0.039 | 0.138 | 0.108 |
| Enterocyte Progenitor) | | | | |
| EP/E (Enterocyte Progenitor/ | 0.153 | 0.080 | 0.201 | 0.132 |
| Enterocyte) | | | | |
| Enterocyte | 0.086 | 0.042 | 0.133 | 0.075 |
| Goblet | 0.106 | 0.042 | 0.087 | 0.079 |
| Paneth | 0.015 | 0.055 | 0.034 | 0.033 |
| Tuft | 0.018 | 0.020 | 0.005 | 0.017 |
| EEC (Enteroendocrine Cell) | 0.019 | 0.013 | 0.015 | 0.016 |
| Total | 1.000 | 1.000 | 1.000 | 1.000 |

Human FAP Tissue Sample

| Cell Type | Normal Tissue | Adenoma Tissue | Combined |
|---|---|---|---|
| Aberrant | 0.062 | 0.381 | 0.324 |
| Intermediate | 0.590 | 0.525 | 0.537 |
| Enterocyte | 0.303 | 0.055 | 0.099 |
| Goblet | 0.044 | 0.039 | 0.040 |
| Total | 1.000 | 1.000 | 1.000 |

TABLE 8

| S. no | Primer | Sequence | Product size (in bp) | SOX9 recombination PCR | |
|---|---|---|---|---|---|
| 1 | SOX9_Unflox_F | TGGCGATGCCTGCT | ~1200 | | |
| | | TGCC | | | |
| 2 | SOX9_Flox_F | TGCACTCACCGACC | ~300 | | |
| | | CCG | | | |
| 3 | SOX9_WT_F | GCTGGGATTCCAGG | 398 | | |
| | | AGAGAGACT | | | |
| 4 | SOX9_Reverse | GGAAGGCTAAGGGA | | | |
| | | CACTCTTG | | | |
| S. no | Primer | Species | Sequence_F | Sequence_R | RT-PCR |
| 1 | Axin2 | Mouse | ATGAGTAGCGCCGTGTTAGTG | GGGCATAGGTTTGGTGGACT | Primers |
| 2 | Ascl2 | Mouse | AAGCACACCTTGACTGGTACG | AAGTGGACGTTTGCACCTTCA | |
| 3 | Lgr5 | Mouse | TCCCCACTGCAATCAAGACA | CGAGGCACCATTCAAAGTCAG | |
| 4 | Myc | Mouse | CAGCTGGAGATGATGACCGA | TTGATGAAGGTCTCGTCGTC | |
| 5 | Prom1 | Mouse | GATCAGGCCAACAACTATGG | CCAGGAGTGTTATGGAATGC | |
| 6 | Lrig1 | Mouse | TAGAGGAGTGGCATCTGGAG | CCATGCGCTAAGGATTAAAA | |
| 7 | Sox9 | Mouse | CCACGGAACAGACTCACATC | CCCTCTCGCTTCAGATCAAC | |
| 8 | Krt20 | Mouse | CGCTCCCTGGAGCAATCCAA | CAATTCGCAGGACGCACTGG | |
| 9 | Muc2 | Mouse | TTCGTCACTTCATCAGCGGG | GGGTGGTCTTGTGGTAGGTG | |
| 10 | Cdx2 | Mouse | GAGTCCTGTGACCTCCTTGC | AGATGCTGTTCGTGGGTAGG | |
| 11 | Ly6a | Mouse | CCCCTACCCTGATGGAGTCT | AGAGGTCTTCCTGGCAACAG | |
| 12 | Gapdh | Mouse | CCCATGTTTGTGATGGGTGT | GTGATGGCATGGACTGTGGT | |
| 13 | Lgr5 | Human | GGTGACAACAGCAGTATGGACGA | GAAGGTGAACACTGCACTGAATGAA | |
| 14 | Sox9 | Human | GTACCCGCACTTGCACAAC | TCTCGCTCTCGTTCAGAAGTC | |
| 15 | Ascl2 | Human | GGCACTGCTGCTCTGCTA | GTTCACGCTCCCTTGAAGA | |
| 16 | Axin2 | Human | CAAGGGCCAGGTCACCAA | CCCCCAACCCATCTTCGT | |
| 17 | Dpp4 | Human | CCTTCTACTCTGATGAGTCACTGC | GTGCCACTAAGCAGTTCCATCTTC | |
| 18 | Krt20 | Human | GCATCTCCAACTCCAGACACA | CACCGAGCATTTTGCAGTTGA | |
| 19 | Ly6e | Human | ATCTTCTTGCCAGTGCTGCT | AGTCACGCAGTAGTTGTCCC | |
| 20 | Trop2 | Human | ACAACGATGGCCTCTACGAC | GTCCAGGTCTGAGTGGTTGAA | |
| 21 | Gapdh | Human | TGTTGCCATCAATGACCCCTT | CTCCACGACGTACTCAGCG | |
| | Name | Sequence | | | |

| | | | |
|---|---|---|---|
| 1 | ATAC_P5_Ad1 | AATGATACGGCGACCACCGAGATCTACA | |
| | | CTCGTCGGCAGCGTCAGATGTG | |
| 2 | ATAC_P7_Ad2.1_ | CAAGCAGAAGACGGCATACGAGATTCGC | Indexed P7 adapter |
| | TAAGGCGA | CTTAGTCTCGTGGGCTCGGAGATGT | |
| 3 | ATAC_P7_Ad2.2_ | CAAGCAGAAGACGGCATACGAGATCTAG | |
| | CGTACTAG | TACGGTCTCGTGGGCTCGGAGATGT | |
| 4 | ATAC_P7_Ad2.3_ | CAAGCAGAAGACGGCATACGAGATTTCT | |
| | AGGCAGAA | GCCTGTCTCGTGGGCTCGGAGATGT | |
| 5 | ATAC_P7_Ad2.4_ | CAAGCAGAAGACGGCATACGAGATGCTC | |
| | TCCTGAGC | AGGAGTCTCGTGGGCTCGGAGATGT | |
| 6 | ATAC_P7_Ad2.5_ | CAAGCAGAAGACGGCATACGAGATAGGA | |
| | GGACTCCT | GTCCGTCTCGTGGGCTCGGAGATGT | |
| 7 | ATAC_P7_Ad2.6_ | CAAGCAGAAGACGGCATACGAGATCATG | |
| | TAGGCATG | CCTAGTCTCGTGGGCTCGGAGATGT | |
| 8 | ATAC_P7_Ad2.7_ | CAAGCAGAAGACGGCATACGAGATGTAG | |
| | CTCTCTAC | AGAGGTCTCGTGGGCTCGGAGATGT | |

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize or be able to ascertain using no more than routine experimentation, many equivalents to the specific embodiments of the invention described herein. Such equivalents are intended to be encompassed by the following claims.

1-51. (canceled)

52. A genetically modified cell, comprising:
a first stably integrated endogenous reporter system co-expressing one or more biomarkers listed in Table 1 and a first signal, wherein an increased level of the first signal corresponds to an endogenous stem cell-like transcriptional activity within the cell; and/or
a second stably integrated endogenous reporter system co-expressing one or more biomarkers listed in Table 2 and a second signal, wherein an increased level of the second signal corresponds to an endogenous intestinal differentiation activity within the cell, optionally further comprising one or more additional stably integrated endogenous reporter systems expressing one or more biomarkers listed in Table 1 or 2 and an additional signal.

53. The genetically modified cell of claim 52, wherein the one or more biomarkers listed in Table 1 comprises SOX9, PROM1, LGR5, and/or ASCL2.

54. The genetically modified cell of claim 52, wherein the one or more biomarkers listed in Table 2 comprises KRT20, MUC2, and/or DPP4.

55. The genetically modified cell of claim 52, wherein the one or more biomarkers listed in Table 1 is SOX9 and the one or more biomarkers listed in Table 2 is KRT20.

56. The genetically modified cell of claim 52, wherein the first signal, the second signal, and/or the additional signal comprise a molecule, a probe, and/or a protein that are fluorescent or radioactive.

57. The genetically modified cell of claim 52, wherein the first signal, the second signal, and/or the additional signal comprise a green fluorescent protein (GFP), a red fluorescent protein (RFP), a yellow fluorescent protein (YFP), and/or a cyan fluorescent protein (CFP), or a derivative or a fragment thereof.

58. (canceled)

59. The genetically modified cell of claim 52, wherein the cell exhibits a phenotype of an intestinal cancer or a CRC, and/or wherein the cell is derived from a model of an intestinal cancer or a CRC, and/or wherein the cell is derived from a cancerous sample from either a subject or a member of the same species to which the subject belongs.

60. (canceled)

61. A stable cell line comprising a plurality of the genetically modified cells of claim 52, wherein the one or more biomarkers listed in Table 1 comprises SOX9, PROM1, LGR5, and/or ASCL2.

62. A method of identifying an agent for activating intestinal cell differentiation and/or treating an intestinal cancer or a CRC, comprising:
a) detecting at a first point in time the first signal and/or the second signal in a sample comprising the genetically modified cell of claim 52;
b) repeating step a) during at least one subsequent point in time after contacting the sample with the agent; and
c) comparing the expression level of the first signal and/or the second signal detected in steps a) and b),
wherein the absence of, or a significant decrease in, the expression level of the first signal in the subsequent sample as compared to the expression level of the first signal in the sample at the first point in time, indicates that the agent effectively activates intestinal cell differentiation and/or treats the intestinal cancer or the CRC, and/or wherein the presence of, or a significant increase in, the expression level of the second signal in the subsequent sample as compared to the expression level of the second signal in the sample at the first point in time, indicates that the agent effectively activates intestinal cell differentiation and/or treats the intestinal cancer or the CRC, optionally wherein the sample is from a subject.

63. The method of claim 62, further comprising detecting at a first point in time the additional signal in a sample comprising the genetically modified cell.

64. The method of claim 62, wherein the agent is a small molecule, CRISPR single-guide RNA (sgRNA), RNA interfering agent, antisense oligonucleotide, peptide or peptidomimetic inhibitor, aptamer, antibody, or intrabody, optionally wherein the RNA interfering agent is a small interfering RNA (siRNA), CRISPR RNA (crRNA), a small hairpin RNA (shRNA), a microRNA (miRNA), or a piwi-interacting RNA (piRNA).

65. (canceled)

66. A method of making an endogenous cell reporter system, comprising:
integrating a first cassette comprising a first signal and an antibiotic resistance into a genomic locus of one or more biomarkers listed in Table 1 in a cell; and
integrating a second cassette comprising a second signal and an antibiotic resistance into a genomic locus of one or more biomarkers listed in Table 2 in a cell,
optionally further comprising integrating one or more additional cassettes comprising an additional signal and an antibiotic resistance into a genomic locus of one or more biomarkers listed in Table 1 or 2.

67. The method of claim 66, wherein the one or more biomarkers listed in Table 1 comprises SOX9, PROM1, LGR5, and/or ASCL2.

68. The method of claim 66, wherein the one or more biomarkers listed in Table 2 comprises KRT20, MUC2, and/or DPP4.

69. The method of claim 66, wherein the one or more biomarkers listed in Table 1 is SOX9 and the one or more biomarkers listed in Table 2 is KRT20.

70. The method of claim 66, further comprising selecting an antibiotic-resistant population of cells, wherein the antibiotic-resistant population of cells indicates successful integration of the first cassette, the second cassette and/or the one or more additional cassettes.

71. The method of claim 66, wherein the first signal, the second signal, and/or the additional signal comprises a polynucleotide encoding a molecule, a probe, and/or a protein that are fluorescent or radioactive.

72. The method of claim 66, wherein the first signal, the second signal, and/or the additional signal comprises a polynucleotide encoding a green fluorescent protein (GFP), a red fluorescent protein (RFP), a yellow fluorescent protein (YFP), and/or a cyan fluorescent protein (CFP), or a derivative or a fragment thereof.

73. (canceled)

74. The method of claim 66, wherein the cell exhibits a phenotype of an intestinal cancer or a CRC, and/or wherein the cell is derived from a model of an intestinal cancer or a CRC, and/or wherein the cell is derived from a cancerous sample from either a subject or a member of the same species to which the subject belongs.

75. The method of claim 70, further comprising developing a stable cell line comprising a plurality of the antibiotic-resistant population of cells.
